(19) United States
(12) Patent Application Publication
(10) Pub. No.: US 2022/0401477 A1
(43) Pub. Date: Dec. 22, 2022

(54) METHOD FOR CONTROLLING VIRAL INFECTIONS THROUGH ADOPTIVE TRANSFER OF A CELL PRODUCT COMPRISING AN EXPANDED AND ENRICHED POPULATION OF SUPERACTIVATED CYTOKINE KILLER CELLS

(71) Applicant: VERDURE BIOTECH, INC., Houston, TX (US)

(72) Inventors: Helen Hao, Piscataway, NJ (US); Lilit Garibyan, Newton, MA (US); Beverly W. Lubit, Kinnelon, NJ (US); Sean O'Connell, Budd Lake, NJ (US)

(73) Assignee: VERDURE BIOTECH, INC., Houston, TX (US)

(21) Appl. No.: 17/746,307

(22) Filed: May 17, 2022

Related U.S. Application Data
(60) Provisional application No. 63/189,842, filed on May 18, 2021.

(51) Int. Cl. A61K 35/17; A61P 37/04

(52) U.S. Cl. CPC A61K 35/17; A61P 37/04; A61K 2035/124

(57) ABSTRACT

The invention of the present disclosure provides a method for treating a viral infection in a recipient subject suffering from or at risk of a viral infection including administering to the recipient subject a pharmaceutical composition comprising a therapeutic amount of superactivated cytokine killer T cells (SCKTCs) and a pharmaceutically acceptable carrier, and mobilizing an immune response of the recipient subject to the viral pathogen. When tested in vitro, the SCKTCs are characterized by a predominant production of T_{H}1 dominant cytokines including IFN-γ; an IFN-γ:IL-4 ratio of at least 500:1; and at least 50% killing of target A549 cells at an effector:target ratio of 20:1. The present disclosure further provides a method of preparing a pharmaceutical composition comprising an enriched population of superactivated cytokine killer T cells (SCKTCs) wherein pulsing steps with monocyte-derived dendritic cells (DCs) loaded with alpha-GalCer achieve at least an 80% pure population of SCKTCs without positive or negative cell separation methods.

### CROSS-REFERENCE TO RELATED APPLICATIONS

This application claims the benefit of priority to U.S. Provisional Application No. 63/189,842, filed May 18, 2021, entitled “A Method for Controlling Viral Infections Through Adoptive Transfer of a Cell Product Comprising An Expanded and Enriched Population of Superactivated Cytokine Killer Cells.” The entire contents of the aforementioned application is incorporated by reference herein in its entirety.

### FIELD OF THE INVENTION

The described invention relates to compositions and methods for treating a viral infection.

### BACKGROUND OF THE INVENTION

Infectious existing and emerging pathogens continue to cause significant morbidity and mortality worldwide. Certain diseases are considered particularly important, e.g., because they had a 100% lethality rate when they emerged, for example, HIV/AIDS; or because the infectious viral agent causes disease beyond the principal person of infection.

In order to cause disease, pathogens must be able to enter the host body, adhere to specific host cells, invade and colonize host tissues, and inflict damage on those tissues. Entrance to the host typically occurs through natural orifices such as the mouth, eyes, or genital openings, or through wounds that breach the skin barrier to pathogens. Although some pathogens can grow at the initial entry site, most must invade areas of the body where they are not typically found by attaching to specific host cells. Some pathogens then multiply between host cells or within body fluids, while others such as viruses and some bacterial species enter the host cells and grow there. Although the growth of pathogens may be enough to cause tissue damage in some cases, damage can also occur due to the production of toxins or destructive enzymes by the pathogen.

### Microorganisms That Cause Infectious Diseases

Classically, there are five major types of infectious agents: bacteria, viruses, fungi, protozoa, and helminths. A brief review of the general characteristics of each of these agents and examples of some diseases they cause follows.

### Bacteria

Bacteria are small, single-celled prokaryotic (lacking a nucleus) organisms that can be classified broadly by their shape as rods (bacilli), spheres (cocci), or spirals (spirillum), and by their cell surface properties. Gram staining, a differential staining procedure in which bacteria can be classified as gram positive or gram negative, depending on whether they retain or lose the primary stain when subjected to treatment with a decolorizing agent; reflects underlying structural differences in the cell walls of gram-positive and gram-negative bacteria. The cell wall of Gram positive organisms is composed of a relatively thick peptidoglycan layer and teichoic acids. Examples of pathogenic gram-positive bacteria include Staphylococcus aureus, which causes skin, respiratory, and wound infections, and Clostridium tetani, which produces a toxin that can be lethal for humans. The cell wall of Gram-negative organisms is composed of a thin peptidoglycan layer, lipoproteins, lipopolysaccharides, phospholipids, and proteins. Exemplary gram-negative pathogenic bacteria include Salmonella typhi, which causes typhoid fever, and Yersinia pestis, which causes plague.

Bacterial virulence factors help bacteria to (1) invade the host; (2) cause disease; and (3) evade host defenses. They may be encoded on chromosomal, plasmid, transposon, or temperate bacteriophage DNA; virulence factor genes on transposons or temperate bacteriophage DNA may integrate into the bacterial genome. Examples of types of virulence factors include adherence factors; invasion factors; bacterial capsules (to protect the bacteria from opsonization and phagocytosis); endotoxins (e.g., the lipopolysaccharide endotoxins on Gram negative bacteria, which can cause lethal shock), exotoxins (e.g., cytotoxins, neurotoxins, and enterotoxins), and siderophores (iron-binding factors that allow some bacteria to compete with the host for iron bound to hemoglobin, transferrin and lactoferrin. In certain infections (e.g., tuberculosis), tissue damage results from toxic mediators released by host lymphoid cells rather than from bacterial toxins.

The capacity of a bacterium to cause disease reflects its relative pathogenicity. When isolated from a patient, frank or primary pathogens are considered to be probable agents of disease. Opportunistic pathogens are those isolated from patients whose host defense mechanisms have been compromised. Bacteria that rarely or never cause human disease are generally considered to be nonpathogens, but that categorization can change because of the adaptability of bacteria and the detrimental effects of therapies on resistance mechanisms. Susceptibility to bacterial infections depends on the physiologic and immunologic condition of the host and on the virulence of the bacterial agent.

### Fungi

Fungi are a group of eukaryotic, heterotrophic organisms that have rigid cellulose- or chitin-based cell walls and reproduce primarily by forming spores. Most fungi are multicellular, although some, such as yeasts, are unicellular. Examples of diseases caused by fungi are ringworm and histoplasmosis. Yeast of the Candida genus are opportunistic pathogens that may cause diseases such as vaginal yeast infections and thrush among susceptible subjects, e.g., that are immunocompromised or undergoing antibiotic therapy.

### Protozoa

Protozoa are unicellular, heterotrophic eukaryotes without a cell wall that include the familiar amoeba and paramecium. They can be acquired through contaminated food or water or by the bite of an infected arthropod (e.g., mosquito). Diarrheal disease in the United States can be caused by two common protozoan parasites, Giardia lamblia and Cryptosporidium parvum. Malaria, a tropical illness that causes 300 million to 500 million cases of disease annually, is caused by several species of the protozoan Plasmodium.

### Helminths

Helminths are simple, invertebrate animals, some of which are infectious parasites. They are multicellular and have differentiated tissues. Drugs that kill helminths are frequently very toxic to human cells.

Many helminths have complex reproductive cycles that include multiple stages, many or all of which require a host. Schistosoma, a flatworm, causes the mild disease swimmer's itch in the United States; another species of Schistosoma causes the much more serious disease schistosomiasis, which is endemic in Africa and Latin America. Schistosome eggs hatch in freshwater, and the resulting larvae infect snails. When the snails shed these larvae, the larvae attach to and penetrate human skin. They feed, grow, and mate in the human bloodstream; the damage to human tissues caused by the accumulating schistosome eggs with their sharp spines results in disease symptoms including diarrhea and abdominal pain. Liver and spleen involvement are common. Another disease due to a helminth is trichinosis, caused by the roundworm Trichinella spiralis. This infectious agent is typically ingested in improperly cooked pork from infected pigs. Early disease symptoms include vomiting, diarrhea, and fever; later symptoms include intense muscle pain because the larvae grow and mature in those tissues. Fatal cases often show congestive heart failure and respiratory paralysis.

### Viruses

A virus particle is composed of a viral genome of nucleic acid that is surrounded by a protein coat (capsid). Many animal viruses are surrounded by an outer lipid envelope, which they acquire from the host cell membrane as they leave the virus-infected cell.

Viral genomes may be double- or single-stranded DNA (a DNA virus), or double- or single-stranded RNA (an RNA virus). The Baltimore classification of viruses [Baltimore, D., 1974Haravey Lecture 70 Series: 57-74) is based on the viral mechanism of mRNA production. Viral genomes may be single-stranded (ss) or double stranded (ds), RNA or DNA, and may or may not use reverse transcriptase. Additionally, single stranded RNA viruses may be either positive sense (+) or negative or antisense (−). This classification places viruses into seven groups, as shown in Table 1:

TABLE 1
Baltimore classification of viruses

| Group | Examples |
|---|---|
| ds DNA viruses | Adenoviruses, Herpesviruses, |
| | Poxviruses |
| ds DNA viruses | + sense DNA (Parvoviruses) |
| dsRNA viruses | Rheoviruses |
| (+) ssRNA viruses | Picornaviruses, Togaviruses |
| (−) ssRNA viruses | Orthomyxoviruses, Rhabdoviruses |
| ssRNA-RT viruses | Retroviruses |
| dsDNA-RT viruses | Hepadnaviruses |

Table 2, taken from https://viralzone.expasy.org/678 (visited Mar. 15, 2021), displays an exemplary list of human viral pathogens, their host, transmission and disease.

TABLE 2

| Virus | Genus, Family | Host | Transmission | Disease |
|---|---|---|---|---|
| Adeno-associated | Dependovirus, | Human, | Respiratory | None |
| virus | Parvoviridae | vertebrates | | |
| Aichi virus | Kobuvirus, | Human | Fecal-oral | Gastroenteritis |
| | Picornaviridae | | | |
| Australian bat | Lyssavirus, | Human, bats | Zoonosis, | Fatal encephalitis |
| lyssavirus | Rhabdoviridae | | animal bite | |
| BK polyomavirus | Polvomavirus, | Human | Respiratory | None |
| | Polyomaviridae | | fluids or urine | |
| Banna virus | Seadornavirus, | Human, cattle, | Zoonosis, | Encephalitis |
| | Reoviridae | pig, mosquitoes | arthropod bite | |
| Barmah forest virus | Alphavirus, | Human, | Zoonosis, | Fever, joint pain |
| | Togaviridae | marsupials, | arthropod bite | |
| | | mosquitoes | | |
| Bunyamwera virus | Orthobunyavirus, | Human, | Zoonosis, | Encephalitis |
| | Bunyaviridae | mosquitoes | arthropod bite | |
| Bunyavirus La Crosse | Orthobunyavirus, | Human, deer, | Zoonosis, | Encephalitis |
| | Bunyaviridae | mosquitoes, | arthropod bite | |
| | | tamias | | |
| Bunyavirus snowshoe | Orthobunyavirus, | Human, rodents, | Zoonosis, | Encephalitis |
| hare | Bunyaviridae | mosquitoes | arthropod bite | |
| Cercopithecine | Lvmphocryptovirus, | Human, | Zoonosis, | Encephalitis |
| herpesvirus | Herpesviridae | monkeys | animal bite | |
| Chandipura virus | Vesiculovirus, | Human, | Zoonosis, | Encephalitis |
| | Rhabdoviridae | sandflies | athropod bite | |
| Chikungunya virus | Alphavirus, | Human, | Zoonosis, | Fever, joint pain |
| | Togaviridae | monkeys, | arthropod bite | |
| | | mosquitoes | | |
| Cosavirus A | Cosavirus, | Human | Fecal-oral | — |
| | Picornaviridae | | (probable) | |
| Cowpox virus | Orthopoxvirus, | Human, | Zoonosis, | None |
| | Poxviridae | mammals | contact | |
| Coxsackievirus | Enterovirus, | Human | Fecal-oral | Meningitis, |
| | Picornaviridae | | | myocarditis, |
| | | | | paralysis |
| Crimean-Congo | Nairovirus, | Human, | Zoonosis, | Hemorrhagic fever |
| hemorrhagic fever | Bunyaviridae | vertebrates, ticks | arthropod bite | |
| virus | | | | |
| Dengue virus | Flavivirus, | Human, | Zoonosis, | Hemorrhagic fever |
| | Flaviviridae | mosquitoes | arthropod bite | |
| Dhori virus | Thogotovirus, | Human, ticks | Zoonosis, | Fever, encephalitis |
| | Orthomyxoviridae | | arthropod bite | |
| Dugbe virus | Nairovirus, | Human, ticks | Zoonosis, | Thrombocytopaenia |
| | Bunyaviridae | | arthropod bite | |
| Duvenhage virus | Lyssavirus, | Human, | Zoonosis, | Fatal encephalitis |
| | Rhabdoviridae | mammals | animal bite | |
| Eastern equine | Alphavirus, | Human, birds, | Zoonosis, | Encephalitis |
| encephalitis virus | Togaviridae | mosquitoes | arthropod bite | |
| Ebolavirus | Ebolavirus, | Human, | Zoonosis, | Hemorrhagic fever |
| | Filoviridae | monkeys, bats | contact | |
| Echovirus | Enterovirus, | Human | Fecal-oral | Common cold |
| | Picornaviridae | | | |
| Encephalomyocarditis | Cardiovirus, | Human, mouse, | Zoonosis | Encephalitis |
| virus | Picornaviridae | rat, pig | | |
| Epstein-Barr virus | Lymphocryptovirus, | Human | Contact, saliva | Mononucleosis |
| | Herpesviridae | | | |
| European bat | Lyssavirus, | Human, bats | Zoonosis, | Fatal encephalitis |
| lyssavirus | Rhabdovirus | | animal bite | |
| GB virus C/Hepatitis | Pegivirus, | Human | Blood, | None |
| G virus | Flaviviridae | | occasionally | |
| | | | sexual | |
| Hantaan virus | Hantavirus, | Human, rodents | Zoonosis, | Renal or respiratory |
| | Bunyaviridae | | urine, saliva | syndrome |
| Hendra virus | Henipavirus, | Human, horse, | Zoonosis, | Encephalitis |
| | paramyxoviridae | bats | animal bite | |
| Hepatitis A virus | Hepatovirus, | Human | Fecal-oral | Hepatitis |
| | picornaviridae | | | |
| Hepatitis B virus | Orthohepadnavirus, | Human, | Sexual contact, | Hepatitis |
| | Hepadnaviridae | Chimpanzees | blood | |
| Hepatitis C virus | Hepacivirus, | Human | Sexual, blood | Hepatitis |
| | Flaviviridae | | | |
| Hepatitis E virus | Hepevirus, | Human, pig, | Zoonosis, food | Hepatitis |
| | Unassigned | monkeys, some | | |
| | | rodents, chicken | | |
| Hepatitis delta virus | Deltavirus, | Human | Sexual contact, | Hepatitis |
| | Unassigned | | blood | |
| Horsepox virus | Orthopoxvirus, | Human, horses | Zoonosis, | None |
| | Poxviridae | | contact | |
| Human adenovirus | Mastadenovirus, | Human | Respiratory, | Respiratory |
| | Adenoviridae | | fecal-oral | |
| Human astrovirus | Mamastrovirus, | Human | Fecal-oral | Gastroenteritis |
| | Astroviridae | | | |
| Human coronavirus | Alphacoronavirus, | Human | Respiratory | Respiratory |
| | Coronaviridae | | | |
| Human | Cytomegalovirus, | Human | Contact, urine, | Mononucleosis, |
| cytomegalovirus | Herpesviridae | | saliva | pneumonia |
| Human enterovirus | Enterovirus, | Human | Fecal-oral | Diarrhea, |
| 68, 70 | Picornaviridae | | | neurological |
| | | | | disorder |
| Human herpesvirus 1 | Simplexvirus, | Human | Sexual contact, | Skin lesions |
| | Herpesviridae | | saliva | |
| Human herpesvirus 2 | Simplexvirus, | Human | Sexual contact, | Skin lesions |
| | Herpesviridae | | saliva | |
| Human herpesvirus 6 | Roseolovirus, | Human | Respiratory, | Skin lesions |
| | Herpesviridae | | contact | |
| Human herpesvirus 7 | Roseolovirus, | Human | Respiratory, | Skin lesions |
| | Herpesviridae | | contact | |
| Human herpesvirus 8 | Rhadinovirus, | Human | Sexual contact, | Skin lymphoma |
| | Herpesviridae | | saliva | |
| Human | Lentivirus, | Human | Sexual contact, | AIDS |
| immunodeficiency | Retroviridae | | blood | |
| virus | | | | |
| Human papillomavirus | Mupapillomavirus, | Human | Contact | Skin warts |
| 1 | Papillomaviridae | | | |
| Human papillomavirus | Alphapapillomavirus, | Human | Contact | Skin warts |
| 2 | Papillomaviridae | | | |
| Human papillomavirus | Alphapapillomavirus, | Human | Sexual | Genital warts, |
| 16, 18 | Papillomaviridae | | | cervical cancer |
| Human parainfluenza | Respirovirus, | Human | Respiratory | Respiratory |
| | Paramyxoviridae | | | |
| Human parvovirus | Erythrovirus, | Human | Respiratory | Skin lesion |
| B19 | Parvoviridae | | | |
| Human respiratory | Orthopneumovirus, | Human | Respiratory | Respiratory |
| syncytial virus | Pneumoviridae | | | |
| Human rhinovirus | Enterovirus, | Human | Respiratory | Respiratory |
| | Picornaviridae | | | |
| Human SARS | Betacoronavirus, | Human, bats, | Zoonosis | Respiratory |
| coronavirus | Coronaviridae | palm civet | | |
| Human | Spumavirus, | Human | Contact, saliva | None |
| spumaretrovirus | Retroviridae | | | |
| Human T- | Deltaretrovirus, | Human | Sexual contact, | Leukemia |
| lymphotropic virus | Retroviridae | | maternal- | |
| | | | neonatal | |
| Human torovirus | Torovirus, | Human | Fecal-oral | Gastroenteritis |
| | Coronaviridae | | | |
| Influenza A virus | Influenzavirus A, | Human, birds, | Respiratory or | Flu |
| | Orthomyxoviridae | pigs | Zoonosis, | |
| | | | animal contact | |
| Influenza B virus | Influenzavirus B, | Human | Respiratory | Flu |
| | Orthomyxoviridae | | | |
| Influenza C virus | Influenzavirus C, | Human | Respiratory | Flu |
| | Orthomyxoviridae | | | |
| Isfahan virus | Vesiculovirus, | Human, | Zoonosis, | Undocumented, |
| | Rhabdoviridae | sandflies, gerbils | arthropod bite | encephalitis? |
| JC polyomavirus | Polvomavirus, | Human | Fecal-oral or | Encephalitis |
| | Polyomaviridae | | urine | |
| Japanese encephalitis | Flavivirus, | Human, horses, | Zoonosis, | Encephalitis |
| virus | Flaviviridae | birds, | arthropod | |
| | | mosquitoes | borne | |
| Junin arenavirus | Arenavirus, | Human, rodents | Zoonosis, | Hemorrhagic fever |
| | Arenaviridae | | fomite | |
| KI Polyomavirus | Polvomavirus, | Human | Fecal-oral or | Encephalitis |
| | Polyomaviridae | | urine | |
| Kunjin virus | Flavivirus, | Human, horses, | Zoonosis, | Encephalitis |
| | Flaviviridae | birds, | arthropod | |
| | | mosquitoes | borne | |
| Lagos bat virus | Lyssavirus, | Human, | Zoonosis, | Fatal encephalitis |
| | Rhabdoviridae | mammals | animal bite | |
| Lake Victoria | Marburgvirus, | Human, | Zoonosis, | Hemorrhagic fever |
| marburgvirus | Filoviridae | monkeys, bats | fomite | |
| Langat virus | Flavivirus, | Human, ticks | Zoonosis, | Encephalitis |
| | Flaviviridae | | arthropod | |
| | | | borne | |
| Lassa virus | Arenavirus, | Human, rats | Zoonosis, | Hemorrhagic fever |
| | Arenaviridae | | fomites | |
| Lordsdale virus | Norovirus, | Human | Fecal-oral | Gastroenteritis |
| | Caliciviridae | | | |
| Louping ill virus | Flavivirus, | Human, | Zoonosis, | Encephalitis |
| | Flaviviridae | mammals, ticks | arthropod bite | |
| Lymphocytic | Arenavirus, | Human, rodents | Zoonosis, | Encephalitis |
| choriomeningitis virus | Arenaviridae | | fomite | |
| Machupo virus | Arenavirus, | Human, | Zoonosis, | Encephalitis |
| | Arenaviridae | monkeys, mouse | fomite | |
| Mayaro virus | Alphavirus, | Human, | Zoonosis, | Fever, joint pain |
| | Togaviridae | mosquitoes | arthropod bite | |
| MERS coronavirus | Betacoronavirus, | Human, Tomb | Zoonosis | Respiratory |
| | Coronaviridae | bat | | |
| Measles virus | Morbilivirus, | Human | Respiratory | Fever, rash |
| | Paramyxoviridae | | | |
| Mengo | Cardiovirus, | Human, mouse, | Zoonosis | Encephalitis |
| encephalomyocarditis | Picornaviridae | rabbit | | |
| virus | | | | |
| Merkel cell | Polvomavirus, | Human | — | Merkel cell |
| polyomavirus | Polyomaviridae | | | carcinoma |
| Mokola virus | Lyssavirus, | Human, rodents, | Zoonosis, | Encephalitis |
| | Rhabdoviridae | cat, dog shrew | animal bite | |
| Molluscum | Molluscipoxvirus, | Human | Contact | Skin lesions |
| contagiosum virus | Poxviridae | | | |
| Monkeypox virus | Orthopoxvirus, | Human, mouse, | Zoonosis, | Skin lesions |
| | Poxviridae | prairie dog | contact | |
| Mumps virus | Rubulavirus, | Human | Respiratory, | Mumps |
| | Paramyxoviridae | | saliva | |
| Murray valley | Flavivirus, | Human, | Zoonosis, | Encephalitis |
| encephalitis virus | Flaviviridae | mosquitoes | arthropod bite | |
| New York virus | Hantavirus, | Human, mouse | Zoonosis, | Hemorrhagic fever |
| | Bunyavirus | | urine, saliva | |
| Nipah virus | Henipavirus, | Human, bats | Zoonosis, | Encephalitis |
| | Paramyxoviridae | | animal bite | |
| Norwalk virus | Norovirus, | Human | Fecal-oral | Gastroenteritis |
| | Caliciviridae | | | |
| O'nyong-nyong virus | Alphavirus, | Human, | Zoonosis, | Fever, joint pain |
| | Togaviridae | mosquitoes | arthropod bite | |
| Orf virus | Parapoxvirus, | Human, | Zoonosis, | Skin lesions |
| | Poxviridae | mammals | contact | |
| Oropouche virus | Orthobunvavirus, | Human, wild | Zoonosis, | Fever, joint pain |
| | Bunyaviridae | animals(sloths) | arthropod bite | |
| Pichinde virus | Arenavirus, | Human, rat, | Zoonosis, | Hemorrhagic fever |
| | Arenaviridae | guinea pig | fomite | |
| Poliovirus | Enterovirus, | Human, | Fecal-oral | Poliomyelitis |
| | Picornaviridae | mammals | | |
| Punta toro phlebovirus | Phlebovirus, | Human, | Zoonosis, | Hemorrhagic fever |
| | Bunyaviridae | sandflies | arthropod bite | |
| Puumala virus | Hantavirus, | Human, bank | Zoonosis, | Hemorrhagic fever |
| | Bunyavirus | vole | urine, saliva | |
| Rabies virus | Lvssavirus, | Human, | Zoonosis, | Fatal encephalitis |
| | Rhabdoviridae | mammals | animal bite | |
| Rift valley fever virus | Phlebovirus, | Human, | Zoonosis, | Hemorrhagic fever |
| | Bunyaviridae | mammals, | arthropod bite | |
| | | mosquitoes, | | |
| | | sandflies | | |
| Rosavirus A | Rosavirus, | Human | | |
| | Picornaviridae | | | |
| Ross river virus | Alphavirus, | Human, | Zoonosis, | Fever, joint pain |
| | Togaviridae | mosquitoes, | arthropod bite | |
| | | marsupials | | |
| Rotavirus A | Rotavirus, | Human | Fecal-oral | Gastroenteritis |
| | Reoviridae | | | |
| Rotavirus B | Rotavirus, | Human | Fecal-oral | Gastroenteritis |
| | Reoviridae | | | |
| Rotavirus C | Rotavirus, | Human | Fecal-oral | Gastroenteritis |
| | Reoviridae | | | |
| Rubella virus | Rubivirus, | Human | Respiratory | Rubella |
| | Togaviridae | | | |
| Sagiyama virus | Alphavirus, | Human, horse, | Zoonosis, | Fever, joint pain |
| | Togaviridae | pig, mosquitoes | arthropod bite | |
| Salivirus A | Salivirus, | Human | | Gastroenteritis |
| | Picornaviridae | | | |
| Sandfly fever Sicilian | Phlebovirus, | Human, | Zoonosis, | Hemorrhagic fever |
| virus | Bunyaviridae | sandflies | arthropod bite | |
| Sapporo virus | Sapovirus, | Human | Fecal-oral | Gastroenteritis |
| | Caliciviridae | | | |
| SARS coronavirus 2 | Betacoronavirus, | Human, bats, | Respiratory | Covid-19 |
| | Coronaviridae | pangolin? | | |
| Semliki forest virus | Alphavirus, | Human, birds, | Zoonosis, | Fever, joint pain |
| | Togaviridae | hedgehog, | arthropod bite | |
| | | mosquitoes | | |
| Seoul virus | Hantavirus, | Human, rats | Zoonosis, | Hemorrhagic fever |
| | Bunyavirus | | urine, saliva | |
| Simian foamy virus | Spumavirus, | Human, | Zoonosis, | None |
| | Retroviridae | monkeys | contact | |
| Simian virus 5 | Rubulavirus, | Human, dog | Zoonosis, | Undocumented |
| | Paramyxoviridae | | contact | |
| Sindbis virus | Alphavirus, | Human, birds, | Zoonosis, | Pogosta_disease |
| | Togaviridae | mosquitoes | arthropod bite | Fever, joint pain |
| Southampton virus | Norovirus, | Human | Fecal-oral | Gastroenteritis |
| | Caliciviridae | | | |
| St. louis encephalitis | Flavivirus, | Human, birds, | Zoonosis, | Encephalitis |
| virus | Flaviviridae | mosquitoes | arthropod bite | |
| Tick-bome powassan | Flavivirus, | Human, ticks | Zoonosis, | Encephalitis |
| virus | Flaviviridae | | arthropod bite | |
| Torque teno virus | Alphatorquevirus, | Human | Sexual, blood | None |
| | Anelloviridae | | | |
| Toscana virus | Phlebovirus, | Human, | Zoonosis, | Hemorrhagic fever |
| | Bunyaviridae | mosquitoes | arthropod bite | |
| Uukuniemi virus | Phlebovirus, | Human, ticks | Zoonosis, | Hemorrhagic fever |
| | Bunyaviridae | | arthropod bite | |
| Vaccinia virus | Orthopoxvirus, | Human, | Contact | None |
| | Poxviridae | mammals | | |
| Varicella-zoster virus | Varicellovirus, | Human | Respiratory, | Varicella |
| | Herpesviridae | | contact | |
| Variola virus | Orthopoxvirus, | Human | Respiratory | Variola |
| | Poxviridae | | | |
| Venezuelan equine | Alphavirus, | Human, rodents, | Zoonosis, | Fever, joint pain |
| encephalitis virus | Togaviridae | mosquitoes | arthropod bite | |
| Vesicular stomatitis | Vesiculovirus, | Human, cattle, | Zoonosis, | Encephalitis |
| virus | Rhabdoviridae | horse, pig, flies | athropod bite | |
| Western equine | Alphavirus, | Human, | Zoonosis, | Fever, joint pain |
| encephalitis virus | Togaviridae | vertebrates, | arthropod bite | |
| | | mosquitoes | | |
| WU polyomavirus | Polvomavirus, | Human | Respiratory | None |
| | Polyomaviridae | | fluids or urine | |
| West Nile virus | Flavivirus, | Human, birds, | Zoonosis, | Encephalitis |
| | Flaviviridae | ticks, | arthropod bite | |
| | | mosquitoes | | |
| Yaba monkey tumor | Orthopoxvirus, | Human, | Zoonosis, | None |
| virus | Poxviridae | monkeys | contact | |
| Yaba-like disease | Orthopoxvirus, | Human, | Zoonosis, | None |
| virus | Poxviridae | monkeys | contact | |
| Yellow fever virus | Flavivirus, | Human, | Zoonosis, | Hemorrhagic fever |
| | Flaviviridae | monkeys, | arthropod bite | |
| | | mosquitoes | | |
| Zika virus | Flavivirus, | Human, | Zoonosis, | Fever, joint pain, |
| | Flaviviridae | monkeys, | arthropod bite | rash |
| | | mosquitoes | | |

In the general process of infection and replication by a DNA virus, a viral particle first attaches to a specific host cell via protein receptors on its outer envelope, or capsid. The viral genome is then inserted into the host cell, where it uses host cell enzymes to replicate its DNA, transcribe the DNA to make messenger RNA, and translate the messenger RNA into viral proteins. The replicated DNA and viral proteins are then assembled into complete viral particles, and the new viruses are released from the host cell. In some cases, virus-derived enzymes destroy the host cell membranes, killing the cell and releasing the new virus particles. In other cases, new virus particles exit the cell by a budding process, weakening but not destroying the cell. Examples of DNA viruses that can cause human disease include, without limitation, herpesviruses that cause chicken pox, cold sores, and painful genital lesions, poxviruses; and Hepadnaviruses (e.g., hepatitis B virus).

In the case of some RNA viruses, the genetic material can be used directly as messenger RNA to produce viral proteins, including a viral RNA polymerase that copies the RNA template to produce the genetic material for new viral particles. Other RNA viruses, called retroviruses, use reverse transcriptase to copy the RNA genome into DNA, which integrates itself into the host cell genome. These viruses frequently exhibit long latent periods in which their genomes are faithfully copied and distributed to progeny cells each time the host cell divides. The human immunodeficiency virus (HIV), which causes AIDS, is a familiar example of a retrovirus. Examples of RNA viruses that cause human disease include, without limitation, rhinoviruses that cause most common colds; myxoviruses and paramyxoviruses that cause influenza, measles, and mumps; rotaviruses that cause gastroenteritis; retroviruses that cause AIDS and several types of cancer; Flaviviruses including Zika virus (ZIKV), dengue virus (DENV), yellow fever virus (YFV), West Nile virus (WNV), Japanese encephalitis virus (JEV), hepatitis C virus (HCV) and tick-borne encephalitis virus (TBEV); Togaviruses (e.g., Chikungunya virus (CHIKV), rubella virus), Filoviruses (e.g., Marburg virus, Ebola virus) Rhabdoviruses (e.g., rabies virus); picomaviruses (e.g., enteroviruses (including polio, PV, and rhinoviruses), foot-and-mouth disease virus (FMDV), and hepatitis A virus (HAV); and Coronaviruses.

The two primary patterns of viral infection are acute infections and persistent infections. In acute infections, some viruses rapidly kill the cell while producing a burst of new infectious particles (cytopathic viruses), while others infect cells and actively produce infectious particle without causing immediate host cell death (noncytopathic viruses). In persistent infections (e.g., latent infections, slow, abortive and transforming infections), some viruses infect, but neither kill the cell nor produce any viral progeny. Instead, replicated viruses remain inert unless they attach to the surface of another compatible host cell. [Principles of Virology. Flint, S J, Enquist L W Q, Krug, R M, Racaniello, V R, Skalka, A M, Eds. (2000) ASM Press, Washington, D.C., Chapter 15, pp. 519-551]

To initiate an infection in an individual host, sufficient virus must be available to initiate infection, the cells at the site of infection must be susceptible and permissive for the virus, and the local host antiviral defense systems must be absent or at least initially ineffective. Common sites of viral entry include the mucosal linings of the respiratory, alimentary and urogenital tracts, the outer surface of the eye (conjunctival membranes or cornea), and the skin. Following replication at the site of entry, virus particles can remain localized or can spread to other tissues. Local replication in the respiratory tract is characteristic of influenza virus, parainfluenza virus, rhinovirus and respiratory CoVs; replication of rotaviruses and enteric corona- and adenoviruses is restricted to the alimentary tract, and replication of some papillomaviruses is confined to the skin. Local spread of the infection in the epithelium occurs when newly released virus infects adjacent cells. An infection that spreads beyond the primary site of infection is said to be disseminated. If many organs become infected, the infection is described as systemic.

A severe virus infection attacks the host on multiple fronts. Some host defenses may be overcome passively by an overwhelming inoculum of virus. Directional release of virus particles from polarized cells at the musosal surface can avoid local host defenses and facilitate spread. For example, since virus particles released from the basolateral surfaces of polarized epithelial cells have been moved away from the defenses of the luminal surface, their release provides access to the underlying tissues and may facilitate systemic spread. Viruses that escape from local defenses to produce a disseminated infection often do so by entering the bloodstream (hematogenous spread). In addition, many viruses have evolved active mechanisms for bypassing or disarming host defenses.

Coronaviruses (CoVs), a large family of single-stranded RNA viruses, can infect a wide variety of animals, including humans, causing respiratory, enteric, hepatic and neurological diseases. [Yin, Y., Wunderink, R G, Respirology (2018) 23 (2): 130-37, citing Weiss, S R, Leibowitz, I L, Coronavirus pathogenesis. Adv. Virus Res. (2011) 81: 85-164]. Human coronaviruses, which were considered to be relatively harmless respiratory pathogens in the past, have now received worldwide attention as important pathogens in respiratory tract infection. As the largest known RNA viruses, CoVs are further divided into four genera: alpha-, beta-, gamma- and delta-coronavirus; the β-coronaviruses are further divided into A, B, C, and D lineages (Woo et al., J Virol. 2012 April; 86(7):3995-4008).

Coronaviruses [“CoVs” ] are enveloped with a non-segmented, positive sense, single strand RNA, with size ranging from 26,000 to 37,000 bases; this is the largest known genome among RNA viruses. [Yang, Y. et al., J. Autoimmunity (2020) doi.org/10.1016/j.jaut.2020.102434, citing Weiss, S R et al. Microbiol. Mol. Biol. Rev. (2005) 69 (4): 635-64]. The viral RNA encodes structural proteins, and genes interspersed within the structural genes, some of which play important roles in viral pathogenesis [Yang, Y. et al., J. Autoimmunity (2020) doi.org/10.1016/j.jaut.2020.102434, citing Fehr, A R, Perlman, S. Methods Mol. Biol. (2015) 1282: 1-23; Zhao, L. et al. Cell Host Microbe (2012) 11(6): 607-16]. The spike protein (S) is responsible for receptor binding and subsequent viral entry into host cells; it consists of S1 and S2 subunits. The membrane (M) and envelope (E) proteins play important roles in viral assembly; the E protein is required for pathogenesis [Id., citing DeDiego, M L, et al. J. Virol. (2007) 81(4): 1701-13; Nieto-Torres, J L et al. PLoS Pathog. (2014) 10(5): e1004077]. The nucleocapsid (N) protein contains two domains, both of which can bind virus RNA genomes via different mechanisms, and are necessary for RNA synthesis and packaging the encapsulated genome into virions. [Yang, Y. et al., J. Autoimmunity (2020) doi.org/10.1016/j.jaut.2020.102434., citing Fehr, A R, Perlman, S. Methods Mol. Biol. (2015) 1282: 1-23; Song, Z. et al. Viruses (2019) 11(1): 59; Chang, C K et al., J. Biomed. Sci. (2006) 13(1): 59-72; Hurst, K R, et al. J. Virol. (2009) 83 (14): 7221-34] The N protein also is an antagonist of interferon and viral encoded repressor (VSR) of RNA interference (RNAi), which benefits viral replication [Id., citing Cui, L. et al. J. Virol. (2015) 89 (17): 9029-43].

CoVs can co-infect humans and other vertebrate animals. Previously, seven CoVs were known to infect humans (HCoVs), including HCoV-229E and HCoV-NL63 in the α-coronaviruses, HCoV-OC43 and HCoV-HKU1 in the β-coronaviruses lineage A, SARS-CoV and SARS-CoV-2 in the β-coronaviruses lineage B (β-B coronaviruses), and MERS-CoV in the β-coronaviruses lineage C. SARS-CoV-2 shares a highly similar gene sequence and behavior pattern with SARS-CoV (Chan et al., Emerg Microbes Infect. 2020; 9(1):221-236). Both SARS-CoV-2 and SARS-CoV are in the coronavirus family, β-coronavirus genera. The genome of SARS-CoV-2 is more than 85% similar to the genome of the SARS-like virus ZC45 (bat-SL-CoVZC45, MG772933.1), and together these types of viruses form a unique Orthocoronavirinae subfamily with another SARS-like virus ZXC21 in the sarbecovirus subgenus [Zhu et al., N Engl J Med. 2020 Feb. 20; 382(8):727-733]. All three viruses show typical β-coronavirus gene structure. Human SARS-CoV and a genetically similar bat coronavirus (bat-SL-CoVZXC21, MG772934) from southwest of China have formed another clade within the sarbecovirus [Zhu et al., Id.]. 229E, OC43, NL63, and HKU1 infections are frequently mild, mostly caused common cold symptoms [Xu, X. et al. Eur. J. Nuclear Medicine & Molec. Imaging (2020) doi.org/10.1007/s00259-020-04735-9, citing Su, S. et al. Trends Microbiol. (2016) 24: 490-502]. Severe acute respiratory syndrome coronavirus (SARS-CoV) and Middle East respiratory syndrome coronavirus (MERS-CoV), have a different pathogenicity and have caused fatal illness [Id., citing Cui, J. et al. Nat. Rev. Microbio. (2019) 17: 181-92].

Beginning on or about December 2019, pneumonia cases of unknown origin were identified in Wuhan, China. The cause has been identified as severe acute respiratory syndrome coronavirus 2 (SARS-CoV-2) and the virus-infected pneumonia was later designated coronavirus disease 2019 (COVID-19) by WHO. SARS-CoV-2 is the eighth member of the coronaviruses that infects humans [Zhu, N. et al. N. Engl. J. Med. (2020) 382: 727-33], WHO reported that as of Mar. 15, 2021, there have been 119,452,269 confirmed cases of COVID-19, including 2,647,662 deaths. [covid19.who.int, visited 15 Mar. 2021].

COVID-19 can present as an asymptomatic carrier state, acute respiratory disease, and pneumonia. Adults represent the population with the highest infection rate; however, neonates, children, and elderly patients can also be infected by SARS-CoV-2. In addition, nosocomial infection of hospitalized patients and healthcare workers, and viral transmission from asymptomatic carriers are possible. The most common finding on chest imaging among patients with pneumonia was ground-glass opacity with bilateral involvement.

The severity of COVID-19 can be roughly categorized into three groups based on the severity of the initial infection. Mild COVID-19, which, along with asymptomatic COVID-19 comprises the majority of cases, is characterized by symptoms such as fever, shortness of breath, gastrointestinal distress, malaise, headaches and a loss of taste and small. Severely ill patients require hospitalization for treatment of the infection, because of respiratory issues. Critical patients are a subset of the severely ill patients who experience respiratory failure that requires mechanical ventilation support. The percentages of patients vary, but mild patients are reported to be approximately 80%, severe cases are 14%, and critical cases are 6%. As many countries prioritize testing only for hospitalized patients, determining the exact percentages of patients in the general population is challenging. [Disser, N P et al. J. Bone Joint Surg. Am. (2020) 102: 1197-204]. Severe cases are more likely to be older patients with underlying comorbidities compared to mild cases. Indeed, age and disease severity may be correlated with the outcomes of COVID-19.

SARSCoV-2 uses the SARS-CoV receptor ACE2 to gain entry into host cells and the serine protease TMPRSS2 for S protein priming. [Hoffman, M. et al. Cell (2020) 181 (2): 271-80] One mechanism for SARS-CoV-2 entry occurs when the spike protein on the surface of SARS-CoV-2 binds to an ACE2 receptor followed by cleavage at two cut sites (“priming”) that causes a conformational change allowing for viral and host membrane fusion. [Shrimp, J H et al. ACS Pharmacol. Trans. Sci. (2020) 3(5): 997-1007]. Angiotensin converting enzyme 2 (ACE2) and dipeptidyl peptidase 4 (DPP4) are known host receptors for SARS-CoV and MERS-CoV respectively [Yang, Y. et al., J. Autoimmunity (2020) doi.org/10.1016/j.jaut.2020.102434, citing Kuhn, J H, et al. Cell Mol. Life Sci. (2004) 61 (21): 2738-43; Raj, V S, et al. Nature (2013) 495 (7440): 251-54].

Although the respiratory system is a primary target of SARS-CoV-2, multiple nonpulmonary manifestations and complications of COVID-19 are being documented on an ongoing basis. For example, bioinformatics analysis of single-cell transcriptosome datasets of lung, esophagus, gastric, ileum and colon tissue reveal that the digestive system is also a potential route of entry for COVID-19. In addition, cardiovascular complications are rapidly emerging as a key threat in COVID-19. [Varga, Z. et al. The Lancet (2020) doi.org/10.1016/S0140-6736(20)30937-5] Endothelial cell involvement across vascular beds of different organs has been demonstrated in a series of patients with COVID-19. [Varga, Z. et al. The Lancet (2020) doi.org/10.1016/S0140-6736(20)30937-5].Early studies also have indicated that there is considerable musculoskeletal dysfunction in some patients with COVID-19, although long-term follow-up studies have not yet been conducted. [Disser, N P et al. J. Bone Joint Surg. Am. (2020) 102: 1197-204; Lopez. M. et al. Am. J. Physical Med. & Rehab. 99 (8) 669-73]. CoVs, which are neuroinvasive and neurotropic, can also be neurovirulent, causing illnesses such as meningitis and encephalitis. In addition, brain tissue is reported to contain ACE2 receptors. [Lopez. M. et al. Am. J. Physical Med. & Rehab. 99 (8) 669-73]. Thrombotic complications have been reported, including pulmonary embolism. Skin manifestations also have been documented. [Lopez. M. et al. Am. J. Physical Med. & Rehab. 99 (8) 669-73]

Just like other infectious agents, viruses cause disease by disrupting normal cell function in a variety of ways. Some viruses make repressor proteins that stop the synthesis of the host cell's proteins, RNA, and DNA. Viral activity may weaken cell membranes and lysosomal membranes, leading to cell autolysis. Some viral proteins are toxic to cells, and the body's immune defenses also may kill virus-infected cells.

### Disease Reservoirs

The reservoir for a disease is the site where the infectious agent survives. For example, humans are the reservoir for the measles virus because it does not infect other organisms.

Animals often serve as reservoirs for diseases that infect humans. For example, the major reservoir for Yersinia pestis is wild rodents. The reservoir for influenza is water fowl. The reservoir for the Sin Nombre hantavirus is the deer mouse (Peromyscus maniculatus), There are also nonliving reservoirs. Soil is the reservoir for many pathogenic fungi as well as some pathogenic bacteria such as Clostridium tetani, which causes tetanus.

### Modes of Transmission

Infectious agents may be transmitted through either direct or indirect contact. Direct contact occurs when an individual is infected by contact with the reservoir, for example, by touching an infected person, ingesting infected meat, or being bitten by an infected animal or insect. Transmission by direct contact also includes inhaling the infectious agent in droplets emitted by sneezing or coughing and contracting the infectious agent through intimate sexual contact. Some diseases that are transmitted primarily by direct contact with the reservoir include ringworm, AIDS, trichinosis, influenza, rabies, and malaria.

Indirect contact occurs when a pathogen can withstand the environment outside its host for a long period of time before infecting another individual. Inanimate objects that are contaminated by direct contact with the reservoir may be the indirect contact for a susceptible individual. Ingesting food and beverages contaminated by contact with a disease reservoir is another example of disease transmission by indirect contact. The fecal-oral route of transmission, in which sewage-contaminated water is used for drinking, washing, or preparing foods, is a significant form of indirect transmission, especially for gastrointestinal diseases such as cholera, rotavirus infection, cryptosporidiosis, and giardiasis.

These modes of transmission are all examples of horizontal transmission because the infectious agent is passed from person to person in a group. Some diseases also are transmitted vertically; that is, they are transmitted from parent to child during the processes of reproduction (through sperm or egg cells), fetal development, or birth. Diseases in which vertical transmission occurs include AIDS and herpes encephalitis (which occurs when an infant contracts the herpes simplex type II virus during vaginal birth).

### Active Immunization: Vaccination

The term “active immunization” as used herein refers to the production of active immunity, meaning immunity resulting from a naturally acquired infection or from intentional vaccination (artificial active immunity). Active immunity can be induced by either natural or artificial mechanisms.

The term “vaccination” as used herein refers to the act of administering a preparation intended for active immunological prophylaxis. Historically, vaccine approaches have been highly successful in providing cost effective measures to prevent disease and to control outbreaks of infection at herd level.

The first vaccine developed was one in which the wild-type disease or the wild-type version of a related disease was “killed” and delivered. While such vaccines were known to work, they carried a significant risk of severe disease or even death in the recipient.

The second type of vaccine developed was attenuated vaccines. This vaccine was based on material obtained from infected rabbit brain attenuated by drying, an uncertain process; vaccines prepared in this way frequently caused serious side effects. Attenuated vaccines are mostly now based on inactivated virus grown in tissue culture. Rabies was the first virus attenuated in a laboratory to create a human vaccine. Acquisition of the ability to grow viruses in tissue culture for an extended period led to the development of attenuated vaccines against measles, poliomyelitis, rubella, influenza, rotavirus, tuberculosis and typhoid. Because the vaccine components are alive, they can spread to non-vaccinated subjects, extending the impact of vaccination to the community at large (See generally, Greenwood B. The contribution of vaccination to global health: past, present and future. (2014). Philosophical transactions of the Royal Society of London. Series B, Biological sciences, 369(1645), 20130433. doi:10.1098/rstb.2013.0433).

Live attenuated vaccines. Live attenuated virus vaccines are a favored vaccination strategy, in part due to their previous success with the yellow fever virus vaccine, YF-17D, in the 1930s. (Ghaffar, K A. et al, “Fast Tracks and Roadblocks for Zika Vaccines,” Vaccines (2018) 6, 77; doi: 10.3390/vaccines040077).A single dose of YF-17D vaccine, for example, is able to induce high titers of neutralizing antibody (nAb) which confer protection on at least 95% of recipients (Id., citing Barrett A. D., Teuwen D. E. Curr. Opin. Immunol. (2009)21: 308-313. doi: 10.1016/j.coi.2009.05.018; Bonaldo, M C et al., Hum. Vaccin. Immunother. (2014) 10: 1256-1265. doi: 10.4161/hv.28117). This strategy has been employed with many other diseases, including polio, measles and mumps (Id., citing Plitnick L. M. Chapter 9-Global Regulatory Guidelines for Vaccines. In: Plitnick L. M., Herzyk D. J., editors. Nonclinical Development of Novel Biologics, Biosimilars, Vaccines and Specialty Biologics. Academic Press; San Diego, Calif., USA: (2013). pp. 225-241). Moreover, the production of attenuated vaccines is cost effective and fairly simple in comparison to other vaccine strategies.

While a live attenuated vaccine has the advantage of being able to elicit immune responses with a single dose, drawbacks include its limited use in immunocompromised or pregnant patients due to the risk of adverse effects. Indeed, because these vaccines contain live virus, mutations may occur in the attenuated vaccine strain with a reversion to virulence, as seen with oral polio vaccine, which causes paralysis in about one in two million recipients. Further, they may cause significant illness in subjects with impaired immunity, as has been seen with the anti-tuberculosis vaccine Bacille Calmette Guérin (BCG) when given to immunodeficient patients, including those with human immunodeficiency virus (HIV) infection.

Killed vaccines. Next, researchers developed killed vaccines where the pathogens were killed and then used. These vaccines were usually poorly immunogenic and often caused significant side effects, so that whole-cell vaccines have largely given way to subunit vaccines, among other types of vaccines. (See generally, Greenwood B. The contribution of vaccination to global health: past, present and future. (2014). Philosophical transactions of the Royal Society of London. Series B, Biological sciences, 369(1645), 20130433. doi:10.1098/rstb.2013.0433). Subunit vaccines comprise a fragment of a pathogen, i.e. a protein, or peptides (Ghaffar, K. A. et al, “Fast Tracks and Roadblocks for Zika Vaccines,” Vaccines (2018) 6, 77; doi: 10.3390/vaccines040077). While subunit vaccines are generally a safer choice, because they tend to be less immunogenic, an adjuvant and/or multiple doses are required.

mRNA-based vaccines. As the minimal genetic construct, mRNA contains only the elements required for expression of the specific encoded protein region. In addition, mRNA is incapable of interacting with the genome, but instead acts only as a transient carrier of information. Other advantages for its use as a vaccine platform include its safety profile (Ghaffar, K. A. et al, “Fast Tracks and Roadblocks for Zika Vaccines,” (2018) Vaccines 6, 77; doi: 10.3390/vaccines040077 citing Plitnick L. M. Chapter 9—Global Regulatory Guidelines for Vaccines. In: Plitnick L. M., Herzyk D. J., editors. Nonclinical Development of Novel Biologics, Biosimilars, Vaccines and Specialty Biologics. Academic Press; San Diego, Calif., USA: (2013). pp. 225-241; Lundstrom, K., Futre Sci. OA (2018) 4: FS0300 doi: 10.4155/fsoa-2017-0151). However, one of the disadvantages of utilizing mRNA as an approach to vaccine design is its potential for rapid degradation by ribonucleases.

DNA vaccines. DNA vaccines were one of the earliest vaccine platforms to be proposed for human clinical trials following the ZIKV outbreak (Id). The use of genetically engineered DNA plasmids encoding various antigens to induce both humoral and cellular responses also has been explored against various infectious diseases caused by parasites (Id., citing Cherif, M S et al, Vaccine (2011)29: 9038-9050; Cheng, P C et al., PLoS Neg. Trop Dis. (2016) 10: e00044594; doi: 10.1371/journal.pntd.0004459), bacteria (Id., citing Li, X. et al., Clin. Vaccine Immunol. 2012; 19:723-730. doi: 10.1128/CVI.05700-11; Albrecht, M T, et al., Med. Microbiol. (2012) 65: 505-509 doi: 10.1111/j.1574-695X.2012.00974.x). and other viruses (Id., citing Donnelly, J J et al., Nature Med. (1995) 1: 583-597 doi: 10.1038/nm0695-583; Porter, K R et al., Vaccine (2012) 30: 36-341 doi: 10.1016/j.vaccine.2011.10.085).

Adenovirus vectors whereby the vector expresses an unknown antigenic protein have been well studied for gene and cancer therapy and vaccines (Id). Apart from its extensive safety profile, the advantages of utilizing an adenovirus vector are that it is relatively stable, easy to attain high titers and able to infect multiple cell lines which attributes to its potency. Even though recombinant adenoviral vectors are widely used today thanks to its high transduction efficiency and transgene expression, there is likelihood for pre-existing immunity against the vector, because most of the population has been exposed to adenovirus (Id). This has been proven detrimental in a human immunodeficiency virus (HIV-1) phase IIb vaccine trial in which the vector-based vaccines provided favorable conditions for HIV-1 replication (Id., citing Smaill, F. et al., Sci. Transl. Med. (2013) 5: 205ra134. doi: 10.1126/scitranslmed.3006843).

### COVID-19 Vaccines

There has been a worldwide effort to respond to the COVID-19 pandemic.

In May 2020, in response to the COVID19 pandemic, the US Department of Health and Human Services (HHS) launched Operation Warp Speed—a partnership between government and industry—with the goal of delivering 300 million doses of a safe and effective vaccine by January 2021. [O'Callaghan, K P, et al. JAMA (2020) 324 (5): 437-38] This ambitious plan initially focused on 125 potential vaccine candidates, but was rapidly narrowed to 14 candidates in May 2020. Several of the vaccines that resulted have secured regulatory approval.

Moderna, a Massachusetts-based biotechnology company, successfully developed mRNA-1273, a lipid nanoparticle-encapsulated mRNA vaccine that encodes a full-length, prefusion stabilized spike (S) protein of SARS-CoV-2. [NCT04405076, visited 8/26/20]. Pfizer, in concert with BioNTech, a German company, developed a second, independent mRNA platform focused on lipid nanoparticle-encapsulated mRNA that encodes SARS-CoV-2 spike (S) protein.[NCT04368728, visited 8/26/20].

Johnson & Johnson's Janssen group has developed a COVID-19 vaccine that leverages the AdVac® vaccine platform also used to develop and manufacture Janssen's European Commission-approved Ebola vaccine regimen and construct its investigational Zika, RSV, and HIV vaccines. On Feb. 27, 2021, the U.S. Food and Drug Administration (FDA) issued an Emergency Use Authorization (EUA) for active immunization to prevent Coronavirus Disease 2019 (COVID-19) in individuals 18 years of age and older. [https://www.jnj.com/johnson-johnson-covid-19-vaccine-authorized-by-u-s-fda-for-emergency-usefirst-single-shot-vaccine-in-fight-against-global-pandemic, visited 15 Mar. 2021]. The AdVac® vaccine platform uses a replication-defective adenovirus type 26 (Ad26) vector that delivers recombinant SARS-CoV-2 spike (S) protein genes to human cells, [https://www.jnj.com/johnson-johnson-announces-acceleration-of-its-covid-19-vaccine-candidate-phase-1-2a-clinical-trial-to-begin-in-second-half-of-july, visited 8/26/20].

COVID-19 Vaccine AstraZeneca was co-invented by the University of Oxford and its spin-out company, Vaccitech. It uses a replication-deficient chimpanzee viral vector based on a weakened version of a common cold virus (adenovirus) that causes infections in chimpanzees and contains the genetic material of the SARS-CoV-2 virus spike protein. After vaccination, the surface spike protein is produced, priming the immune system to attack the SARS-CoV-2 virus if it later infects the body. The vaccine has been granted a conditional marketing authorization or emergency use in more than 70 countries across six continents; the recent Emergency Use Listing granted by the World Health Organization accelerates the pathway to access in up to 142 countries through the COVAX Facility. [https://www.astrazeneca.comn/media-centre/press-releases/2021/update-on-the-safety-of-covid-19-vaccine-astrazeneca.html]

WHO reported that as of 10 Mar. 2021, a total of 300,002,228 vaccine doses have been administered. [covid19.who.int, visited 15 Mar. 2021]. The ultimate success of this herculean effort to stem the COVID-19 pandemic remains to be determined. There is no evidence to date that vaccination will lead to long term immunity or herd immunity.

### Vaccination Mediated Protection and its Shortcomings

Vaccine-induced immune effectors are essentially antibodies, produced by B lymphocytes, which are capable of binding specifically to a toxin or a pathogen. Other potential effectors are cytotoxic CD8+ T lymphocytes that may limit the spread of infectious agents by recognizing and killing infected cells or secreting specific antiviral cytokines and CD4+ T-helper (T_{H}) lymphocytes. These T_{H} cells may contribute to protection through cytokine production and provide support to the generation and maintenance of B and CD8+ T-cell responses. Effector CD4+ T_{H} cells were initially subdivided into T-helper 1 (T_{H}1) or T-helper 2 (T_{H}2) subsets depending on their main cytokine production (interferon-γ or interleukin 4 (IL-4), respectively. T_{H} cells include a large number of subsets with distinct cytokine-producing and homing capacities. For example, follicular T-helper (T_{FH}) cells are specially equipped and positioned in the lymph nodes to support potent B-cell activation and differentiation into antibody secreting cells; they were identified as directly controlling antibody responses and mediating adjuvanticity. T-helper 17 (T_{H}17) cells essentially defend against extracellular bacteria that colonize the skin and mucosa, recruiting neutrophils and promoting local inflammation. These effectors are controlled by regulatory T cells (Tregs) involved in maintaining immune tolerance.

Although the nature of a vaccine exerts a direct influence on the type of immune effectors that are elicited, the induction of antigen-specific immune effectors (and/or immune memory cells) by an immunization process does not imply that the resulting antibodies, cells, or cytokines represent surrogates, or even correlates, of vaccine efficacy (Rueckert C, Guzmán CA (2012) Vaccines: From Empirical Development to Rational Design. PLoS Pathog 8(11): e1003001. doi:10.1371/journal.ppat.1003001).

The protection provided by current vaccination efforts is largely dependent on the induction of neutralizing antibodies. Antibody-mediated neutralization of viruses is the direct inhibition of viral infectivity resulting from antibody docking to virus particles. Neutralization occurs when the process of virion binding to the cell surface receptors is inhibited, or when the fusion process of virion with cellular endosomal or plasma membranes is disrupted. Neutralizing antibodies precisely target specific antigens. In addition to directly interfering with virus entry into cells, antibodies can further counteract viral infection through their Fc fragments, triggering immune regulatory mechanisms, including antibody-dependent cell cytotoxicity (ADCC), antibody-dependent cellular phagocytosis (ADCP), and complement-dependent cell cytotoxicity (CDCC) or complement dependent cytotoxicity (CDC).

However, neutralizing antibody protection has its limitations. First, there are a number of issues with the process of vaccine development itself, such as animal model unavailability. For example, in targeting Flaviviridae viruses, there is currently no perfect small animal model for pre-clinical testing; so far, non-human primates (NHP), one of the natural reservoirs of the virus, are the best pre-clinical models [Lee, C Y P, Ng, L F P, (2018) Microbes Infect. doi: 10.1016/j.micinf.2018.02.009].

Second, pathogens present themselves in numerous variants and may undergo mutations to enable immune escape. For example, influenza viruses utilize RNA-dependent RNA polymerase (RdRp) to catalyze the replication cycle; RdRp is prone to error thus accumulating new mutations and changing the genome over time in a process termed “antigenic drift.” (Fermin, Gustavo, and Paula Tennant. (2018) Viruses: Molecular Biology, Host Interactions and Applications to Biotechnology, edited by Jerome E. Foster, Elsevier Science & Technology. ProQuest Ebook Central, https://ebookcentral.proquest.com/lib/jhu/detail.action?docID=5322098). Antigenic shift also allows some influenza strains to adapt to new species, such as has been observed in recent times with avian influenza. Further, segmented genomes, such as those of Orthomyxovirus. can undergo genome reassortment. Moreover, when the protein antigens are highly variable among the different strains, the antibody produced is non-neutralizing. (Stanley A. Plotkin, (2015) Increasing Complexity of Vaccine Development, The Journal of Infectious Diseases, Volume 212, Issue Suppl_1, Pages S12-S16, https://doi.org/10.1093/infdis/jiu568). The extreme diversity of human immunodeficiency virus (HIV), a member of the family Retroviridae is a major obstacle to vaccine development, since strains belonging to different subtypes can differ by up to 35% in some of their proteins, such as the env proteins. Therefore, while some vaccines may be effective against some virus clades, they may not be effective against other clades (see Hsu, D. et al, (2017) “Progress in HIV vaccine development” Human Vaccines & Immunotherapeutics 13(5): 1018-1030).

Third, vaccine induced immunity may not be effective enough to confer long-term immunity. While improving humoral immunity to viral infection is the target of many current conventional vaccines, for example influenza vaccines, such vaccines are generally not cross-protective. Developments in Herpesviridae vaccine design have resulted in vaccines that only have partial efficacy. (Sandgren, K., et al., (2016) “Understanding natural herpes simplex virus immunity to inform next-generation vaccine design.” Clinical & Translational Immunology 5(7)).

Fourth, despite experimental models, some vaccines may not invoke the desired functional response. In a macaque model, some sera from patients who eventually died of SARS-CoV and that displayed faster neutralizing antibody responses to the CoV Spike proteins caused severe acute lung injury in productively infected lungs by skewing macrophage responses during the acute phase of infection. [Liu, L. et al. J. Clin. Insight (2019) 4 (40): e123158]. Antibody-dependent enhancement (ADE) of ZIKV by dengue and West Nile immune sera has been shown in vitro and induced in immunosuppressed mice by dengue and West Nile immune sera due to the sequence and antigenic similarity between them. Sairol, C A et al (2018), Trends in Microbiol. 26(3): 186-190. During this phenomenon, cells bearing Fc receptors (FcR) can uptake and internalize antibody-coated viruses and be further infected (Yang, C. et al., (2019) Development of neutralizing antibodies against Zika virus based on its envelope protein structure,” Virologica Sinica 34: 168-174, citing Dowd, K A and Pierson, T C, (2011) Virology. 411: 306-315. doi: 10.1016/j.virol.2010.12.020).

Fifth, there are a number of population specific challenges that may alter the immune response to the vaccine. For example, early life antibody responses markedly differ from those elicited in mature hosts, and the capacity to induce protective antibody at a sufficient titer to prevent infection declines significantly with age. In individuals 65 years or older, influenza and hepatitis B vaccines induce protective antibody titers in less than half of recipients. [Siegrist, C-A and Aspinall, R. Nature Reviews Immunology (2009) 9: 185-194, citing Hannoun, C. et al. Virus Res. (2004) 4: 553-64; Looney, R J et al, J. Clin. Immunol. (2001) 21: 30-36]. Also, vaccine immunogenicity may be low in immunocompromised persons, including those with solid organ transplants, hematopoietic stem cell transplants, solid cancers and hematologic malignancy as well as those with autoimmune conditions receiving biological therapies [Bosaeed, M. and Kumar, D. Hum. Vaccin. Immunother. (2018) 14 (6): 1311-22].

Sixth, there is insufficient information about the mechanisms of protection, as well as the antigens/epitopes required for sufficient activation of the targeted mechanism. For example, recent data showed that the structure of dengue virus differs according to the temperature at which virus replication takes place. In cell culture or in the human at 37° C., the structure of the virus is expanded, whereas at lower temperatures in the mosquito, the particle is more compact It has been suggested that epitopes exposed on the vaccine virus may not be exposed on the mosquito challenge virus, which is therefore able to enter cells without being neutralized. (Stanley A. Plotkin, (2015) Increasing Complexity of Vaccine Development, The Journal of Infectious Diseases, Volume 212, Issue Suppl_1, Pages S12-S16, https://doi.org/10.1093/infdis/jiu568).

In summary, despite the development of vaccines, morbidity and mortality from pathogens worldwide has not truly decreased. There are no universally accepted strategies and tools to rationally design vaccines, and vaccine development generally is still a tedious and costly empiric process. In many cases, rationally designed vaccines have not been successful, due to insufficient knowledge about the mechanisms of protection. Although the repertoire of immune clearance mechanisms to fight pathogens is known, the specific contributions of different effector mechanisms are well-characterized for only a few pathogens. It is also largely unclear what determines the immunogenicity and selection of particular epitopes among all possible antigenic options offered by a pathogen. For example, it is not known which factors determine dominant or balanced immune responses, and what are the mechanisms leading to long-term protection for each individual pathogen (Dye C. (2014). After 2015: infectious diseases in a new era of health and development. Philosophical transactions of the Royal Society of London. Series B, Biological sciences, 369(1645), 20130426. doi:10.1098/rstb.2013.0426).

### The Host Immune Response to Viral Infection

The human immune system is a complex arrangement of cells and molecules that maintain immune homeostasis to preserve the integrity of the organism by elimination of all elements judged to be dangerous. Responses in the immune system may generally be divided into two arms, referred to as “innate immunity” and “adaptive immunity.” The two arms of immunity do not operate independently of each other, but rather work together to elicit effective immune responses.

### Innate Immune Response

The innate arm of the immune system is a nonspecific fast response to pathogens that is predominantly responsible for an initial inflammatory response via a number of soluble factors, including the complement system and the chemokine/cytokine system; and a number of specialized cell types, including mast cells, macrophages, dendritic cells (DCs), and natural killer cells (NKs).

### Complement Activation

The complement system is a system of soluble pattern recognition receptors (PRRs) and effector molecules that detect and destroy microorganisms. In the presence of pathogens or of antibody bound to pathogens, soluble plasma proteins that in the absence of infection circulate in an inactive form becomes activated, so that particular complement proteins interact with each other to form the pathways of complement activation, which are initiated in different ways. As shown schematically in FIG. 1, complement is activated through the classical pathway (CP), the lectin pathway (LP), and the alternative pathway (AP). In the classical and lectin pathways, binding of soluble pattern recognition molecules (PRMs) to a pathogen-associated molecular pattern (PAMP) or a damage-associated molecular pattern (DAMP) (the activator) activates zymogen proteases in complex with the PRMs. The classical pathway is initiated when complement component C1, which comprises a recognition protein (C1q) associated with proteases (C1r and C1s) either recognizes a microbial surface directly or binds to antibodies already bound to a pathogen. Exemplary C1q ligands include antigen-antibody complexes, molecular patterns on certain bacteria, viruses, parasites, and mycoplasma, C-reactive protein (CRP) in complex with exposed phosphocholine residues on bacteria; pentraxin-3 (PTX-3), serum amyloid P component, β-amyloid fibrils, as well as tissue damage elements such as DNA and mitochondrial membranes [Bajik, G., et al. EMBO J. (2015) 34 (22) 2735-57, citing Kang, Y H et al. Adv Exp Med Biol (2009) 653: 117-128], and DAMPS such as DNA, histones, and annexins A2 and A5 exposed by apoptotic cells [Id., citing Martin, M. et al. J Biol Chem (2012) 287: 33733-33744]. The proteins SCARF1 and LAIR have been implicated as immunomodulatory receptors for C1q-opsonized apoptotic cells. [Id., citing Son, M. et al. Proc Natl Acad Sci USA (2012) 109: E3160-E3167; Ramirez-Ortiz et al, Nat Immunol (2013) 14: 917-926]. Following C1q-ligand binding, C1r autoactivates and subsequently cleaves C1s, which may then cleave C4 into the fragments C4a and C4b. The nascent C4b can be covalently bound to the activator via an exposed internal thioester leading to irreversible tagging of the activator. C2 binds activator-bound C4b and is cleaved by C1s to generate the active serine protease C2a bound to C4b resulting in the CP C3 convertase C4b2a [Id., citing Muller-Eberhard, H J et al, J Exp Med (1967) 125: 359-380 1967]. The C3 convertase cleaves C3 into the anaphylatoxin C3a and the major opsonin of the complement system, C3b, which like C4b, becomes covalently coupled to the activator through its exposed thioester [Id., citing Law S K, Dodds A W. Protein Sci (1997) 6: 263-274).

The lectin pathway is initiated by soluble carbohydrate-binding proteins—mannose-binding lectin (MBL) and the ficolins—that bind to particular carbohydrate structures on microbial surfaces. MBL-associated serine proteases (MASPs), which associate with the recognition proteins, then trigger cleavage of complement proteins and activation of the pathway.

Activation of the lectin pathway (LP) is initiated by the collectins MBL and CL-LK or one of three ficolins. MBL and CL-LK harbor Ca2+-dependent carbohydrate-recognition domains (CRDs) and collagen-like regions through which they trimerize. Such trimers oligomerize in larger complexes, allowing high-avidity binding (K_{D}≈10⁻⁹ M) based on multiple low-affinity interactions of their CRDs (K_{D}≈10⁻³ M) [Id., citing Kawasaki et al. J Biochem (1983) 94: 937-947; Degn S E, Thiel S. Scand J Immunol (2013) 78: 181-193]. Ficolins are structurally similar to collectins, but instead of C-type lectin domains they possess fibrinogen (FBG)-like domains for PAMP recognition [Id., citing Matsushita M. Ficolins in complement activation. Mol Immunol (2013)55: 22-26]. Ficolins recognize motifs containing acetylated groups, including non-sugars such as N-acetyl-glycine, N-acetyl-cysteine, and acetylcholine. Besides conferring avidity, the oligomerization of collectins and ficolins allows these PRMs to discriminate not only specific monosaccharides or acetylated groups but also specific patterns of sugars and acetyl groups characteristic to pathogens. The LP PRMs form complexes with MBL-associated serine proteases (MASPs), which are always present as dimers. MASP-1 and MASP-2 are structural and functional homologs of C1r and C1s from the CP, but there are important differences between PRM-protease complexes from the two pathways. Whereas the C1 complex has a defined stoichiometry (a hexamer of the heterotrimeric C1q subunit in complex with a C1r2s2 tetramer), the LP PRMs are polydisperse oligomers of trimers. For MBL, a tetramer is the most abundant oligomer and this carries only a single MASP-1 or MASP-2 dimer, but the more rare, larger oligomers may simultaneously carry both dimers [Id., citing Dahl M R, et al. Immunity (2001) 15: 127-135; Teillet, F. et al, J Immunol (2005) 174: 2870-2877; Degn, S E et al J Immunol (2013) 191: 1334-1345]. MASP-1 in complex with an activator-bound PRM autoactivates and cleaves MASP-2 as well as C2, whereas activated MASP-2 cleaves C4 and C2 resulting in the same C3 convertase as in the CP, that is, C4b2a [Id., citing Matsushita, M et al. J Immunol (2000) 165: 2637-2642; Rossi, V. et al. J Biol Chem (2001) 276: 40880-40887; Chen C B, Wallis R J Biol Chem (2004)279: 26058-26065].

The alternative pathway (AP) can be initiated by spontaneous hydrolysis and activation of complement component C3, which can then bind directly to microbial surfaces. Activation through the CP and LP results in deposition of C3b on the activator, which recruits factor B (FB) in the first step of the AP. The resulting proconvertase C3bB is subsequently cleaved by factor D (FD), generating the AP C3 convertase C3bBb (Id., citing Fearon, D T et al. J Exp Med (1973) 138: 1305-1313], which is functionally homologous to the CP C3 convertase C4b2a. A positive feedback amplification loop is initiated as multiple copies of C3b are deposited on the activator leading to further assembly of the AP C3 convertase. Regardless of the initiating pathway, up to 90% of the deposited C3b molecules are generated through the AP [Id., citing Harboe, M. et al, Clin Exp Immunol (2004) 138: 439-446; Harboe, M. et al. Mol Immunol 47: 373-380]. This amplification is rapidly terminated on host cells by various regulators, but proceeds on pathogens and altered host tissues lacking such regulators.

The three pathways converge at the step whereby enzymatic activity of a C3 convertase is generated. Cleavage of C3 is the critical step in complement activation and leads directly or indirectly to all the effector activities of the complement system. The C3 convertase is bound covalently to the pathogen surface, where it cleaves C3 to generate large amounts of C3b, the main effector molecule of the complement system, and C3a, a small peptide that binds to specific receptors and helps induce inflammation.

The terminal pathway (TP) of complement is initiated when a threshold density of C3b molecules on an activator has been reached. The C3 convertases can recruit another C3b molecule to form C3bBb3b [Id., citing Medicus, R G et al. J Exp Med (1976) 144: 1076-1093] and C4b2a3b [Id., citing Takata, Y et al. J Exp Med (1987) 165: 1494-1507], the AP and CP C5 convertases, respectively. Through cleavage of C5, they generate the potent chemoattractant C5a and C5b. The latter forms the lytic membrane attack complex (MAC, also called C5b-9) together with C6, C7, C8, and multiple C9 molecules in membranes of pathogens lacking a protective cell wall like Gram-negative bacteria [Id., citing Laursen, N S et al. Curr Mol Med (2012) 12: 1083-1097; Berends, E T et al. FEMS Microbiol Rev (2014) 38: 1146-1171].

All three pathways have the final outcome of killing the pathogen, either directly or by facilitating its phagocytosis, and inducing inflammatory responses that help to fight infection.

The anaphylatoxins C3a and C5a, which are released when the convertases cleave C3 and C5, exert their biological functions upon binding to seven-transmembrane domain (7TM) receptors in the membranes of host cells. Two of these receptors, C3aR and C5aR1 (CD88), are G protein-coupled receptors (GPCR), whereas the third, C5aR2 (previously known as C5L2), is structurally similar to C5aR1 but does not couple to heterotrimeric G proteins [Id., citing Li, R. et al. FASEB J (2013) 27: 855-864, 2013]. C5aR2 was first considered as a decoy receptor, limiting the availability of the C5a and C5adesArg ligands to C5aR1. Decoy receptors do not undergo ligand-induced internalization but are rather continuously recycled between the cell membrane and the intracellular compartments, thereby removing their extracellular ligand [Id., citing Weber, M. et al. Mol Biol Cell (2004) 15: 2492-2508]. Thus, it has been suggested that C5aR2 may reduce the cellular responses to pro-inflammatory molecules and thereby actively regulate inflammatory processes [Id., citing Rittirsch, D. et al. Nat Med (2008) 14: 551-557]. Additionally, some studies report concerted action of C5aR1 and C5aR2 in adipocyte metabolism and immunity as well as formation of C5aR1/C5aR2 heterocomplexes [Id., citing Bamberg, C E et al. Adv Exp Med Biol (2010) 632: 117-142; Poursharifi, P. et al. Mol Cell Endocrinol (2014) 382: 325-333].

Signaling through C3aR and C5aR1 triggers chemotaxis, oxidative burst, histamine release, and leukotriene and interleukin synthesis [Id., citing Klos, A. et al, Mol Immunol (2009) 46: 2753-2766].

There are five known C3b receptors on the surface of cells, especially immune cells. Complement receptor 1 (CR1, CD35) is a large CCP module-based glycoprotein expressed on almost all peripheral blood cells except NK and T cells [Id., citing Fearon, D T. J Exp Med (1980) 152: 20-30; Tedder, T F et al, J Immunol (1983) 130: 1668-1673]. CR1 binds C3b and C4b with high affinity and iC3b and C3d with a lower affinity [Id., citing Reynes, M. et al J Immunol (1985)135: 2687-2694]. CR1 on erythrocytes may bind C3b-containing immune complexes as part of removal processes, whereas on phagocytic cells it promotes C3b/C4b− coated particle uptake. CR1 also plays an important role in the germinal centers of lymph nodes where it is found on follicular dendritic cells (FDCs) capturing complement-opsonized antigens that serve to stimulate B cells [Id., citing Heesters, B A et al. (2013) Nat Rev Immunol 14: 495-504]. CR2 (CD21), also possessing a CCP architecture, is primarily present on B cells and FDCs. It is important in trapping of C3-opsonized antigens by FDCs in the germinal centers and stimulating B cells for affinity maturation, isotype switching, and memory [Id., citing Fang, Y. et al. (1998) J Immunol 160: 5273-5279; Carroll, M C (2000) Adv Immunol 74: 61-88]. CR2 binds C3b, iC3b, and C3d with the same affinity in agreement with the crystal structure of the CR2-C3d complex revealing recognition of a surface patch on the thioester (TE) domain accessible in all three ligands but concealed in C3 prior to cleavage [Id.].

CR3 and CR4 are integrin-type heterodimeric receptors (CD11b/CD18 and CD11c/CD18) having distinct α-chains, αM and αX, respectively, but sharing a common β2-chain. Both are phagocytic receptors expressed on myeloid leukocytes and NK cells and share iC3b as ligand [Id., citing Metlay, J P et al. (1990) J Exp Med 171: 1753-1771; Ross, G D (2000) Crit Rev Immunol 20: 197-222]. However, structural studies indicate that the receptors bind to different epitopes of iC3b. CR3 was shown to recognize the thioester (TE) domain of iC3b [Id., citing Bajic, G. et al. (2013) Proc Natl Acad Sci USA 110: 16426-16431], whereas CR4 binds quite far from this in the C3c moiety of iC3b [Id., citing Chen, X. et al. (2012) Proc Natl Acad Sci USA 109: 4586-4591]. CR3 and CR2 may bind simultaneously to the iC3b TE domain [Id., citing Bajic, G. et al (2013) Proc Natl Acad Sci USA 110: 16426-16431], and since CR3 is expressed on subcapsular sinus macrophages (SSMs), it is plausible that complement-bearing immune complexes could be conveyed from CR3-positive SSMs to CR2-positive naïve B cells within lymph nodes [Id., citing Phan, T G et al. (2007) Nat Immunol 8: 992-1000; Bajic, G. et al. (2013) Proc Natl Acad Sci USA 110: 16426-16431; Heesters, B A et al. (2014) Nat Rev Immunol 14: 495-504]. SSM are poorly endocytic, and appear to retain ICs on their surface during the IC shuttling from the sinus-lining to the follicular side [Id., citing Phan, T G et al. (2009) Nat Immunol 10: 786-793]. The fifth C3b receptor is CRIg (VSIG4), an immunoglobulin-type receptor expressed on liver-resident macrophages (Kupffer cells), which plays an important role in the clearance of pathogens from the circulation through interaction with surface-bound C3b and iC3b opsonins [Id., citing Helmy, K Y et al. (2006) Cell 124: 915-927]. The binding of CRIg to C3b selectively inhibits the interaction of C3 and C5 with the AP, but not with the CP convertases.

Besides acting in innate immunity, the complement system also influences adaptive immunity. For example, opsonization of pathogens (meaning the coating of the surface of a pathogen that makes it more easily ingested by phagocytes) by complement facilitates their uptake by phagocytic APCs that express complement receptors, which enhances presentation of pathogen antigens to T cells. B cells express receptors for complement proteins that enhance their responses to complement-coated antigens. Several complement fragments also can act to influence cytokine production by APCs, thereby influencing the direction and extent of the subsequent adaptive immune response. [Janeway's Immunology, 9th Ed. (2017) Garland Science, New York, Chapter 2, 49-51].

Complement fragments can be generated by other means besides the three canonical activation routes. The cross-talk with the coagulation system has regained attention due to studies indicating that thrombin, coagulation factors XIa, Xa, and IXa, and plasmin effectively cleave C3 and C5 and generate C3a and C5a [Bajik, G. et al. EMBO J. (2015) 34(22): 2735-57, citing Huber-Lang, M. et al. (2006) Nat Med 12: 682-687; Amara U. et al. (2010) J Immunol 185: 5628-5636; Berends, E T et al. (2014) FEMS Microbiol Rev 38: 1146-1171]. C3 can also be produced intracellularly by CD4+ T cells. Thus C3 is processed by the T-cell lysosomal protease cathepsin L, yielding biologically active C3a and C3b [Id., citing Liszewski, M K et al. (2013) Immunity 39: 1143-1157]. Tonic intracellular C3a generation is required for homeostatic T-cell survival, whereas shuttling of this intracellular C3 activation system to the cell surface upon T-cell stimulation additionally induces autocrine proinflammatory cytokine production. Thus, C3aR activation via intrinsic generation of C3a appears to be an integral part of human T_{H}1 immunity [Id., citing Ghannam, A. et al, (2014) Mol Immunol 58: 98-107]. Thrombin slowly cleaves C5 and generates C5a, but under conditions with normal convertase activity, this is possibly not a physiologically significant reaction. Clotting-induced production of thrombin instead leads to cleavage of C5 or C5b in the CUB domain. C5a can be released from such CUB-digested C5 by the conventional C5 convertases, and the combined action of thrombin and convertases appears to enhance the efficiency of the lytic pathway [Id., citing Krisinger, M J et al. (2012) Blood 120: 1717-17251. Conversely, MASP-1 has been reported to activate coagulation [Id., citing Takahashi, K. et al. (2011) Immunobiology 216: 96-102; La Bonte, L R et al. (2012) J Immunol 188: 885-8911 and to initiate endothelial cell signaling via cleavage of protease-activated receptor 4 [Id., citing Megyeri, M. et al. (2009) J Immunol 183: 3409-3416].

The complement system has been implicated as a contributor to the observed tissue damage that occurs in such severe virus infections as influenza A virus H1N1 [Wang, R. et al. Emerging Microbes and Infections (2015) 4: e28, citing Garcia, C C. et al. PLoS One (2013) 8: e64443; Berdal, J E et al., J. Infect. (2011) 63: 308-16], H5N1 [Id., citing Sun, S. et al. Am. J. Respir. Cell Mol. Biol. (2013) 49: 221-230], H7N9 [Id., citing Sun, S. et al. Clin. Infect. Dis. (2014) 60: 586-95], SARS-CoV [Id., citing Huang, K J, et al. J. Med. Virol. (2005) 75: 185-94]; and MERS-CoV [Id., citing Zhou, J. et al. J. Infect. Dis. (2014) 209: 1331-1442]. Studies suggest the synthesis of complement components by human alveolar macrophages and synthesis and secretion of complement components by pulmonary alveolar type II epithelial cells. [Id., citing Ackerman, S K et al., Immunology (1978) 35: 369-72; Coi, F S, et al., Clin. Immunol. Immunopathol. (1983) 27: 153-59; Strunk, R C et al., J. Clin. Invest. (1988) 81: 1419-146].

Among the complement activation products, the anaphylatoxin C5a is one of the most potent inflammatory peptides. [Id. citing Marc, M M, et al. Am. J. Respir. Cell Mol. Biol. (2004) 31: 216-19]. It is a strong chemoattractant for neutrophils and monocytes and activates these cells to generate oxidative bursts with release of reactive oxygen species (ROS), especially O₂ and H₂O₂. [Id., citing Guo, R F, Ward, P A. Annu. Rev. Imunol. (2005) 23: 821-52]. C5a mediates neutrophil attraction, aggregation, activation and subsequent pulmonary endothelial damage. [Id., citing Stevens, J H, Raffom. T A. Postgrad. Med. J. (1984) 60: 505-513; Tate, R M, Repine, J E. Am. Rev. Respir. Dis. (1983) 128: 802-806; Craddock, P R et al., J. Clin. Invest. (1977) 60: 260-64; Sacks, T. et al. J. Clin. Invest. (1978) 61: 1161-67]. C5a activates macrophages and endothelial cells and promotes vascular leakage and the release of Neutrophil Extracellular Traps (NETs). [Id., citing Guo, R F, Ward, P A. Annu. Rev. Immunol; (2005) 23: 821-52]. NETs are primarily composed of DNA from neutrophils, which bind pathogens with anti-microbial proteins, and increase the permeability of the alveolar-capillary barrier by cleaving endothelial actin cytoskeleton, E-cadherin and VE-cadherin. [Id., citing Saffarzadeh, M. et al. PLoS One (2012) 7: e32366]. The antimicrobial peptide LL-37 in NET structures is cytotoxic and pro-apoptotic toward endothelial and epithelial cells [Id., citing Aarbiou, J. et al. Inflamm. Res. (2006) 55: 119-127]. NETs also induce the release of proinflammatory cytokines. [Id., citing Saffarzadeh, M. et al. PLoS One (2012) 7: e32366].

C5a is also a potent chemoattractant for T cells [Id., citing Nataf, S., e al., J. Immunol. (1999) 162: 4018-23; Tsuji, R F et al. J. Immunol. (2000) 165: 1588-98], B cells [Id., citing Ottonello, L, et al. J. Immunol. (1999) 162: 6510-17], and dendritic cells (DCs) [Id., citing Morelli, A., et al., Immunology (1996) 89: 126-34; Sozzani, S. et al, J. Immunol. (1995) 155: 3292-95; Mrowietz, U. et al. Exp. Dermatol. (2001) 10: 238-45; Yang, D. et al. J. Immunol. (2000) 165: 2694-2702], which release cytokines, such as TNF-α, IL-1β, IL-6, and IL-8 [Id., citing Hopken, U., et al. Eur. J. Immunol. (1996)26: 1103-09; Strieter, R M et al. Am. J. Pathol. (1992) 141: 397-407]. DCs can then take up antigen and are primed for T cell help. [Id., citing Kim, A H, et al. J. Immunol. (2004) 173: 2524-29]. The process of leukocyte adhesion to endothelial cells is the first critical step in neutrophil migration into an area of inflammation. C5a induces upregulation of CD11b/CD18 expression on neutrophils. [Id., citing Guo, R F, Ward, P A. Annu. Rev. Imunol. (2005) 23: 821-52]. IL-8 levels have been found to correlate with neutrophil numbers and the degree of lung dysfunction. [Id., citing Williams, T J, Jose, P J. Novartis Found Symp. (2001) 234: 136-41]. C5a directly activates endothelial cells to upregulate adhesion molecules, such as P-selectin, and C5a and TNF-α cooperate to enhance upregulation of intracellular adhesion molecule 1 and E-selectin [Id., citing Ward, P A. Ann. NY Acad. Sci. (1996) 796: 104-112].

Strategies of Innate Immunity that Defend Against Intracellular Pathogens

Viruses are obligate intracellular pathogens—they must invade host cells to replicate. Two strategies of innate immunity defend against intracellular pathogens.

One is to destroy pathogens before they infect cells. To this end, innate immunity includes soluble defenses, such as antimicrobial peptides (e.g., defensins, athelicidins, and histatins) and phagocytic cells (macrophages, neutrophils and dendritic cells) that can engulf and destroy pathogens before they become intracellular. Macrophages and neutrophils constitutively express cell-surface receptors that stimulate the phagocytosis and intracellular killing of microbes bound to them, although some also signal through other pathways to trigger other responses, e.g., cytokine production. These phagocytic receptors include several members of the C-type lectin-like family (e.g., Dectin-1, and the mannose receptor (MR)); scavenger receptors that recognize various anionic polymers and acetylated low density lipoproteins; and complement receptors and Fc receptors that bind to complement coated microbes or to antibodies bound to the surface of microbes that facilitate phagocytosis.

The nucleic acid sensing toll like receptors (TLRs)—TLR3, TLR-7, TLR-8 and TLR-9, are endosomal nucleotide sensors involved in the recognition of viruses. TLR-3 is expressed by macrophages, conventional dendritic cells, and intestinal epithelial cells; it recognizes double-stranded RNA which is a replicative intermediate of many types of viruses. TLR-7 and TLR-9 are expressed by plasmacytoid dendritic cells, B cells and eosinophils; TLR-8 is expressed primarily by monocytes and macrophages. TLR-7 and TLR-8 are activated by single-stranded RNA. The virus genome for example of orthomyxoviruses (such as influenza) and flaviviruses (such as West Nile virus) consist of single stranded RNA. When extracellular particles of these viruses are endocytosed by macrophages or dendritic cells, they are uncoated in the acidic environment of endosomes and lysosomes, exposing the ssRNA genome for recognition by TLR-7. TLR-8 is physiologically most similar to TLR7, recognizes viral ssRNA, and is predominantly expressed in monocytes. [Petrasek, J. et al., Advances in Clin. Chem. (2013) 59: 255-201]. TLR-9 recognizes unmethylated CpG nucleotides; in the genomes of bacteria and many viruses, CpG dinucleotides remain unmethylated. [Janeway's Immunology, 9th Ed. (2017) Garland Science, New York, at 91]

Macrophages and neutrophils secrete lipid mediators of inflammation—prostaglandins, leukotrienes, and platelet-activating factor (PAF)—which are rapidly produced by enzymatic pathways that degrade membrane phospholipids. Signaling by mammalian TLRs in various cell types induces a diverse range of intracellular responses that together result in the production of inflammatory cytokines, chemotactic factors, antimicrobial peptides, and the antiviral cytokines interferon α and interferon β. [Janeway's Immunology, 9th Ed. (2017) Garland Science, New York, at 92]

Viral RNAs produced within a cell are sensed by Retinoic acid-Inducible Gene I (RIG-1) like receptors (RLR), which bind to viral RNA using an RNA helicase-like domain in their carboxy terminal, which has a DexH tetrapeptide amino acid motif and is a subgroup of DEAD-box family proteins. The RLR proteins also contain two amino terminal CARD domains that interact with adaptor proteins and activate signaling to produce type 1 interferons when viral RNAs are bound. RIG-1 discriminates between host and viral RNA by sensing differences at the 5′ end of single stranded RNA transcripts—most RNA viruses do not replicate in the nucleus where addition of a 7-methylguanosine to the 5′triphosphate (called capping) occurs, and their RNA genomes do not undergo this modification. RIG-1 senses the unmodified 5′-triphosphate end of the ssRNA viral genome. MDA-5 (melanoma differentiation-associated 5), also called hellicard, is similar in structure to RIG-1, but it senses dsRNA. The RLR family member LGP2 (encoded by DHX58) retains a helicase domain but lacks CARD domains. It appears to cooperate with RIG-1 and MDA-5 in the recognition of viral RNA. Before infection by viruses, RIG-1 and MDA-5 are in the cytoplasm in an auto-inhibited configuration that is stabilized by interactions between the CARD and helicase domains. [Id.]

Sensing of viral RNAs activates signaling by RIG-1 and MDA-5, which leads to type 1 interferon production. Upon infection, viral RNA associates with the helicase domains of RIG-1 or MDA-5, freeing the two CARD domains for other interactions. The more amino-proximal portion of the two CARD domains can then recruit E3 ligases, including TRIM25 and RIPLET, which initiate K63-linked polyubiquitin scaffolds, which appear to help RIG-1 and MDA-5 interact with a downstream adaptor protein called mitochondrial antiviral signaling protein (MAVS). MAVS is attached to the outer mitochondrial membrane and contains a CARD domain that may bind RIG-1 and MDA-5. This aggregation of CARD domains may initiate aggregation of MAVs, which propagates signals by recruiting various TRAF family E3 ubiquitin ligases, including TRAF2, TRAF3, TRAF5, and TRAF6. Their further production of K63-linked polyubiquitin leads to activation of TBK1 and IRF3 and production of type 1 interferons as described for TLR-3 signaling, and to activation of Nf-κB. [Janeway's Immunology, 9th Ed. (2017) Garland Science, New York, at 102-103].

Alternatively, the innate immune system can recognize and kill cells infected by some pathogens. Natural killer cells (NK cells), the only cytotoxic population of innate lymphoid cells (ILCs) [Jiao, Y. et al., Front. Immunol. (2020) 11: 282] are instrumental in keeping certain viral infections in check before cytotoxic T cells of the adaptive immune response become functional. Virus-infected cells can become susceptible to being killed by NK cells by a variety of mechanisms. First, since some viruses inhibit all protein synthesis in their host cells; synthesis of MHC class I proteins would be blocked in infected cells, which would make them correspondingly less able to inhibit NK cells through their MHC-specific receptors, and they would become more susceptible to being killed. Second, many viruses can selectively prevent the export of MHC class I molecules to the cell surface, or induce their degradation once there. Virally infected cells can still be killed by NK cells even if the cells do not downregulate MHC, provided that ligands for activating receptors are induced. Viruses that target ligands for the activating receptors on NK cells can thwart NK cell recognition and killing of virus-infected cells. NK cells also express receptors that more directly sense the presence of infection or other perturb ations in a cell. Activating receptors include the natural cytotoxicity receptors (NCRs) NKp30, NKp44, and NKp46, which are immunoglobulin-like receptors, and the C-type lectin-like family members LY49H and NKG2D. Recognition by NKG2D acts as a generalized ‘danger’ signal to the immune system. Besides being expressed by a subset of NK cells, NKG2D is expressed by various T cells, including all human CD8 T cells, γδ T cells, activated murine CD8 T cells and invariant NKT cells. In these cells, recognition of NKG2D ligands provides a potent co-stimulatory signal that enhances their effector functions. [Janeway's Immunology, 9th Ed. (2017) Garland Science, New York, pp 125-130]

The conventional NK (cNK) cell pool consists of a circulating compartment and a tissue-resident compartment in the gut intraepithelial layer and lamina propria layer. [Jiao, Y. et al., Front Immunol. (2020) 11: 282] cNK cells are able to sense pathogens, oncogenesis and tissue damage signals. Activation and turnover of cNK cells relies on the overall signal input of activating signals, inhibitory signals, and exogenous cytokine signals, which further leads to the alteration of specific transcription factors and a group of pro-apoptotic proteins and ultimately determines the fate of cNK cells. [Id., citing Viant C, et al. J Exp Med. (2017) 214:491-510]. Upon activation, cNK cells exert their cytotoxicity function by releasing the pore forming cytolytic protein-perforin and the cytotoxic protein-granzyme. cNK cells also utilize tumor necrosis factor (TNF)-related apoptosis-inducing ligand (TRAIL) pathways and antibody-dependent cellular cytotoxicity (ADCC) (Id., citing Caligiuri M A. Blood. (2008) 112:461-9). At the same time, cNK cells possess strong cytokine production ability, including TNF, IFN-γ, and granulocyte-macrophage colony-stimulating factor (GM-CSF) (Id., citing Souza-Fonseca-Guimaraes F, et al. J Biol Chem. (2013) 288:10715-21).

The theory of cNK education posits that the threshold of activation of cNKs throughout their development is modulated by adjusting the expression level of their activating receptors and inhibitory receptors. The processes of cNK cell arming (meaning the downregulation of inhibitory receptors that could upregulate the threshold of activation) and cNK cell licensing (meaning the scenario where activating receptors are downregulated to endow cNK cells with increased receptivity to activating signals) ensure the appropriate activation strategy, namely, to limit self-reaction of cNK cells that do not recognize self MHC class I molecules by inhibitory receptors. Generally, educated cNK cells, marked by the elevated expression of the activating receptor DNAM-1, exhibit higher reactivity to missing-self targets with increased degranulation and cytokine production capability [Id., citing Enqvist M, et al. J Immunol. (2015) 194:4518-27]. It has been hypothesized that the gut may be one of the centers for cNK cells to obtain normal function and acquire education. Gain of cytotoxic function of cNK cells is dependent on the priming step by commensal bacteria in a dendritic cell dependent manner [Id., citing Ganal S C, et al. Immunity. (2012) 37:171-86] and commensal lactic acid bacteria are a key regulator in the cross-talk between cNK cells. Lactic acid bacteria activate immature dendritic cells in the gut to produce key cytokines, including IL-12 and IL-15, and to favor the activation and proliferation of cNK cells [Id., citing Rizzello V, et al. BioMed Res Int. (2011) 2011:473097].

### Human Immunodeficiency Virus

The innate immune response has a major role in the control of HIV-1 infection. At best, it induces host restriction factors that suppress the replication and spread of HIV-1 and activates innate immune cells for HIV-1 control. Among the processes of innate immune activation, the effective licensing of NK cells is essential to facilitate killing of HIV-1 infected cells. At worst, the innate immune response can promote CD4+ T cell death and chronic immune activation linked with HIV-1 disease progression. [Altfeld, M. & Gale, Jr., M. Nature Immunol. (2015) 16 (6): 554-562].

HIV enters target cells by binding via viral surface glycoproteins to the CD4 receptor; it interacts with the coreceptors CCR5 and CXCR4 to facilitate the entry process. For HIV-1 infection, pathogen sensing and innate immune induction typically occur in CD4+ target cells of infection, including innate immune cells and CD4+ T cells. Sensing of HIV occurs in response to the interaction between the whole virion and the cell, to capsid interactions, and to interactions of viral genome RNA with various PRRs. For example, cyclic GMP-AMP synthase (cGAS), a bifunctional protein that contains amino-terminal DNA-binding domains followed by a nucleotidyltransferase domain, is a cytosolic DNA-binding protein and a PRR for HIV and other retroviruses [Id., citing Gao, D. et al. Science (2013) 341: 903-6]. cGAS can bind to dsDNA, including cytosolic DNA of host origin, to produce cGAMP (Id., citing Sun, L. et al. Science (2013) 339: 786-91; Wu et al., Science (2013) 339: 826-30). cGAMP then functions as a second messenger to bind synthase-stimulator of interferon genes (cGAS-STING), thereby activating TBK1 and downstream IRF3 and IRF7 to drive the cell-intrinsic innate immune response. [Id., citing Zhang, X. et al. Mol. Cell (2013) 51: 226-35].

The HIV envelope glycoprotein gp120 can be recognized by TLR3 and TLR4 on the surface of mucosal epithelial cells [Id., citing Nazli, A. et al. J. Immunol. (2013) 191: 4246-58]. Although epithelial cells are not themselves targets of HIV infection, the virion-induced gp120-TLR interaction results in signaling in epithelial cells that triggers proinflammatory cytokine and chemokine production to activate nearby innate immune cells and recruit immune cells to the site of virus encounter. Moreover, HIV-1 genomic RNA is recognized by endosomal TLR7 and TLR8, which program plasmacytoid dendritic cells and specific myeloid cells, respectively, to respond to HIV infection. [Id., citing Schlaepfer, E. et al. J. Immunol. (2006) 176: 2888-95]. The cytosolic PRR and RNA helicase RIG-I (Id., citing Loo, Y M and Gale, M., Jr. Immunity (2011) 34: 680-92) can also recognize HIV genomic RNA and induce innate immune signaling in HIV target cells. [Id., citing Wang, Y. et al. J. Leukoc. Biol. (2013) 94: 337-41; Berg, R K et al. PLoS One (2012) 7: e29291].

As a result of early PRR signaling after HIV infection, innate immune activation produces a local environment high in IFN and other cytokines that induce ISG expression. This response increases the abundance of the PRRs and produces an inflammatory state that is amplified by additional rounds of PRR signaling actions. Tetherin (CD137), an ISG product expressed on the surface of cells in response to IFN; tethers newly produced HIV virions to the cell surface to abrogate virus release and the cell-to cell spread of infection. [Id., citing Tokarev, A., et al. AIDS Res. Hum. Retroviruses (2009) 25: 1197-1210; Perez-Cabellero, D. et al. Cell (2009) 139: 599-511; Neil, S J et al. Nature (2008) 451: 425-30]. After interaction with HIV-1, tetherin also acts as a PRR and initiates an intracellular signaling cascade downstream that activates the transcription factor NF-κB and drives proinflammatory cytokine production [Id., citing Hotter, D. et al. J. Mol. Biol. (2013) 425: 4956-64]. Similarly, interferon inducible protein 16 (IFI16) serves as a PRR for HIV and drives the production of type I IFN and inflammatory cell death or pyroptosis of CD4+ cells [Id., citing Thompson, M R et al. J. Biol. Chem. (2014) 289: 23568-81; Monroe, K M et al. Science (2014) 343: 428-32]. Overall, the sensing of HIV-1 infection by PRRs results in the innate immune activation of both infected cells and bystander cells, accompanied by the induction and production of proinflammatory cytokines and chemokines. This leads to the consecutive activation of innate immune cells, starting with macrophages and dendritic cells, and progressing to activation of NK cells. Virus-host interactions at mucosal sites of virus exposure and in lymphoid tissues mediate innate immune activation to determine outcomes of immune responses, virus controls, inflammation and immune pathology.

### Dengue Virus

Dengue virus (DENV) belongs to the genus Flavivirus of Flaviviridae and is the leading cause of mosquito-borne viral diseases. The DENV virion harbors a messenger-sense, single-stranded RNA (ssRNA) genome that contains a 5′ cap but lacks a 3′ poly-A tail. The DENV invasion starts with cell-surface attachment and receptor binding. After internalization, the nucleocapsid is uncoated, and the virus genome then releases to the cytoplasm. The DENV RNA genome is similar to cellular mRNA, translating a polyprotein precursor in a cap-dependent manner. Viral and cellular proteases then process the polyprotein into three structural proteins (capsid [C], precursor membrane [prM], and envelope [E]) and seven non-structural (NS) proteins (NS1, NS2A, NS2B, NS3, NS4A, NS4B, and NS5). After that, viral RNA is replicated by the viral RNA-dependent RNA polymerase NS5 in the replication complex. Structural proteins are assembled with the DENV RNA genome in the endoplasmic reticulum (ER) and then transmitted to the Golgi apparatus. Ultimately, the mature and infectious virions are secreted into the extracellular space and await the next round of infection (19, 20).

With dengue virus (DENV) infection, the body's antiviral actions start with the recognition of PAMPs derived from the virus, then proceeds with ultimately turning on particular transcription factors to generate antiviral interferons or pro-inflammatory cytokines via fine-tuned signing cascades. Its viral RNA is recognized by the host RNA sensors, mainly retinoic acid inducible gene-I (RIG-I)-like receptors (RLRs) and toll-like receptors. DENV infection also activates the cyclic GMP-AMP synthase-stimulator of interferon genes (cGAS-STING)-mediated DNA-sensing pathway despite the absence of a DNA stage in the DENV lifecycle.

DENV passively escapes from innate immunity surveillance and also actively subverts the innate immune system at multiple steps. DENV targets both RNA-triggered RLR-mitochondrial antiviral signaling protein (RLR-MAVS) and DNA-triggered cGAS-STING signaling to reduce IFN production in infected cells. It also blocks IFN action by inhibiting IFN regulatory factor- and signal transducer and activator of transcription-mediated signaling. [Kao, Y-T et al. Front. Immunol. (2018) 9: 2860].

### ILCs

Innate lymphoid cells (ILCs) are the innate counterparts of T lymphocytes. They lack adaptive antigen receptors generated by the recombination of genetic elements. [Vivier, E. et al. Cell (2018) 174: 1054-66, citing Spits, et al. Nat. Rev. Immunol. (2013) 13: 145-49; Eberl, G et al. Science (2015) 348: aaa6566; Artis, D. and Spits, H. Nature (2015) 517: 293-301]. All ILCs express interleukin-7 receptorα (CD127). ILCs may be activated by signals from other cells around them upon exposure to foreign antigens (including microbes), rather than by being directly activated by foreign antigens. Some ILCs express TLRs that recognize microbes, and the cells may be directly activated by the PAMPs of microbes. However, there have been some reports showing that ILCs express various kinds of receptors for cytokines, danger signals, neuropeptides and lipid mediators that are more dominant than TLRs. [Id.]

ILCs are generally thought to be tissue-resident cells that differentiate into mature effector cells in tissues, and show minimal movement between organs. Instead, they have functional plasticity that enables them to respond promptly to microenvironmental changes, thereby precluding any need for differentiation and/or migration of new ILC subsets adapted to a new environment. For example, transdifferentiation has been shown between ILC1s and ILC3s [Id., citing Bemink J H, et al. Immunity (2015) 43:146-160, Bemink J H, et al. Nat Immunol (2013) 14:221-229], between ILC1s and ILC2 [Id., citing Bal S M, et al. Nat Immunol (2016) 17:636-645; Silver J S, et al. Nat Immunol 2016; 17:626-635; Ohne Y, et al. Nat Immunol (2016) 17:646-655], and between ILC2s and ILC3s [Id. citing Bemink J H, et al. Nat Immunol (2019) 20:992-1003, Golebski K, et al. Nat Commun (2019) 10:2162].Group 1 ILCs currently are divided into 3 different subtypes, according to their expression of cytokines and transcription factors: group 1 ILCs (ILC1s), group 2 ILCs (ILC2s), and group 3 ILCs (ILC3s).

ILC1s are defined as ILCs that express T box-expressed in T cells (T-bet) and produce interferon (IFN-γ); they include conventional natural killer cells (cNK) and are considered to be involved in anti-viral immunity, like T_{H}1 cells. [Orimo, K. et al., Allergy Asthma Immunol. Res. (2020) 19(3): 381-98]

ICL2s are defined as ILCs that express GATA-binding protein 3 and produce such cytokines as IL-4, IL-5, IL-9, and IL-13, as well as the epidermal growth factor, amphiregulin; like T_{H}2 cells, they are considered to be involved in anti-helminth immunity. [Id.]

ILC3s are defined as ILCs that express retinoic acid receptor-related orphan receptor-γt and produce cytokines, such as IL-17A, IL-22 and GM-CSF; they include both natural cytotoxicity receptor (NCR)-ILC3s and NCR+ ILC3s, and are considered to be involved in antibacterial immunity, like T_{H}17 cells. [Id.]

In humans, ILC3s are the predominant population in mucosal tissues, including the lung and gut, whereas the proportion of ILC2s is a little higher in the skin compared to mucosal tissues [Id., citing Bal S M, et al. Nat Immunol (2016) 17:636-645). The proportion of the ILC subsets is influenced by age; although ILC3s are the predominant population in the fetal human lung, their proportion decreases while the proportions of ILC1s and ILC2s increase with age in the adult human lung. [Id., citing Bal S M, et al. Nat Immunol (2016) 17:636-645]. There is substantial heterogeneity in each subset of ILCs. Moreover ILCs show different phenotypes depending on the organ. (Id., citing Ricardo-Gonzalez R R, et al. (2018) 19:1093-1099). For example, although ILC2s from different organs share canonical markers such as GATA3 and IL-7R, expression of IL-33R, IL-25R, and IL-18R1 differs depending on the organ. [Id., citing Ricardo-Gonzalez R R, et al. Nat Immunol (2018) 19:1093-1099].

Another ILC subset, called regulatory ILCs (ILCregs), which resemble regulatory T cells (Tregs) and have regulatory functions, has been reported. [Id., citing Morita H, et al. Allergy Clin Immunol (2019) 143:2190-2201.e9; Wang S, et al. Cell (2017) 171:201-216.e18; Seehus C R, et al. Nat Commun (2017) 8:1900]. ILCregs produce regulatory cytokines such as IL-10 and/or TGFβ, but they do not express FOXP3, the canonical transcription factor of Tregs. It remains controversial wither ILCregs represents an independent effector subset, or just a temporary state of ILCs.

There is increasing evidence to suggest that like T helper cell subsets, ILC subsets also display a certain degree of plasticity, which enables them to adjust to their microenvironment. Thus, ILC subsets can change their phenotype and functional capacities. For example, although ILC2s from different organs share canonical markers such as GATA3 and IL-7R, expression of IL-33R, IL-25R, and IL-18R1 differs depending on the organ. [Id., citing Ricardo-Gonzalez R R, et al. Tissue signals imprint ILC2 identity with anticipatory function. Nat Immunol (2018) 19:1093-1099]. This requires accessible polarizing signals in the tissue in which conversion occurs, together with the expression of cognate cytokine receptors and key transcription factors in the responding ILCs. [Vivier, E. et al. Cell (2018) 174: 1054-66].

### Airway ILCs

### ILC1

### IL-12

IL-12 is a major activator of ILC1s and promotes their secretion of IFN-γ. [Orimo, K. et al. Allergy Asthma Immunol. Res. (2020) 12 (3): 381-98, citing Bemink J H, et al. Nat Immunol (2013) 14:221-229]. The major physiological producers of IL-12 are APCs, such as dendritic cells and macrophages. In the mouse lung, INF-γ produced by ILC1s in response to DC-derived IL-12 during viral infection suppresses early viral growth, suggesting that the IL-12-ILC1 axis may be involved in anti-viral immunity. [Id., citing Weizman O E, et al. Cell (2017) 171:795-808.e12]. Furthermore, IL-12 mediates the transdifferentiation of ILC2s [Id., citing Bal S M, et al. Nat Immunol (2016) 17:636-645; Silver J S, et al. Nat Immunol (2016) 17:626-635; Ohne Y, et al. Nat Immunol (2016) 17:646-655] and ILC3s [Id., citing Bemink J H, et al. Immunity (2015) 43:146-160] into INF-γ producing ILC1s, a mechanism that may be involved in immune responses to viral infections and in the pathophysiology of COPD.

### IL-15

Like IL-12, IL-15 activates ILC1s to produce IFN-γ. IL-15 is known to be produced by APCs, a subset of thymic epithelial cells, and by stromal cells. In the airways, human bronchial epithelial cells produce IL-15 in response to respiratory syncytial virus infection [Id., citing Zdrenghea M T, et al. Eur Respir J 2012; 39:712-720]. In human airway diseases, IL-15-positive cells have been reported to be increased in patients with sarcoidosis, tuberculosis or chronic bronchitis compared to asthmatic patients and healthy subjects, [Id., citing Muro S, et al. Allergy Clin Immunol (2001) 108:970-975] suggesting the involvement of IL-15 in the pathophysiology of these diseases.

### IL-18

IL-18 also activates ILC2s and ILC3s to produce their signature cytokines, (Id., citing 12, 16) suggesting that IL-18 may be a pan-activator of ILCs. Furthermore, IL-18 and IL-12 together promote conversion of ILC2s to ILC1s. [Id., citing Silver J S, et al. Nat Immunol (2016) 17:626-635]. IL-18 is produced by APCs such as macrophages and DCs. In regard to the airways, IL-18 was shown to be released from human bronchial epithelial cells upon human rhinovirus infection [Id., citing Briend E, et al. Respir Res 2017; 18:159] and Alternaria extract stimulation [Id., citing Murai H, et al. Biochem Biophys Res Commun (2015) 464:969-974] in vitro. In addition, cigarette smoke exposure induced IL-18 production by alveolar macrophages in the mouse lungs. [Id., citing Kang M J, et al. J Immunol (2007) 178:1948-1959]. In humans, the levels of IL-18 in bronchoalveolar lavage fluids (BALFs) were significantly higher in patients with COPD than in healthy subjects, and even higher in patients with acute exacerbations of COPD. [Id., citing Wang H, et al. Inflammation (2018) 41:1321-1333]. In addition, the expression of IL-18 in lung epithelial cells was significantly increased in patients with severe COPD compared to healthy individuals who never smoked. [Id., citing Briend E, et al. Respir Res 2017; 18:159]. These findings suggest that IL-18 may be involved in the pathophysiology of COPD.

### ILC2s

### IL-25

IL-25 activates ILC2s and promotes type 2 cytokine production. Various kinds of immune cells, such as macrophages, eosinophils and T cells, have been shown to produce IL-25. Recently, bottle-shaped epithelial-lineage cells expressing taste receptors, named tuft cells-including intestinal tuft cells, brush cells in the lower airways and solitary chemosensory cells (SCCs) in nasopharyngeal tissue—have attracted broad attention as major sources of IL-25. [Id., citing Schneider C, et al. Nat Rev Immunol (2019) 19:584-593]. In mice, intestinal tuft cells produce IL-25 after sensing microbial metabolites through succinate receptors or taste receptors during protozoan and helminth infections, which results in activation of ILC2s and promotion of an anti-helminth response. [Id., citing Schneider C, et al. Nat Rev Immunol (2019) 19:584-593]. Similarly, recent findings have suggested that SCCs in the human upper respiratory tract [Id., citing Kohanski M A, et al. J Allergy Clin Immunol (2018) 142:460-469.e7] and brush cells in the murine lower respiratory tract [Id., citing Bankova L G, et al. Sci Immunol (2018) 3:eaat9453] are major producers of IL-25 in the airways. Besides allergic disorders, the concentration of IL-25 and the number of ILC2s were increased in BALF from patients with idiopathic pulmonary fibrosis and in the lungs of mice with helminth-induced lung fibrosis compared to controls, [Id., citing Hams E, et al. Proc Natl Acad Sci USA (2014) 111:367-372] suggesting possible involvement of the IL-25-ILC2 axis in lung fibrosis as well.

### IL-33

Unlike other cytokines that are newly synthesized upon stimulation and secreted via the endoplasmic reticulum/Golgi pathway, IL-33 is constitutively expressed in cells at the mucosal barrier and released from the nucleus in active form in response to tissue damage. [Id., citing Cayrol C, Girard J P. Immunol Rev (2018) 281:154-168]. It is believed to be one of the “alarmins” that gather components of the repair response to the sites of injury. However, several studies suggest that IL-33 may be actively secreted from live cells, including bronchial epithelial cells [Id., citing Hristova M, et al. J Allergy Clin Immunol (2016) 137:1545-1556.e111] and fibroblasts, even in the absence of necrosis. Although the mechanisms of IL-33 secretion are not fully understood, adenosine triphosphate-induced purinoceptor-dependent activation of epithelial nicotinamide adenine dinucleotide phosphate oxidase, i.e., dual oxidase 1, may be involved. [Id., citing Hristova M, et al. J Allergy Clin Immunol (2016) 137:1545-1556.e111]. IL-33 is recognized as one of the major activators of ILC2s that induce production of type 2 cytokines. In mice, IL-33 is released from alveolar epithelial cells in response to tissue damage caused by fungi such as Alternaria and Aspergillus and viruses such as respiratory syncytial virus (RSV) and rhinovirus (RV). [Id., citing Cayrol C, Girard J P. Immunol Rev (2018) 281:154-168]. Meanwhile, in humans, IL-33 is released from bronchial epithelial cells located more centrally, [Id., citing Cayrol C, Girard J P. Immunol Rev (2018) 281:154-168] similar to IL-25 and thymic stromal lymphopoietin (TSLP).

The expression of IL-33 in the lungs peaks during infancy, and declines with age. The number of ILC2s in the lungs also peaks in infancy. [Id., citing de Kleer I M, et al. Immunity (2016) 45:1285-1298]. These findings suggest that IL-33 may play a major role in the developing phase of acquired immunity and that epithelial damage may induce more severe allergic airway inflammation during infancy than during adulthood through the IL-33-ILC2s axis. In addition to epithelial cells, stromal cells, [Id., citing Dahlgren M W, et al. Immunity (2019) 50:707-722.e6] endothelial cells, fibroblasts [Id., citing Cayrol C, Girard J P. Immunol Rev 2018; 281:154-168] and platelets [Id., citing Takeda T, et al. J Allergy Clin Immunol (2016) 138:1395-1403.e6] may produce IL-33.

### Thymic Stromal Lymphopoietin (TSLP)

Like other epithelial-derived cytokines such as IL-33 and IL-25, TSLP is recognized as a major activator of ILC2s that induces production of type 2 cytokines. However, unlike other epithelial-derived cytokines, TSLP was shown to induce corticosteroid resistance in murine ILC2s through activation of an intracellular signaling molecule, signal transducer and activator of transcription 5. [Id., citing Kabata H, et al. Nat Commun 2013; 4:2675]. TSLP is produced by various kinds of cells including DCs, vascular endothelial cells, macrophages and mast cells. In the airways, similar to IL-25 and IL-33, TSLP is produced mainly by airway epithelial cells in response to exposure to bacteria, fungi and viruses. [Id., citing Varricchi G, et al. Front Immunol 2018; 9:1595]. Adventitial stromal cells localize with ILC2s in adventitial niches around the lung bronchi and large vessels, and support ILC2s through constitutive expression of TSLP and IL-33. [Id., citing Dahlgren M W, et al. Immunity (2019) 50:707-722.e6].

### IL-27

IL-27 is generally produced by DCs and macrophages. In mice, IL-27 suppresses the proliferation and cytokine production of ILC2 cells in vitro, [Id., citing Moro K, et al. Nat Immunol (2016) 17:76-86, Duerr C U, et al. Nat Immunol (2016)17:65-75] and it also suppresses Alternaria-induced eosinophilic airway inflammation by regulating ILC2 activation in vivo. [Id., citing Moro K, et al. Nat Immunol (2016) 17:76-86].

### Interferons

IFNs are divided into types 1 (α/β), 2 (γ) and 3 (λ).

Type I IFNs combat viral infection both directly by inhibiting viral replication in infected cells and indirectly by stimulating the adaptive immune system [Zhou, Z. et al. J. Virology (2007) 81 (14): 7749-58, citing Biron, C. A. (1994) Curr. Opin. Immunol. 6:530-538, Ida-Hosonuma, M., et al (2005) J. Virol. 79:4460-4469, Sen, G. C., and P. Lengyel. (1992). J. Biol. Chem. 267:5017-5020, Stark, G. R., et al. (1998) Annu. Rev. Biochem. 67:227-264]. The IFN-α family of 12 closely related human genes and IFN-β, the product of a single gene, are best understood; less well studied are IFN-κ, IFN-ε, and IFN-ω.

The engagement of type I IFNs and their cell surface receptors (IFN-α-receptors, or IFNARs) activates Janus kinase (JAK)-signal transducer and activator of transcription (STAT)-signaling, which promotes the transcription of a large array of IFN-stimulated genes (ISGs) to exert antiviral activities [He, Y. et a., Sci. Signal. 13(2020) eaaz3381]. Although all type I IFNs can bind to IFNARs, their antiviral activities appear to be distinct. For example, IFN-ω exhibits increased anti-influenza activity in cultured cells compared to INF-α2, albeit less than IFN-β1a [Id., citing Skorvanoa, L. et al. Acta Virol. 2015] 59: 413-17]. IFN-κ was first identified in human keratinocytes and then in dendritic cells and monocytes [Id., citing Nardelli, B. et al. J. Immunol. (2002) 169: 4822-30]. While it is primarily viewed as a keratinocyte-specific IFN dedicated to skin immune responses, it can also induce antiviral responses in other human cell types. [Id., citing LaFleur, D W, et al. J. Biol. Chem. (2001) 276: 39765-71].

IFNβ is induced earlier than IFN-α in cultured cells in response to virus infection [Id., citing Honda, K., et al. Immunity (2006) 25: 349-60]; many virus-encoded proteins interfere with the production of IFN through various mechanisms [Id., citing Garda-Sastre, A. Cell Host Microbe (2017) 22: 176-84]. For example, the NS1 proteins of some IAVs are capable of inhibiting the 3′ end processing of cellular pre-mRNAs by binding to cleavage and polyadenylation specific factor (CPSF30) and accordingly blocking the production of mature mRNAs, including those of IFN-α and IFN-β [Id., citing Krug, R M. Curr. Opin. Virol. (2015) 12: 106]

He et al. reported a study in which they analyzed the expression of genes encoding different type I IFNs during infection of epidemic-causing H7N9 virus and an H9N2 virus in a mouse model. They identified Ifnk, the gene that encodes IFN-κ, as the most differentially expressed type I IFN gene in the early phase of infection, being the only one increased after H9N2 infection, but decreased after H7N9 infection. They then used cultured human cells to study the action of IFN-κ against IAV infection. On the basis of the identification of a mutant IFNK gene in human lung epithelial A549 cells and subsequent demonstration that wild-type IFN-κ, but not the mutant, failed to contain IAV in cultured human cells, they pinned down chromodomain helicase DNA binding protein 6 (CHD6) as the major effector molecule mediating the anti-influenza activity of IFN-κ. Compared to its induction by IFN-κ, CHD-6 was less induced by IFN-α and IFN-β [collectively IFN-α/β] and was dispensable for IFN-α/β-mediated inhibition of IAV replication. They also identified the upstream signaling required by IFN-κ to stimulate CHD6 expression. Unlike IFN-α/β, which transduce antiviral signal preferentially through IFNAR1, IFN-κ required the engagement of both IFNAR1 and IFNAR2. The binding by IFN-κ to the individual IFNARs is weaker than the binding of IFN-α/β [Id., citing Harris, B D et al. J. Biol. Chem. (2018) 293: 16057-068], suggesting different modes of action between IFN-α/β and IFN-κ. IFN-κ also was distinct in that it induced CHD6 through a p38-cFos axis, rather than the canonical JAK-STAT pathway. IFNα/β therefore use multiple signaling pathways to activate a diverse array of ISGs, exerting profound effects on both virus and cells, while IFN-κ instead exhibited a selective use of downstream signaling, resulting in a relatively narrower spectrum of downstream targets, among which some effector genes are preferentially stimulated, as seen for CHD6. Such focused strategy, underlying the observed dominance of a single effector molecule in the antiviral activity of IFN-κ-CHD6 for influenza as shown here, and Sp100 for human papillomavirus (HPV) as shown in a previous study [Id., citing Habiger, C. et al. J. Virol. (2016) 90: 694-704] may bestow a benefit on the host by constraining responding cells from overreacting. Last, they showed that preapplication of IFN-κ protected mice against lethal IAV infection with H7N9.

IFN-γ is the sole type II interferon; the dominating biological role of IFN-γ seems to be stimulation of the adaptive immune system, primarily activation of T cells [Zhou, Z. et al. J. Virology (2007) 81 (14): 7749-58., citing Biron, C. A. (1994). Curr. Opin. Immunol. 6:530-538, Muller, U., et al. (1994) Science 264:1918-1921]. Type III interferons are the products of three IFN-γ genes, IL-28A, IL-28B, and IL-29, which bind a heterodimeric IFN-λ receptor composed on a unique IL-28Rα subunit and the β subunit of the IL-10 receptor. Unlike the ubiquitously expressed type I IFNR complex, the type III IFNR has a more restricted tissue distribution pattern. Although the IL10R2 chain is ubiquitously expressed in all tissues and cells, the expression of the IFNλR1 varies widely between different organs and at the cellular level is restricted to epithelial cells. [Zhou, P. et al., PLoS One (2011) 6(9): e15385, citing Witte K, Witte E, et al. Cytokine Growth Factor Rev. (2010) 21:237-251, Witte K, et al. Genes Immun. (2009) 10:702-14; Sommereyns C, et al. PLoS Pathog. (2008) 4:e1000017; Donnelly R P, et al. J Leukoc Biol. (2004) 76:314-21.

Type I interferons are inducible and are synthesized by many cell types after infection by diverse viruses. Almost all types of cells can produce IFN-α and IFN-β in response to activation of several innate sensors. For example, type I interferons are induced by RIG-1 and MDA-5 (the sensors of cytoplasmic viral RNA) downstream of MAVs, and by signaling from cGAS (the sensor of cytoplasmic DNA) downstream of STING. Plasmacytoid dendritic cells (pDCs), also called interferon-producing cells (IPCs) or natural interferon-producing cells, make abundant type I interferons, which may result from the efficient coupling of viral recognition by TLRs to the pathways of interferon production. pDCs express a subset of TLRs that includes TLR-7 and TLR-9, which are endosomal sensors of viral RNA and of the nonmethylated CpG residues present in the genomes of many DNA viruses. pDCs express CXCR3, a receptor for chemokines CXCL9, CXCL10, and CXCR11, which are produced by T cells, which allows pDCs to migrate from the blood into lymph nodes in which there is an ongoing inflammatory response to a pathogen. [Janeway's Immunology, 9th Ed. (2017) Garland Science, New York, pp 122-125]

Interferons help defend against viral infections in several ways. IFN-β induces cells to make IFN-α, thus amplifying the interferon response. Interferons act to induce a state of resistance to viral replication in all cells. IFN-α and IFN-β bind to a common cell surface receptor, the interferon-a receptor (IFNAR), which uses the JAK and STAT pathways. IFNAR uses the kinases Tyk2 and Jak1 to activate the factors STAT1 and STAT2, which can interact with IRF9 and form a complex called ISGF4, which binds to the promoters of many interferon stimulated genes (ISGs). [Janeway's Immunology, 9th Ed. (2017) Garland Science, New York, pp 122-125]

One ISG encodes the enzyme oligoadenylate synthetase, which polymerizes ATP into 2′-5′ linked oligomers, which activate an endoribonuclase that then degrades viral RNA. A second protein induced by IFN-α and IFN-β is protein kinase R (PKR), a dsRNA-dependent protein kinase, which phosphorylates the a subunit of eukaryotic initiation factor 2 (eIF2α), thus suppressing protein translation and contributing to the inhibition of viral replication. Mx (myxoma resistant) proteins also are induced by type I interferons. Mx1 and Mx2 are GTPases belong to the dynamin protein family; how they interfere with virus replication is not understood. [Janeway's Immunology, 9th Ed. (2017) Garland Science, New York, pp 122-125]

The interferon-induced protein with tetratricoid repeats (IFIT) family contains four human and three mouse proteins that function in restraining the translation of viral RNA into proteins. IFIT1 and IFIT2 can suppress the translation of normal capped mRNAs by binding to subunits of the eukaryotic initiation factor 3 (eIF3) complex, which prevents eIF3 from interacting with eIF2 to form the 43S pre-initiation complex. Mice lacking IFIT1 or IFIT2 show increased susceptibility to infection by certain viruses, e.g., vesicular stomatitis virus. IFIT1 also suppresses translation of viral RNA that lacks a normal host modification of the 5′ cap. Many viruses, e.g., West Nile virus, and SARS coronavirus, have acquired a 2′e-O-methyltransferase (MTase) that produce cap-1 or cap-2 on their viral transcripts, thus evading restriction by IFIT1. [Janeway's Immunology, 9th Ed. (2017) Garland Science, New York, at 122-125]

Members of the interferon-induced transmembrane protein (IFITM) family are strongly induced by type I interferons. There are four functional IFITM genes in humans and in mice; these encode protein that have two transmembrane domains and are localized to various vesicular compartments of the cell. IFITM protein act to inhibit, or restrict, viruses at early steps of infection. IFITM1 appears to interfere with the fusion of viral membranes with the membrane of the lysosome, which is required for introducing some viral genomes into the cytoplasm. Viruses that must undergo this fusion event in lysosomes, e.g., Ebola virus, are restricted by IFITM1. IFITM2 interferes with membrane fusion in late endosomes, and so restricts the influenza A virus, which undergoes fusion there. [Janeway's Immunology, 9th Ed. (2017) Garland Science, New York, at 122-125]

Interferons stimulate production of the chemokines CXCL9, CXCL10, and CXCL11, which recruit lymphocytes to sites of infection, and increases expression of MHC class I molecules on all types of cells. [Janeway's Immunology, 9th Ed. (2017) Garland Science, New York, at 122-125]

Type 1 and 2 IFNs have been shown to suppress type 2 cytokine production by ILC2s, both in vitro and in vivo. [Id., citing Moro K, et al. Nat Immunol (2016) 17:76-86, Duerr C U, et al. Nat Immunol (2016) 17:65-75]. The major producers of IFN-α and -β are macrophages and DCs. IFN-γ is produced by activated T_{H}1 cells and ILC1s, including NK cells, which are activated mainly through TLRs. In mice, the deficiency of type 1 IFN during influenza virus and helminth infections results in severe or prolonged eosinophilic airway inflammation mediated by activated ILC2s. In humans, dozens of reports have shown impaired production of type 1 and 3 IFNs by cultured primary bronchial epithelial cells, BAL cells, peripheral blood mononuclear cells (PBMCs) and plasmacytoid DCs in response to infection with viruses such as RSV and rhinovirus (RV) in patients with asthma compared to healthy individuals. [Id., citing Edwards M R, et al. J Allergy Clin Immunol (2017) 140:909-920]. Therefore, dysregulation of ILC2 activity by type 1 and 3 IFNs during viral infection in asthmatic patients may result in the development and exacerbation of allergic airway inflammation.

### Lipid Inflammatory Mediators

Although lipids are primarily involved in the formation of cell membranes of organs, various reports have shown that bioactive lipids or lipid mediators also play crucial roles in immune responses and the maintenance of homeostasis. Cysteinyl leukotrienes (CysLTs) as well as prostaglandin (PG) D2 are products of arachidonic acid and were known to be major pro-inflammatory lipid mediators of allergic disorders from early days. Mast cells activated by immunoglobulin (Ig) E-crosslinking are the major source of PGD2 in terms of quantity, but other leukocytes, including eosinophils, T_{H2} cells, DCs and cytokine-activated ILC2s [Id., citing Maric J, et al. J Allergy Clin Immunol (2019) 143:2202-2214.e5] also produce PGD2. Since human ILC2s are identified as lineage-negative cells expressing chemoattractant receptor-homologous molecules on T_{H}2 cells (CRTH2), [Id., citing Mjösberg J M, et al. Nat Immunol (2011) 12:1055-1062] which is the PGD2 receptor, PGD2 influences ILC2s in a variety of ways, including their migration [Id., citing Winkler C, et al. J Allergy Clin Immunol (2019) 144:61-69.e7] and production of IL-13. [Id., citing Doherty T A, Broide D H. J Allergy Clin Immunol (2018) 141:1587-1589]. CysLTs are generally produced by leukocytes such as eosinophils, mast cells, macrophages and basophils. CysLTs act directly on ILC2s to enhance their ability to produce type 2 cytokines, both in vivo and in vitro. [Id., citing Doherty T A, Broide D H. J Allergy Clin Immunol (2018) 141:1587-1589]. There are some lipid molecules that inhibit ILC2 activation. PGI2, PGE2 and lipoxin A4—also products of arachidonic acid-suppress ILC2s' cytokine production and proliferation, in vitro and in vivo. [Id., citing Doherty T A, Broide D H. J Allergy Clin Immunol (2018) 141:1587-1589]. LTE4 and PGD2 reportedly induce T_{H}2 cytokines, including IL-4, synergistically in purified human peripheral blood ILC2s. [Id., citing Salimi M, et al. J Allergy Clin Immunol (2017) 140:1090-1100.e111].

### Neuropeptides

Neuropeptides are peptides that are expressed in the nervous system and exhibit physiological activity. They are present not only in the central nervous system, but also in the nervous system of peripheral tissues such as the lungs, and they function as signal transmitters between cells. Among several neuropeptides known to act on ILC2s, vasoactive intestinal peptide (VIP) was the first one shown to modulate ILC2 activation. VIP belongs to the glucagon/secretin family and is highly expressed in intestinal neurons, where it coordinates pancreatic secretion with smooth muscle relaxation in response to feeding. Both lung and intestinal ILC2s express VIP receptors, including VIP receptor type 1 and type 2, and VIP simulation induces IL-5 production by the cells. The IL-5 produced in turn activates sensory neurons to produce VIP [Id., citing Talbot S, et al. Neuron (2015) 87:341-354], which may exacerbate allergic airway inflammation. Lung ILC2s also express receptors for another neuropeptide, called neuromedin U (NMU), whereas ILC1s and ILC3s do not. NMU is thought to directly activate lung ILC2s to proliferate and produce type 2 cytokines. [Id., citing Wallrapp A, et al. Nature (2017) 549:351-356]. Calcitonin gene-related peptide (CGRP) is a calcitonin gene product, like the thyroid hormone calcitonin and it is involved in the regulation of blood calcium levels. CGRP is widely distributed in the central and peripheral nervous systems; It was also produced by non-neuronal cells in the airways-called pulmonary neuroendocrine cells (PNECs)—after OVA challenge in an OVA-sensitized mouse model. [Id., citing Sui P, et al. Science (2018) 360:eaan8546]. It has been reported that ILC2s are localized in close proximity to PNECs and that CGRP enhances type 2 cytokine production by lung ILC2s in the presence of IL-33 or IL-25, [Id., citing Sui P, et al. Science (2018) 360:eaan8546] suggesting that interaction between PNECs and ILC2s may be involved in allergic airway inflammation. Besides the neuropeptides that induce activation of ILC2s, there is also a neuropeptide that regulates activation of ILC2s. Both lung and intestinal ILC2s express the β2-adrenergic receptor (β2-AR), which is a receptor for epinephrine released by sympathetic nerve stimulation. Treatment with a β2-AR agonist, salmeterol, suppressed proliferation and type 2 cytokine production by ILC2s in an IL-33-induced airway inflammation model. (Id., citing Moriyama S, et al. Science (2018) 359:1056-1061). These findings suggest that β2-AR agonists used as therapeutic agents for asthma may work not only as a bronchodilator, but also as a suppressor of type 2 inflammation induced by ILC2s.

### Sex Steroids

Sex steroids, such as estrogen and androgen, are steroid hormones that are produced mainly by the reproductive organs and modulate reproductive functions. In addition to their effects on the reproductive organs, sex steroids have recently been shown to have effects on immune cells, including ILC2s in peripheral tissues. Androgen receptors are expressed on lung ILC2s [Id., citing Cephus J Y, et al. Cell Reports (2017) 21:2487-2499] as well as ILC2 progenitors (ILC2Ps) in bone marrow (BM), [Id., citing Laffont S, et al. J Exp Med 2017; 214:1581-1592], whereas estrogen receptors are expressed on lung ILC2s and uterine ILC2s [Id., citing Bartemes K, et al. J Immunol (2018) 200:229-236], but not on ILC2Ps in BM. [Id., citing Laffont S, et al. J Exp Med (2017) 214:1581-1592]. These findings indicate that androgens may influence both the development of ILC2s in BM and the activation of ILC2s in peripheral tissues, whereas estrogens may influence mainly ILC2s in peripheral tissues.

Androgens and estrogens are thought to exert opposite effects on ILC2s. Androgen signaling inhibits differentiation of ILC2Ps into ILC2s [Id., citing Laffont S, et al. J Exp Med (2017) 214:1581-1592] and also activation of ILC2s. (Id., citing Cephus J Y, et al. Cell Reports (2017) 21:2487-2499, Laffont S, et al. J Exp Med (2017) 214:1581-1592). In contrast, estrogen has been suggested to have supportive effects on ILC2s. [Id., citing Bartemes K, et al. J Immunol (2018) 200:229-236]. Indeed, the numbers of lung ILC2s [Id., citing Laffont S, et al. J Exp Med (2017) 214:1581-1592] and BM ILC2Ps are significantly lower in adult male mice than in adult female mice in the steady state. [Id., citing Laffont S, et al. J Exp Med (2017) 214:1581-1592].

### ILC3s

### IL-23

IL-23 is a major activator of ILC3s that induces production of inflammatory cytokines such as IL-17 and IL-22. IL-23 also induces conversion of ILC1s to ILC3s in conjunction with IL-1β and retinoic acid [Id., citing Bernink J H, et al. Immunity (2015) 43:146-160], and ILC2s to ILC3s in conjunction with IL-1β and TGF-β. [Id., citing Bemink J H, et al. Nat Immunol (2019) 20:992-1003, Golebski K, et al. Nat Commun (2019) 10:2162]. IL-23 is generally produced by DCs and macrophages.

### IL-1β

IL-1β is a major activator of ILC3s that induces IL-17A production. [Id., citing Kim H Y, et al. Nat Med (2014) 20:54-61] While IL-1β is a potent activator of ILC2s that induce type 2 cytokine production [Id., citing Bal S M, et al. Nat Immunol (2016) 17:636-645], it also induces conversion of ILC2s to ILC1Ss together with IL-12, [Id., citing Bal S M, et al. Nat Immunol (2016) 17:636-645, Ohne Y, et al. Nat Immunol (2016) 17:646-655] and to ILC3s together with IL-23 and TGF-β. [Id., citing Golebski K, et al. Nat Commun (2019) 10:2162]. In the airways, IL-1β is produced by DCs in response to exposure to chitin and IL-33 [Id., citing Arae, K. et al. Sci. Rep. (2018) 8: 11721] and by nasal epithelial cells exposed to Staphylococcus aureus or Pseudomonas aeruginosa [Id., citing Golebski K, et al. Nat Commun (2019) 10:2162].

### Vitamins

Retinoic acid (RA)—which is a metabolite of vitamin A (Vit A)—and vitamin D (Vit D) is known to regulate ILCs. [Id., citing Morita H, et al. J Allergy Clin Immunol (2019) 143:2190-2201.e9, Seehus C R, et al. Nat Commun (2017) 8:1900, Bernink J H, et al. Immunity (2015) 43:146-160, Golebski K, et al. Nat Commun (2019) 10:2162, Konya V, et al. J Allergy Clin Immunol (2018) 141:279-292]. RA is synthesized from a Vit A metabolite, retinal, by cells having enzymes such as retinaldehyde dehydrogenase (ALDH)1A1, ALDH1A2 and ALDH1A3. RA is generally synthesized by CD103+ DCs, intestinal epithelial cells and lamina propria stromal cells in the gut that express ALDHs. In the airways, bronchial epithelial cells express ALDHs in response to IL-13 stimulation [Id., citing Morita H, et al. J Allergy Clin Immunol (2019) 143:2190-2201.e9], suggesting that these cells could be the source of RA during allergic airway inflammation. Vit D can be absorbed by oral intake, but it is synthesized mainly in the skin upon exposure to ultraviolet light from the sun. RA enhances activation of ILC3s by IL-1β and IL-23 to increase production of IL-22, and it also induces conversion of ILC1s to ILC3s in conjunction with IL-1β and IL-23. [Id., citing Bernink J H, et al. Immunity (2015) 43:146-160]. In addition, RA inhibits development of ILC2s from ILC2Ps in mouse BM69 and induces conversion of ILC2s to IL-10-producing ILCregs in both humans and mice. [Id., citing Morita H, et al. J Allergy Clin Immunol (2019) 143:2190-2201.e9, Seehus C R, et al. Nat Commun (2017) 8:1900]. In contrast to the positive effects of RA on ILC3s, Vit D suppresses production of cytokines such as IL-22, IL-17F and GM-CSF by ILC3s by down-regulating the IL-23/IL-23R pathway, [Id., citing Konya V, et al. J Allergy Clin Immunol (2018) 141:279-292] and it also prevents IL-1β-, IL-23- and TGF-β-induced conversion of ILC2s to ILC3s. [Id., citing Golebski K, et al. Nat Commun (2019) 10:2162].

ILC3s are emerging as key orchestrators and regulators of adaptive immune responses, either through indirect modulation of bystander cells that subsequently modulate the adaptive immune response or directly via both soluble mediators and cell contact-dependent interactions with adaptive lymphocytes. [Domingues, R G, Hepworth, M R. Front. Immunol. (2020) 11:116]. In addition to their function as tissue-resident cytokine producing cells, ILC3s have the capacity to participate in multiple cellular circuits through direct cell-cell modulation of T cell responses, as well as the release of soluble mediators that augment adaptive immune function and development. For example, ILC3s can control the magnitude and quality of the CD4+ T cell response via antigen presentation in the context of MHC class II. At steady state, ILC3s lack co-stimulatory molecule expression and appear to limit CD4+ T cell responses; however, this interaction may be altered in inflammatory scenarios via upregulation of costimulatory molecules such as CD4, CD80, and CD86, which favor the promotion of a T cell response. Further, ILCs act to modulate the survival of recirculating memory CD4+ T cells via interactions via OX40L and CD30L. In addition, ILC3 regulation of T follicular helper (T_{FH}) cell responses has consequences for the priming of germinal center B cells and the induction of T dependent IgA responses toward colon-dwelling commensal microbes. ILC3s also can modulate adaptive immune cells through the production of regulatory cytokines and growth factors. For example ILC3 directly support B cell responses in the spleen through provision of critical growth factors such as BAFF/APRIL. Similarly they modulate the magnitude of the T cell response within the intestinal tract through production of soluble mediators. For example, ILC3-derived IL-22 induces epithelial serum amyloid A (SAA) protein, which subsequently promotes local T_{H}17 responses and acts to limit colonization with segmented filamentous bacteria (SGF) via induction of antimicrobial peptides. In addition, ILC3 facilitate the establishment of a regulatory and tolerogenic environment in the gut by promoting Treg responses. Finally ILC subsets are a potent source of IL-2 in the small intestine which provide survival signals for Tregs. [Domingues, R G, Hepworth, M R. Front. Immunol. (2020) 11:116].

The immune response to invading pathogens requires the successful activation of innate immunity, which informs the development of the subsequent adaptive immune response. While most pathogens can overcome innate immune responses, the adaptive immune response is required to eliminate them and to prevent subsequent reinfection.

### Adaptive Immune Response

The adaptive arm of the immune response involves a specific, delayed and longer-lasting response by various types of cells that create long-term immunological memory against a specific antigen. It can be further subdivided into cellular and humoral branches, the former largely mediated by T cells and the latter by B cells. This arm further encompasses cell lineage members of the adaptive arm that have effector functions in the innate arm, thereby bridging the gap between the innate and adaptive immune response.

Generally speaking, these immune responses are initiated by an encounter between an individual and a foreign substance, e.g., an infectious microorganism. The infected individual rapidly responds with both a humoral immune response with the production of antibody molecules specific for the antigenic determinants/epitopes of the immunogen, and a cell mediated immune response with the expansion and differentiation of antigen-specific regulatory and effector T-lymphocytes, including cells that produce cytokines and killer T cells, capable of lysing infected cells. Primary immunization with a given microorganism evokes antibodies and T cells that are specific for the antigenic determinants/epitopes found on that microorganism; these usually fail to recognize or recognize only poorly antigenic determinants expressed by unrelated microbes [Paul, W. E., “Chapter 1: The immune system: an introduction,” Fundamental Immunology, 4th Edition, Ed. Paul, W. E., Lippincott-Raven Publishers, Philadelphia, (1999), at p. 102].

As a consequence of this initial response, the immunized individual develops a state of immunologic memory. If the same or a closely related microorganism is encountered again, a secondary response ensues. This secondary response generally consists of an antibody response that is more rapid, greater in magnitude and composed of antibodies that bind to the antigen with greater affinity and that are more effective in clearing the microbe from the body, and a similarly enhanced and often more effective T-cell response. However, immune responses against infectious agents do not always lead to elimination of the pathogen [Paul, W. E., “Chapter 1: The immune system: an introduction,” Fundamental Immunology, 4th Edition, Ed. Paul, W. E., Lippincott-Raven Publishers, Philadelphia, (1999), at p. 102].

### Lymphocytes

Lymphocytes are a type of white blood cell involved in immune system regulation. Lymphocytes are much more common in the lymphatic system, and include B cells, T cells, killer T-cells, and natural killer (NK) cells. There are two broad categories of lymphocytes, namely T cells and B cells. T-cells are responsible for cell-mediated immunity whereas B-cells are responsible for humoral immunity (relating to antibodies). T-cells are so-named such because these lymphocytes mature in the thymus; B-cells mature in bone marrow. B cells make antibodies that bind to pathogens to enable their destruction. CD4+(helper) T cells coordinate the immune response. CD8+(cytotoxic) T cells and Natural Killer (NK) cells are able to kill cells of the body that are, e.g., infected by a virus or display an antigenic sequence.

### Compartmentalization of the Immune System

The periphery of the immune system—as opposed to the central lymphoid organs—contains inhomogeneously distributed B and T cells whose phenotype, repertoire, developmental origin, and function are highly divergent. Nonconventional lymphocytes bearing a phenotype that is rare in the blood, spleen, or lymph nodes of undiseased individuals are encountered at high frequency in different localizations, e.g., alpha/beta TCR+CD4−CD8− cells in the bone marrow and gut epithelium, particular invariant gamma/delta TCR+CD4−CD8 alpha+CD8 beta− and gamma/delta TCR+CD4−CD8 alpha−CD8 beta− T cells in various epithelia, or CD5+ B cells in the peritoneum. The antigen receptor repertoire is different in each localization. Thus, different gamma/delta TCR gene products dominant in each site, and the proportion of cells expressing transgenic and endogenous alpha/beta TCR and immunoglobulin gene products follows a gradient, with a maximum of endogenous gene expression in the peritoneum, intermediate values in other peripheral lymphoid organs (spleen, lymph nodes), and minimum values in thymus and bone marrow. Forbidden T cells that bear self-superantigen-reactive V beta gene products are physiologically detected among alpha/beta TCR+CD4−CD8− lymphocytes of the bone marrow, as well as in the gut. Violating previous ideas on self-tolerance preservation, self-peptide-specific gamma/delta T cells are present among intestinal intraepithelial lymphocytes, and CD5+ B cells produce low-affinity cross-reactive autoantibodies in a physiological fashion. It appears that, in contrast to the bulk of T and B lymphocytes, certain gamma/delta and alpha/beta T cells found in the periphery, as well as most CD5+ B cells, do not depend on the thymus or bone marrow for their development, respectively, but arise from different, nonconventional lineages. In addition to divergent lineages that are targeted to different organs guided by a spatiotemporal sequence of tissue-specific homing receptors, local induction or selection processes may be important in the diversification of peripheral lymphocyte compartments. Selection may be exerted by local antigens, antigen-presenting cells whose function varies in each anatomical localization, cytokines, and cell-matrix interactions, thus leading to the expansion and maintenance of some clones, whereas others are diluted out or deleted.

In multicellular organisms, cells that are specialized to perform common functions are usually organized into cooperative assemblies embedded in a complex network of secreted extracellular macromolecules, the extracellular matrix (ECM), to form specialized tissue compartments. Individual cells in such tissue compartments are in contact with ECM macromolecules. The ECM helps hold the cells and compartments together and provides an organized lattice or scaffold within which cells can migrate and interact with one another. In many cases, cells in a compartment can be held in place by direct cell-cell adhesions. In vertebrates, such compartments include four major types, a connective tissue (CT) compartment, an epithelial tissue (ET) compartment, a muscle tissue (MT) compartment and a nervous tissue (NT) compartment, which are derived from three embryonic germ layers: ectoderm, mesoderm and endoderm. The NT and portions of the ET compartments are differentiated from the ectoderm; the CT, MT and certain portions of the ET compartments are derived from the mesoderm; and further portions of the ET compartment are derived from the endoderm.

The lifelong production of blood cells depends on hematopoietic stem cells (HSC) and their ability to self-renew and to differentiate into all blood lineages. Hematopoietic stem cells (HSCs) develop during embryogenesis in a complex process that involves multiple anatomical sites (the yolk sac, the aorta-gonadmesonephros region, the placenta and the fetal liver), Once HSC precursors have been specified from mesoderm, they have to mature into functional HSCs and undergo self-renewing divisions to generate a pool of HSCs. During this process, developing HSCs migrate through various embryonic niches, which provide signals for their establishment and the conservation of their self-renewal ability. [Mikkola, H K A, Orkin, S H. Development (2006) 133: 3733-44].

B & T lymphocytes have to receive contact-dependent activation signals from immobile cells in situ, and exert the majority of their functions via direct intercellular interactions. Lymphocytes are influenced in their behavior by local antigens and metabolites and are embedded in a complex network of interactions with neighboring accessory cells (e.g., B cells and macrophages). ECM proteins continuously interact with signal-transducing receptors on lymphoid cells. [Kroemer, G. et al. Adv. Immunol. (1993) 53: 157-216].

Lymphocytes located outside of the thymus and bone marrow are considered as peripheral cells. Peripheral lymphocytes are contained in the classic lymphoid organs (spleen, lymph nodes, tonsils and Peyer's patches), the epidermis, the mucosae of the gastrointestinal, respiratory, and female reproductive tracts, and in mesoderm derivatives (e.g., the pleuroperitoneal cavity. Lymphocytes in the lung interstitium are as numerous as those of the circulating blood pool. [Kroemer, G. et al. Adv. Immunol. (1993) 53: 157-216, citing Pabst, R. (1992) Immunology Today 13: 119-22].

T cells are extremely heterogeneous in specificity, activation requirements, life span, and functional properties. T cells produce a nearly infinite antigen receptor repertoire via somatic diversification processes, including gene rearrangements and somatic mutation. They can also be classified into subpopulations that differ in the expression of classes of the T cell receptor (TCR; α/β or γ/δ heterodimers) and CD antigens, in the activation state, or in functional terms. For example, the differentiation antigens CD4 and CD8 are found on mutually exclusive α/β T lymphocyte subsets in the periphery. CD4+α/β T cells are predominantly of the helper phenotype, whereas CD8 (usually a heterodimer composed of CD8α, and CD8β) is mainly expressed on cytotoxic and suppressor T cells. This functional distinction is not absolute, because some CD4− T lymphocytes can effect cytotoxicity and suppression, and a more stringent correlation exists between CD4/CD8 expression and MHC gene products expressed by target or antigen presenting cells (APC). CD4+ T cells interact with cells expressing MHC class II; whereas CD8+ T cells are class I restricted [Id., citing Moller, G. (Ed) Immunol. Rev. (1989) 109: 5-153, Pames, J R. Adv. Immunol. 44: 265-311; Bierer, B E et al (1989) Annu. Rev. Immunol. (1989) 7: 579-99; Auffray, C. et al. Trends Biotechnol. (1991) 9: 124-30). A majority of the γ/δ do not express either CD4 or CD8; however a significant fraction displays CD8 and exerts a suppressor or cytotoxic function [Id., citing Bandeira, A. et al. Proc. Natl Acad. Sci. USA (1991) 88: 43-47]. A minor population that expresses CD4 exhibits a helper phenotype (Id., citing Morita, C T et al. Eur. J. Immunol. (1991) 21: 2999-3007). T cells may differ in their activation state, which may or may not be reflected by the expression of activation markers. [Id. citing Crabtree, G R. Science (1989) 243: 355-361].

The best characterized lymphocyte populations in humans are those contained in the peripheral blood. Peripheral blood lymphocytes (PBLs), which are mature lymphocytes that circulate in the blood rather than being localized to organs, include B cells, T cells and natural killer cells. [Chiu, Po-Llin, et al. Scientific Reports (2019) 9: article 8145].

By analogy to T lymphocytes, the distribution of B cells follows a nonrandom pattern. Surface IgA-bearing lymphocytes are highly represented in mucosa-associated lymphoid structures (e.g., lamina propria and Peyer's patches), the nonkeratinizing external surfaces of the body (gut and exocrine glands, including the lactating mammary gland, urogenital epithelia and upper respiratory tract) attract predominantly IgA-secreting plasma cells. In nonmucosal sites (peripheral lymph nodes, spleen and skin), IgA secreting cells are infrequent and most plasma cells secrete IgM or IgG. Similarly, distinct differentiation and activation stages of B lymphocytes are discontinuously distributed in different zones of lymphoid follicles (a lymphoid follicle is a compartment of primarily B cells, which represents a unique microenvironment). The expression of different V_{H} gene families is also inhomogeneous [Kroemer, G. et al. Adv. Immunol. (1993) 53: 157-216, citing, Freitas, A. A. et al. Int. Immunol. (1989) 1: 342-54]. B cells may be divided into two classes according to the expression of CD5, a signal-transducing receptor [Id., citing Alberola-Ila, J. et al. J. Immunol. (1992) 148: 1287-93] that interacts with the B cell surface marker CD72/Lub-2 [Id., citing Van de Velde, H. et al. Nature (London) (1992) 148: 1287-93]. B1 cells represent the CD5+(Ly-1+) subset, and have the phenotype IgM^{high}IgD^{low}-Mac-1 (CD11b/CD18)+CD45^{low}FceR-IL-5R+. B2 “conventional” cells have a similar phenotype except that they lack CD5 (Id. citing Hayakawa, K., et al. J. Exp. Med. (1983) 157: 202-15; Wetzel, G D. Eur. J. Immunol. (1989) 19: 1701-08; Herzenberg, L A, et al. Immunol. Rev. (1986) 93: 81-109; Waldschmidt, T J et al. Int. Immunol. (1991) 3: 305-315; Marcos, M A R, et al. Scand. J. Immunol. (1991) 34: 129-35; Kasaian, M T et al. J. Immunol. (1992) 148: 2690-2702]. CD5+ B cells are endowed with the capacity of self-renewal, i.e., they may expand in the absence of any cell input from IgM-precursors, unlike conventional B cells [Id., citing Herzenberg, L A, et al. Immunol. Rev. (1986) 93: 81-109, Hayakawa, K. et al. Eur. J. Immunol. (1986) 4: 243-52; Forster, I., Rajewsky, K. Eur. J. Immunol. (1987) 17: 521-28].

A large proportion of mature lymphocytes continuously traffic from the bloodstream into lymphoid organs and tissue, then to the collecting efferent lymphatics, and eventually back to the bloodstream. Lymphocyte migration follows a nonrandom pattern. Naïve T cells migrate into lymph nodes, whereas memory T cells traffic preferentially into nonlymphoid tissue [Id., citing Mackay, C F (1991) Immunol. Today (1991) 12: 189-92; Pober, J S, Cotran, R S Adv. Immunol. (1991) 50: 261-302; Dustin, M L, Springer, T A Annu. Rev. Immunol. 9: 27-66; Oppenheimer-Marks, N. et al. J. Immunol. (1990) 145: 140-48].

### Memory T Cells

The vast majority of human memory T cells reside in tissue sites, including lymphoid tissues, intestines, lungs and skin. By the end of puberty, lymphoid tissues, mucosal sites and the skin are populated predominantly by memory T cells, which persist throughout adult life and represent the most abundant lymphocyte population throughout the body.

Memory T cells in humans are classically distinguished by the phenotype CD45RO+CD45RA−, and comprise heterogeneous populations of memory T cell subsets. [Farber, D L, et al. Nat. Rev. Immunol. (2014) 14(1): 24-35] Naïve T cells uniformly express CCR7, reflecting their predominant residence in lymphoid tissue. Memory T cells are subdivided into CD45RA−CCR7+ central memory T (T_{CM}) cells, which traffic to lymphoid tissues, and CD45RA−CCR7− effector memory T (T_{EM}) cells, which can migrate to multiple peripheral tissue sites. Functionally, both T_{CM} and T_{EM} cell subsets produce effector cytokines in response to viruses, antigens and other stimuli [Id., citing Wang A, et al. Sci Transl Med. (2012) 4:149ra12030-33; Pedron B, et al. Pediatr Res. (2011) 69:106-111; Champagne P, et al. Nature. (2001) 410:106-111; Ellefsen K, et al. Eur J Immunol. (2002) 32:3756-3764], although T_{CM} cells exhibit a higher proliferative capacity. (Id. citing Wang A, et al. Sci Transl Med. (2012) 4:149ra120, Fearon D T, et al. Immunol Rev. 2006; 211:104-118). A new subset, T memory stem (T_{SCM}) cells, which resemble naïve T cells in that they are CD45RA+CD45RO− and express high levels of the co-stimulatory receptors CD27 and CD28, IL-7 receptor α chain (IL7Rα), CD62L and CCR7, have high proliferative capacity and are both self-renewing and multipotent in that they can further differentiate into other subsets, including T_{CM} and T_{EM} cells [Id. citing Gattinoni L, et al. Nat Med. (2011) 17:1290-1297, Gattinoni L, et al. Clin Cancer Res. (2010) 16:4695-4701]. A progressive differentiation pathway based on signal strength and/or extent of activation places naïve (T_{N}), T_{SCM}, T_{CM} and T_{EM} cells in a differentiation hierarchy, serving as precursors for effector T cells [Id. citing Gattinoni L, et al. Nat Rev Cancer. (2012) 12:671-684; Klebanoff C A, et al. Immunol Rev. (2006) 211:214-224; Lanzavecchia A, Sallusto F. Nat Rev Immunol. (2002) 2:982-987].

In mice, tissue resident memory T (T_{RM}) cells are a non-circulating subset that resides in peripheral tissue sites and, in some cases, elicits rapid in situ protective responses. Mouse CD4+T_{RM} cells can be generated in the lungs from adoptive transfer or activated (effector) T cells [Id., citing Teijaro J R, et al. J Immunol. (2011) 187:5510-5514] or following respiratory virus infection [Id., citing Turner, D L, et al. Mucosal Immunol. (2014) 7 (3): 501-510], and are distinguished from splenic and circulating memory T cells by their upregulation of the early activation marker CD69, their tissue-specific retention in niches of the lung [Id., citing Turner, D L, et al. Mucosal Immunol. (2014) 7 (3): 501-510] and their enhanced ability to mediate protection to influenza virus infection compared to circulating memory CD4+ T cells [Id. citing Teijaro J R, et al. J Immunol. (2011) 187:5510-5514]. An analogous non-circulating CD4+ T_{RM} cell subset has been identified in the bone marrow of mice following systemic virus infection that exhibits enhanced helper functions. [Id., citing Hemdler-Brandstetter D, et al. J Immunol. (2011) 186:6965-6971]. CD8+T_{RM} cells generated following infection have been identified in multiple mouse tissues, including skin [Id., citing Clark R A, et al. Sci Transl Med. (2012) 4:117ra117; Liu L, et al. Nat Med. (2010) 16:224-227], vaginal mucosa [Id., citing Mackay L K, et al. Proc Natl Acad Sci USA. (2012) 109:7037-7042, Shin H, Iwasaki A. Nature. (2012) 491:463-467], intestine [Id., citing Klonowski K D, et al. Immunity. (2004) 20:551-562, Masopust D, et al. J Exp Med. (2010) 207:553-564, Masopust D, et al. J Immunol. (2006) 176:2079-2083], lungs [Id., citing Turner, D L, et al. Mucosal Immunol. (2014) 7 (3): 501-510, Anderson K G, et al. J Immunol. (2012) 189:2702-2706] and brain [Id., citing Wakim L M, et al. Proc Natl Acad Sci USA. (2010) 107:17872-17879]. They are distinguished from splenic and circulating memory CD8+ T cells by their increased expression of CD69 and by expression of the epithelial cell binding integrin αEβ7 (also known as CD103 [Id, citing Mueller S N, et al. Annu Rev Immunol. (2013) 31:137-161, Mackay L K, et al. Proc Natl Acad Sci USA. (2012) 109:7037-7042, Casey K A, et al. J Immunol. (2012) 188:4866-4875; Masopust D, Picker U. J Immunol. (2012) 188:5811-5817; Gebhardt T, Mackay L K. Front Immunol. (2012) 3:340].

In humans, memory CD4+ T cells predominate throughout the body and persist as CCR7+ or CCR7− subsets localized to lymphoid tissues and mucosal sites, respectively, whereas memory CD8+ T cells persist as mainly CCR7− subsets in all sites, with low numbers of CD8 T_{CM} cells in lymphoid tissues and negligible numbers of these cells in other sites [Id., citing Sathaliyawala T, et al. Immunity (2013) 38:187-197]. Most memory T cells in human mucosal, lymphoid and peripheral tissue sites such as skin express the putative T_{RM} cell marker CD69 [Id., citing Goronzy J J, Weyand C M. Nat Immunol. (2013) 14:428-436; Nikolich-Zugich J, Rudd B D. Curr Opin Immunol. (2010) 22:535-540; Clark R A, et al. J Immunol. (2006) 176:4431-4439, Mueller S N, et al. Annu Rev Immunol. (2013) 31:137-161, Casey K A, et al. J Immunol. (2012) 188:4866-4875], whereas circulating blood memory T cells uniformly lack CD69 expression. [Id., citing Sathaliyawala T, et al. Immunity (2013) 38:187-197].

Human T_{RM} cells also exhibit tissue-specific properties, suggesting in situ influences. For example, memory T cells in the small intestine and colon express the gut-homing receptor CCR9 [Id., citing Kunkel E J, et al. J Exp Med. (2000) 192:761-768] and the integrin a407 [Id., citing Agace W W. Trends Immunol. (2008) 29:514-522], and memory T cells in the lungs upregulate CCR6 expression [Id., citing Purwar R, et al. PLoS One. (2011) 6:e16245]. There is also evidence for crosstalk between mucosal sites, such as lung and intestines. For example lung dendritic cells induce migration of protective T cells to the gastrointestinal tract. [Id. citing Ruane D, et al. J Exp Med. (2013) 210:1871-1888].

There is evidence that Tim can be multifunctional and also exhibit qualitative functional differences. A substantial fraction of human lung T_{RM} cells produce multiple pro-inflammatory cytokines [Id., citing Purwar R, et al. PLoS One. 2011; 6:e16245], and human intestinal T_{RM} cells are also multifunctional [Id. citing Sathaliyawala T, et al. Immunity. 2013; 38:187-197]. Other functions appear to be confined to specific subsets and/or tissue sites. For example, IL-17 is produced by a subset of CD4+ T_{RM} cells in mucosal sites, particularly in intestines in healthy individuals [Id., citing Sathaliyawala T, et al. Immunity (2013) 38:187-197], by CCR6+ memory T cells in peripheral blood [Id., citing Singh S P, et al. J Immunol. (2008) 180:214-221, Wan Q, et al. J Exp Med. (2011) 208:1875-1887], and by a subset of CD161+ T cells in inflamed tissue, such as the skin of patients with psoriasis [Id., citing Cosmi L, et al. J Exp Med. 2008; 205:1903-1916]. Thus, while predominant memory T cell functions, such as IFNγ production, are broadly distributed among multiple memory T cell subsets and tissues, T_{RM} cells in tissue sites can adopt multiple or distinct functional attributes, which may also depend on tissue-specific inflammation.

Despite their specificity, human memory T cells exhibit cross-reactivity to antigenic epitopes not previously encountered, which may be due to intrinsic properties of TCR recognition [Id., citing Sewell A K. Nat Rev Immunol. 2012; 12:669-677] and to the range and breadth of human antigenic experience. Memory CD4+ and CD8+ T cells specific for unique epitopes of avian influenza strain H5N1 were detected in healthy individuals that were not exposed to H5N1 infection assessed by serology [Id., citing Lee L Y, et al. J Clin Invest. (2008) 118 (10): 3478-90; Roti M, et al. J Immunol. (2008) 180:1758-1768]. In addition, HIV-specific memory T cells have been identified in HIV-negative individuals [Id., citing Su, L F et al. Immunity (2013) 38: 373-83]. Virus-specific memory T cells also show cross-reactivity to alloantigens, autoantigens and unrelated pathogens [Id., citing D'Orsogna L J, et al. Transpl Immunol. (2010) 23:149-155, Wucherpfennig K W. Mol Immunol. (2004) 40:1009-1017]: EBV-specific human memory T cells generated in HLA-B8 individuals exhibit allogeneic cross-reactivity to HLA-B44 [Id., citing Burrows S R, et al. J Exp Med. (1994) 179:1155-11611, and influenza virus- and HIV-specific memory CD4+ T cells recognize epitopes from unrelated microbial pathogens [Id., citing Su L F, et al. Immunity. (2013) 38:373-3831. Furthermore, T cells specific for the autoantigen myelin basic protein (MBP) recognized multiple epitopes from viral and bacterial pathogens [Id., citing Wucherpfennig K W. Mol Immunol. (2004) 40:1009-1017, Wucherpfennig K W, Strominger J L. Cell. (1995) 80:695-7051. This cross-reactivity may enable memory T cells to mediate protection without initial disease—a phenomenon known as heterologous immunity [Id., citing Welsh R M, Selin L K. Nat Rev Immunol. (2002) 2:417-4261. Heterologous immunity has been demonstrated in humans where EBV infection expanded clones of influenza virus-specific T cells [Id., citing Clute S C, et al. J Clin Invest. 2005; 115:3602-3612].

Analysis of human samples has revealed that influenza-specific Ti can be found in substantial numbers in lung tissue, highlighting their role in natural infection. Despite expressing low levels of granzyme B and CD107a, these CD8+ T_{RM} had a diverse T cell receptor (TCR) repertoire, high proliferative capacities, and were polyfunctional [Muruganandah, V., et al. (2018). Front. Immunol., 9, 1574. doi:10.3389/fimmu.2018.01574]. Influenza infection history suggests a greater level of protection against re-infections is likely due to the accumulation of CD8+ T_{RM} in the lungs. Furthermore, the natural immune response to influenza A virus infection in a rhesus monkey model demonstrated that a large portion of influenza-specific CD8+ T cells generated in the lungs were phenotypically confirmed as CD69+CD103+ T_{RM}. Unlike lung parenchymal T_{RM}, airway CD8+ T_{RM} are poorly cytolytic and participate in early viral replication control by producing a rapid and robust IFN-γ response. Bystander CD8+ T_{RM} may also take part in the early immune response to infection through antigen non-specific, NKG2D-mediated immunity. The generation of functional T_{RM} that protect against heterosubtypic influenza infection appear to be dependent on signals from CD4+ T cells. [Muruganandah, V., et al. (2018). Frontiers in Immunology, 9, 1574. doi:10.3389/fimmu.2018.01574].

According to the paradigm of a typical CD8+ T cell response to acute viruses, CD8+ T cells are effectors when an antigen is present and become memory when the antigen is eliminated. However, it has become apparent that in viral infections, a memory T cell population comprises multiple subtypes of cells, distributed in diverse anatomic compartments and possibly recirculating among them. The memory CD8+ T cell response to most viruses is diverse in phenotype and function and undergoes dynamic changes during its development and maintenance in vivo. This heterogeneity is related to the nature of the infecting virus, its cellular tropism, the anatomic location of the infection, and the location of the CD8+ T cells. In resolved acute infections, the presence of memory CD8+ T cells at the site of the original virus entry and replication is crucial for a rapid response to a secondary infection. In latent infections, the presence of memory CD8+ T cells at sites of virus persistence is important for immune surveillance of virus reactivation. [Racanelli, V. et al., Rev. Med. Virol. (2011) 21 (6): 347-357].

### Mucosal Immune System

While the mucosal surfaces of the body have a protective barrier of mucus, they are highly vulnerable to infection and possess a complex array of innate and adaptive mechanisms of immunity. The adaptive immune system of the mucosa-associated lymphoid tissues differs from that of the rest of the peripheral lymphoid system in several respects. The types and distribution of T cells differ, with significantly greater numbers of γ:δ T cells in the gut mucosa compared with peripheral lymph nodes and blood. In addition, thee major antibody type secreted across the epithelial cells lining mucosal surfaces is different—it is secretory polymeric IgA. [Immunobiology: The Immune System in Health and disease. Janeway, C A et al Eds., 5^{th} Ed. (2001), Garland Publishing, New York, Ch. 10, p. 482-493].

The mucosal immune system protects internal mucosal surfaces, such as the linings of the gut, respiratory tract and urogenital tracts, which are the site of entry for virtually all pathogens and other antigens. The mucosa-associated lymphoid tissues lining the gut are known as gut-associated lymphoid tissue or GALT. The tonsils and adenoids, which form a ring, known as Waldeyees ring, at the back of the mouth at the entrance of the gut and airways, represent large aggregates of mucosal lymphoid tissue, which often become extremely enlarged in childhood because of recurrent infections. The other principal sites within the gut mucosal immune system for the induction of immune responses are the Peyer's patches of the small intestine, the appendix, and solitary lymphoid follicles of the large intestine and rectum. Peyer's patches are an important site for the induction of immune responses in the small intestine and have a distinctive structure, forming domelike structures extending into the lumen of the intestine. The overlying layer of follicle-associated epithelium of the Peyer's patches contains specialized epithelial cells (microfold cells or M cells) that have microfolds on their luminal surface, instead of the microvilli present on the absorptive epithelial cells of the intestine. They are much less prominent than the absorptive gut epithelial cells, known as enterocytes, and form a membrane overlying the lymphoid tissue within the Peyer's patch. Since M cells lack a thick surface glycocalyx and do not secrete mucus, they are adapted to interact directly with molecules and particles within the lumen of the gut. M cells take up molecules and particles from the gut lumen by endocytosis or phagocytosis. This material is then transported through the interior of the cell in vesicles to the basal cell membrane, where it is released into the extracellular space by transcytosis. At their basal surface, the cell membrane of M cells is extensively folded around underlying lymphocytes and antigen-presenting cells, which take up the transported material released from the M cells and process it for antigen presentation. [Immunobiology: The Immune System in Health and disease. Janeway, C A et al Eds., 5^{th} Ed. (2001) Garland Publishing, New York, Ch. 10, p. 482-493].

In addition to the organized lymphoid tissue in which induction of immune responses occurs within the mucosal immune system, small foci of lymphocytes and plasma cells, which are scattered widely throughout the lamina propria of the gut wall, represent the effector cells of the gut mucosal immune system. As naive lymphocytes, these cells emerge from the primary lymphoid organs of bone marrow and thymus to enter the inductive lymphoid tissue of the mucosal immune system via the bloodstream where they may encounter foreign antigens presented within the organized lymphoid tissue of the mucosal immune system and become activated to effector status. From these sites, the activated lymphocytes traffic via the lymphatics draining the intestines, pass through mesenteric lymph nodes, and eventually wind up in the thoracic duct, from which they circulate in the blood throughout the entire body. They reenter the mucosal tissues from the small blood vessels lining the gut wall and other sites of mucosa-associated lymphoid tissue (MALT), such as the respiratory or reproductive mucosa, and the lactating breast; these small vessels express the mucosal adressin MAdCAM-1. In this way, an immune response that may be started by foreign antigens presented in a limited number of Peyer's patches is disseminated throughout the mucosa of the body. This pathway of lymphocyte trafficking is distinct from and parallel to that of lymphocytes in the rest of the peripheral lymphoid system. [Immunobiology: The Immune System in Health and disease. Janeway, C A et al Eds., 5^{th} Ed. (2001) Garland Publishing, New York, Ch. 10, p. 482-493].

The distinctiveness of the mucosal immune system from the rest of the peripheral lymphoid system is further underlined by the different lymphocyte repertoires in the different compartments. The T cells of the gut can be divided into two types. One type bears the conventional α:β T-cell receptors in conjunction with either CD4 or CD8, and participates in conventional T-cell responses to foreign antigens. The second class is made up of T cells with unusual surface phenotypes such as TCRγ:δ and CD8α:α TCRα:β. The receptors of these T cells do not bind to the normal MHC:peptide ligands. Instead, they bind to a number of different ligands, including MHC class IB molecules. These highly specialized T cells are abundant in the epithelium of the gut and have a restricted repertoire of T-cell receptor specificities. Unlike conventional T cells, many of these cells do not undergo positive and negative selection in the thymus, and express receptors with sequences that have undergone no or minimal divergence from their germline-encoded sequences. These cells may be classified in phylogenetic terms as being at the interface between innate and adaptive immunity. [Immunobiology: The Immune System in Health and disease. Janeway, C A et al Eds., 5^{th} Ed. (2001) Garland Publishing, New York, Ch. 10, p. 482-493].

T cells bearing a γ:δ receptor are especially abundant in the gut mucosa compared with other lymphoid tissues. One subset of these γ:δ T cells in humans, which expresses a T-cell receptor that uses the Vδ1 gene segment, carries an activating C-type lectin NK receptor, NKG2D. NKG2D binds to two MHC-like molecules-MIC-A and MIC-B—that are expressed on intestinal epithelial cells in response to cellular injury and stress. The injured cells may then be recognized and killed by the subset of γ:δ T cells. The Vδ1-containing receptor on these T cells may also play a part in allowing them to survey tissues for injured cells. Some human T cells expressing this receptor bind to CD1c, one of the isotypes of the CD1 family of MHC class I-like molecules. This protein, which shows increased expression on activated monocytes and dendritic cells, presents endogenous lipid and glycolipid antigens to some types of T cell. In response to antigen presentation by CD1c, these T cells secrete IFN-γ, which may have an important role in polarizing the response of conventional T cells bearing α:β receptors toward a T_{H}1 response. This is closely analogous, although opposite in effect, to the polarization toward T_{H}2 cells induced by secretion of IL-4 by NK 1.1+ T cells (NK1+ T cells) responding to CD1d. [Immunobiology: The Immune System in Health and disease. Janeway, C A et al Eds., 5^{th} Ed. (2001) Garland Publishing, New York, Ch. 10, p. 482-493].

### Involvement of γδ T Cells in Viral Lung Infections

Lung-resident γδ T cells play critical roles in anti-viral immune responses and are involved in virus-induced lung inflammation and injury. Respiratory syncytial virus (RSV), one of many (˜200) viruses known as a common cold virus, predominately affects infants and leads to long-term lung disease. [Cheng, M., and Hu, Shilian. Immunology (2017) 151: 375-84]. The contribution of γδ T cells to RSV infection has been tested in mice infected with RSV with or without immunization with a live vaccine vector expressing RSV F protein. Vγ4+γδ T cells were enhanced in the lungs and produced IFN-γ, RANTES, IL-10, IL-4 and IL-5 in a time-dependent manner after challenge of sensitized mice. Depletion of γδ T cells reduced lung inflammation and disease severity and slightly increased peak viral replication without compromising viral clearance during secondary challenge in vaccinated mice. [Id., citing Dodd J, et al. J Immunol (2009) 182:1174-81]. Using a neonatal mouse model of RSV, it was found that neonates failed to develop IL-17A responses of the type observed in adult mice. In adults, γδ T cells are the main producers of IL-17A. Exogenous IL-17A administration decreases inflammation in RSV-infected neonates, whereas neutralization of IL-17A increases lung inflammation and airway mucus in RSV-infected adults. Hence, RSV disease severity is in part mediated by a lack of IL-17A+γδ T cells in the lungs of neonates. [Id., citing Huang H, et al. Immunol Cell Biol (2015) 93:126-35]. Additionally, RSV infection elevates T_{H}1 cytokine- and suppresses T_{H}2 cytokine-expression in lung γδ T cells. Ovalbumin (OVA) challenge induces a large influx of γδ T cells into the lungs. When mice were previously infected with RSV, the OVA-induced infiltration and activation of γδ T cells were inhibited, suggesting that RSV protected against subsequent OVA-induced allergic responses by inhibiting T_{H}2-type γδ T cells. [Id., citing Zhang L, et al. J Med Virol (2013) 85:149-56]

During influenza virus infection, RORγt-positive αβ and γδ T cells, as well as innate lymphoid cells, express enhanced IL-22 as early as 2 days post-infection. Although IL-22 plays no role in the control of influenza A virus replication, IL-22 is beneficial during sublethal influenza A virus infection but not lethal influenza A virus infection, which limits lung inflammation and injury after a secondary challenge with S. pneumoniae. [Id., citing Ivanov S, et al. J Virol (2013) 87:6911-24] Type I interferon induction during influenza virus infection increases susceptibility to secondary S. pneumoniae infection by negative regulation of γδ T cells with decreased IL-17 production. [Id., citing Li W, et al. J Virol (2012) 86:12304-12]. Human Vγ9Vδ2 T cells that are activated in vitro by aminobisphosphonate pamidronate efficiently kill influenza virus-infected lung alveolar epithelial cells and inhibit virus replication in a cell-to-cell contact manner. The cytotoxic activity of Vγ9Vδ2 T cells requires NKG2D activation and involves perforin/granzyme B, TRAIL and FasL. [Id., citing Li H, et al. Cell Mol Immunol (2013) 10:159-64, Tu W, et al. J Exp Med (2011) 208:1511-22]

### Natural Killer T (NKT) Cells

Natural killer T (NKT) cells are a heterogeneous subset of specialized T cells (Brennan et al., Nat Rev Immunol. 2013 February; 13(2):101-17). These cells exhibit an innate cell-like feature of quick response to antigenic exposure in combination with an adaptive cell's precision of antigenic recognition and diverse effector responses (Salio et al., Annu Rev Immunol. 2014; 320:323-66). Like conventional T cells, NKT cells undergo thymic development and selection and possess T cell receptors (TCRs) to recognize antigens (Berzins et al., Immunol Cell Biol. 2004 June; 82(3):269-75).

Natural killer T (NKT) cells represent a small population of T lymphocytes defined by the expression of both αβ T-cell receptors (TCR) and some lineage markers of NK cells. However, unlike conventional T cells, the TCRs expressed by NKT cells recognize lipid antigens presented by the conserved and non-polymorphic MHC class 1 like molecule CD1d (Godfrey et al., Nat Immunol. 2015 November; 16(11):1114-23). In addition to TCRs, NKT cells also possess receptors for cytokines such as IL-12, IL-18, IL-25, and IL-23 similar to innate cells such as NK and innate lymphoid cells (Cohen et al., Nat Immunol. 2013 January; 14(1):90-9). These cytokine receptors can be activated by steady state expression of these inflammatory cytokines even in the absence of TCR signals. Thus, NKT cells can amalgamate signals from both TCR-mediated stimulation and inflammatory cytokines to generate promptly release of an array of cytokines (Kohlgruber et al., Immunogenetics. 2016 August; 68(8):649-63), which, in turn, can modulate different immune cells present in a tumor microenvironment (TME) thus influencing host immune responses to cancer.

As shown in Table 3, there are a number of subtypes of NKT cells, which can be determined through their T cell receptor (TCR) usage, cytokine production, expression of specific surface molecules and reactivity.

TABLE 3

| NKT Cell Subset | | Mouse | Human |
|---|---|---|---|
| Type I | TCR | Vα14-Jα18; | Vα24-Jα18; |
| | | Vβ8.2/7/2 | Vβ11 |
| | Subsets | CD4+, DN | CD4+, CD8+, |
| | | | DN |
| | Ligand | αGalCer | αGalCer |
| | Restriction | CD1d | CD1d |
| | NK Receptors | NK1.1+/− | CD161+/− |
| Type II | TCR | Vα3.2-Jα9 or | Diverse |
| | | Vα8; VP8 | |
| | Subsets | CD4+, DN | CD4+, CD8+ |
| | Ligand | Sulfatide, | Sulfatide, |
| | | lysosulfatide, | lysosulfatide, |
| | | lysophosphatidyl- | lysophosphatidyl- |
| | | choline | choline |
| | Restriction | CD1d | CD1d |
| | NK Receptors | NK1.1+/− | CD161+ |

### Type-I NKT Cells

Broadly, CD1d-restricted NKT cells can be divided into two main subsets based on their TCR diversity and antigen specificities. The most extensively characterized subtype of NKT cells are the type-I or invariant natural killer T cell (iNKT cells) (Matsuda et al, Curr Opin Immunol, 20: 358-68, 2008). Type-I (invariant) NKT cells (iNKT cells), so named because of their limited TCR repertoire, express a semi-invariant TCR (iTCR) α chain (Vα14-Jα18 in mice, Vα24-Jα18 in humans) paired with a heterogeneous VO chain repertoire (V β 2,7 or 8.2 in mice and V β 11 in humans) (Brennan et al., Nat Rev Immunol. 2013 February; 13(2):101-17; Salio et al., Annu Rev Immunol. 2014; 32( ):323-66). The prototypic antigen for type-I NKT cells is galactosylceramide (α-GalCer or KRN 7000), which was isolated from a marine sponge as part of an antitumor screen (Kawano et al., Science. 1997 Nov. 28; 278(5343):1626-9). α-GalCer is a potent activator of type-I NKT cells, inducing them to release large amounts of interferon-γ (IFN-γ), which helps activate both CD8+ T cells and antigen presenting cells (APCs) (Kronenberg, Nat Rev Immunol. 2002 August; 2(8):557-68). The primary techniques used to study type-I NKT cells include staining and identification of type-I NKT cells using CD1d-loaded α-GalCer tetramers, administering α-GalCer to activate and study the functions of type-I NKT cells, and finally using CD1d deficient mice (that lack both type-I and type-II NKT) or Jα18-deficient mice (lacking only type-I NKT) (Berzins et al., Immunol Cell Biol. 2004 June; 82(3):269-75). It has been reported that Jα18-deficient mice in addition to having deletion in the Traj18 gene segment (essential for type-I NKT cell development), also exhibited an overall lower TCR repertoire caused by influence of the transgene on rearrangements of several Ja segments upstream Traj18, complicating interpretation of data obtained from the Jα18-deficient mice (Bedel et al., Nat Immunol. 2012 Jul. 19; 13(8):705-6). To overcome this drawback, a new strain of Jα18-deficient mice lacking type-I NKT cells while maintaining the overall TCR repertoire has been generated to facilitate future studies on type-I NKT cells (Chandra et al., Nat Immunol. 2015 August; 16(8):799-80). Type-I NKT cells can be further subdivided based on the surface expression of CD4 and CD8 into CD4+ and CD4-CD8-(double-negative, or DN) subsets and a small fraction of CD8+ cells found in humans (Bendelac et al., Science. 1994 Mar. 25; 263(5154):1774-8; Lee et al., J Exp Med. 2002 Mar. 4; 195(5):637-41). Type-I NKT cells are present in different tissues in both mice and humans, but at higher frequency in mice (Arrenberg et al., J Cell Physiol. 2009 February; 218(2):246-50).

Type-I NKT cells possess dual reactivity to both self and foreign lipids. Even at steady state, type-I NKT cell have an activated/memory phenotype (Bendelac et al., Annu Rev Immunol. 2007; 25( ):297-336; Godfrey et al., Nat Immunol. 2010 March; 11(3):197-206).

Functionally distinct subsets of NKT cells analogous to T_{H}1, T_{H}2, T_{H}17, and T_{FH} subsets of conventional T cells have been described. These subsets express the corresponding cytokines, transcription factors and surface markers of their conventional T cell counterparts (Lee et al., Immunity. 2015 Sep. 15; 43(3):566-78). Type-I NKT cells have a unique developmental program that is regulated by a number of transcription factors (Das et al., Immunol Rev. 2010 November; 238(1):195-215). Transcriptionally, one of the key regulators of type-I NKT cell development and activated memory phenotype is the transcription factor promyelocytic leukemia zinc finger (PLZF). In fact, PLZF deficient mice show profound deficiency of type-I NKT cells and cytokine production (Kovalovsky D, et al., Nat Immunol (2008) 9:1055-64.10.1038/ni.164; Savage A K et al., Immunity (2008) 29:391-403). Other transcription factors that are known to impact type-I NKT cell differentiation are c-Myc (Dose et al., Proc Natl Acad Sci USA. 2009 May 26; 106(21):8641-6), RORγt (Michel et al., Proc Natl Acad Sci USA. 2008 Dec. 16; 105(50):19845-50), c-Myb (Hu et al., Nat Immunol. 2010 May; 11(5):435-41), Elf-1 (Choi et al., Blood. 2011 Feb. 10; 117(6):1880-7), and Runx1 (Egawa et al., Immunity. 2005 June; 22(6):705-16). Furthermore, transcription factors that control conventional T cell differentiation, such as T_{H}i lineage specific transcription factor T-bet and T_{H}2 specific transcription factor GATA-3, can also affect type-I NKT cell development (Kim et al., J Immunol. 2006 Nov. 15; 177(10):6650-9; Townsend et al., Immunity. 2004 April; 20(4):477-94). Aside from transcription factors, SLAM-associated protein (SAP) signaling pathway can also selectively control expansion and differentiation of type-I NKT cells (Nichols et al., Nat Med. 2005 March; 11(3):340-5). Type-I NKT cells have been shown to respond to both self and foreign α and β linked glycosphingolipids (GSL), ceramides, and phospholipids (Macho-Femandez et al., Front Immunol. 2015; 6: 362). Type-I NKT cells have been reported to mostly aid in mounting an effective immune response against tumors (McEwen-Smith et al., Cancer Immunol Res. 2015 May; 3(5):425-35; Robertson et al., Front Immunol. 2014; 5( ):543; Ambrosino et al., J Immunol. 2007 Oct. 15; 179(8):5126-36).

### Type-II NKT Cells

Type-II NKT cells, also called diverse or variant NKT cells, are CD1d-restricted T cells that express more diverse alpha-beta TCRs and do not recognize α-GalCer (Cardell et al., J Exp Med. 1995 Oct. 1; 182(4):993-1004). Type-II NKT cells are a major subset in humans with higher frequency compared to type-I NKT cells. Due to an absence of specific markers and agonistic antigens to identify all type-II NKT cells, characterization of these cells has been challenging. Different methodologies employed to characterize type-II NKT cells include, comparing immune responses between Jα18−/− (lacking only type-I NKT) and CD1d−/− (lacking both type I and type-II NKT) mice, using 24 αβ TCR transgenic mice (that overexpress Vα3.2N9 TCR from type-II NKT cell hybridoma VIII24), using a Jα18-deficient IL-4 reporter mouse model, staining with antigen-loaded CD1d tetramer and assessing binding to type-II NKT hybridomas [reviewed in Macho-Fernandez, Front Immunol. 2015; 6:362)].

The first major antigen identified for self-glycolipid reactive type-II NKT cells in mice was myelin derived glycolipid sulfatide (Arrenberg et al., J Cell Physiol. 2009 February; 218(2):246-50; Jahng et al., J Exp Med. 2001 Dec. 17; 194(12):1789-99). Subsequently, sulfatide and lysosulfatide reactive CD1d-restricted human type-II NKT cells have been reported (Shamshiev et al., J. Exp. Med. 2002; 195:1013-1021; Blomqvist et al., Eur J Immunol. 2009 July; 39(7): 1726-1735). Sulfatide specific type-II NKT cells predominantly exhibit an oligoclonal (meaning cloned or derived from one or a few cells) TCR repertoire (V a 3/V a 1-J α 7/J α 9 and V β 8.1V β 3.1-J β 2.7) (Arrenberg et al., J Cell Physiol. 2009 February; 218(2):246-50). Other self-glycolipids such as β GcCer and β GalCer have been shown to activate murine type-II NKT cells (Rhost et al., Scand J Immunol. 2012 September; 76(3):246-55; Nair et al., Blood. 2015 Feb. 19; 125(8):1256-71). It was reported that two major sphingolipids accumulated in Gaucher disease (GD), β-glucosylceramide (β GlcCer) and its deacylated product glucosylsphingosine, are recognized by murine and human type-II NKT cells (Nair et al., Blood. 2015 Feb. 19; 125(8):1256-71). In an earlier study, it was shown that lysophosphatidylcholine (LPC), a lysophospholipid markedly upregulated in myeloma patients, was an antigen for human type-II NKT cells (Chang et al., Blood. 2008 Aug. 15; 112(4):1308-16).

Type-II NKT cells can be distinguished from type-I NKT cells by their predominance in humans versus mice, TCR binding and distinct antigen specificities (J Immunol. 2017 Feb. 1; 198(3):1015-1021).

Crystal structures of type-II NKT TCR-sulfatide/CD1d complex and type-I NKT TCR-α-GalCer/CD1d complex provide insights into the mechanisms by which NKT TCRs recognize antigen (Girardi et al., Immunol Rev. 2012 November; 250(1):167-79). The type-I NKT TCR was found to bind α-GalCer/CD1d complex in a rigid, parallel configuration mainly involving the a-chain. The key residues within the complementarity-determining region (CDR) CDR2β, CDR3α, and CDR1α loops of the semi-iTCR of type-I NKT cells were determined to be involved in the detection of the α-GalCer/CD1d complex (Pellicci et al, Immunity. 2009 Jul. 17; 31(1):47-59). On the other hand, type-II NKT TCRs contact their ligands primarily via their CDR30 loop rather than CDR3 α loops in an antiparallel fashion very similar to binding observed in some of the conventional MHC-restricted T cells (Griardi et al., Nat Immunol. 2012 September; 13(9):851-6). Ternary structure of sulfatide-reactive TCR molecules revealed that CDR3 α loop primarily contacted CD1d and the CDR30 determined the specificity of sulfatide antigen (Patel et al., Nat Immunol. 2012 September; 13(9):857-63). The flexibility in binding of type-II NKT TCR to its antigens akin to TCR-peptide-MHC complex resonates with its greater TCR diversity and ability to respond to wide range of ligands.

However, despite striking differences between the two subsets, similarities among the two subsets have also been reported. For example, both type-I and type-II NKT cells are autoreactive and depend on the transcriptional regulators PLZF and SAP for their development (Rhost et al., Scand J Immunol. 2012 September; 76(3):246-55). Although, many type-II NKT cells seem to have activated/memory phenotype like type-I NKT cells, in other studies, a subset of type-II NKT cells also displayed naïve T cell phenotype (CD45RA+, CD45RO−, CD62high, and CD69−/low) (Arrenberg et al., Proc Natl Acad Sci USA. 2010 Jun. 15; 107(24):10984-9). Type-II NKT cells are activated mainly by TCR signaling following recognition of lipid/CD1d complex (Roy et al., J Immunol. 2008 Mar. 1; 180(5):2942-50) independent of either TLR signaling or presence of IL-12 (Zeissig et al., Ann N Y Acad Sci. 2012 February; 1250:14-24).

### T Cell Development

As T cells develop in the thymus, TCR signals provide critical checkpoints as cells transit through the various stages of maturation. (See Huang, E. Y., et al, J. Immunol. (2003) 171: 2296-2304). For example, a pre-TCR signal is necessary for the most immature thymocyte subset, termed double negative (DN), to develop into double-positive (DP) thymocytes, expressing both CD4 and CD8. Id. The assembly and surface expression of CD3, pre Tα, and a functionally rearranged TCRβ-chain mediate this checkpoint, termed β selection. Id. After successful pre-TCR signaling, DN thymocytes undergo many rounds of division and multiple phenotypic changes. Id. In addition to genes that encode pre-TCR components, a number of other genes, which either affect pre-TCR signaling indirectly or are required for the numerous cellular changes seen during the DN to DP transition, regulate maturation. Id.

### Type-I NKT Cell Development

In both mice and humans, Type-I NKT cells segregate from conventional T cells during development at the double-positive (CD4+CD8+, DP) thymocyte stage, coincident with TCR αβ expression (Godfrey D I, Berzins S P Nat Rev Immunol. 2007 July; 7(7):505-18). Generation of the canonical TCRα used by type-I NKT cells is widely believed to be a random event, for although the amino acids which define the invariant Vα14-Jα18 rearrangement never vary, sequencing analysis has revealed that the nucleotides used to code for these amino acids are diverse (Lantz O, Bendelac A J Exp Med. 1994 Sep. 1; 180(3):1097-106). Due to structural constraints on recombination events in the TCRα locus, the numerous Vα and Jα gene segments become accessible for recombination as a function of their relative location in the locus. As a result, the Vα 14 gene segment only starts rearranging with Jα18 within a 24-48 h window before birth (Hager E. et al. J Immunol. 2007 Aug. 15; 179(4):2228-34). This explains the relatively late appearance of NKT cells in the thymus and is consistent with random generation of the canonical Vα14-Jα18 rearrangement within a common T cell progenitor pool. Furthermore, the frequency of the earliest identified NKT cell precursor was estimated to be 1 cell per 106 thymocytes (Benlagha K. et al. J Exp Med. 2005 Aug. 15; 202(4):485-92). Together, these data support the notion that Vα14-Jα18 rearrangement occurs randomly at very low frequency.

As with conventional T cells, type-I NKT cell development requires recognition of self. The restriction element CD1d is expressed by both DP thymocytes and epithelial cells in the thymus. However, early studies revealed that type-I NKT cells are selected at the DP stage by CD1d-expressing DP cells themselves as opposed to epithelial cells that drive the selection of conventional T cells. Such a mode of selection was hypothesized to impart the unique developmental program of type-I NKT cells to the selected thymocytes. Recently, it was demonstrated that homotypic interactions across the DP-DP synapse generated “second signals” that are mediated by the cooperative engagement of the homophilic receptors of at least two members of the signaling lymphocytic-activation molecule (SLAM) family (Slamf1 [SLAM] and Slamf6 [Ly108]) [8λλ-10λλ]. Such engagements lead to the downstream recruitment of the adaptor SLAM-associated protein (SAP) and the Src kinase Fyn, which were previously recognized as essential for the expansion and differentiation of the type-I NKT cell lineage (Godfrey D I, 2007).

Once type-I NKT cells have been positively selected, they expand in the thymus and undergo an orchestrated maturation process that ultimately leads to the acquisition of their activated NK-like phenotype. This process relies on the proper expression of cytokine receptors, signal transduction molecules (e.g. Fyn, SAP), transcription factors (e.g. NFκB, T-bet, Ets1, Runx1, RORγ, Itk, Rlk, AP-1) (see Godfrey D I, 2007 for reviews), and co-stimulatory molecules such as CD28 and ICOS (Hayakawa et al., J Immunol. 2001 May 15; 166(10):6012-8; Akbari et al., J Immunol. 2008 Apr. 15; 180(8): 5448-5456). Most type-I NKT cells leave the thymus in an immature stage (as defined by the absence of expression of NK receptors such as NK1.1) and fulfill their terminal maturation in the periphery (Benlagha K. et al., Science. 2002 Apr. 19; 296(5567):553-5; McNab F W et al., J Immunol. 2005 Sep. 15; 175(6):3762-8). However, a sizeable fraction of these NK1.1—type-I NKT cells in the peripheral organs do not acquire expression of NK markers and in fact represent mature cells that are functionally distinct from their NK1.1+ thymic counterpart (McNab et al., J Immunol. 2007; 179:6630-6637).

The egress of type-I NKT cells from the thymus to the periphery requires lymphotoxin (LT) αβ signaling through the LTD receptor expressed by thymic stromal cells (Franki A S et al., Proc Natl Acad Sci USA. 2006 Jun. 13; 103(24):9160-5). Such signaling in turn regulates thymic medullary chemokine secretion (Zhu M. et al., J Immunol. 2007 Dec. 15; 179(12):8069-75). Establishment of type-I NKT cells tissue residency in the periphery requires expression of the Sphingosinel-Phosphate 1 receptor (S1P1R) by type-I NKT cells (Allende M L et al., FASEB J. 2008 January; 22(1):307-15) and more specifically expression of CxCR6 for liver localization (Geissmann F. et al., PLoS Biol. 2005 April; 3(4):e113).

However, many type-I NKT cells remain in the thymus, mature to the NK1.1+ phenotype there, and become long-lived residents (Berzins S P et al. J Immunol. 2006 Apr. 1; 176(7):4059-65). The mechanisms responsible for the export/retention of type-I NKT cells from the thymus at various developmental stages are unknown.

### Type-I NKT Cell Activity

Type-I NKT cells have been shown to have many different activities during an immune response. Not only do they have the capacity to rapidly and robustly produce cytokines and chemokines, they also have the ability, as their name would suggest, to kill other cells. In addition, they have been shown to influence the behavior of many other immune cells. In this section, the multitude of functional properties that have been attributed to type-I NKT cells is described.

### Cytokine and Chemokine Production

Type-I NKT cells were originally identified as an unusual T cell population with NK markers that had the unique capacity to rapidly and robustly produce IL-4 upon the injection of anti-CD3 antibodies in mice. Later studies revealed that while this robust IL-4 production was a signature of Type-I NKT cells, it was not the only cytokine type-I NKT cells can produce. Type-I NKT cells have been shown to produce IFN-γ and IL-4, as well as IL-2, IL-5, IL-6, IL-10, IL-13, IL-17, IL-21, TNF-α, TGF-β and GM-CSF (Bendelac A. et al., Annu Rev Immunol. 2007; 250:297-336; Gumperz J E et al., J Exp Med. 2002 Mar. 4; 195(5):625-36). Type-I NKT cells are also known to produce an array of chemokines (Chang Y J et al., Proc Natl Acad Sci USA. 2007 Jun. 19; 104(25):10299-304).

The rapid and dual production of IL-4 and IFNγ by type-I NKT cells in vivo following administration of the α-GalCer antigen has become a trademark feature of type-I NKT cells. In fact, within 2 h of in vivo exposure to antigen, intracellular analysis of ex vivo type-I NKT cells from naïve mice revealed that the majority of type-I NKT cells in the liver produced both IL-4 and IFNγ (Matsuda J L et al., J Exp Med. 2000 Sep. 4; 192(5):741-54). How type-I NKT cells from unsensitized mice produce cytokines so rapidly when activated is unclear. However, the observation that resting type-I NKT cells have high levels of IL-4 and IFNγ mRNAs provides one potential mechanism (Matsuda J L et al., Proc Natl Acad Sci USA. 2003 Jul. 8; 100(14):8395-400; Stetson D B et al., J Exp Med. 2003 Oct. 6; 198(7):1069-76).

Type-I NKT cells also regulate their cytokine production at the transcriptional level. Several transcription factors known to regulate cytokine gene transcription in conventional T cells (T-bet, GATA-3, NFκB, c-Rel, NFAT, AP-1, STATs, Itk) have also been implicated in type-I NKT cells. For example, type-I NKT cells appear to co-express both T-bet and GATA-3 transcription factors leading to the transcription of both IFNγ and IL-4 mRNAs. This is in contrast to conventional T cells where T-bet has been shown to repress the expression of GATA-3 and vice versa.

### Cytolytic Activity of Type-I NKT Cells

Type-I NKT cells express high levels of granzyme B, perforin, and FasL, consistent with a cytolytic function for these cells. In vitro assays have demonstrated that type-I NKT cells have the ability to kill antigen-pulsed APCs in a CD1d-dependent manner. In addition, several mouse models have revealed that type-I NKT cells play an important role in tumor surveillance and tumor rejection. In some tumor models, IFNγ production by type-I NKT cells is instrumental in the activation of NK cells, which in turn mount a robust anti-tumor response (Crowe N Y et al., J Exp Med. 2002 Jul. 1; 196(1):119-27). Similarly, type-I NKT cells have been shown to recognize and respond to bacterial antigens and participate in bacterial clearance (Mattner et al., Nature. 2005 Mar. 24; 434(7032):525-9; Ranson et al., J Immunol. 2005 Jul. 15; 175(2):1137-44).

### Regulation of Other Immune Cells

Early studies demonstrated that type-I NKT cell-derived cytokines can activate several other cell types, including NK cells, conventional CD4+ and CD8+ T cells, macrophages and B cells, and recruit myeloid dendritic cells (Kronenberg M, Gapin L Nat Rev Immunol. 2002 August; 2(8):557-68). Type-I NKT cells can also modulate the recruitment of neutrophils through their secretion of IFNγ (Nakamatsu M. et al., Microbes Infect. 2007 March; 9(3):364-74). Further, cross-talk between CD4+CD25+ regulatory T cells (Treg) and type-I NKT cells has been described, where activated type-I NKT cells quantitatively and qualitatively modulate Treg function through an IL-2 dependent mechanism, while Treg can suppress type-I NKT cell functions by cell-contact-dependent mechanisms (LaCava A. et al., Trends Immunol. 2006 July; 27(7):322-7). A similar cross-regulation between type-I NKT cells and other CD1d-restricted NKT cells that do not express the invariant TCR-α chain that characterize type-I NKT cells (type-II NKT cells), has also been observed (Ambrosino E. et al., J Immunol. 2007 Oct. 15; 179(8):5126-36). Type-I NKT cells have also been reported to synergize with γδ T cells in a model of allergic airway hyper-responsiveness (Jin N. et al., J Immunol. 2007 Sep. 1; 179(5):2961-8). Finally, it has been recognized for some time that systemic type-I NKT cell activation by α-GalCer injection induces activation of B cells non-specifically. Data show that purified type-I NKT cells from lupus-prone NZB/W F1 mice can spontaneously increase antibody secretion by B-1 and marginal zone B cells but not follicular zone B cells (Takahashi T, Strober S Eur J Immunol. 2008 January; 38(1):156-65). Direct interactions between type-I NKT cells and the B cell subsets were necessary and the effect could be blocked by anti-CD1d and anti-CD40L mAbs (Takahashi T, 2008). C57BL/6 mice immunized with proteins and α-GalCer developed antibody titers 1-2 logs higher than those induced by proteins alone and increased the frequency of memory B cells generated (Galli G et al., Proc Natl Acad Sci USA. 2007; 104:3984-3989). The mechanism was mediated through the combined action of CD40-CD40L interactions and cytokine secretion. CD1d expression by B cells is also required for the type-I NKT cell enhanced response, suggesting cognate interaction between type-I NKT cells and B cells (Lang G A et al., Blood. 2008 Feb. 15; 111(4):2158-62).

### Antigens Recognized by Type-I NKT Cells

The first described type-I NKT cell ligand was α-Galactosylceramide (α-GalCer), which was identified from a panel of marine extracts for its anti-tumor activity (Kawano T. et al., Science. 1997 Nov. 28; 278(5343):1626-9). Since then, many more type-I NKT cell antigens have been discovered, including both endogenous and exogenous antigens. Unlike conventional T cell antigens that are predominantly peptides presented by MHC molecules, type-I NKT cell antigens have a distinct lipid component to them. Most type-I NKT cell antigens defined to date share a common structure: a lipid tail that is buried into CD1d and a sugar head group that protrudes out of CD1d and makes contact with the NKT TCR. The main exception to this is the type-I NKT antigen phosphatidylethanolamine, which lacks a sugar head group.

### Recognition of Antigens by NKT Cells

The unique antigen specificity of type-I NKT cells is dictated by the expression of the semi-invariant TCR. How this TCR, which was known to have a similar overall structure to known peptide/MHC reactive TCRs, might instead recognize glycolipid antigens in the context of CD1d was the subject of constant speculation. Crystallographic success and mutational analyses have exposed how this TCR recognizes CD1d/glycolipid complexes. The crystal structure of a human type-I NKT TCR in complex with CD1d/α-GalCer revealed a unique docking strategy that differed from known TCR/MHC/peptide interactions (Borg et al., Nature. 2007; 448:44-49). Compared with conventional TCR-MHC interactions, where TCR engages the distal portion of the MHC in a diagonal orientation, the type-I NKT TCR docked at the very end of, and parallel to, the CD1d-α-Galcer complex. In the structure, the binding surface between the type-I NKT TCR and CD1d-α-GalCer complex was composed primarily of three out of the six complementarity-determining region (CDR) loops: CDR1α, CDR3α and CDR2β, with the invariant TCRα chain dominating the interaction with both the glycolipid and CD1d, while the role of the TCRα chain was restricted to the CDR20 loop interacting with the α1 helix of CD1d. CDR3β, the only hypervariable region of the type-I NKT TCR, which usually mediates antigen specificity together with CDR3α for conventional TCR, did not make any contact with the antigen. Thus, recognition of α-Galcer-CD1d by the type-I NKT TCR is entirely mediated by germline-encoded surface on the type-INKT TCR.

These results were confirmed and extended through an extensive mutational analyses of both mouse and human type-I NKT TCRs (Browne et al., Nat Immunol. 2007; 8: 1105-1113). The results confirmed an energetic ‘hot-spot’ formed by residues within the CDR1α, CDR3α and CDR2β loops of the TCR that were critical for the recognition of the α-GalCer-CD1d complex and provided the basis for the extremely biased TCR repertoire of type-I NKT cells. In the mouse system, this ‘hot-spot’ was similarly required for recognition of structurally different glycolipid antigens such as α-GalCer and iGb3. Because recognition of diverse glycolipid antigens used the same germline-encoded residues, these observations suggest that the type-I NKT TCR functions as a pattern-recognition receptor (Browne et al., Nat Immunol. 2007; 8: 1105-1113). In this way, different NKT cell clones have overlapping antigen specificity despite diversity in the TCRβ chain.

### Activation of Type-I NKT Cells

### Cognate Recognition and Activation of Type-I NKT Cells by Foreign Antigen

Microbial glycolipids presented as cognate antigens that activate type-I NKT cells have been identified. Type-I NKT cells have been shown to directly recognize α-linked glycosphingolipids and diacylglycerol antigens that are expressed by bacteria such as Sphingomonas, Ehrlichia and Borrelia burgdorferi in a CD1d-dependent manner (Mattner J. et al., Nature. 2005 Mar. 24; 434(7032):525-9; Kinjo Y. et al., Nature. 2005 Mar. 24; 434(7032):520-5). The biological response to these glycolipid antigens includes the production of IFNγ and IL-4 by type-I NKT cells.

### Indirect Recognition and Activation of Type-I NKT Cells

Even though no cognate glycolipid antigens that are recognized by type-I NKT cell TCRs have been found in the main Gram-negative and Gram-positive bacterial pathogens that are prominent in human disease, alternative modes of type-I NKT cell activation have been reported for such bacteria. For example, LPS-positive bacteria like Salmonella or Escherichia have been shown to activate type-I NKT cells indirectly. These indirect means of recognition fall into two main groups: those that depend, at least partially, upon CD1d/TCR interactions in conjunction with the activation of antigen presenting cells, and those that appear to be CD1d-independent.

First, it was shown that Gram-negative bacteria (such as Salmonella typhimurium) or Gram-positive bacteria (such as Staphylococcus aureus) cultured with dendritic cells can stimulate type-I NKT cells in absence of specific cognate foreign glycolipids (Mattner J. et al., Nature. 2005 Mar. 24; 434(7032):525-9; Brigl M et al., Nat Immunol. 2003 December; 4(12):1230-7). Such stimulation is blocked by either anti-CD1d or anti-IL-12 mAbs in vitro and in vivo. These results suggest that a vast array of microorganisms might be able to induce type-I NKT activation indirectly through APC stimulation. This mechanism is dependent on TLR engagement of the APC as S. typhimurium-exposed wild-type derived bone marrow-derived dendritic cells (DCs), but not TLR-signaling molecules-deficient DCs, were able to stimulate type-I NKT cells in vitro (Mattner J. et al., Nature. 2005 Mar. 24; 434 (7032): 525-9). It is also likely dependent upon recognition of a self-glycolipid by the type-I NKT TCR because CD1-deficient DCs are unable to stimulate type-I NKT cells when stimulated similarly. Furthermore, APC activation by TLR ligands was shown to modulate the lipid biosynthetic pathway and to induce the specific upregulation of CD1d-bound ligand(s), as demonstrated using multimeric type-I NKT TCRs as a staining reagent (Salio M. et al., Proc Natl Acad Sci USA. 2007; 104: 20490-20495). In contrast with these results, it was reported that Escherichia coli LPS induces the stimulation of type-I NKT cells in an APC-dependent but CD1d-independent manner (Nagarajan N A. et al., J Immunol. 2007; 178:2706-2716). In these experiments, IFNγ-production by type-I NKT cells did not require the CD1d-mediated presentation of an endogeneous antigen, and exposure to a combination of IL-12 and IL-18 was sufficient to activate them.

Finally, it was reported that in addition to the LPS-detecting sensor TLR4, activation of the nucleic acid sensors TLR7 and TLR9 in DCs also leads to the stimulation of type-I NKT cells, as measured by their production of IFNγ (Paget C. et al., Immunity. 2007; 27:597-609).

### Type-I NKT Cells in Disease

Although type-I NKT cells represent a relatively low frequency of peripheral blood T cells in humans, their limited TCR diversity means that they respond at high frequency following activation. As such, type-I NKT cells are uniquely positioned to shape adaptive immune responses and have been demonstrated to play a modulatory role in a wide variety of diseases such as cancer, autoimmunity, inflammatory disorders, tissue transplant-related disorders, and infection (Terabe & Berzofsky, Ch. 8, Adv Cancer Res, 101: 277-348, 2008; Wu & van Kaer, Curr Mol Med, 9: 4-14, 2009; Tessmer et al, Expert Opin Ther Targets, 13: 153-162, 2009). For example, mice deficient in NKT cells are susceptible to the development of chemically induced tumors, whereas wild-type mice are protected (Guerra et al, Immunity 28: 571-80, 2008). These experimental findings correlate with clinical data showing that patients with advanced cancer have decreased type-I NKT cell numbers in peripheral blood (Gilfillan et al, J Exp Med, 205: 2965-73, 2008).

Type-I NKT cells constitute <0.1% of peripheral blood and <1% of bone marrow T cells in humans, but despite their relative scarcity, they exert potent immune regulation via production of IL-2, T_{H}1-type (IFN-γ, TNF-α), T_{H}2-type (IL-4, IL-13), IL-10, and IL-17 cytokines. (Lee et al, J Exp Med, 2002; 195: 637-641; Bendelac et al, Annu Rev Immunol, 2007; 178: 58-66; Burrows et al, Nat Immunol, 2009; 10(7): 669-71). Type-I NKT cells are characterized by a highly restricted (invariant) T-cell receptor (TCR)-Vα chain (Vα24 in humans). Their TCR is unique in that it recognizes altered glycolipids of cell membranes presented in context of a ubiquitous HLA-like molecule, CD1d. (Zajonc & Kronenberg, Immunol Rev, 2009; 230 (1): 188-200). CD1d is expressed at high levels on many epithelial and hematopoietic tissues and on numerous tumor targets, and is known to specifically bind only the type-I NKT TCR. (Borg et al, Nature, 2007, 448: 44-49).

Like NK cells, type-I NKT cells play a major role in tumor immunosurveillance, via direct cytotoxicity mediated through perforin/Granzyme B, Fas/FasL, and TRAIL pathways. (Brutkiewicz & Sriram, Crit Rev Oncol Hematol, 2002; 41: 287-298; Smyth et al, J. Exp. Med. 2002; 191: 661-8; Wilson & Delovitch, Nat Rev Immunol, 2003; 3: 211-222; Molling et al, Clinical Immunology, 2008; 129: 182-194; Smyth et al, J Exp Med, 2005; 201 (12):1973-1985; Godfrey et al, Nat Rev Immunol, 2004, 4: 231-237). In mice, type-I NKT cells protect against GVHD, while enhancing cytotoxicity of many cell populations including NK cells. Unlike NK cells, type-I NKT cells are not known to be inhibited by ligands such as Class I MHC, making them useful adjuncts in settings of tumor escape from NK cytotoxicity via Class I upregulation. (Brutkiewicz & Sriram, Crit Rev Oncol Hematol, 2002; 41: 287-298; Smyth et al, J Exp Med 2002; 191: 661-8; Wilson & Delovitch, Nat Rev Immunol, 2003; 3: 211-222; Molling et al, Clinical Immunology, 2008; 129: 182-194; Smyth et al, J Exp Med, 2005; 201 (12):1973-1985; Godfrey et al, Nat Rev Immunol, 2004, 4: 231-237).

Further evidence supporting a role for type-I NKT cells in antitumor immunity is provided in studies using Jα18 gene-targeted knockout mice that exclusively lack type-I NKT cells (Smyth et al, J Exp Med, 191: 661-668, 2000). For example, type-I NKT-deficient mice exhibited significantly increased susceptibility to methylcholanthrene-induced sarcomas and melanoma tumors, an effect reversed by the administration of liver-derived type-I NKT cells during the early stages of tumor growth (Crowe et al, J Exp Med, 196: 119-127, 2002).

At least one contribution of type-I NKT cells to antitumor immunity occurs indirectly via the activation of type-I NKT cells by DCs. Activated type-I NKT cells can initiate a series of cytokine cascades—including production of interferon gamma (IFN-γ)—that helps boost the priming phase of the antitumor immune response (Terabe &. Berzofsky, Ch 8, Adv Cancer Res, 101: 277-348, 2008). IFN-γ production by type-I NKT cells, as well as NK cells and CD8+ effectors, has been shown to be important in tumor rejection (Smyth et al, Blood, 99: 1259-1266, 2002). The underlying mechanisms are well characterized (Uemura et al, J Imm, 183: 201-208, 2009).

Further, type-I NKT cells have been shown to specifically target the killing of CD1d-positive tumor-associated macrophages (TAMs), a highly plastic subset of inflammatory cells derived from circulating monocytes that perform immunosuppressive functions (Sica & Bronte, J Clin Invest, 117: 1155-1166, 2007). TAMs are known to be a major producer of interleukin-6 (IL-6) that promotes proliferation of many solid tumors, including neuroblastomas and breast and prostate carcinomas (Song et al., J Clin Invest, 119: 1524-1536, 2009; Hong et al, Cancer, 110: 1911-1928, 2007). Direct CD1d-dependent cytotoxic activity of type-I NKT cells against TAMs suggests that important alternative indirect pathways exist by which type-I NKT cells can mediate antitumor immunity, especially against solid tumors that do not express CD1d.

In humans, type-I NKT cells home to neuroblastoma cells (Metelitsa et al, J Exp Med 2004; 199 (9):1213-1221) and B cell targets (Wilson & Delovitch, Nat Rev Immunol 2003; 3: 211-222; Molling et al, Clinical Immunology, 2008; 129: 182-194) both of which express high levels of CD1d. Type-I NKT cell cytokines may increase NK cytotoxicity. IFN-γ enhances NK cell proliferation and direct cytotoxicity, whereas IL-10 potently increases TIA-1, a molecule within NK cytotoxic granules which has direct DNA cleavage effects (Tian et al, Cell, 1991; 67 (3): 629-39) and can regulate mRNA splicing in NK cell targets, favoring expression of membrane-bound Fas on targets. (Izquierdo et al, Mol Cell, 2005; 19 (4): 475-84). IL-10 further enhances tumor target susceptibility to NK lysis by inducing tumor downregulation of Class I MHC, a major inhibitory ligand for NK cells. (Kundu & Fulton, Cell Immunol, 1997; 180:55-61).

Evidence supporting an important role for type-I NKT cells in the treatment of inflammatory diseases and/or autoimmune diseases comes from studies using murine autoimmune disease models. For example, in mouse models of type I diabetes (M. Falcone et al, J Immunol, 172: 5908-5916, 2004; Mizuno et al, J Autoimmun, 23: 293-300, 2004), rheumatoid arthritis (Kaieda et al, Arthritis and Rheumatism, 56: 1836-1845, 2007; Miellot-Gafsou et al, Immunology, 130: 296-306, 2010), autoimmune colitis (Crohn's disease and ulcerative colitis models DSS-induced colitis and autoimmune T cell-mediated colitis; Geremia et al., Autoimmun Rev. 13(1):3-10, 2014 doi: 10.1016/j.autrev.2013.06.004. Epub 2013 Jun. 15. Katsurada et al., PLoS One, 7(9):e44113, 2012; Fuss and Strober, Mucosal Immunol., 1 Suppl 1:S31-3, 2008), and experimental autoimmune encephalitis (EAE) (van de Keere & Tonegawa, J Exp Med, 188: 1875-1882, 1998; Singh et al, J Exp Med, 194:1801-1811, 2001; Miyamoto et al, Nature, 413: 531-534, 2001), type-I NKT cells played key roles in establishing immune tolerance and preventing autoimmune pathology.

Type-I NKT cells are also activated and participate in responses to transplanted tissue. Without subscribing exclusively to any one theory, evidence supports an important role for type-I NKT cells in transplantation-related disorders. For example, type-I NKT cells have been shown to infiltrate both cardiac and skin allografts prior to rejection and have been found in expanded numbers in peripheral lymphoid tissue following transplantation (Maier et al, Nat Med, 7: 557-62, 2001; Oh et al, J Immunol, 174: 2030-6, 2005; Jiang et al, J Immunol, 175: 2051-5, 2005). Type-I NKT cells are not only activated, but also influence the ensuing immune response (Jukes et al, Transplantation, 84: 679-81, 2007). For example, it has been found consistently that animals deficient in either total NKT cells or type-I NKT cells are resistant to the induction of tolerance by co-stimulatory/co-receptor molecule blockade (Seino et al, Proc Natl Acad Sci USA, 98: 2577-81, 2001; Jiang et al, J Immunol, 175: 2051-5, 2005; Jiang et al, Am J Transplant, 7: 1482-90, 2007). Notably, the adoptive transfer of NKT cells into such mice restores tolerance, which is dependent on interferon (IFN)-γ, IL-10 and/or CXCL16 (Seino et al, Proc Natl Acad Sci USA, 98: 2577-81, 2001; Oh et al, J Immunol, 174: 2030-6, 2005; Jiang et al, J Immunol, 175: 2051-5, 2005; Jiang et al, Am J Transplant, 7: 1482-90, 2007; Ikehara et al, J Clin Invest, 105: 1761-7, 2000). In addition, type-I NKT cells have proved to be essential for the induction of tolerance to corneal allografts and have been demonstrated to prevent graft-versus-host disease in an IL-4-dependent manner (Sonoda et al, J Immunol, 168: 2028-34, 2002; Zeng et al, J Exp Med, 189: 1073-81 1999; Pillai et al, Blood. 2009; 113:4458-4467; Leveson-Gower et al, Blood, 117: 3220-9, 2011).

Type-I NKT cell responses may depend on the type of transplant carried out, for example, following either vascularized (heart) or non-vascularized (skin) grafts, as the alloantigen drains to type-I NKT cells residing in the spleen or axillary lymph nodes, respectively. Further, type-I NKT cell responses can be manipulated, for example, by manipulating type-I NKT cells to release IL-10 through multiple injections of α-GalCer, which can prolong skin graft survival (Oh et al, J Immunol, 174: 2030-6, 2005).

Achievement of allogeneic immune tolerance while maintaining graft-versus-tumor (GVT) activity has previously remained an elusive goal of allogeneic hematopoietic cell transplantation (HCT). Immune regulatory cell populations including NKT cells and CD4⁺Foxp3⁺ regulatory T (Treg) cells are thought to play a key role in determining tolerance and GVT. To this end, reduced intensity conditioning methods which enrich for NKT and Treg cells have recently been applied with some measure of success. Specifically, a regimen of total lymphoid irradiation (TLI) and anti-thymocyte globulin (ATG) has resulted in engraftment and protection from graft-versus-host disease (GVHD) in both children and adults (Lowsky et al, New England Journal of Medicine. 2005, 353:1321-1331; Kohrt et al, Blood. 2009; 114:1099-1109; Kohrt et al, European Journal of Immunology. 2010; 40:1862-1869; Pillai et al, Pediatric Transplantation. 2011; 15:628-634) and GVT appeared to be maintained in adult patients whose disease features rendered them at high risk for relapse (Lowsky et al, The New England Journal of Medicine. 2005, 353:1321-1331; Kohrt et al, Blood. 2009; 114:1099-1109; Kohrt et al, European Journal of Immunology. 2010; 40:1862-1869).

Murine pre-clinical modeling of this regimen showed that GVHD protection is dependent upon the IL-4 secretion and regulatory capacity of type-I NKT cells, and that these cells regulate GVHD while maintaining GVT (Pillai et al, Journal of Immunology. 2007; 178:6242-6251). Further, type-I NKT derived IL-4 results can drive the potent in vivo expansion of regulatory CD4⁺CD25⁺Foxp3⁺ Treg cells, which themselves regulate effector CD8⁺ T cells within the donor to prevent lethal acute GVHD (Pillai et al, Blood. 2009; 113:4458-4467). It has been shown that type-I NKT cell-dependent immune deviation results in the development and augmentation of function of regulatory myeloid dendritic cells, which in turn induce the potent in vivo expansion of regulatory CD4+CD25+ Foxp3+ Treg cells and further enhance protection from deleterious T cell responses (van der Merwe et al, J. Immunol., 2013; Nov. 4, 2013).

In response to infection, the immune system relies upon a complex network of signals through the activation of receptors for PAMPs, such as the Toll-like receptors (TLRs) expressed on antigen-presenting cells (APC), consequently promoting antigen-specific T cell responses (Medzhitov & Janeway Jr, Science 296: 298-300, 2002). For example, during such responses, type-I NKT cells respond through the recognition of microbial-derived lipid antigens, or through APC-derived cytokines following TLR ligation, in combination with, and without the presentation of, self- or microbial-derived lipids. Bacterial antigens can also directly stimulate type-I NKT cells when bound to CD1d, acting independently of TLR-mediated activation of APC (Kinjo et al, Nat Immunol, 7: 978-86, 2006; Kinjo et al, Nature, 434:520-5, 2005; Mattner et al, Nature, 434: 525-9, 2005; Wang et al, Proc Natl Acad Sci USA, 107: 1535-40, 2010).

Further, NKT (CD1d−/−) and type-I NKT (Jα18−/−) cell-deficient mice have been shown to be highly susceptible to influenza compared with wild-type mice (De Santo et al, J Clin Invest, 118: 4036-48, 2008). In this model, type-I NKT cells were found to suppress the expansion of myeloid-derived suppressor cells (MDSC) which were expanded in CD1d and Jα18−/− mice (Id.). Importantly, although the exact mechanism of type-I NKT cell activation was not determined, the authors suggest that type-I NKT cells required TCR-CD1d interactions, as the adoptive transfer of type-I NKT cells to Jα18−/− but not CD1d−/− mice suppressed MDSC expansion following infection with PR8 (De Santo et al, J Clin Invest, 118:4036-48, 2008). Thus another application of type-I NKT cells is in augmentation of immune responses to pathogens (e.g., bacterial, viral, protozoal, and helminth pathogens).

Finally, type-I NKT cells have been shown to play a critical role in regulating and/or augmenting the allergic immune response, both through secretion of cytokines and through modulation of other immune subsets including regulatory Foxp3+ cells, APCs, and NK cells (Robinson, J Allergy Clin Immunol., 126(6):1081-91, 2010; Carvalho et al., Parasite Immunol., 28(10):525-34, 2006; Koh et al., Hum Immunol., 71(2):186-91, 2010). This includes evidence in atopic dermatitis models (Simon et al., Allergy, 64(11):1681-4, 2009).

However, a major obstacle to application of human innate regulatory type-I NKT cells in immunotherapy is their relative scarcity in common cellular therapy cell products including human peripheral blood (Berzins et al, Nature Reviews Immunology. 2011; 11:131-142; Exley et al, Current Protocols in Immunology, 2010; Chapter 14: Unit 14-11; Exley & Nakayama, Clinical Immunology, 2011; 140:117-118) and the lack of clear phenotypic and functional data on ex vivo expanded human type-I NKT cells to validate the potential application of post-expansion human type-I NKT cells therapeutically.

### Passive Immunization

The term “passive immunization” as used herein refers to the production of passive immunity, meaning immunity acquired from transfer of antibodies either naturally, as from mother to fetus, or by intentional inoculation (artificial passive immunity). Passive immunity can be induced by either natural or artificial mechanisms. Where antibodies are transferred, the passive immunity, with respect to the particular antibodies transferred, is specific.

### Convalescent Plasma and COVID-19

In COVID-19 infection, convalescent plasma has been tested only in small trials without the statistical power to provide firm conclusions of efficacy. The idea is that plasma contains antibodies, some of which might have helped the donor to recover from their infection, and proteins involved in regulating immune responses. However, such plasma varies widely in antibody concentration. As part of the US FDA expanded access program for COVID 19 convalescent plasma, key safety metrics were analyzed in a study of 500 patients; early indicators suggested that transfusion of convalescent plasma is safe in hospitalized patients with COVID-19. [Joyner, M J, et al. J. Clin. Invest. (2020) doi 10.1172/JC1140200]

### Antibody Cocktails

Regeneron's REGN-COV2, an antibody cocktail containing two noncompeting, neutralizing human IgG1 antibodies that target the receptor-binding domain (RBD) of the SARS-CoV-2 spike protein, thereby preventing viral entry into human cells through the angiotensin-converting enzyme 2 (ACE2) receptor, is being studied in an ongoing double-blind phase 1-3 trial involving nonhospitalized patients with COVID-19 to determine whether reducing viral burden leads to clinical benefit. [Weinreich, D M et al. N. Engl. J. Med. (2021) 384: 238-51; citing Baum, A. et al. Science (2020) 369: 1014-181 Hansen, J. et al. Science (2020) 369: 1010-141. The “cocktail” approach is being pursued because of previous experience with the emergence of treatment-resistant mutant virus when a single antibody, suptavumab, was used to target respiratory syncytial virus. [Id., citing Simoes, E A F et al. Clin. Infect. Dis. 2020 September 81. Preclinical studies confirmed that the REGN-COV2 cocktail protects against the rapid emergence of such mutants seen with either single antibody. [Id., citing Baum, A. et al. Science (2020) 369: 1014-181. In vivo studies in nonhuman primates showed profound antiviral activity of REGN-COV2 in reducing viral load when given in a prophylactic context and in improving viral clearance when given in a therapeutic context.[Id., citing Baum, A. et al. Science (2020) 370: 1110-151.

Lilly has reported that bamlanivimab (LY-CoV555) 2800 mg and etesevimab (LY-CoVO16) 2800 mg together significantly reduced COVID-19-related hospitalizations and deaths (collectively, “events”) in high-risk patients recently diagnosed with COVID-19, meeting the primary endpoint of the Phase 3 BLAZE-1 trial. [https:/investor.lilly.com/news-releases/news-release-details/new-data-show-treatment-lillys-neutralizing-antibodies, visited 15 Mar. 20211. Bamlanivimab is a recombinant, neutralizing human IgG1 monoclonal antibody (mAb) directed against the spike protein of SARS-CoV-2. It is designed to block viral attachment and entry into human cells, thus neutralizing the virus, potentially treating COVID-19. Bamlanivimab emerged from a collaboration between Lilly and AbCellera to create antibody therapies for the prevention and treatment of COVID-19. Etesevimab (LY-CoV016, also known as JS016) is a recombinant fully human monoclonal neutralizing antibody, which specifically binds to the SARS-CoV-2 surface spike protein receptor binding domain (RBD) with high affinity and can block the binding of the virus to the ACE2 host cell surface receptor. Point mutations were introduced into the native human IgG1 antibody to mitigate effector function. Lilly licensed etesevimab from Junshi Biosciences after it was jointly developed by Junshi Biosciences and Institute of Microbiology, Chinese Academy of Science (IMCAS).

The US FDA has ordered both Regeneron and Eli Lilly to monitor new variants of SARSCoV-2 and conduct additional tests of their antibody cocktails against these variants, in an expression of concern that emerging variants can pose a threat of reduced antibody potency. (https://www.fiercepharmia.com/pharma/fda-revises-covid-19-euas-requiring-regeneron-lilly-to-monitor-efficacy-against-coronavirus?mkt_tok=Mjk0LU1RRi0wNTYAAAF727ldR_IRI11ilsjWUAoZU6A6Lbor6FQ6 uh07v1DrdkM-1QYYzQEBhukmzMiZmqVzPTlwBaaTgbHq8Cij0SCRKnLNcuDxr4oAUYzgtHOdmWiK-YdYJTg&mrkid=49260466, Mar. 16, 2021).

### Passive Cell-Mediated Immunity

Passive cell-mediated immunity (also referred to as adoptive immunity or acquired immunity) is produced by the transfer of living lymphoid cells from an immune cell source.

Cells like invariant NKT cells are attractive candidates for adoptive immunity, because of their ability to rapidly and robustly produce cytokines and chemotoxins, their ability to kill other cells, their ability to influence the behavior of other immune cells, e.g., APCs, NK cells, CD4+ T cells, CD8+ T cells, macrophages, B cells, myeloid dendritic cells and neutrophils, their responsiveness at high frequency upon activation while preventing autoimmune pathology, and their potential to augment an immune response against invaders by suppressing expansion of myeloid derived suppressor cells without invoking a GvHD response.

iNKT Adoptive Therapy in Cancer

Adoptive transfer of activated iNKT cells to restore iNKT cell numbers and potentially iNKT cell function in cancer patients has been tested in preclinical models of melanoma and lung cancer and shown to be more effective than the i.v. administration of a-GalCer [Wolf, B J et al. Front. Immunol. (2018) 9: 384, citing 50]. Trials of iNKT-enriched PBMC have supported direct use of iNKT with evidence for immunological and objective clinical responses [Id., citing Motohashi, S. et al. Clin. Cancer Res. (2006) 12: 6079-86; Kunii, N. et al. Cancer Sci. (2009) 100 (6): 1092-8; Yamasaki, K. et al. Clin. Immunol. (2011) 138 (3): 255-65; Exley, M A et al. Clin. Cancer Res. (2017) 23 (14): 3510-9].

The first of these adoptive iNKT cell therapies targeted six patients with non-small cell lung cancer [Id., citing Motohashi, S. et al. Clin. Cancer Res. (2006) 12: 6079-86]. To grow out iNKT cells, bulk PBMCs were stimulated two to three times via addition of a-GalCer to the cultured cells. These iNKT cell-enriched products were then infused back into the patient, and the iNKT cell numbers, persistence, and phenotype were measured. In most patients, there was a transient but not long-term increase in iNKT cell number within the blood, which coincided with the ability to detect IFNγ production ex vivo via α-GalCer stimulation of PBMCs. Only minor adverse effects were seen in this first trial, demonstrating that adoptive cell therapy of iNKT cells is likely to be safe. No partial or complete responses were seen [Id., citing Motohashi, S. et al. Clin. Cancer Res. (2006) 12: 6079-86].

The next adoptive iNKT cell-based therapy studies combined autologous iNKT cell-enriched product with in vivo boosting. In a Phase I and subsequent Phase II study, the trial group first treated head and neck squamous cell carcinoma (HNSCC) patients with two doses of α-GalCer-loaded DCs followed by an iNKT cell infusion [Id., citing Kunii, N. et al. Cancer Sci. (2009) 100 (6): 1092-8]. In the Phase I trial, three patients showed partial responses, four had stable disease, and one had progressive disease Kunii, N. et al. Cancer Sci. (2009) 100 (6): 1092-8. Of the eight patients, only one had grade 3 adverse events and that patient also had a partial response: a fistula formed within the tumor apparently due to rapid tumor killing [Kunii, N. et al. Cancer Sci. (2009) 100 (6): 1092-8]. In the follow-up Phase II trial for 10 patients with HNSCC, patients were first given nasal submucosal administration of α-GalCer loaded DCs followed by iNKT cell infusion directly into the tumor-feeding arteries, so that iNKT cells were more likely to end up in the tumor site Id., citing Yamasaki, K. et al. Clin. Immunol. (2011) 138 (3): 255-65]. Adverse events were minimal and limited to grade 2 or below, five patients had a partial response, and five patients had stable disease [Id., citing Yamasaki, K. et al. Clin. Immunol. (2011) 138 (3): 255-65]. iNKT cell numbers within the tumor and in the peripheral blood were measured, and while iNKT cell numbers in the blood did increase in 9 of 10 patients post-treatment, this did not correlate with outcome. Instead, a high number of tumor-infiltrating iNKT cells correlated with an objective response of patients [Id., citing Yamasaki, K. et al. Clin. Immunol. (2011) 138 (3): 255-65].

A Phase I clinical trial of autologous purified [with the iNKTCR mAb 6B11; Id., citing Exley, M A et al. Clin. Cancer Res. (2017) 23 (14): 3510-9] and expanded iNKT cells was performed in nine melanoma cancer patients [Id., citing Exley, M A et al. Clin. Cancer Res. (2017) 23 (14): 3510-9]. iNKT cells were isolated from PBMCs with a protocol based on a monoclonal antibody that specifically recognizes the invariant TCR of iNKT cells and then expanded in vitro with plate bound anti-CD3 antibody [Id., citing Exley, M A et al. Clin. Cancer Res. (2017) 23 (14): 3510-9; Exley, M A et al. Eur. J. Imunol. (2008) 20: 1756; Exley, M A et al. Curr. Protoc. Immunol. (2017) 119: 1-14]. Compared to previous studies using α-GalCer stimulated PBMCs as a source of iNKT cells [Motohashi, S. et al. Clin. Cancer Res. (2006) 12: 6079-86; Kunii, N. et al. Cancer Sci. (2009) 100 (6): 1092-8; Yamasaki, K. et al. Clin. Immunol. (2011) 138 (3): 255-65], this study transferred in generally higher purity and/or larger numbers of iNKT cells (3 doses at up to 250 million iNKT cells per dose). Since iNKT cells are activated via interaction with CD1d on APC, after the first three patients had no significant toxicities, subsequent patients were pre-treated with GM-CSF to enhance DC functions before iNKT infusion cycles 2 and 3. Like in the other studies, a transient increase in circulating iNKT cell numbers following adoptive cell transfer and increased activation of other T cells and myeloid cells in some patients was noted; and toxicities were minor and readily treatable (Grade 1 & 2 only) [Exley, M A et al. Clin. Cancer Res. (2017) 23 (14): 3510-9]. At the end of the study, three patients had no evidence of disease or stable disease, three eventually progressed and responded to subsequent treatment, and three died of disease (one removed from study after infusions, two at 2 or more years post-treatment). Overall, the trial confirmed that iNKT cell adoptive therapy is safe and well-tolerated.

Phase I clinical trials of iNKT based immunotherapy conducted for non-small cell lung cancer, and head and neck cancer have been reported. Eleven patients with stage IV or recurrent NSCLC were enrolled in the study and 9 completed the treatment. Safety profiles of α-GalCer pulsed APCs were examined at level 1 (5×10⁷; Level 2: 2.5×10⁸; and level 3: 1×10⁹ cells/m2. Patients received four i.v. injections of the α-GalCer pulsed APCs over 3 months. While objective clinical responses were not observed in all cases, patients who received the level 3 does exhibited iNKT cell expansion in the periphery and showed long term survival for over one year. In a phase I-II clinical trial, 23 patients were enrolled and 17 completed the protocol treatment. Patients were either stage IIIB, stage IV or recurrent NSCLC patients who received standard therapy. All patients received two courses of α-GalCer-pulsed APCs with four injections of 1×10⁹ cells. 10 patients had a greater than two-fold increase of IFN-γ producing cells in the periphery after administration (good responders) whereas 7 patients showed mild or no increase of IFN-γ producing cells (poor responders). The increase of IFN-γ producing cells in the periphery correlated with the median survival time; good responders showed a longer MST compared with poor responders. A third clinical trial targeted four patients diagnosed as stage IIB and IIIA NSCLC who underwent surgical treatment compared with 6 patient controls. Patients received as single i.v. injection of 1×10⁹ α-Gal Cer-pulsed APCs seven days prior to surgery and characteristic tumor infiltration was analyzed from surgically removed tumor tissue specimens. The systemic administration of α-GalCer pulsed APCs led to local iNKT cell accumulation in the tumor microenvironment and induced immune responses by producing IFN-γ. Lastly the administration of in vitro expanded NKT cells was performed as a phase I clinical trial in 6 patients with recurrent lung cancer. iNKT cells were prepared in vitro from PBMCs cultured in the presence of α-GalCer and IL-2. In vitro expanded iNKT cells (level 1: 1×10⁷ cells; level 2: 5×10⁷ cells per injection) were transferred to patients i.v. iNKT cells derived from patients in this study expanded and produced T_{H}1 dominant cytokines including IFNγ along with tumoricidal activity ex vivo. In cases of advanced head and neck squamous cell carcinoma, the increased accumulation of iNKT cells in the tumor microenvironment was correlated with objective clinical responses. [Takami, M. et al. Front. Immunol. (2018) 9: 2021].

iNKT Adoptive Therapy in Infectious Disease

The natural role of iNKT cells in antiviral immunity and in the control of viral replication and pathology has been studied using Jα18^{-/-} a mice, which lack iNKT cells. It was reported that the role of iNKT cells during experimental viral infection can vary according to the virus and experimental conditions. [Paget, C. et al., J. Immunol. (2011) 186: 5590-5602, citing Diana, J. and Lehuen, A. Eur. J. Immunol. (2009) 39: 3283-91; Tessmer, M. et al. Expert Opin. Ther. Targets (2009) 13: 153-62]. For example, during HSV type 1 and 2 and lymphocytic choriomeningitis virus infections iNKT cells play a positive role in the antiviral immune responses and virus-associated pathology [Id., citing Ashkar, A. A. and Rosenthal, K L. J. Virol. (2003) 10168-71; Diana, J. et al. Immunity (2009) 30: 289-99; Grubor-Bauk, B. et al. (2008) J. Virol. (2008) J. Immunol. 170: 1430-34], whereas they appear to be deleterious during Sendai virus [Id., citing Kim, E Y et al. Nat. Med. (2008) 14: 633-408], HSV type 2 (only in aged mice, Id., citing Stout-Delgado, H W, et al. Cell Host Microbe (2009) 6: 446-56) and Dengue virus serotype 2. Studies using CD1d-deficient mice, which not only lack iNKT cells but also non-iNKT cells, have suggested that NKT cells positively contribute to the immune response to respiratory syncytial virus [Id., citing Johnson, T R et al. J. Virol. (2002) 76: 4294-4303], encephalomyocarditis virus [Id., citing Exley, M A et al. Immunology 110: 519-26], murine CMV [Id., citing Wesley, J D et al. PLoS Pathog. (2008) 4: e10000106], and Theiler's murine encephalomyocarditis virus [Id., citing Tsunoda, I. et al. J. Virol. (2008) 82: 10279-89], while being deleterious during coxsackie virus infection [Id., citing Huber, S. et al. J. Immunol. (2003) 170: 3147-53]. Although suspected [Id., citing Levy, O. et al. J. Infect. Dis. (2003) 188: 948-53; Nichols, K E et al. Nat. Med. (2005) 11: 340-45; Rigaud, S. et al. Nature (2006) 444: 110-14]], the role of iNKT cells in human viral infections is not entirely clear.

As shown in an in vivo mouse model, iNKT cells play a role in the control of pneumonia as well as in the development of the CD8+ T cell response during the early stage of acute influenza A virus (IAV) H3N2 infection. A virulent, mouse adapted IACH3N2 strain was used to infect C57BL/6 mice deficient in iNKT cells (Jα18^{-/-} mice). Upon infection with a lethal dose of the virus, iNKT cells became activated in the lungs and bronchoalveolar space to become rapidly anergic to further re-stimulation [see Id., citing 43-45]. Relative to wide type animals, the Jα18^{-/-} a mice developed a more severe bronchopneumonia and had an accelerated fatal outcome; this was reversed by the adoptive transfer of 1×10⁶ NKT cells prior to infection. For adoptive transfer experiments, and to prevent activation of iNKT cells, NKT cells were purified from the livers of naïve animals using CD5 (allophycocyanin conjugated) and NK1.1 (PE-conjugated antibodies (BD Pharmingen), but not on the basis of PPBS-57 glycolipid-loaded Cd1d tetramer and TCRβ or CD3 staining; labeled cells were isolated using a FACSAria and BDFACSDiva software. The enhanced pathology was not associated with either reduced or delayed viral clearance in the lungs or with a defective local NK cell response. Jα18^{-/-} mice displayed a dramatically reduced virus-specific CD8+ T cell response in the lungs and in lung-draining mediastinal lymph nodes. This defective CD8+ T response correlates with an altered accumulation and maturation of pulmonary CD103+, but not CD11b^{high}, dendritic cells in the mediastinal lymph nodes.

The present disclosure provides a cell product comprising superactivated cytokine killer cells that, when administered to a subject in need, supplements the body's natural response either directly by killing of virus infected cells or indirectly by mobilizing other immune cells to fight against the viral infection. It also provides a method for effective control of a viral infection through adoptive transfer of superactivated cytokine killer cells.

### SUMMARY OF THE INVENTION

According to one aspect, the present disclosure provides a method for treating a viral infection in a recipient subject suffering from or at risk of the viral infection comprising: administering to the recipient subject a pharmaceutical composition comprising a cell product containing a therapeutic amount of superactivated cytokine killer T cells (SCKTCs) and a pharmaceutically acceptable carrier, and mobilizing an immune response of the recipient subject to the viral pathogen; wherein the therapeutic amount is at least 0.2×109 SCKTCs per 30 day treatment cycle; and wherein when tested in vitro, the SCKTCs predominantly produce TH1 dominant cytokines including IFN-γ; or an IFN-γ:IL-4 ratio of the SCKTC population when tested in vitro is at least 500: 1 with IL-12 stimulation; and at an effector:target ratio of 20:1, cytotoxicity against A549 target cells is ≥50%.

According to some embodiments of the method, the immune response of the recipient subject comprises stimulating activation of one or more immune cell population of the recipient subject. According to some embodiments the immune cell population of the recipient subject comprises one or more of a dendritic cell population; a CD8+ T cell population; an NK cell population; or an MHC-restricted T cell population. According to some embodiments the MHC-restricted T cell population comprises an invariant NKT population. According to some embodiments the therapeutic amount stimulates an effector function of the immune cells of the recipient subject. According to some embodiments the effector function includes one or more of cytokine secretion, cytotoxicity, or antibody-mediated clearance of the pathogen. According to some embodiments the viral infection is characterized by virus-infected cells. According to some embodiments the therapeutic amount destroys virus-infected cells through direct lysis, by effecting destruction of the infected cells indirectly or both. According to some embodiments, destruction of the infected cells indirectly comprises mobilizing attracting cell cytotoxicity agents through secretion of cytokines. According to some embodiments the virus infection is an infection with a respiratory virus. According to some embodiments the respiratory virus is a respiratory syncytial virus (RSV), an Ebola virus, a cytomegalovirus, a Hanta virus, an influenza virus, a coronavirus, a Zika virus, a West Nile virus, a dengue virus, a Japanese encephalitis virus, a tick-borne encephalitis virus, a yellow fever virus, a rhinovirus, an adenovirus, a herpes virus, an Epstein Barr virus, a measles virus, a mumps virus, a rotavirus, a coxsackie virus, a norovirus, or an encephalomyocarditis virus (EMCV). According to some embodiments the coronavirus is SARS-CoV-1, SARS-CoV-2 or MERS. According to some embodiments, the therapeutic amount reduces risk of the virus infection; or the therapeutic amount reduces signs, symptoms, or both signs and symptoms of the viral infection; or the therapeutic amount reduces extent of the viral infection where symptoms are not yet clinically recognized; or the therapeutic amount reduces worsening or progression of the viral infection; or the therapeutic amount reduces severity of the viral infection, compared to an untreated subject; or the therapeutic amount improves progression-free survival; or the therapeutic amount improves overall survival. According to some embodiments, the superactivated cytokine killer T cells (SCKTCs) are derived from blood; or the SCKTCs are derived from a leukapheresis; or the SCKTCs are derived from hematopoietic stem cells; or the SCKTCs are derived from hematopoietic stem cells derived from adult bone marrow, umbilical cord, umbilical cord blood, placental tissue or fetal liver.

According to some embodiments the pharmaceutical composition further comprises an enriched differentiated and expanded population of NK cells. According to some embodiments the population of SCKTCs is autologous to the recipient subject. According to some embodiments the population of SCKTCs is allogeneic to the recipient subject. According to some embodiments the NK cells are derived from CD34+ hematopoietic stem cells of a donor. According to some embodiments the population of NK cells of the donor is autologous to the recipient subject. According to some embodiments the population of NK cells of the donor is allogeneic to the recipient subject. According to some embodiments the population of NK cells is depleted of CD3+ T cells, CD19 B cells or both.

According to some embodiments, the method further comprises administering the pharmaceutical composition comprising the cell product containing the population of SCKTCs with a supportive therapy or an additional compatible therapeutic agent. According to some embodiments the supportive therapy reduces viral load. According to some embodiments the additional compatible therapeutic agent is one or more of an immunomodulatory agent, an anti-inflammatory agent, an anti-infective agent, an anti-malarial agent, an anti-viral agent or an anti-fibrotic agent. According to some embodiments, the immunomodulatory agent comprises one or more of methotrexate; a glucocorticoid, cyclosporine, tacrolimus and sirolimus; a recombinant interferon selected from IFN-α; IFN-α-2b, IFN-β, IFN-γ, IFN-κ, IFN-ω; a recombinant IL-2 receptor inhibitor; a PDE4 inhibitor; a hyperimmune globulin prepared from a donor with high titers of a desired antibody; a TNFα inhibitor/antagonist; an IL-1β inhibitor; a chimeric IL-1Ra; an IL-6 inhibitor; an IL-12/IL-23 inhibitor selected from ustekinumab, briakinumab; an IL-23 inhibitor selected from guselkumab, tildrakizumab; a compound that targets TLR4 signaling; a p38 MAPK inhibitor, a Janus kinase signaling inhibitor; a compound that targets cell adhesion molecules to reduce leukocyte recruitment; a checkpoint inhibitor, or a recombinant anti-inflammatory cytokine; or the anti-infective agent is amoxicillin, doxycycline, demeclocycline; eravacycline, minocycline, ormadacycline, tetracycline, cephalexin, defotaxime, cetazidime, cefuroxime, ceftaroline; ciprofloxacin, levofloxacin, moxifloxacin, clindamycin, lincomycin, metronidazole, azithromycin; clarithromycin, erythromycin, sulfamethoxazle and trimethoprim; sulfasalazine, amoxicillin and clavulanate; vancomycin, dalbavancin, oritavancin, telavancin, gentamycin, tobramycin, amikacin, imipenem and cilastatin, meropenem, doripenem, or ertapenem; or the anti-viral agent is selected from acyclovir, gancidovir, foscamet; ribavirin; amantadine, azidodeoxythymidine/zidovudine), nevirapine, a tetrahydroimidazobenzodiazepinone (TIBO) compound; efavirenz; remdecivir, lopinavir/ritonavir, umifenovir, favipiravir, ivermectin, and delavirdine; or the anti-fibrotic agent is selected from nintedanib, pirfenidone, and combinations thereof. According to some embodiments, the immunomodulatory agent comprises recombinant IL-37, recombinant CD24, or both. According to some embodiments, the anti-viral agent is an agent that inhibits viral entry and decreases viral load. According to some embodiments the checkpoint inhibitor is YERVOY™ (Ipilimumab; CTLA-4 antagonist), OPDIVO™ (Nivolumab; PD-1 antagonist) or KEYTRUDA™ (Pembrolizumab; PD-1 antagonist).

According to another aspect, the present disclosure provides a method for preparing a pharmaceutical composition comprising an enriched population of superactivated cytokine killer T cells (SCKTCs) comprising, in order (a) isolating a population of mononuclear cells (MCs) comprising a population of cytokine killer T cells (CKTCs); (b) transporting the preparation of (a) to a processing facility under sterile conditions; (c) on day 0, placing the population of MCs in a suspension culture system comprising a serum-free culture medium; (d) on day 6, contacting the culture system of step (c) with the serum-free culture medium containing IL-2 and IL-7, wherein the contacting stimulates CKTC activation; (e) on day 7, pulsing the CKTCs of step (d) with an enriched population of CD1d− expressing antigen presenting cells (APCs) derived from the MCs in (a) loaded with α-GalCer; (f) replenishing the serum-free culture medium every 1-3 days from day 7 to day 14; (g) on day 14, adding CD1d expressing APCs loaded with α-GalCer; (h) replenishing the serum-free culture medium of the cells every 1-3 days; (i) on day 14+7 days, replenishing the culture medium of the culture and pulsing with CD1d expressing APCs loaded with α-GalCer; (j) on day 14+14 days, a replenishing the culture medium of the culture and pulsing with CD1d-expressing APCs loaded with α-GalCer; (k) on day 14+21 days, replenishing the culture medium of the culture and adding IL-12; (l) on Day 14+22 harvesting the amplified enriched superactivated population of SCKTCs from the culture system to form a SCKTC cell product; and (m) filling and finishing the SCKTC cell product into a container; and (n) optionally cryopreserving the SCKTC cell product in the vapor phase of a liquid nitrogen freezer in a serum-free cryo freezing medium. According to some embodiments of the method, the population of MCs comprising the population of CKTCs: is derived from hematopoietic stem cells derived from adult bone marrow, umbilical cord, umbilical cord blood, placental tissue, or fetal liver; or is derived from leukapheresis of a donor subject allogeneic to a recipient subject; or is derived from leukapheresis of a donor subject autologous to a recipient subject.

According to some embodiments, in step (a) frequency of the population of CKTCs from the donor represents <0.5% of the total MNC population. According to some embodiments the population of MCs comprises subpopulations of T lymphocytes, NK cells, B lymphocytes, and monocytes. According to some embodiments the subpopulation of T lymphocytes comprises NKT cells, CD4+ T cells, and CD8+ T cells. According to some embodiments, the CD1d− expressing antigen presenting cells (APCs) derived from the MCs comprises CD14+ monocytes; or the CD1d− expressing antigen presenting cells (APCs) derived from the MCs comprise an irradiated population of PBMCs. According to some embodiments the CD1d-expressing population of APCs loaded with alpha-GalCer is a population of monocyte-derived dendritic cells. According to some embodiments at least 30% of the monocyte derived population of DCs constitutively expresses CD1d. According to some embodiments the pulsing steps with DCs loaded with alpha-GalCer achieve at least an 80% pure population of SCKTCs without positive or negative cell separation methods. According to some embodiments the population of dendritic cells loaded with αGalCer is prepared by a method comprising: (i) isolating a population of mononuclear cells (MCs) comprising CD14+ monocytes; (ii) inducing differentiation of the CD14+ monocytes into dendritic cells by culturing the population of CD14+ monocytes in a culture system; and (iii) contacting the culture system with αGalCer, wherein the contacting is sufficient to load the monocyte-derived dendritic cells with αGalCer. According to some embodiments minimum acceptable specifications of the SCKTC cell product when tested in vitro include: (i) cytokine production comprising IL-4 low, IL-5 low, IL-6 low, IL-10 low, IFNγ high, and (ii) a ratio of IFN-γ:IL-4 in culture supernatants of at least 500: 1; and (iii) at an effector:target cell ratio of 20:1 greater than or equal to 50% cytotoxicity against A549 cells; and (iv) a therapeutic dose of the cell product per treatment cycle of 30 days comprising about 0.2×10⁹ activated SCKTCs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic showing the three pathways for complement activation.

FIG. 2A show a flow chart depicting process flow in tissue culture flasks for stimulation of superactivated cytokine killer cells in Run 5. FIG. 2B shows a flow chart depicting the process flow for dendritic cell culture in Run 5.

FIG. 3 shows representative morphology of the Run 5 cultures at day 7, day 10, day 12, day 14, day 18 and day 20. Cell morphology shows obvious cell colonies beginning from day 7.

FIG. 4 shows representative growth curves of total viable cells vs. days in culture for Run 5. After 21 days in culture total number of cells is about 1.5×10⁹.

FIGS. 5A-5D show forward (FSC) and side scatter (SSC) plots for size and granularity from multicolor flow cytometry experiments for Run 5 on day 20 for cell identity. Fresh cultured Run 5 day 20 cells were used for staining. FIG. 5A shows FSC/SSC plot of the total cell population. FIG. 5B shows Vβ11 v. Vα24; FIG. 5C shows CD8 v. CD4; Gating was on Vα24+Vβ11+ cells; FIG. 5D shows CD56 v. CD3. In this culture, SCKTC purity achieved was about 81.6% of total viable cells.

FIGS. 6A-6C show representative bar graphs depicting cytokine production of Run 5 cultures plotting concentration in culture supernatant (pg/ml), y-axis for IFN-γ (FIG. 6A), IL-4 (FIG. 6B) and the ratio of IFNγ to IL-4 (FIG. 6C). Cytokines were measured by Cytometric Bead Array (CBA) assay [BD, Human IFNγ Flex Set; Human IL-4 Flex Set]. The data show that IL-12 can strongly stimulate IFN-γ secretion, while having no obvious effect on IL-4 secretion.

FIG. 7 shows in vitro cytotoxicity of Run 5 cultures on A549 cells. Cytotoxicity was determined by LDH cytotoxicity assay kit (Dojindo Molecular Technologies #CK12-05). The results show that IL-12 stimulation can slightly increase in vitro cytotoxicity of SCKTCs on A549 target cells.

FIG. 8A shows a flow chart depicting process flow for stimulation of superactivated cytokine killer cells in Run 14 using gas-permeable immobile tissue culture bags. FIG. 8B shows a flow chart depicting the process flow for dendritic cell culture in Run 14.

FIG. 9 shows a representative growth curve of total viable cells vs. days in culture for an aliquot cell culture of the Run 14 supercell cultures. On day 14+22, the total viable cell number is about 1.68×10¹⁰.

FIGS. 10A-10D show forward (FSC) and side scatter (SSC) plots for size and granularity from multicolor flow cytometry experiments for cell identity of the Run 14 supercell cultures. Fresh cultured cells were used for staining of cells on day 14+22. FIG. 10A shows an FSC/SSC plot of the total cell population. FIG. 10B shows Vβ11 v. Vα24; FIG. 10C shows CD8 v. CD4; Gating was on Vα24+Vβ11+ cells; FIG. 10D shows CD56 v. CD3. SCKTC purity achieved was about 92.5% of total viable cells.

FIGS. 11A-11F show representative bar graphs depicting cytokine production by the Run 14 supercell cultures. Row 1 shows supercell stimulation with IL-12. Row 2 shows super-cell stimulation with DCs. The bar graphs plot concentration in culture supernatant (pg/ml), y-axis for IFN-γ (FIG. 11A, FIG. 11D), IL-4 (FIG. 11B, FIG. 11E) and the ratio of IFNγ to IL-4 (FIG. 11C, FIG. 11F). Cytokines were measured by Cytometric Bead Array (CBA) assay [BD, Human IFNγ Flex Set; Human IL-4 Flex Set]. The results show that both IL-12 (FIG. 11A) and DCs (FIG. 11D) can strongly stimulate IFNγ secretion. As for IL-4, IL-12 stimulated IL-4 secretion (FIG. 11B) and increased the ratio of IFNγ/IL-4 (FIG. 11C). While DCs could also robustly stimulate IL-4 secretion (FIG. 11E), this stimulation caused a decrease in the ratio of IFNγ/IL-4 (FIG. 11F).

FIGS. 12A-12B show representative bar graphs depicting in vitro cytotoxicity of the Run 14 supercell cultures on A549 target cells. FIG. 12A shows cytotoxicity with and without IL-12 stimulation at an effector:target cell ratio of (from left to right) 5:1, 10:1, and 20:1. FIG. 12B shows cytotoxicity of Run 14 supercell cultures on A549 target cells comparing SCKTCs only (SCKTC:A549 cell ratio: 10:1), DCs only (DC:A549 cell ratio, 1:1), and DC-stimulated SCKTCs+ DCs (SCKTC:DC=10:1). Cytotoxicity was determined by an LDH cytotoxicity assay kit (Dojindo Molecular Technologies (#CK12-05). The results show that both IL-12 and DC stimulation can strongly activate in vitro cytotoxicity of SCKTCs on target A549 cells.

FIG. 13 is a schematic illustrating that IL-37 is a dual function cytokine with intracellular/endogenous activity and extracellular/exogenous activity. Taken from Cavallli, G. and Dinarello, C A. Immunological Reviews (2018) 281 (1): 179-90.

### DETAILED DESCRIPTION OF THE INVENTION

### Definitions

As used in the specification and the appended claims, the singular forms “a,” “an” and “the” include plural referents unless the context clearly dictates otherwise.

The term “about” as used herein when referring to a measurable value such as an amount, a temporal duration, and the like, is meant to encompass variations of ±20%, ±10%, ±5%, ±1%, ±0.9%, ±0.8%, ±0.7%, ±0.6%, ±0.5%, ±0.4%, ±0.3%, ±0.2% or ±0.1% from the specified value, as such variations are appropriate to perform the disclosed methods.

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer. According to some embodiments, to A without B (optionally including elements other than B); According to some embodiments, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, “either,” “one of,” “only one of,” or “exactly one of” “Consisting essentially of,” when used in the claims, shall have its ordinary meaning as used in the field of patent law.

As used herein, the phrase “integer from X to Y” means any integer that includes the endpoints. That is, where a range is disclosed, each integer in the range including the endpoints is disclosed. For example, the phrase “integer from X to Y” discloses 1, 2, 3, 4, or 5 as well as the range 1 to 5.

As used herein, when used to define products, compositions and methods, the term “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are open-ended and do not exclude additional, unrecited elements or method steps. Thus, a polypeptide “comprises” an amino acid sequence when the amino acid sequence might be part of the final amino acid sequence of the polypeptide. Such a polypeptide can have up to several hundred additional amino acids residues (e.g. tag and targeting peptides as mentioned herein). “Consisting essentially of” means excluding other components or steps of any essential significance. Thus, a composition consisting essentially of the recited components would not exclude trace contaminants and pharmaceutically acceptable carriers. A polypeptide “consists essentially of” an amino acid sequence when such an amino acid sequence is present with eventually only a few additional amino acid residues. “Consisting of” means excluding more than trace elements of other components or steps. For example, a polypeptide “consists of” an amino acid sequence when the polypeptide does not contain any amino acids but the recited amino acid sequence.

As used herein, “substantially equal” means within a range known to be correlated to an abnormal or normal range at a given measured metric. For example, if a control sample is from a diseased patient, substantially equal is within an abnormal range. If a control sample is from a patient known not to have the condition being tested, substantially equal is within a normal range for that given metric.

The terms “activate,” “stimulate,” “enhance” “increase” and/or “induce” (and like terms) are used interchangeably to generally refer to the act of improving or increasing, either directly or indirectly, a concentration, level, function, activity, or behavior relative to the natural, expected, or average, or relative to a control condition. “Activate” refers to a primary response induced by ligation of a cell surface moiety. For example, in the context of receptors, such stimulation entails the ligation of a receptor and a subsequent signal transduction event. Further, the stimulation event may activate a cell and upregulate or downregulate expression or secretion of a molecule. Thus, ligation of cell surface moieties, even in the absence of a direct signal transduction event, may result in the reorganization of cytoskeletal structures, or in the coalescing of cell surface moieties, each of which could serve to enhance, modify, or alter subsequent cellular responses.

As used herein, the terms “activating or activated cytokine killer T cells” or “CKTC1 activation” is meant to refer to a process causing or resulting in one or more cellular responses of CKTCs, including: proliferation, differentiation, cytokine secretion, cytotoxic effector molecule release, cytotoxic activity, and expression of activation markers. As used herein, an “activated cytokine killer T cell” refers to a cytokine killer T cell that has received an activating signal, and thus demonstrates one or more cellular responses, including proliferation, differentiation, cytokine secretion, cytotoxic effector molecule release, cytotoxic activity, and expression of activation markers. The activating of the CKTC can comprise one or more of inducing secretion of a cytokine from the CKTC, stimulating proliferation of the CKTC, and upregulating expression of a cell surface marker on the CKTC. The cytokine can be one or more of IL-1, IL-2, IL-4, IL-5, IL-6, IL-10, IL-13, IL-15, TNF-α, TNF-β, and IFN-γ. According to certain embodiments, activating of a CKTC can comprise secretion of one or more of, IL-4, IL-5, Il-6, IL-10, or IFN-γ. Suitable assays to measure CKTC activation are known in the art and are described herein.

The term “active” refers to the ingredient, component or constituent of the pharmaceutical compositions of the described invention responsible for an intended therapeutic effect.

The term “administration” and its various grammatical forms as it applies to a mammal, cell, tissue, organ, or biological fluid, as used herein is meant to refer without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods. “Administration” also encompasses in vitro and ex vivo treatments, e.g., of a cell, by a reagent, diagnostic, binding composition, or by another cell. It should be understood that “administration” includes co-formulation (meaning formulated together) as well as administration via one or more pharmaceutical compositions administered concurrently (meaning at the same time, including, e.g., co-administration) or sequentially (meaning coming after in time or order).

Acute lung injury (ALI) and acute respiratory distress syndrome (ARDS). ALI and its more severe form, ARDS, are syndromes of acute respiratory failure that result from acute pulmonary edema and inflammation. ALI/ARDS is a cause of acute respiratory failure that develops in patients of all ages from a variety of clinical disorders, including sepsis (pulmonary and nonpulmonary), pneumonia (bacterial, viral, and fungal), aspiration of gastric and oropharyngeal contents, major trauma, and several other clinical disorders, including severe acute pancreatitis, drug over dose, and blood products [Ware, L. and Matthay, M., N Engl J Med, (2000) 342:1334-1349,]. Most patients require assisted ventilation with positive pressure. The primary physiologic abnormalities are severe arterial hypoxemia as well as a marked increase in minute ventilation secondary to a sharp increase in pulmonary dead space fraction. Patients with ALI/ARDS develop protein-rich pulmonary edema resulting from exudation of fluid into the interstitial and airspace compartments of the lung secondary to increased permeability of the barrier. Additional pathologic changes indicate that the mechanisms involved in lung edema are complex and that edema is only one of the pathophysiologic events in ALI/ARDS. One physiologic consequence is a significant decrease in lung compliance that results in an increased work of breathing [Nuckton T. et al., N Engl J Med. (2002) 346:1281-1286,], one of the reasons why assisted ventilation is required to support most patients. It has been reported that soon after onset of respiratory distress from COVID, patients initially retain relatively good compliance despite very poor oxygenation. [Marini, J J and Gattinoni, L., JAMA Insights (2020) doi: 10.1001/jama.2020.6825, citing Grasselli, G. et al., JAMA (2020) doi: 10.1001/jama.2020.5394; Arentz, M. et al. JAMA (2020) doi: 10.1001/jama.2020.4326]. Minute ventilation is characteristically high. Infiltrates are often limited in extent and, initially, are usually characterized by a ground-glass pattern on CT that signifies interstitial rather than alveolar edema. Many patients do not appear overtly dyspneic. These patients can be assigned, in a simplified model, to “type L,” characterized by low lung elastance (high compliance), lower lung weight as estimated by CT scan, and low response to PEEP. (Id., citing Gattinoni, L. et al. Intensive Care Med. (2020) doi: 10.1007/s00134-020-06033-2). For many patients, the disease may stabilize at this stage without deterioration while others, either because of disease severity and host response or suboptimal management, may transition to a clinical picture more characteristic of typical ARDS. These can be defined as “type H,” with extensive CT consolidations, high elastance (low compliance), higher lung weight, and high PEEP response. Types L and H are the conceptual extremes of a spectrum that includes intermediate stages, in which their characteristics may overlap.

As used herein, the term “adaptive cellular therapy” or “adaptive transfer” refer to a treatment used to help the immune system fight diseases by which T cells collected from a patient are expanded (grown in a laboratory in culture) to increase the number of T cells able to fight the disease. These T cells then are given back to the patient.

The term “adaptor molecule” as used herein refers to a specialized protein that links protein components of a signaling pathway, thereby aiding intracellular signal transduction.

As used herein the term “allogeneic” is meant to refer to being derived from two genetically different individuals.

The term “alveolar type II cells (AT2 cells)” as used herein refers to the progenitors for alveolar type I cells. Alveolar type I cells cover 95 percent of the alveolar surface of the lung; they comprise the major gas exchange surface of the alveolus and are integral to the maintenance of the permeability barrier function of the alveolar membrane. AT2 cells are the only pulmonary cells that synthesize, store, and secrete all components of pulmonary surfactant important to regulate surface tension, preventing atelectasis and maintaining alveolar fluid balance within the alveolus.

The terms “amino acid residue” or “amino acid” or “residue” are used interchangeably to refer to an amino acid that is incorporated into a protein, a polypeptide, or a peptide, including, but not limited to, a naturally occurring amino acid and known analogs of natural amino acids that can function in a similar manner as naturally occurring amino acids. The amino acids may be L- or D-amino acids. An amino acid may be replaced by a synthetic amino acid, which is altered so as to increase the half-life of the peptide, increase the potency of the peptide, or increase the bioavailability of the peptide.

The single letter designation for amino acids is used predominately herein. As is well known by one of skill in the art, such single letter designations are as follows: A is alanine; C is cysteine; D is aspartic acid; E is glutamic acid; F is phenylalanine; G is glycine; H is histidine; I is isoleucine; K is lysine; L is leucine; M is methionine; N is asparagine; P is proline; Q is glutamine; R is arginine; S is serine; T is threonine; V is valine; W is tryptophan; and Y is tyrosine.

The following represents groups of amino acids that are conservative substitutions for one another: 1) Alanine (A), Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The term “anergy” as used herein refers to a state of lymphocyte nonresponsiveness to specific antigen induced by an encounter of the lymphocyte with cognate antigen under less than optimal conditions, such as in the absence of costimulation.

The term “angiotensin-converting enzyme 2” or “ACE2” as used herein refers to a type 1 integral membrane glycoprotein [Tikellils, C. and Thomas M C. Intl J. Peptides (2012) 256294, citing Tipnis, S R et al. J. Biol. Chem. (2000) 275 (43): 33238-43] that is expressed and active in most tissues. The highest expression of ACE2 is observed in the kidney, the endothelium, the lungs, and in the heart [Id., citing Donoghue, M. et al. Cir. Res. (2000) 87 (5): E1-E9, Tipnis, S R et al. J. Biol. Chem. (2000) 275 (43): 33238-43]. The extracellular domain of ACE2 enzyme contains a single catalytic metallopeptidase unit that shares 42% sequence identity and 61% sequence similarity with the catalytic domain of ACE [[Id., citing Donoghue, M. et al. Cir. Res. (2000) 87 (5): E1-E9]. However, unlike ACE, it functions as a carboxypeptidase, rather than a dipeptidase, and ACE2 activity is not antagonized by conventional ACE inhibitors [Id., citing Rice, G I et al. Biochemical J. (2004) 383 (1): 45-51]. The major substrate for ACE2 appears to be (Ang II) [Id., citing Donoghue, M. et al. Circulation Res. (2000) 87 (5): E1-E9; turner, AJ and Hooper N M, Trends in Pharmcological Sci. (2002) 23 (4): 177-83; Rice, G I et al. Biochemical J. (2004) 383 (1): 45-51], although other peptides may also be degraded by ACE2, albeit at lower affinity. For example, ACE2 is able to cleave the C-terminal amino acid from angiotensin I, vasoactive bradykinin, des-Arg-kallidin (also known as des-Arg10 Lys-bradykinin), Apelin-13 and Apelin-36 [Id., citing Kuba, K. et al. Circulation Res. (2007) 101 (4): e32-e42] as well as other possible targets [Id., citing Vickers, C. et al. J. Biol. Chem. (2002) 277 (17): 14838-43]. The noncatalytic C-terminal domain of ACE2 shows 48% sequence identity with collectrin [Id., citing Zhang, H. et al. J. Biol. Chem. (2001) 276 (20): 17132-39], a protein shown to have an important role in neutral amino acid reabsorption from the intestine and the kidney [Id., citing Kowalczuk, S. et al. The FASEB J. (2008) 22 (8): 2880-87]; the removed amino acid then becomes available for reabsorption. The cytoplasmic tail of ACE2 also contains calmodulin-binding sites [Id., citing D W Lambert, et al. FEBS Letters (2008) 582 (2): 385-90] which may influence shedding of its catalytic ectodomain. In addition, ACE2 has also been associated with integrin function, independent of its angiotensinase activity.

As used herein, the term “antibody” includes, by way of example, both naturally occurring and non-naturally occurring antibodies. Specifically, the term “antibody” includes polyclonal antibodies and monoclonal antibodies, and fragments thereof. Furthermore, the term “antibody” includes chimeric antibodies and wholly synthetic antibodies, and fragments thereof.

As used herein, the term “antibody” is used in the broadest sense and encompasses various antibody structures, including but not limited to monoclonal antibodies, polyclonal antibodies, antibody fragments, chimeric antibodies and wholly synthetic antibodies as long as they exhibit the desired antigen-binding activity. In nature, antibodies are serum proteins the molecules of which possess small areas of their surface that are complementary to small chemical groupings on their targets. These complementary regions (referred to as the antibody combining sites or antigen binding sites) of which there are at least two per whole antibody molecule, and in some types of antibody molecules ten, eight, or in some species as many as 12, may react with their corresponding complementary region on an antigen (the antigenic determinant or epitope) to link several molecules of multivalent antigen together to form a lattice. The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

The basic structural unit of a whole antibody molecule consists of four polypeptide chains, two identical light (L) chains (each containing about 220 amino acids) and two identical heavy (H) chains (each usually containing about 440 amino acids). The two heavy chains and two light chains are held together by a combination of noncovalent and covalent (disulfide) bonds. The molecule is composed of two identical halves, each with an identical antigen-binding site composed of the N-terminal region of a light chain and the N-terminal region of a heavy chain. Both light and heavy chains usually cooperate to form the antigen binding surface.

Human antibodies show two kinds of light chains, κ and λ; individual molecules of immunoglobulin generally are only one or the other. In mammals, there are five classes of antibodies, IgA, IgD, IgE, IgG, and IgM, each with its own class of heavy chain. All five immunoglobulin classes differ from other serum proteins in that they show a broad range of electrophoretic mobility and are not homogeneous. This heterogeneity—that individual IgG molecules, for example, differ from one another in net charge—is an intrinsic property of the immunoglobulins.

The principle of complementarity, which often is compared to the fitting of a key in a lock, involves relatively weak binding forces (hydrophobic and hydrogen bonds, van der Waals forces, and ionic interactions), which are able to act effectively only when the two reacting molecules can approach very closely to each other and indeed so closely that the projecting constituent atoms or groups of atoms of one molecule can fit into complementary depressions or recesses in the other. Antigen-antibody interactions show a high degree of specificity, which is manifest at many levels. Brought down to the molecular level, specificity means that the combining sites of antibodies to an antigen have a complementarity not at all similar to the antigenic determinants of an unrelated antigen. Whenever antigenic determinants of two different antigens have some structural similarity, some degree of fitting of one determinant into the combining site of some antibodies to the other may occur, and that this phenomenon gives rise to cross-reactions. Cross reactions are of major importance in understanding the complementarity or specificity of antigen-antibody reactions. Immunological specificity or complementarity makes possible the detection of small amounts of impurities/contaminations among antigens.

Monoclonal antibodies (mAbs) can be generated by fusing mouse spleen cells from an immunized donor with a mouse myeloma cell line to yield established mouse hybridoma clones that grow in selective media. A hybridoma cell is an immortalized hybrid cell resulting from the in vitro fusion of an antibody-secreting B cell with a myeloma cell. In vitro immunization, which refers to primary activation of antigen-specific B cells in culture, is another well-established means of producing mouse monoclonal antibodies.

Diverse libraries of immunoglobulin heavy (VH) and light (Vκ and Vλ) chain variable genes from peripheral blood lymphocytes also can be amplified by polymerase chain reaction (PCR) amplification. Genes encoding single polypeptide chains in which the heavy and light chain variable domains are linked by a polypeptide spacer (single chain Fv or scFv) can be made by randomly combining heavy and light chain V-genes using PCR. A combinatorial library then can be cloned for display on the surface of filamentous bacteriophage by fusion to a minor coat protein at the tip of the phage.

The technique of guided selection is based on human immunoglobulin V gene shuffling with rodent immunoglobulin V genes. The method entails (i) shuffling a repertoire of human V_{L} chains with the heavy chain variable region (V_{H}) domain of a mouse monoclonal antibody reactive with an antigen of interest; (ii) selecting half-human Fabs on that antigen (iii) using the selected V L genes as “docking domains” for a library of human heavy chains in a second shuffle to isolate clone Fab fragments having human light chain genes; (v) transfecting mouse myeloma cells by electroporation with mammalian cell expression vectors containing the genes; and (vi) expressing the V genes of the Fab reactive with the antigen as a complete IgG1 antibody molecule in the mouse myeloma.

An antibody may be an oligoclonal antibody, a polyclonal antibody, a monoclonal antibody, a chimeric antibody, a CDR-grafted antibody, a multi-specific antibody, a bi-specific antibody, a catalytic antibody, a chimeric antibody, a humanized antibody, a fully human antibody, an anti-idiotypic antibody, and an antibody that can be labeled in soluble or bound form, as well as fragments, variants or derivatives thereof, either alone or in combination with other amino acid sequences provided by known techniques.

An antibody may be from any species. The term antibody also includes binding fragments of the antibodies of the invention. Binding fragments of an antibody can be produced by recombinant DNA techniques, or by enzymatic or chemical cleavage of intact antibodies. Exemplary fragments include Fv, Fab, Fab′, single stranded antibody (svFC), dimeric variable region (Diabody) and di-sulphide stabilized variable region (dsFv). Structural and functional domains can be identified by comparison of the nucleotide and/or amino acid sequence data to public or proprietary sequence databases. For example, computerized comparison methods can be used to identify sequence motifs or predicted protein conformation domains that occur in other proteins of known structure and/or function. Methods to identify protein sequences that fold into a known three-dimensional structure are known. See, for example, Bowie et al. Science 253:164 (1991), which is incorporated by reference in its entirety. An antibody other than a “bispecific” or “bifunctional” antibody is understood to have each of its binding sites identical.

The term “antibodyconstruct” as used herein refers to a polypeptide comprising one or more the antigen-binding portions of the invention linked to a linker polypeptide or an immunoglobulin constant domain. Linker polypeptides comprise two or more amino acid residues joined by peptide bonds and are used to link one or more antigen-binding portions. Such linker polypeptides are well known in the art (see e.g., Holliger, P., et al. (1993) Proc. Natl. Acad. Sci. USA 90:6444-6448; Poljak, R. J., et al. (1994) Structure 2:1121-1123). An immunoglobulin constant domain refers to a heavy or light chain constant domain. Human IgG heavy chain and light chain constant domain amino acid sequences are known in the art. Antibody portions, such as Fab and F(ab′)2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques.

The term “antibody-dependent cellular cytotoxicity” or ADCC, also called antibody-dependent cell-mediated cytotoxicity, is an immune mechanism through which Fc receptor-bearing effector cells can recognize and kill antibody-coated target cells expressing tumor- or pathogen-derived antigens on their surface. It is mediated by the recruitment of cytotoxic effector cells, such as natural killer (NK) cells, macrophages, and polymorphonuclear leukocytes (PMNs), that express Fc gamma receptors (FcγRs) on their surface.

The term “antibody-dependent cellular phagocytosis” or ADCP is a potent mechanism of elimination of antibody-coated foreign particles such microbes or tumor cells. Engagement of FcγRIIa and FcγRI expressed on macrophages triggers a signaling cascade leading to the engulfment of the IgG-opsonized particle.

The term “antigen” as used herein, is meant to refer to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 7, 8, 9, 10, 11, 12, 13, 14, or 15 amino acids. The term includes polypeptides which include modifications, such as deletions, additions and substitutions (generally conservative in nature) as compared to a native sequence, as long as the protein maintains the ability to elicit an immunological response, as defined herein. These modifications may be deliberate, as through site-directed mutagenesis, or may be accidental, such as through mutations of hosts which produce the antigens.

The term “antigenic drift” as used herein refers to subtle modification of pathogen antigens through random point mutations; it usually involves surface proteins that would normally be the target of neutralizing antibodies.

The term “antigenic shift” as used herein refers to dramatic modification of viral antigens due to reassortment of genomic segments of two different strains of a virus that simultaneously infect the same individual to generate progeny virions with new combinations of genome segments and thus new proteins.

The term “antigen presentation” as used herein, generally refers to the display of antigen on the surface of a cell, e.g., in the form of peptide fragments bound to MHC molecules.

As used herein, the term “antigen presenting cell (APC)” refers to a class of cells capable of displaying on its surface (“presenting”) one or more antigens in the form of peptide-MHC complex recognizable by specific effector cells of the immune system, and thereby inducing an effective cellular immune response against the antigen or antigens being presented. Examples of professional APCs are dendritic cells and macrophages, though any cell expressing MHC Class I or II molecules can potentially present peptide antigen. An APC can be an irradiated population of PBMCs. An APC can be an “artificial APC,” meaning a cell that is engineered to present one or more antigens. Before a T cell can recognize a foreign protein, the protein has to be processed inside an antigen presenting cell or target cell so that it can be displayed as peptide-MHC complexes on the cell surface.

As used herein the term “antigen processing” refers to the intracellular degradation of foreign proteins into peptides that can bind to MHC molecules for presentation to T cells.

The term “apheresis” as used herein refers to a medical technology in which the blood of a donor or patient is passed through an apparatus that separates out one particular constituent and returns the remainder back to the donor or patient's circulation. Leukapheresis is one type of apheresis where leukocytes (white blood cells) are selectively removed.

The terms “apoptosis” or “programmed cell death” refer to a highly regulated and active process that contributes to biologic homeostasis comprised of a series of biochemical events that lead to a variety of morphological changes, including blebbing, changes to the cell membrane, such as loss of membrane asymmetry and attachment, cell shrinkage, nuclear fragmentation, chromatin condensation, and chromosomal DNA fragmentation, without damaging the organism.

Apoptotic cell death is induced by many different factors and involves numerous signaling pathways, some dependent on caspase proteases (a class of cysteine proteases) and others that are caspase independent. It can be triggered by many different cellular stimuli, including cell surface receptors, mitochondrial response to stress, and cytotoxic T cells, resulting in activation of apoptotic signaling pathways

The caspases involved in apoptosis convey the apoptotic signal in a proteolytic cascade, with caspases cleaving and activating other caspases that then degrade other cellular targets that lead to cell death. The caspases at the upper end of the cascade include caspase-8 and caspase-9. Caspase-8 is the initial caspase involved in response to receptors with a death domain (DD) like Fas.

Receptors in the TNF receptor family are associated with the induction of apoptosis, as well as inflammatory signaling. The Fas receptor (CD95) mediates apoptotic signaling by Fas-ligand expressed on the surface of other cells. The Fas-FasL interaction plays an important role in the immune system and lack of this system leads to autoimmunity, indicating that Fas-mediated apoptosis removes self-reactive lymphocytes. Fas signaling also is involved in immune surveillance to remove transformed cells and virus infected cells. Binding of Fas to oligimerized FasL on another cell activates apoptotic signaling through a cytoplasmic domain termed the death domain (DD) that interacts with signaling adaptors including FAF, FADD and DAX to activate the caspase proteolytic cascade. Caspase-8 and caspase-10 first are activated to then cleave and activate downstream caspases and a variety of cellular substrates that lead to cell death.

Mitochondria participate in apoptotic signaling pathways through the release of mitochondrial proteins into the cytoplasm. Cytochrome c, a key protein in electron transport, is released from mitochondria in response to apoptotic signals, and activates Apaf-1, a protease released from mitochondria. Activated Apaf-1 activates caspase-9 and the rest of the caspase pathway. Smac/DIABLO is released from mitochondria and inhibits IAP proteins that normally interact with caspase-9 to inhibit apoptosis. Apoptosis regulation by Bcl-2 family proteins occurs as family members form complexes that enter the mitochondrial membrane, regulating the release of cytochrome c and other proteins. TNF family receptors that cause apoptosis directly activate the caspase cascade, but can also activate Bid, a Bcl-2 family member, which activates mitochondria-mediated apoptosis. Bax, another Bcl-2 family member, is activated by this pathway to localize to the mitochondrial membrane and increase its permeability, releasing cytochrome c and other mitochondrial proteins. Bcl-2 and Bcl-xL prevent pore formation, blocking apoptosis. Like cytochrome c, AIF (apoptosis-inducing factor) is a protein found in mitochondria that is released from mitochondria by apoptotic stimuli. While cytochrome C is linked to caspase-dependent apoptotic signaling, AIF release stimulates caspase-independent apoptosis, moving into the nucleus where it binds DNA. DNA binding by AIF stimulates chromatin condensation, and DNA fragmentation, perhaps through recruitment of nucleases.

The mitochondrial stress pathway begins with the release of cytochrome c from mitochondria, which then interacts with Apaf-1, causing self-cleavage and activation of caspase-9. Caspase-3, -6 and -7 are downstream caspases that are activated by the upstream proteases and act themselves to cleave cellular targets.

Granzyme B and perforin proteins released by cytotoxic T cells induce apoptosis in target cells, forming transmembrane pores, and triggering apoptosis, perhaps through cleavage of caspases, although caspase-independent mechanisms of Granzyme B mediated apoptosis have been suggested.

Fragmentation of the nuclear genome by multiple nucleases activated by apoptotic signaling pathways to create a nucleosomal ladder is a cellular response characteristic of apoptosis. One nuclease involved in apoptosis is DNA fragmentation factor (DFF), a caspase-activated DNAse (CAD). DFF/CAD is activated through cleavage of its associated inhibitor ICAD by caspases proteases during apoptosis. DFF/CAD interacts with chromatin components such as topoisomerase II and histone H1 to condense chromatin structure and perhaps recruit CAD to chromatin. Another apoptosis activated protease is endonuclease G (EndoG). EndoG is encoded in the nuclear genome but is localized to mitochondria in normal cells. EndoG may play a role in the replication of the mitochondrial genome, as well as in apoptosis. Apoptotic signaling causes the release of EndoG from mitochondria. The EndoG and DFF/CAD pathways are independent since the EndoG pathway still occurs in cells lacking DFF.

Hypoxia, as well as hypoxia followed by reoxygenation can trigger cytochrome c release and apoptosis. Glycogen synthase kinase (GSK-3) a serine-threonine kinase ubiquitously expressed in most cell types, appears to mediate or potentiate apoptosis due to many stimuli that activate the mitochondrial cell death pathway. Loberg, R D, et al., J. Biol. Chem. 277 (44): 41667-673 (2002). It has been demonstrated to induce caspase 3 activation and to activate the proapoptotic tumor suppressor gene p53. It also has been suggested that GSK-3 promotes activation and translocation of the proapoptotic Bcl-2 family member, Bax, which, upon aggregation and mitochondrial localization, induces cytochrome c release. Akt is a critical regulator of GSK-3, and phosphorylation and inactivation of GSK-3 may mediate some of the antiapoptotic effects of Akt.

The term “attenuate” as used herein refers to render less virulent, to weaken or reduce in force, intensity, effect or quantity.

As used herein, the term “autologous” is meant to refer to being derived from the same individual.

As used herein, the term “autophagy” refers to the digestion and breakdown by a cell of its own organelles and proteins in lysosomes.

The terms “B lymphocyte” or “B cell” are used interchangeably to refer to a broad class of lymphocytes, which are precursors of antibody-secreting cells, that express clonally diverse cell surface immunoglobulin (Ig) receptors (BCRs) recognizing specific antigenic epitopes. Mammalian B-cell development encompasses a continuum of stages that begin in primary lymphoid tissue (e.g., human fetal liver and fetal/adult marrow), with subsequent functional maturation in secondary lymphoid tissue (e.g., human lymph nodes and spleen). The functional/protective end point is antibody production by terminally differentiated plasma cells. A mature B cell can be activated by an encounter with an antigen that expresses epitopes that are recognized by its cell surface immunoglobulin (Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”).[LeBien, T W & T F Tedder, B lymphocytes: how they develop and function. Blood (2008) 112 (5): 1570-80].

The term “B cell receptor” or “BCR” as used herein refers to the antigen-receptor complex of B lineage cells, which is composed of a membrane bound Ig (mIg) monomer plus the Igα/Igβ complex required for intracellular signaling.

The term “beta2-microglobulin” as used herein refers to the light chain of the MHC class I proteins, encoded outside the MHC. It binds noncovalently to the heavy or a, chain.

The term “binding” and its various grammatical forms means a lasting attraction between chemical substances. Binding specificity involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “binding specificity” as used herein involves both binding to a specific partner and not binding to other molecules. Functionally important binding may occur at a range of affinities from low to high, and design elements may suppress undesired cross-interactions. Post-translational modifications also can alter the chemistry and structure of interactions. “Promiscuous binding” may involve degrees of structural plasticity, which may result in different subsets of residues being important for binding to different partners. “Relative binding specificity” is a characteristic whereby in a biochemical system a molecule interacts with its targets or partners differentially, thereby impacting them distinctively depending on the identity of individual targets or partners.

The term “bioavailable” and its other grammatical forms as used herein refers to the ability of a substance to be absorbed and sued by the body.

The term “biocompatible” as used herein refers to a material that is generally non-toxic to the recipient and does not possess any significant untoward effects to the subject and, further, that any metabolites or degradation products of the material are non-toxic to the subject. Typically a substance that is “biocompatible” causes no clinically relevant tissue irritation, injury, toxic reaction, or immunological reaction to living tissue.

The term “biodegradable” as used herein refers to a material that will erode to soluble species or that will degrade under physiologic conditions to smaller units or chemical species that are, themselves, non-toxic.

As used herein, the term “biomarker” (or “biosignature”) refers to a peptide, protein, nucleic acid, antibody, gene, metabolite, or any other substance used as an indicator of a biologic state. It is a characteristic that is measured objectively and evaluated as a cellular or molecular indicator of normal biologic processes, pathogenic processes, or pharmacologic responses to a therapeutic intervention. The term “indicator” as used herein refers to any substance, number or ratio derived from a series of observed facts that may reveal relative changes as a function of time; or a signal, sign, mark, note or symptom that is visible or evidence of the existence or presence thereof. Once a proposed biomarker has been validated, it may be used to diagnose disease risk, presence of disease in an individual, or to tailor treatments for the disease in an individual (choices of drug treatment or administration regimes). In evaluating potential drug therapies, a biomarker may be used as a surrogate for a natural endpoint, such as survival or irreversible morbidity. If a treatment alters the biomarker, and that alteration has a direct connection to improved health, the biomarker may serve as a surrogate endpoint for evaluating clinical benefit. Clinical endpoints are variables that can be used to measure how patients feel, function or survive. Surrogate endpoints are biomarkers that are intended to substitute for a clinical endpoint; these biomarkers are demonstrated to predict a clinical endpoint with a confidence level acceptable to regulators and the clinical community.

As used herein the term “CD1d” is meant to refer to a family of transmembrane glycoproteins, which are structurally related to the MHC proteins and form heterodimers with beta-2-microglobulins that mediate the presentation of primarily lipid and glycolipid antigens of self or microbial origin to T cells.

The term “CARD domain” as used herein refers to the family subclass of the caspase recruitment domain. The formation of apoptotic and inflammatory multiprotein complexes together with defined signaling episodes in innate immunity heavily relies on members of the death domain family and particularly on the family subclass of the caspase recruitment domain (CARD). [Palacios-Rodriguez, Y. et al., Polypeptide Modulators of Caspase Recruitment Domain (CARD)-Card-mediated protein-protein interactions. J. Biol. Chem. (2011) 286 (52): 44457-66, citing Varfolomeev, E. et al. Cell (2007) 131: 669-81] The interaction between the CARD of Apaf-1 (apoptotic protease-activating factor) and the CARD of procaspase-9 (PC9) in the mitochondria-mediated apoptotic intrinsic pathway is essential for the recruitment of PC9 into the apoptosome and its subsequent activation [Id., citing Acehan, D., et al. Mol. Cell (2002) 9: 423-32]. On the other hand, proteins like those of the NOD-like receptor (NLR) family (in particular NOD-1, NOD-2, and NLRP-1) act as intracellular scrutiny devices and signaling initiators to face microbial aggressions [Id., citing Proell, M. et al. PLoS One (2008) 3: e2119]. The NLR proteins utilize the CARD for binding to downstream signaling molecules through CARD-CARD interactions in order to ultimately initiate the innate immune and inflammatory responses [Id., citing Inohara N., Nufiez G. Nat. Rev. Immunol. (2003) 3, 371-382; Park, H H, et al. Annu. Rev. Immunol. (2007) 25, 561-586].

The term “carrier” as used herein describes a material that does not cause significant irritation to an organism and does not abrogate the biological activity and properties of the active compound of the composition of the described invention. Carriers must be of sufficiently high purity and of sufficiently low toxicity to render them suitable for administration to the mammal being treated. The carrier can be inert, or it can possess pharmaceutical benefits, cosmetic benefits or both. The terms “excipient”, “carrier”, or “vehicle” are used interchangeably to refer to carrier materials suitable for formulation and administration of pharmaceutically acceptable compositions described herein. Carriers and vehicles useful herein include any such materials know in the art which are nontoxic and do not interact with other components. The carrier can be liquid or solid and is selected, with the planned manner of administration in mind, to provide for the desired bulk, consistency, etc., when combined with an active agent and other components of a given composition.

The term “CD40” as used herein refers to a tumor necrosis factor receptor (TNFR) superfamily member expressed on APCs, such as dendritic cells (DC), B cells, and monocytes as well as many non-immune cells and a wide range of tumors. Interaction with its trimeric ligand CD40 Ligand (CD40L) on activated T helper cells results in APC activation, required for the induction of adaptive immunity. CD40 on B cells and CD40 ligand on activated helper T cells are co-stimulatory molecules whose interaction is required for the proliferation and class switching of antigen activated naïve B cells. CD40 is also expressed by dendritic cells; where the CD40-CD40L interaction provides co-stimulatory signals to naïve T cells.

As used herein, the term “cell growth” is the process by which cells accumulate mass and increase in physical size. There are many different examples in nature of how cells can grow. In some cases, cell size is proportional to DNA content. For instance, continued DNA replication in the absence of cell division (called endoreplication) results in increased cell size. Megakaryoblasts, which mature into granular megakaryocytes, the platelet-producing cells of bone marrow, typically grow this way. By a different strategy, adipocytes can grow to approximately 85 to 120 pm by accumulating intracellular lipids. In contrast to endoreplication or lipid accumulation, some terminally differentiated cells, such as neurons and cardiac muscle cells, cease dividing and grow without increasing their DNA content. These cells proportionately increase their macromolecule content (largely protein) to a point necessary to perform their specialized functions. This involves coordination between extracellular cues from nutrients and growth factors and intracellular signaling networks responsible for controlling cellular energy availability and macromolecular synthesis. Perhaps the most tightly regulated cell growth occurs in dividing cells, where cell growth and cell division are clearly separable processes. Dividing cells generally must increase in size with each passage through the cell division cycle to ensure that a consistent average cell size is maintained. For a typical dividing mammalian cell, growth occurs in the G1 phase of the cell cycle and is tightly coordinated with S phase (DNA synthesis) and M phase (mitosis). The combined influence of growth factors, hormones, and nutrient availability provides the external cues for cells to grow. [Guertin, D. A., Sabatini, D. M., “Cell Growth,” in The Molecular Basis of Cancer (4^{th} Edn) Mendelsohn, J. et al Eds, Saunders (2015), 179-190].

As used herein, the term “cell proliferation” is meant to refer to the process that results in an increase of the number of cells, and is defined by the balance between cell divisions and cell loss through cell death or differentiation.

As used herein, the term “chemokine” is meant to refer to a class of chemotactic cytokines that signal leukocytes to move in a specific direction.

The term “clade” as used herein refers to related organisms descended from a common ancestor.

The term “class switching”, “isotype switching” or “class switch recombination” as used herein refers to a somatic gene recombination process in activated B cells that replaces one heavy chain constant region with one of a different isotype, switching the isotype of antibodies from IgM to IgG, IgA or IgE. This affects the antibody effector functions but not their antigen specificity.

As used herein, the term “cognate help” is meant to refer to a process that occurs most efficiently in the context of an intimate interaction with a helper T cell.

The term “complement” as used herein refers to a system of over 30 soluble and membrane-bound proteins that act through a tightly regulated cascade of pro-protein cleavage and activation to mediate cell lysis through assembly of the membrane attack complex (MAC) composed of complement components C5b, C6, C7, C8, and C9 in a target cell membrane. Intermediates in the complement cascade play a variety of roles in antigen clearance. The activation of complement can lead to lysis of an antibody-opsonized cell by complement-dependent cytotoxicity (CDC) or complement-dependent cell-mediated cytotoxicity (CDCC) [Meyer, S. et al. MAbs (2014) 6 (5): 1133-44].

The term “component” as used herein, is meant to refer to a constituent part, element or ingredient.

The term “composition” as used herein, is meant to refer to a material formed by a mixture of two or more substances.

As used herein, the term “condition” as used herein, is meant to refer to a variety of health states and is meant to include disorders or diseases caused by any underlying mechanism or disorder.

As used herein, the term “contact” and its various grammatical forms is meant to refer to a state or condition of touching or of immediate or local proximity. Contacting a composition to a target destination may occur by any means of administration known to the skilled artisan.

The term “costimulation” as used herein refers to the second signal required for completion of lymphocyte activation and prevention of anergy, which is supplied by engagement of CD28 by CD80 and CD86 (T cells) and of CD40 by CD40 Ligand (B cells).

The term “costimulatory molecule” as used herein refers to molecules that are displayed on the cell surface that have a role in enhancing the activation of a T cell that is already being stimulated through its TCR. For example, HLA proteins, which present foreign antigen to the T cell receptor, require costimulatory proteins which bind to complementary receptors on the T cell's surface to result in enhanced activation of the T cell. The term “co-stimulatory molecules” as used herein refers to highly active immunomodulatory proteins that play a critical role in the development and maintenance of an adaptive immune response (Kaufman and Wolchok eds., General Principles of Tumor Immunotherapy, Chpt 5, 67-121 (2007)). The two signal hypothesis of T cell response involves the interaction between an antigen bound to an HLA molecule and with its cognate T cell receptor (TCR), and an interaction of a co-stimulatory molecule and its ligand. Specialized APCs, which are carriers of a co-stimulatory second signal, are able to activate T cell responses following binding of the HLA molecule with TCR. By contrast, somatic tissues do not express the second signal and thereby induce T cell unresponsiveness (Id.). Many of the co-stimulatory molecules involved in the two-signal model can be blocked by co-inhibitory molecules that are expressed by normal tissue (Id.). In fact, many types of interacting immunomodulatory molecules expressed on a wide variety of tissues may exert both stimulatory and inhibitory functions depending on the immunologic context (Id.).As used herein the term “co-stimulatory receptor” is meant to refer to a cell surface receptor on naïve lymphocytes through which they receive signals additional to those received through the antigen receptor, and which are necessary for the full activation of the lymphocyte. Examples are CD30 and CD40 on B cells, and CD27 and CD28 on T cells.

As used herein, the term “cross-protection” is used to describe immunity against at least two subgroups, subtypes, strains and/or variants of a virus, bacteria, parasite or other pathogen with a single inoculation with one subgroup, subtype, strain and/or variant thereof.

The term “culture” and its other grammatical forms as used herein, is meant to refer to a process whereby a population of cells is grown and proliferated on a substrate in an artificial medium.

The term “cytokine” as used herein refers to small soluble protein substances secreted by cells which have a variety of effects on other cells. Cytokines mediate many important physiological functions including growth, development, wound healing, and the immune response. They act by binding to their cell-specific receptors located in the cell membrane, which allows a distinct signal transduction cascade to start in the cell, which eventually will lead to biochemical and phenotypic changes in target cells. Generally, cytokines act locally. They include type I cytokines, which encompass many of the interleukins, as well as several hematopoietic growth factors; type II cytokines, including the interferons and interleukin-10; tumor necrosis factor (“TNF”)-related molecules, including TNFα and lymphotoxin; immunoglobulin super-family members, including interleukin 1 (“IL-1”); and the chemokines, a family of molecules that play a critical role in a wide variety of immune and inflammatory functions. The same cytokine can have different effects on a cell depending on the state of the cell. Cytokines often regulate the expression of, and trigger cascades of, other cytokines. Non-limiting examples of cytokines include e.g., IL-1, IL-2, IL-3, IL-4, IL-5, IL-6, IL-7, IL-8, IL-9, IL-10, IL-11, IL-12/IL-23 P40, IL13, IL-15, IL-15/IL15-RA, IL-17, IL-18, IL-21, IL-23, TGF-β, IFNγ, GM-CSF, Groα, MCP-1 and TNF-α.

The term “cytotoxic T lymphocytes” (CTLs) as used herein, is meant to refer to effector CD8+ T cells. Cytotoxic T cells kill by inducing their targets to undergo apoptosis. They induce target cells to undergo programmed cell death via extrinsic and intrinsic pathways.

The term damage-associated molecular patterns” or “DAMPS” as used herein refers to molecules released by stressed or dying cells that bind to pattern recognition molecules (PRMs) and induce inflammation.

The term “dendritic cells (DC)” as used herein refers to professional antigen presenting cells, which induce naïve T cell activation and effector differentiation. [Patente, T A, et al., Frontiers Immunol. (2019) doi.org/10.3389/fimmu.2018.03176]. Human DC are identified by their high expression of major histocompatibility complex (MHC) class II molecules (MHC-II) and of CD11c, both of which are found on other cells, like lymphocytes, monocytes and macrophages [Id., citing Carlens J, et al. J Immunol. (2009) 183:5600-7; Drutman S B, et al. J Immunol. (2012) 188:3603-10; Hochweller K, S et al. Eur J Immunol. (2008) 38:2776-83; Huleatt J W, Lefrangois L. J Immunol. (1995) 154:5684-93; Rubtsov A V, et al. Blood (2011) 118:1305-15; Probst H C, et al. Clin Exp Immunol. (2005) 141:398-404; Vermaelen K, Pauwels R. Cytometry (2004) 61A:170-7]. DC express many other molecules which allow their classification into various subtypes. Although some of the DC subtypes were originally described as macrophages, DC and macrophages have distinct characteristics [Id., citing Delamarre L, Science (2005) 307:1630-4; Geissmann F, et al. Science (2010) 327:656-61; van Montfoort N, et al. Proc Natl Acad Sci USA. (2009) 106:6730-5] and ontogeny, so that, currently, little doubt remains that they belong to distinct lineages [Id., citing Haniffa M, et al. (2013) 120:1-49; Hashimoto D, et al. Immunity (2013) 38:792-804; Hettinger J, et al. Nat Immunol. (2013) 14:821-30; McGovern N, et al. Immunity (2014) 41:465-77; Naik S H, et al. Nature (2013) 496:229-32; Schulz C, et al. Science (2012) 336:86-90; Schraml B U, et al. Cell (2013) 154:843-58; Wang J, et al. Mol Med Rep. (2017) 16:6787-93; Yona S, et al. Immunity (2013) 38:79-91]. DC are found in two different functional states, “mature” and “immature”. These are distinguished by many features, but the ability to activate antigen-specific naïve T cells in secondary lymphoid organs is the hallmark of mature DC [Id., citing Hawiger D, Inaba K, et al. J Exp Med. (2001) 194:769-79; Steinman R M, et al. Ann NY Acad Sci. (2003) 987:15-25; Worbs T, et al. Nat Rev Immunol. (2017) 17:30-48]. DC maturation is triggered by tissue homeostasis disturbances, detected by the recognition of pathogen-associated molecular patterns (PAMP) or damage-associated molecular patterns (DAMPs) [Id., citing Hemmi H, et al. Chem Immunol Aller. (2005) 86:120-135, Cerboni S, et al. Adv Immunol. (2013) 120:211-237]. Maturation turns on metabolic, cellular, and gene transcription programs allowing DC to migrate from peripheral tissues to T-dependent areas in secondary lymphoid organs, where T lymphocyte-activating antigen presentation may occur [Id., citing Alvarez D, et al. Immunity (2008) 29:325-42; Dong H, Bullock T N J. Front Immunol. (2014) 5:24; Friedl P, Gunzer M. Trends Immunol. (2001) 22:187-91; Henderson R A, et al. J Immunol. (1997) 159:635-43; Randolph G J, et al. Nature Rev Immunol. (2005) 5:617-28 Imai Y, et al. Histol Histopathol. (1998) 13:469-510]. During maturation, DC lose adhesive structures, reorganize the cytoskeleton and increase their motility [Id., citing Winzler C, et al. J Exp Med. (1997) 185:317-28). DC maturation also leads to a decrease in their endocytic activity but increased expression of MHC-II and co-stimulatory molecules [Id., citing Reis e Sousa C. Nature Rev Immunol. (2006) 6:476-83; Steinman R M. Annu Rev Immunol. (2012) 30:1-22; Trombetta E S, Mellman I. Annu Rev Immunol. (2005) 23:975-1028]. Mature DC express higher levels of the chemokine receptor, CCR7 [Id., citing Forster R, et al. Cell (1999) 99:23-33; Ohl L, et al. Immunity (2004) 21:279-88; Sallusto F, et al. Eur J Immunol. (1998) 28:2760-9; Steinman R M. The control of immunity and tolerance by dendritic cell. Pathol Biol. (2003) 51:59-60] and secrete cytokines, essential for T-cell activation [Id., citing Reis e Sousa C. Nature Rev Immunol. (2006) 6:476-83, Caux C, et al. J Exp Med. (1994) 180:1263-72; Jensen S S, Gad M. J Inflamm (Lond) (2010) 7:37; Tan J K H, O'Neill H C. J Leukocyte Biol. (2005) 78:319-324; Iwasaki A, Medzhitov R. Nat Immunol. (2015) 16:343-353]. Thus, the interaction between mature DC and antigen-specific T cells is the trigger of antigen-specific immune responses [Id., citing Luft T., Blood (2006) 107:4763-9, Jonuleit H. Arch Dermatol Res. (1996) 289:1-8]. When interacting with CD4+ T cells, DC may induce their differentiation into different T helper (T_{H}) subsets [Id., citing Iwasaki A, Medzhitov R. Nat Immunol. (2015) 16:343-353] such as T_{H}1 [Amsen D, et al. Cell (2004) 117:515-26; Constant S, et al. J Exp Med (1995) 182:1591-6; Hosken N A, et al. J Exp Med. (1995) 182:1579-84; Kadowaki N. Allergol Int. (2007) 56:193-9; Maekawa Y, et al. Immunity (2003) 19:549-59; Pulendran B, et al. Proc Natl Acad Sci USA. (1999) 96:1036-41, Th2 [Id., citing Constant S, et al. J Exp Med (1995) 182:1591-6, Hosken N A, et al. J Exp Med. (1995) 182:1579-84, Jenkins S J, P. et al. J Immunol. (2007) 179:3515-23, Soumelis V, et al. Nat Immunol. (2002) 3:673-6801, T_{H}17 [Id., citing Bailey S L, Nat Immunol. (2007) 8:172-80; Iezzi G, et al. Proc Natl Acad Sci USA. (2009) 106:876-81; Huang G, et al. Cell Mol Immunol. (2012) 9:287-951, or other CD4+ T cell subtypes [Id., citing Levings M K, et al. Blood (2005) 105:1162-91. T cell differentiation in each subtype is a complex phenomenon, that can be influenced by the cytokines in the DC tissue of origin [Id., citing Rescigno M. Dendritic cell-epithelial cell crosstalk in the gut. Immunol Rev. (2014) 260:118-281, their maturation state [Id., citing Reis e Sousa C. Nature Rev Immunol. (2006) 6:476-831 and cause of tissue imbalance [Id., citing Vega-Ramos J, et al. Curr Opin Pharmacol. (2014) 17:64-701. DCs present a unique characteristic: the ability to perform cross-presentation [Id., citing Coulon P-G, et al. J Immunol. (2016) 197:517-32; Delamarre L, Mellman I. Semin Immunol. (2011) 23:2-11; Jung S, et al. Immunity (2002) 17:211-20; Segura E, Amigorena S. Adv Immunol. (2015) 127:1-31; Segura E, Villadangos J A. Curr Opin Immunol. (2009) 21:105-1101, defined as the presentation, in the context of class I MHC molecules (MHC-I), of antigens captured from the extracellular milieu. This feature allows DC to trigger responses against intracellular antigens from other cell types, thus providing means for the system to deal with threats that avoid professional APC [Id., citing Coulon P-G, et al. J Immunol. (2016) 197:517-32, Bevan M J. Cross-priming for a secondary cytotoxic response to minor H antigens with H-2 congenic cells which do not cross-react in the cytotoxic assay. J Exp Med. (1976) 143:1283-8, Sinchez-Paulete A R, et al. Ann Oncol. (2017) 28:xii74. doi: 10.1093/annonc/mdx727] and, even, to prime CD8+ lymphocytes in the absence of CD4+ T cells [Id., citing McCoy K D, et al. J Exp Med. (1999) 189:1157-62, Young J W, Steinman R M. J Exp Med. (1990) 171:1315-321. Cross-presentation is involved also in the induction of tolerance to intracellular self-antigens that are not expressed by APC and, then, called, cross-tolerance [Kurts C, et al. J Exp Med. (1997) 186:239-45, Rock K L, Shen L. Immunol Rev. (2005) 207:166-831.

Before receiving maturation stimuli, DC are said to be in an “immature state.” Immature DC are poor inducers of naïve lymphocyte effector responses, since they have low surface expression of co-stimulatory molecules, low expression of chemokine receptors, and do not release immunostimulatory cytokines [Id., citing Trombetta E S, Mellman I. Annu Rev Immunol. (2005) 23:975-1028, Steinman R M, Swanson J. J Exp Med. (1995) 182:283-81. These “immature” cells, though, are very efficient in antigen capture due to their high endocytic capacity, via receptor-mediated endocytosis, including lectin-[Id., citing Geijtenbeek T B, et al. Cell (2000) 100:575-585; Sallusto F, et al. J Exp Med. (1995) 182:389-400; Valladeau J, et al. Cell Immunol. (1994) 159:323-30; Medzhitov R, et al. Nature (1997) 388:394-7; Muzio M, et al. J Immunol. (2000) 164:5998-6004], FC- and complement receptors [Id., citing Muzio M, et al. J Immunol. (2000) 164:5998-6004) and macropinocytosis (Id., citing Sallusto F, et al. J Exp Med. (1995) 182:389-400). Thus, immature DCs act not only as sentinels against invading pathogens [Id., citing Worbs T, et al. Nat Rev Immunol. (2017) 17:30-48, Wilson N S, et al. Blood (2004) 103:2187-95], but also as tissue scavengers, capturing apoptotic and necrotic cells [Id., citing Albert M L, et al. Nature (1998) 392:86-9).

This latter feature confers to immature DC an essential role in the induction and maintenance of immune tolerance [Id., citing Steinman R M, et al. Ann NY Acad Sci. (2003) 987:15-25, Castellano G, et al. Mol Immunol. (2004) 41:133-40; Deluce-Kakwata-Nkor N, et al. Transfus Clin Biol. (2018) 25:90-5; Liu J, Cao X. J Autoimmun. (2015) 63:1-12; Shiokawa A, et al. Immunology (2017) 152:52-64]. Apoptotic cells that arise in consequence of natural tissue turnover [Id., citing Huang F P, et al. J Exp Med. (2000) 191:435-44, Steinman R M, et al. J Exp Med. (2000) 191:411-416] are internalized by DCs but do not induce their maturation [Id., citing Steinman R M, et al. Ann NY Acad Sci. (2003) 987:15-25, Liu K, et al. J Exp Med. (2002) 196:1091-1097; Stuart L M, et al. J Immunol. (2002) 168:1627-35; Wallet M A, et al. J Exper Med. (2008) 205:219-32]. Thus, their antigens are presented to T cells without the activating co-stimulatory signals that a mature DC would deliver, resulting in T cell apoptosis [Id., citing Kurts C, et al. J Exp Med. (1997) 186:239-45, Hong J, et al. Chin Med J. (2013) 126:2139-44], anergy [Id., citing Manicassamy S, Pulendran B. Immunol Rev. (2011) 241:206-27, Zhu H-C, et al. Cell Immunol. (2012) 274:12-8] or development into Tregs [Id., citing Saito M, et al. J Exper Med. (2011) 208:235-49, Sela U, et al., PLoS ONE (2016) 11:e0146412).

These “tolerogenic DC” express less co-stimulatory molecules and proinflammatory cytokines, but upregulate the expression of inhibitory molecules (like PD-L1 and CTLA-4), secrete anti-inflammatory cytokines (IL-10, for example) [Id., citing Manicassamy S, Pulendran B. Immunol Rev. (2011) 241:206-27, Grohmann U, et al. Nat Immunol. (2002) 3:1097-101; Morelli A E, Thomson A W. Nature Rev Immunol. (2007) 7:610-21; Sakaguchi S, et al. Nat Rev Immunol. (2010) 10:490-500] and are essential to prevent responses against healthy tissues [Id., citing Hawiger D. J Exp Med. (2001) 194:769-79, Steinman R M, et al. Ann NY Acad Sci. (2003) 987:15-25, Idoyaga J, et al. J Clin Invest. (2013) 123:844-54; Mahnke K, et al. Blood (2003) 101:4862-9; Yates S F, et al. J Immunol (2007) 179:967-76; Yogev N, et al. Immunity (2012) 37:264-75].

However, in some contexts, immature DC can be harmful to the body. It is known that DC that are unable to induce lymphocyte effector responses may contribute to the immune system's failure to fight infections [Id., citing Campanelli A P, et al. J Infect Dis. (2006) 193:1313-22, Montagnoli C, et al. J Immunol. (2002) 169:6298-308] or tumors [Id., citing Baleeiro R B, et al. Cancer Immunol Immunother (2008) 57:1335-45; Almand B, et al. Clin Cancer Res. (2000) 6:1755-66; Bella S D, et al. Br J Cancer (2003) 89:1463-72; Dunn G P, et al. Immunity (2004) 21:137-48; Johnson D J, Ohashi P S. Anna NY Acad Sci. (2013) 1284:46-51; Vicari A P, et al. Semin Cancer Biol. (2002) 12:33-42]. In these situations, DC, even after recognition of pathogens or other changes in microenvironment, fail to increase the co-stimulatory molecules required to activate T cells, thus allowing the disease to “escape” immune control.

The term “DAD” as used herein refers to diffuse alveolar damage (DAD), which is manifested by injury to alveolar lining and endothelial cells, pulmonary edema, hyaline membrane formation and later by proliferative changes involving alveolar and bronchiolar lining cells and interstitial cells (Katzenstein, A L et al. Am J Pathol (1976) 85:209).

The term “derived from” as used herein, is meant to encompasses any method for receiving, obtaining, or modifying something from a source of origin.

The term “detectable marker” encompasses both selectable markers and assay markers. The term “selectable markers” refers to a variety of gene products to which cells transformed with an expression construct can be selected or screened, including drug-resistance markers, antigenic markers useful in fluorescence-activated cell sorting, adherence markers such as receptors for adherence ligands allowing selective adherence, and the like.

The term “detectable response” as used herein, is meant to refer to any signal or response that may be detected in an assay, which may be performed with or without a detection reagent. Detectable responses include, but are not limited to, radioactive decay and energy (e.g., fluorescent, ultraviolet, infrared, visible) emission, absorption, polarization, fluorescence, phosphorescence, transmission, reflection or resonance transfer. Detectable responses also include chromatographic mobility, turbidity, electrophoretic mobility, mass spectrum, ultraviolet spectrum, infrared spectrum, nuclear magnetic resonance spectrum and x-ray diffraction. Alternatively, a detectable response may be the result of an assay to measure one or more properties of a biologic material, such as melting point, density, conductivity, surface acoustic waves, catalytic activity or elemental composition. A “detection reagent” is any molecule that generates a detectable response indicative of the presence or absence of a substance of interest. Detection reagents include any of a variety of molecules, such as antibodies, nucleic acid sequences and enzymes. To facilitate detection, a detection reagent may comprise a marker.

The term “differentiate” and its various grammatical forms as used herein, are meant to refer to the process of development with an increase in the level of organization or complexity of a cell or tissue, accompanied with a more specialized function.

The terms “disease” or “disorder” as used herein refer to an impairment of health or a condition of abnormal functioning.

The term “dose” as used herein, is meant to refer to the quantity of a therapeutic substance prescribed to be taken at one time. The term “maximum tolerated dose” as used herein is meant to refer to the highest dose of a drug or treatment that does not cause unacceptable side effects.

The term “dye” (also referred to as “fluorochrome” or “fluorophore”) as used herein refers to a component of a molecule which causes the molecule to be fluorescent. The component is a functional group in the molecule that absorbs energy of a specific wavelength and re-emits energy at a different (but equally specific) wavelength. The amount and wavelength of the emitted energy depend on both the dye and the chemical environment of the dye. Many dyes are known, including, but not limited to, FITC, R-phycoerythrin (PE), PE-Texas Red Tandem, PE-Cy5 Tandem, propidium iodem, EGFP, EYGP, ECF, DsRed, allophycocyanin (APC), PerCp, SYTOX Green, courmarin, Alexa Fluors (350, 430, 488, 532, 546, 555, 568, 594, 633, 647, 660, 680, 700, 750), Cy2, Cy3, Cy3.5, Cy5, Cy5.5, Cy7, Hoechst 33342, DAPI, Hoechst 33258, SYTOX Blue, chromomycin A3, mithramycin, YOYO-1, SYTOX Orange, ethidium bromide, 7-AAD, acridine orange, TOTO-1, TO-PRO-1, thiazole orange, TOTO-3, TO-PRO-3, thiazole orange, propidium iodide (PI), LDS 751, Indo-1, Fluo-3, DCFH, DHR, SNARF, Y66F, Y66H, EBFP, GFPuv, ECFP, GFP, AmCyan1, Y77W, S65A, S65C, S65L, S65T, ZsGreen1, ZsYellow1, DsRed2, DsRed monomer, AsRed2, mRFP1, HcRed1, monochlorobimane, calcein, the DyLight Fluors, cyanine, hydroxycoumarin, aminocoumarin, methoxycoumarin, Cascade Blue, Lucifer Yellow, NBD, PE-Cy5 conjugates, PE-Cy7 conjugates, APC-Cy7 conjugates, Red 613, fluorescein, FluorX, BODIDY-FL, TRITC, X¬rhodamine, Lissamine Rhodamine B, Texas Red, TruRed, and derivatives thereof.

The term “ECOG performance status scale” as used herein refers to a scale used to assess how a patient's disease is progressing, assess how the disease affects the daily living abilities of the patient, and determine appropriate treatment and prognosis.

The term “effective dose” as used herein, generally refers to that amount of an immunogen comprising an internal conserved protein, or an immunogenic fragment thereof, of an infectious agent or pathogen described herein, or a vaccine comprising the immunogen, sufficient to induce immunity, to control and/or ameliorate an infection or to reduce at least one symptom of an infection and/or to enhance the efficacy of another dose of a immunogen or vaccine comprising the immunogen. An effective dose may refer to the amount of immunogen or vaccine comprising the immunogen sufficient to delay or minimize the onset of an infection. An effective dose may also refer to the amount of immunogen or vaccine comprising the immunogen that provides a therapeutic benefit in the treatment or management of an infection. Further, an effective dose is the amount with respect to an immunogen or vaccine comprising the immunogen of the disclosure alone, or in combination with other therapies, that provides a therapeutic benefit in the treatment or management of an infection. An effective dose may also be the amount sufficient to enhance a subject's (e.g., a human's) own immune response against a subsequent exposure to an infectious agent. Levels of immunity can be monitored, e.g., by measuring amounts of neutralizing secretory and/or serum antibodies, e.g., by plaque neutralization, complement fixation, enzyme-linked immunosorbent, or microneutralization assay. In the case of a vaccine, an “effective dose” is one that prevents disease and/or reduces the severity of symptoms.

The term “effective amount” as used herein, is meant to refer to an amount of immunogen or vaccine comprising the immunogen necessary or sufficient to realize a desired biologic effect. An effective amount of the composition would be the amount that achieves a selected result, and such an amount could be determined as a matter of routine experimentation by a person skilled in the art. For example, an effective amount for preventing, controlling, treating and/or ameliorating an infection could be that amount necessary to cause activation of the immune system, resulting in the development of an antigen specific immune response upon exposure to immunogens or vaccines comprising the immunogen of the disclosure. The term is also synonymous with “sufficient amount”

The term “effector cell” as used herein refers to a cell that carries out a final response or function. The main effector cells of the immune system, for example, are activated lymphocytes and phagocytes.

The term “effector functions” as used herein refers to the actions taken by effector cells and antibodies to eliminate foreign entities, and includes, without limitation, cytokine secretion, cytotoxicity, and antibody-mediated clearance.

The term “endogenous” as used herein refers to any material from or produced inside an organism, cell, tissue or system.

The term “enrich” as used herein refers to increasing the proportion of a desired substance, for example, to increase the relative frequency of a subtype of cell compared to its natural frequency in a cell population. Positive selection, negative selection, or both are generally considered necessary to any enrichment scheme. Selection methods include, without limitation, magnetic separation and FACS. Regardless of the specific technology used for enrichment, the specific markers used in the selection process are critical, since developmental stages and activation-specific responses can change a cell's antigenic profile.

As used herein, the terms “expanding a population of cytokine killer T cells (CKTCs)” or “cytokine killer T cell (CKTC)expansion” are meant to refer to a process wherein a population of cytokine killer T cells undergoes a series of cell divisions and thereby expands in cell number (for example, by in vitro culture). The term “expanded superactivated cytokine killer T cells” relates to superactivated cytokine killer T cells obtained through cell expansion.

As used herein, the term “expression” is meant to encompass production of an observable phenotype by a gene, usually b directing the synthesis of a protein. It includes the biosynthesis of mRNA, polypeptide biosynthesis, polypeptide activation, e.g., by post-translational modification, or an activation of expression by changing the subcellular location or by recruitment to chromatin.

As used herein the term “Fas” is meant to refer to a type 2 membrane protein found on lymphocytes that belongs to the TNF superfamily. In cells that express Fas, engagement of the cell death receptor Fas by Fas ligand (FasL) results in apoptotic cell death, mediated by caspase activation.

The term “flow cytometry” as used herein, is meant to refer to a tool for interrogating the phenotype and characteristics of cells. It senses cells or particles as they move in a liquid stream through a laser (light amplification by stimulated emission of radiation)/light beam past a sensing area. The relative light-scattering and color-discriminated fluorescence of the microscopic particles is measured. Flow analysis and differentiation of the cells is based on size, granularity, and whether the cell is carrying fluorescent molecules in the form of either antibodies or dyes. As the cell passes through the laser beam, light is scattered in all directions, and the light scattered in the forward direction at low angles (0.5-10°) from the axis is proportional to the square of the radius of a sphere and so to the size of the cell or particle. Light may enter the cell; thus, the 90° light (right-angled, side) scatter may be labeled with fluorochrome-linked antibodies or stained with fluorescent membrane, cytoplasmic, or nuclear dyes. Thus, the differentiation of cell types, the presence of membrane receptors and antigens, membrane potential, pH, enzyme activity, and DNA content may be facilitated. Flow cytometers are multiparameter, recording several measurements on each cell; therefore, it is possible to identify a homogeneous subpopulation within a heterogeneous population (Marion G. Macey, Flow cytometry: principles and applications, Humana Press, 2007). Fluorescence-activated cell sorting (FACS), which allows isolation of distinct cell populations too similar in physical characteristics to be separated by size or density, uses fluorescent tags to detect surface proteins that are differentially expressed, allowing fine distinctions to be made among physically homogeneous populations of cells.

The terms “follicular helper T cell” (“T_{H}F”), and “circulatory follicular helper CD4+ T cells” [“cT_{H}F” ] as used herein are used interchangeably to refer to a type of effector CD4 T cell that resides in lymphoid follicles and provides help to B cells for antibody production.

As used herein, the terms “formulation” and “composition” are used interchangeably herein to refer to a product of the present disclosure that comprises all active and inert ingredients. The terms “pharmaceutical formulation” or “pharmaceutical composition” as used herein refer to a formulation or composition that is employed to prevent, reduce in intensity, cure or otherwise treat a target condition or disease.

The terms “functional equivalent” and “functionally equivalent” are used interchangeably herein to refer to substances, molecules, polynucleotides, proteins, peptides, or polypeptides having similar or identical effects or use.

The term “GALTs” as used herein refers to gut-associated lymphoid tissues, which are part of the mucosa-associated lymphoid tissues (MALTs). The histological components of GALTs mainly includes Peyer's patches, crypt patches, isolated lymphoid follicles (ILFs) appendix and mesenteric lymph nodes (mLNs). [Jiao, Y. et al., Crosstalk between gut microbiota and innate immunity and its implication in autoimmune disease. Front. Immunol. (2020) 11: 282; citing Brandtzaeg, P. et al. Terminology: nomenclature of mucosa-associated lymphoid tissue. Mucosal Immunol. (2008) 1: 31; Mowat, A M. Anatomical basis of tolerance and immunity to intestinal antigens. Nat. Rev. Immunol. (2003) 3: 331-41] constituent cells of GALTs include microfold (M) cells, which are capable of transferring antigens but not processing or presenting them [Id., citing Mabbott, N A et al. Microfold (M) cells: important immunosurveillance posts in the intestinal epithelium, Mucosal Immunol. (2013) 6:666]. Conventional lymphocytes, such as helper T cells (T_{H} cells) (Id., citing Dunkley, M., Husband, A. Distribution and functional characteristics of antigen-specific helper T cells arising after Peyer's patch immunization. J. Immunol. (1987) 61: 475; Kiyono, H. e al. Murine Peyer's patch T cell clones. Characterization of antigen-specific helper T cells for immunoglobulin A responses. J. Exp. Med. (1982) 156: 1115-30]; Tregs (Id., citing Coombes, J L., et al. A functionally specialized population of mucosal CD103+ DCs induces Foxp3+ regulatory T cells via a TGFβ-and-retinoid acid-dependent mechanism. J. Exp. Med. (2007) 204: 1757-64; Siddiqui, K., Powrie, F. CD103+ GALT DCs promote Foxp3+ regulatory T cells. Mucosal Immunol. (2008) 1 (Suppl. 1): 534-8), cytotoxic T lymphocytes (Id. citing Nelson, D L et al. Cytotoxic effector cell function in organized gut-associated lymphoid tissue (GALT). Cell Immunol. (1976) 22: 65-75), IgA producing B cells (Id., citing Mora, J R et al. Generation of gut-homing IgA-secreting B cells by intestinal dendritic cells. Science (2006) 314: 1157-60), phagocytes, including dendritic cells (Id., citing Coombes, J L., et al. A functionally specialized population of mucosal CD103+ DCs induces Foxp3+ regulatory T cells via a TGFβ-and-retinoid acid-dependent mechanism. J. Exp. Med. (2007) 204: 1757-64; Siddiqui, K., Powrie, F. CD103+ GALT DCs promote Foxp3+ regulatory T cells. Mucosal Immunol. (2008) 1 (Suppl. 1): 534-8), macrophages, and other nonconventional lymphocytes, such as innate lymphoid cells (ILCs) (Id., citing Pearson, C. et al. Lymphoid microenvironments and innate lymphoid cells in the gut. Trends Immunol. (2012) 33: 289-96; Wojno, E D T; Artis, D. Innate lymphoid cells: balancing immunity, inflammation and tissue repair in the intestine. Cell Host Microbe (2012) 12: 445-57). The gut microbiota shapes the structural development of GALTs and primes its immune response to initiate host defense and to maintain tolerance against commensal bacteria via PRR-PAMP recognition and epigenetic modulators like short-chain fatty acids (SCFAs). [Id.]

The terms “GATA-3” and “GATA binding protein 3” are used interchangeably to refer to a member of the GATA family of conserved zinc-finger transcription factors, several of which are involved in hematopoiesis. GATA-3 is highly expressed in T cells and a wide variety of other tissues, including the CNS and fetal liver. In T cells, Gata3 acts at multiple stages of thymocyte differentiation. It is indispensable for early thymic progenitor differentiation [Hosoya, T. et al., J Exp Med. 2009 206(13):2987-3000] and for thymocytes to pass through beta selection and T cell commitment. Gata3 is also necessary for single-positive CD4 thymocyte development as well as for T_{H}1-T_{H}2 lineage commitment [Ting, C N et al., Nature. (1996) 384(6608):474-8; Thang, D H et al., J Biol Chem. (1997) 272(34):21597-603; Zheng W, Flavell R A. Cell. (1997) 89(4):587-96; Zhang, D H et al., J Immunol. (1998) 161(8):3817-21; Pai, S Y et al. Immunity (2003) 19(6):863-753]. As master regulator of Th2 lineage commitment, GATA3 acts either as a transcriptional activator or repressor through direct action at many critical loci encoding cytokines, cytokine receptors, signaling molecules as well as transcription factors that are involved in the regulation of T(h)1 and T(h)2 differentiation [Jenner, R G et al., Proc Natl Acad Sci USA. (2009) 106(42):17876-81]. For example, it regulates the expression of 7b2 lineage specific cytokine gene such as IL5 and represses the T_{H}1 lineage specific genes IL-12 receptor β2 and STAT4 as well as neutralizing RUNX3 function through protein-protein interaction. Mice lacking Gata3 produce IFN-gamma rather than T_{H}2 cytokines (IL5 and IL13) in response to infection [Zhu, J et al., Nat Immunol. (2004) 5(11):1157-65]. It acts in mutual opposition to the transcription factor T-bet, as T-bet promotes whereas GATA3 represses Fut7 transcription [Hwang, E S et al., Science. (2005) 21; 307(5708):430-3]. It also acts with Tbx21 to regulate cell lineage-specific expression of lymphocyte homing receptors and cytokine in both T_{H}1 and T_{H}2 lymphocyte subsets [Chen, G Y et al., Proc Natl Acad Sci USA. (2006) 103(45):16894-9]. Enforced expression of Gata3 during T cell development induced CD4(+)CD8(+) double-positive (DP) T cell lymphoma [Nawijn, M C et al., J Immunol. (2001) 167(2):724-32a; Nawijn, M C et al., J Immunol. (2001) 167(2):715-23]. Gata3 is essential for the expression of the cytokines IL-4, IL-5 and IL-13 that mediate allergic inflammation. Gata3 overexpression causes enhanced allergen-induced airway inflammation and airway remodeling, including subepithelial fibrosis, and smooth muscle cell hyperplasia [Kiwamoto, T et al., Am J Respir Crit Care Med. (2006) 174(2):142-51]. It additionally has a critical function in regulatory T cells and immune tolerance since deletion of Gata3 specifically in regulatory T cells led to a spontaneous inflammatory disorder in mice [Wang, Y et al., Immunity (2011) 35(3):337-48].

The term “graft-versus-host disease” or “GvHD”, as used herein refers to a complication of allogeneic transplantation in which donated T cells from a non-identical donor view the recipient's body as foreign and the donated cells attack the tissues of the recipient.

The term “granulocyte-macrophage colony-stimulating factor (GM-CSF) as used herein refers to a cytokine involved in the growth and differentiation of cells of the myeloid lineage, including dendritic cells, monocytes and tissue macrophages, and granulocytes.

The term “granulocytes” as used herein refers to myeloid leukocytes that harbor large intracellular granules containing microbe-destroying hydrolytic enzymes, and includes neutrophils, basophils and eosinophils. In synergy with other cytokines such as stem cell factor, IL-3, erythropoietin, and thrombopoietin, it also stimulates erythroid and megakaryocyte progenitor cells (Barreda, D R, et al, Developmental & Comparative Immunol. (2004) 28(50: 509-554). GM-CSF is produced by multiple cell types, including stromal cells, Paneth cells, macrophages, dendritic cells (DCs), endothelial cells, smooth muscle cells, fibroblasts, chondrocytes, and T_{H}1 and T_{H}17 T cells (Francisco-Cruz, A. et al, Medical Oncology (2014) 31: 774 et al.).

The term “helper T cells” or “T_{H}” cells as used herein refers to effector CD4 T cells that stimulate or “help” B cells to make antibody in response to antigenic challenge. T_{H}2, T_{H}1 and the T_{H}F subsets of effector CD4 T cells can perform this function.

The term “homeostatic proliferation” as used herein refers to a process of activation and proliferation of leukocytes in a lymphopenic environment. T cell homeostatic proliferation is driven by T cell receptor interactions with self-peptide-MHC complexes and responsiveness to homeostatic cytokines, such as IL7, IL-15, and possibly IL-21. [Gattinoni, L. et al. Natur Revs. Cancer 12: 671-684].

The term “herd immunity” as used herein refers to protection conferred to unvaccinated individuals in a population produced by vaccination of others and reduction in the natural reservoir for infection.

The term “heterologous immunity” as used herein refers to an immunity that can develop to one pathogen after a host has had exposure to non-identical pathogens.

The term “heterosubtypic immunity” (“HSI”) as used herein refers to immunity based on immune recognition of antigens conserved across all viral strains.

The term “heterotypic” as used herein is used to refer to being of a different or unusual type or form (e.g., different subgroup, subtype, strain and/or variant of a virus, bacteria, parasite or other pathogen).

The term “homotypic” as used herein is used to refer to being of the same type or form, e.g., same subgroup, subtype, strain and/or variant of a virus, bacteria, parasite or other pathogen.

The term “IL-4Rα” as used herein refers to the cytokine-binding receptor chain for IL-4.

The terms “immune response” and “immune-mediated” are used interchangeably herein to refer to any functional expression of a subject's immune system, against either foreign or self-antigens, whether the consequences of these reactions are beneficial or harmful to the subject. The term “immunological response” to an antigen or composition as used herein, is meant to refer to the development in a subject of a humoral and/or a cellular immune response to an antigen present in the composition of interest. For purposes of the present disclosure, a “humoral immune response” refers to an immune response mediated by antibody molecules, while a “cellular immune response” is one mediated by T-lymphocytes and/or other white blood cells. One aspect of cellular immunity involves an antigen-specific response by cytolytic T-cells (“CTL”s). CTLs have specificity for peptide antigens that are presented in association with proteins encoded by the major histocompatibility complex (MHC) and expressed on the surfaces of cells. CTLs help induce and promote the destruction of intracellular microbes or the lysis of cells infected with such microbes. Another aspect of cellular immunity involves an antigen-specific response by helper T-cells. Helper T-cells act to help stimulate the function, and focus the activity of, nonspecific effector cells against cells displaying peptide antigens in association with MHC molecules on their surface. A “cellular immune response” also refers to the production of cytokines, chemokines and other such molecules produced by activated T-cells and/or other white blood cells, including those derived from CD4+ and CD8+ T-cells. Hence, an immunological response may include one or more of the following effects: the production of antibodies by B-cells; and/or the activation of suppressor T-cells and/or γδ T-cells directed specifically to an antigen or antigens present in the composition or vaccine of interest. These responses may serve to neutralize infectivity, and/or mediate antibody-complement, or antibody dependent cell cytotoxicity (ADCC) to provide protection to an immunized host. Such responses can be determined using standard immunoassays and neutralization assays, well known in the art.

The term “immune phenotype” or “immunotype” as used herein refers to the collective frequency of various immune cell populations and their functional responses to stimuli (cell signaling and antibody responses). [See Kaczorowski, K J et al. Proc. Nat. Acad. Sci. USA (2017) doi/10.1073/pnas.1705065114]

The terms “immune surveillance” or “immunological surveillance” are used interchangeably to refer to a monitoring process by the immune system to detect and destroy virally infected and neoplastically transformed cells in the body.

The term “immune system” as used herein refers to the body's system of defenses against disease, which comprises the innate immune system and the adaptive immune system. The innate immune system provides a non-specific first line of defense against pathogens. It comprises physical barriers (e.g. the skin) and both cellular (granulocytes, natural killer cells) and humoral (complement system) defense mechanisms. The reaction of the innate immune system is immediate, but unlike the adaptive immune system, it does not provide permanent immunity against pathogens. The adaptive immune response is the response of the vertebrate immune system to a specific antigen that typically generates immunological memory.

The term “immunocompromised” as used herein refers to having a weakened immune system and a reduced ability to fight infections and other diseases. The term “immunocompromised” as used herein refers to having a weakened immune system and a reduced ability to fight infections and other diseases. Immunocompromised subjects include patients receiving long-term (>3 months) or high-dose (>0.5 mg/kg/day) steroids or other immunosuppressant drugs, organ or bone marrow transplant recipients, patients with a solid tumor requiring chemotherapy in the last 5 years or with a hematological malignancy whatever the time since diagnosis and who received treatments, patients with leukemia or lymphoma, patients with primary immune deficiency; patients with HIV or AIDS; patients with autoimmune conditions, patients with asthma, which causes the immune system to overreact to harmless substances); patients of advanced age; and smokers.

The term “immunological repertoire” refers to the collection of transmembrane antigen-receptor proteins located on the surface of T and B cells. [Benichou, J. et al. Immunology (2011) 135: 183-191)] The combinatorial mechanism that is responsible for encoding the receptors does so by reshuffling the genetic code, with a potential to generate more than 10¹⁸ different T cell receptors (TCRs) in humans [Id., citing Venturi, Y. et al. Nat. Rev. Immunol. (2008) 8: 231-8] and a much more diverse B-cell repertoire. These sequences, in turn, will be transcribed and then translated into protein to be presented on the cell surface. The recombination process that rearranges the gene segments for the construction of the receptors is key to the development of the immune response, and the correct formation of the rearranged receptors is critical to their future binding affinity to antigen. For example, diversity of the TCR gene is generated by rearrangement of the V and J gene segments during T cell development in the thymus. (Makino, Y., et al (1993) J. Exptl Med. 177: 1399-1408). The TCR V and J gene segments, like Ig genes, possess recombination signals in which heptamer and nonamer sequences, separated by a 12/23 bp spacer, are flanked by germline V and J gene segments. Id.

The term “immunogen” and its various grammatical forms as used herein is used interchangeably with the term “antigen”.

The terms “immunomodulatory”, “immune modulator”, “immunomodulatory,” and “immune modulatory” are used interchangeably herein to refer to a substance, agent, or cell that is capable of augmenting or diminishing immune responses directly or indirectly, e.g., by expressing chemokines, cytokines and other mediators of immune responses.

As used herein, the term “immunostimulatory amount” refers to an amount of an immunogenic composition that stimulates an immune response by a measurable amount, for example, as measured by ELISPOT assay (cellular immune response), ICS (intracellular cytokine staining assay) and major histocompatibility complex (MHC) tetramer assay.

As used herein the term “immunosuppressive amount” refers to an amount of an immunosuppressive composition that suppresses an immune response, for example, as measured by ELISPOT assay (cellular immune response), ICS (intracellular cytokine staining assay) and major histocompatibility complex (MHC) tetramer assay.

The term “inflammasome” as used herein refers to a pro-inflammatory protein complex that is formed after stimulation of the intracellular NOD-like receptors. Production of an active caspase in the complex processes cytokine proteins into active cytokines.

The term “inflammation” as used herein refers to the physiologic process by which vascularized tissues respond to injury. See, e.g., FUNDAMENTAL IMMUNOLOGY, 4th Ed., William E. Paul, ed. Lippincott-Raven Publishers, Philadelphia (1999) at 1051-1053, incorporated herein by reference. During the inflammatory process, cells involved in detoxification and repair are mobilized to the compromised site by inflammatory mediators. Inflammation is often characterized by a strong infiltration of leukocytes at the site of inflammation, particularly neutrophils (polymorphonuclear cells). These cells promote tissue damage by releasing toxic substances at the vascular wall or in uninjured tissue. Traditionally, inflammation has been divided into acute and chronic responses.

The term “acute inflammation” as used herein refers to the rapid, short-lived (minutes to days), relatively uniform response to acute injury characterized by accumulations of fluid, plasma proteins, and neutrophilic leukocytes. Examples of injurious agents that cause acute inflammation include, but are not limited to, pathogens (e.g., bacteria, viruses, parasites), foreign bodies from exogenous (e.g. asbestos) or endogenous (e.g., urate crystals, immune complexes), sources, and physical (e.g., burns) or chemical (e.g., caustics) agents.

The term “chronic inflammation” as used herein refers to inflammation that is of longer duration and which has a vague and indefinite termination. Chronic inflammation takes over when acute inflammation persists, either through incomplete clearance of the initial inflammatory agent or as a result of multiple acute events occurring in the same location. Chronic inflammation, which includes the influx of lymphocytes and macrophages and fibroblast growth, may result in tissue scarring at sites of prolonged or repeated inflammatory activity.

The term “inflammatory mediators” or “inflammatory cytokines” as used herein refers to the molecular mediators of the inflammatory process. These soluble, diffusible molecules act both locally at the site of tissue damage and infection and at more distant sites. Some inflammatory mediators are activated by the inflammatory process, while others are synthesized and/or released from cellular sources in response to acute inflammation or by other soluble inflammatory mediators. Examples of inflammatory mediators of the inflammatory response include, but are not limited to, plasma proteases, complement, kinins, clotting and fibrinolytic proteins, lipid mediators, prostaglandins, leukotrienes, platelet-activating factor (PAF), peptides and amines, including, but not limited to, histamine, serotonin, and neuropeptides, and proinflammatory cytokines, including, but not limited to, interleukin-1-beta (IL-1β), interleukin-4 (IL-4), interleukin-6 (IL-6), interleukin-8 (IL-8), tumor necrosis factor-alpha (TNF-α), interferon-gamma (IF-γ), and interleukin-12 (IL-12). Among the pro-inflammatory mediators, IL-1, IL-6, and TNF-α are known to activate hepatocytes in an acute phase response to synthesize acute-phase proteins that activate complement.

The term “infusion” as used herein refers to the introduction of fluid other than blood into a vein.

The terms “inhibiting”, “inhibit” or “inhibition” are used herein to refer to reducing the amount or rate of a process, to stopping the process entirely, or to decreasing, limiting, or blocking the action or function thereof. Inhibition may include a reduction or decrease of the amount, rate, action function, or process of a substance by at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, at least 85%, at least 90%, at least 95%, at least 98%, or at least 99%. The term “injury” as used herein refers to damage or harm to a structure or function of the body caused by an outside agent or force, which may be physical or chemical. As used herein, the term “interferon gamma” (IFN-y) is meant to refer to a soluble cytokine that is a member of the type II interferon class, which is secreted by cells of both the innate and adaptive immune systems. The active protein is a homodimer that binds to the interferon gamma receptor, which triggers a cellular response to viral and microbial infections.

The term “interleukin (IL)” as used herein refers to a cytokine secreted by, and acting on, leukocytes. Interleukins regulate cell growth, differentiation, and motility, and stimulates immune responses, such as inflammation. Examples of interleukins include, without limitation, interleukin-1 (IL-1), interleukin-1β (IL-1β), interleukin-2 (IL-2), interleukin-6 (IL-6), interleukin-7 (IL-7), interleukin-8 (IL-8), interleukin-12 (IL-12), interleukin-15 (IL-15), and interleukin 37 (IL-37).

As used herein, the term “interleukin-2” (IL-2) is meant to refer to a type of cytokine made by a type of T-lymphocyte that increases the growth and activity of other T lymphocytes and B lymphocytes and affects the development of the immune system. IL-2 made in the laboratory is called aldesleukin.

As used herein, the term “interleukin 4” (IL-4) is a pleiotropic cytokine whose actions are generally antagonistic to those of interferon gamma. Because IL-4R is widely expressed, IL-4 influences almost all cell types. In T cells, IL-4 is crucial for the differentiation and growth of the Th2 subset. As such, IL-4 promotes the establishment of the humoral response necessary to combat pathogens that live and reproduce extracellularly. In B cells, IL-4 stimulates growth and differentiation and induces upregulation of MHC class II and FcεRII (CD23). IL-4 also promotes isotype switching in murine B cells to IgG1 and IgE but inhibits switching to IgG2a, IgG2b, and IgG3. IL-4 is a growth factor for mast cells and plays a major regulatory role in allergic responses since these involve IgE-mediated mast cell degranulation. IL-4 is also important for defense against helminth worms because the IgE production promoted by IL-4 allows eosinophils bearing FcεRIIB to carry out efficient ADCC. In macrophages, IL-4 inhibits the secretion of pro-inflammatory chemokines and cytokines such as TNF and IL-1β, impairs the ability of these cells to produce reactive oxygen and nitrogen intermediates, and blocks IFNγ-induced expression of cellular adhesion molecules such as ICAM and E-selectin. However, IL-4 can also induce DCs and macrophages to upregulate their synthesis of IL-12, supplying a negative feedback mechanism to regulate the Th2 response. Mak, T W, Saunders, M E, Chapter 17, “Cytokines and Cytokine Receptors,” in The Immune Response, Basic and Clinical Principles (2006), Academic Press, pp. 463-516).

As used herein, the terms “interleukin-7” (IL-7) also known as “lymphopoietin-1”, are meant to refer to a type of cytokine made by cells that cover and support organs, glands and other structures in the body that causes the growth of T lymphocytes and B lymphocytes.

As used herein, the term “interleukin-12” (IL-12) is meant to refer to a type of cytokine made mainly by B lymphocytes and macrophages that causes other immune cells to make cytokines and increase the growth of T lymphocytes. It may also block the growth of new blood vessels.

As used herein, the term “interleukin-15” (IL-15) is meant to refer to a type of cytokine that acts through its specific receptor, IL-15Rα, which is expressed on antigen-presenting dendritic cells, monocytes and macrophages. IL-15 regulates T and natural killer cell activation and proliferation. IL-15 and IL-2 share many biological activities. They are found to bind common hematopoietin receptor subunits, and may compete for the same receptor, and thus negatively regulate each other's activity. The number of CD8+ memory cells is shown to be controlled by a balance between IL-15 and IL2. IL-15 induces the activation of JAK kinases, as well as the phosphorylation and activation of transcription activators STAT3, STAT5, and STAT6. Studies of the mouse counterpart suggested that IL-15 may increase the expression of apoptosis inhibitor BCL2L1/BCL-x(L), possibly through the transcription activation activity of STAT6, and thus prevent apoptosis.

The term “isolated” is used herein to refer to material, such as, but not limited to, a nucleic acid, peptide, polypeptide, protein, or cell which is: (1) substantially or essentially free from components that normally accompany or interact with it as found in its naturally occurring environment. For example, a naturally-occurring polynucleotide or polypeptide present in a living animal is not isolated, but the same polynucleotide or polypeptide, separated from some or all of the coexisting materials in the natural system, is isolated. The terms “substantially free” or “essentially free” are used herein to refer to considerably or significantly free of, or more than about 95% free of, or more than about 99% free of such components. The isolated material optionally comprises material not found with the material in its natural environment; or (2) if the material is in its natural environment, the material has been synthetically (non-naturally) altered by deliberate human intervention to a composition and/or placed at a location in the cell (e.g., genome or subcellular organelle) not native to a material found in that environment.

The term “labeling” as used herein refers to a process of distinguishing a compound, structure, protein, peptide, antibody, cell or cell component by introducing a traceable constituent. Common traceable constituents include, but are not limited to, a fluorescent antibody, a fluorophore, a dye or a fluorescent dye, a stain or a fluorescent stain, a marker, a fluorescent marker, a chemical stain, a differential stain, a differential label, and a radioisotope.

The term “lectin” as used herein refers to a carbohydrate-binding protein.

The term “lymphocyte” refers to a small white blood cell formed in lymphatic tissue throughout the body and in normal adults making up about 22-28% of the total number of leukocytes in the circulating blood that plays a large role in defending the body against disease. Individual lymphocytes are specialized in that they are committed to respond to a limited set of structurally related antigens. This commitment, which exists before the first contact of the immune system with a given antigen, is expressed by the presence on the lymphocyte's surface membrane of receptors specific for determinants (epitopes) on the antigen. Each lymphocyte possesses a population of receptors, all of which have identical combining sites. One set, or clone, of lymphocytes differs from another clone in the structure of the combining region of its receptors and thus differs in the epitopes that it can recognize. Lymphocytes differ from each other not only in the specificity of their receptors, but also in their functions. Two broad classes of lymphocytes are recognized: the B-lymphocytes (B-cells), which are precursors of antibody-secreting cells, and T-lymphocytes (T-cells),

The term “lymphocyte activation” refers to stimulation of lymphocytes by specific antigens, nonspecific mitogens, or allogeneic cells resulting in synthesis of RNA, protein and DNA and production of lymphokines; it is followed by proliferation and differentiation of various effector and memory cells. For example, a mature B cell can be activated by an encounter with an antigen that expresses epitopes that are recognized by its cell surface immunoglobulin Ig). The activation process may be a direct one, dependent on cross-linkage of membrane Ig molecules by the antigen (cross-linkage-dependent B cell activation) or an indirect one, occurring most efficiently in the context of an intimate interaction with a helper T cell (“cognate help process”). T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC). The soluble product of an activated B lymphocyte is immunoglobulins (antibodies). The soluble product of an activated T lymphocyte is lymphokines (meaning cytokines produced by lymphocytes).

The term “macrophage” as used herein refers to a mononuclear, actively phagocytic cell arising from monocyte stem cells in the bone marrow. These cells are widely distributed in the body and vary in morphology and motility. Phagocytic activity is typically mediated by serum recognition factors, including certain immunoglobulins and components of the complement system, but also may be nonspecific. Macrophages also are involved in both the production of antibodies and in cell-mediated immune responses, particularly in presenting antigens to lymphocytes. They secrete a variety of immunoregulatory molecules.

The terms “Major Histocompatibility Complex (MHC), MHC-like molecule” and “HLA” are used interchangeably herein to refer to cell-surface molecules that display a molecular fraction known as an epitope or an antigen and mediate interactions of leukocytes with other leukocyte or body cells. MHCs are encoded by a large gene group and can be organized into three subgroups—class I, class II, and class III. In humans, the MHC gene complex is called HLA (“Human leukocyte antigen”); in mice, it is called H-2 (for “histocompatibility”). Both species have three main MHC class I genes, which are called HLA-A, HLA-B, and HLA-C in humans, and H2-K, H2-D and H2-L in the mouse. These encode the α chain of the respective MHC class I proteins. The other subunit of an MHC class I molecule is β2-microglobulin. The class II region includes the genes for the α and β chains (designated A and B) of the MHC class II molecules HLA-DR, HLA-DP, and HLA-DQ in humans. Also in the MHC class II region are the genes for the TAP:TAP2 peptide transporter, the PSMB (or LMP) genes that encode proteasome subunits, the genes encoding the DMα and BMO chains (DMA and DMB), the genes encoding the α and β chains of the DO molecule (DOA and DOB, respectively), and the gene encoding tapasin (TAPBP). The class II genes encode various other proteins with functions in immunity. The DMA and DMB genes encoding the subunits of the HLA-DM molecule that catalyzes peptide binding to MHC class II molecules are related to the MHC class II genes, as are the DOA and DOB genes that encode the subunits of the regulatory HLA-DO molecule. [Janeways Immunobiology. 9th ed., G S, Garland Science, Taylor & Francis Group, 2017. pps. 232-233]. In humans, there are three MHC class II isotypes: HLA-DR, HLA-DP, and HLA-DQ, encoded by α and β chain genes within the Human Leukocyte Antigen (HLA) locus on chromosome 6 [Wosen, J E et al. Front. Immunol. (2018) doi.10.3389/fimmu.2018.02144].

The term “MHC restriction” as used herein refers to the requirement that APC or target cells express MHC molecules that a T cell recognizes as self in order for T cell to respond to the antigen presented by that APC or target cell (T cells will only recognize antigens presented by their own MHC molecules). For example, CD8 T cells bind class I MHC which are expressed on most cells in the body, and CD4 T cells bind class II MHC which are only expressed on specialized APCs.

MHC-like molecules, while not encoded by the same gene group as true MHCs, have the same folding and overall structure of MHCs, specifically MHC class I molecules, and thus possesses similar biological functions, such as antigen presentation.

MR1, a nonclassical histocompatibility molecule which activates MAIT cells, and the CD1 family of molecules are examples of MHC-like molecules.

CD1 consists of two groups based on amino acid homology: group 1, which includes CD1a, b, and c; and group 2, which consists of CD1d. Exemplary CD1 antigenic ligands include, without limitation, dideoxymycobactin (Cd1a); glycose monomycolate (Cd1b), sulfatide (CD1a-d)mycolic acid, (CD1b), mannosyl-β1-phosphodolicol (Cd1c) alpha-galactosylceramide (alpha-GalCer, CD1d); phenylpentamethyl dihydrobenzofuransulphonate (CD1d), isoglobotriheoxylceramide (CD1d); palmitoyl-oleoyl-sn-glycero-3-phosphoethanolamine (CD1d); and α-galacturonosyl ceramide (CD1d). Group 1 CD1 molecules can present antigens to a wide variety of T cells, whereas CD1d presents antigens mostly to NKT cells. (Brutkiewicz. “CD1d Ligands: The Good, the Bad, and the Ugly.” The Journal of Immunology (2006) 177 (2) 769-775). While CD1d structurally resembles MHC Class I molecules, it traffics through the endosome of the exogenous antigen presentation pathway. The binding groove of the CD1d molecule tethers the lipid tail of a glycolipid antigen, while the carbohydrate head group of the antigen projects out of the groove for recognition by the TCR of the NKT cell. (Wah, MakTak, et al. “Chapter 11: NK, γδ T and NKT Cells.” Primer to the Immune Response. Elsevier, 2014).

CD1 molecules are glycosylated heterodimers composed of a heavy chain polypeptide noncovalently associated with β2-microglobulin (β2m). Group I and II CD1 proteins are mainly expressed on cortical thymocytes, B-cells (CD1c) and antigen presenting cells (APC), such as dendritic cells (DC). The group II isoform, CD1d, is additionally expressed on macrophages, epithelial cells and hepatocytes [Id., citing Brigl, M.; Brenner, M B. Annu. Rev. Immunol. (2004) 22 (1): 817-90].

CD1 mediates T-cell responses through the presentation of self and foreign lipids, glycolipids, lipopeptides, or amphipathic small molecules to TCRs [Wu, D. et al. “Glycolipids as immunostimulating agents.” Bioorg. Med. Chem. (2008) 16 (3): 1073-83, citing Brigl, M., Brenner, M B. Annu Rev. Immunol. (2004) 22 (1): 817-90; Porcelli, S A, Modlin, R L. Annu. Rev. Immunol. (1999) 17 (1): 297-329; Savage, P B et al. Chemm. Socy Rev. (2007) 35 (9): 771-9].CD1d presents lipid antigens, and requires the presence of particular mechanisms to induce uptake of these molecules by APCs and subsequent loading onto CD1d molecules. Lipid transfer protein such as apolipoprotein E and fatty acid amide hydrolase (FAAH) have been shown to enhance the presentation of certain antigens by CD1d. Loading efficiency can be enhanced by specific proteins, such as saposins and microsomal triglyceride transfer protein, present in the endosomal and lysosomal compartments of cells by promoting lipid antigen exchange. Similar to MHC antigens, lipid antigens can also be processed by lysosomal enzymes to yield active compounds, as demonstrated in the case of CD1d for synthetic antigens, microbial antigens, and self-antigens. [Giradi and Zajonc (2012). “Molecular basis of lipid antigen presentation by CD1d and recognition by natural killer T cells.” Immunol Rev. 250(1): 167-179.]

MHC tetramers are used for the detection of antigen-specific T cell populations. CD1d tetramer is a reagent prepared by tetramerization of complexes of CD1d and β2m by PE- or APC-labeled streptavidin. Binding this reagent to α-GalCer enables highly sensitive detection of CD1d-restricted NKT cells. PBMCs are incubated at room temperature for 5 minutes with 40 μl of Clear Back (Human Fc receptor blocking reagent, MBL code no. MTG-001). CD3-FITC and human CD1d tetramer-PE (with or without binding of α-GalCer) are added and incubated for 30 minutes at 4° C. protected from light. Cells are analyzed by flow cytometry.

As used herein, the terms “marker” or “cell surface marker” are used interchangeably herein to refer to an antigenic determinant or epitope found on the surface of a specific type of cell. Cell surface markers can facilitate the characterization of a cell type, its identification, and eventually its isolation. Cell sorting techniques are based on cellular biomarkers where a cell surface marker(s) may be used for either positive selection or negative selection, i.e., for inclusion or exclusion, from a cell population.

The term “mediated” and its various grammatical forms as used herein refers to depending on, acting by or connected through some intervening agency.

The term “memory cells” as used herein refers to B and T lymphocytes generated during a primary immune response that remain in a quiescent state until fully activated by a subsequent exposure to specific antigen (secondary immune response). Memory cells generally are more sensitive than naïve lymphocytes to antigen and respond rapidly on reexposure to the antigen that originally induced them. During an immune response, naïve T cells (T_{N}) are primed by antigen-presenting cells (APCs). Depending on the strength and quality of stimulatory signals, proliferating T cells progress along a differentiation pathway that culminates in the generation of terminally differentiated short-lived effector T (T_{EFF}) cells. When antigenic and inflammatory stimuli cease, primed T cells become quiescent and enter into the memory stem cell (T_{SCM}), central memory (T_{CM}) cell or effector memory (T_{EM}) cell pools, depending on the signal strength received. T_{SCM} cells possess stem cell-like attributes to a greater extent than any other memory lymphocyte population. Although both T_{CM} and T_{EM} cells can also undergo self-renewal, the capacity to form diverse progeny is progressively restricted, so that only T_{SCM} cells are capable of generating all three memory subsets and Tu cells; T_{CM} cells can give rise to T_{CM}, T_{EM} and T_{EFF} cells, and T_{EM} cells can only produce themselves and T_{EFF} cells. [Gattinoni, L. et al. Nature Revs. Cancer 12 (2012) 671-84].

The term “mitogen” as used herein refers to a substance that stimulates mitosis.

The term “mobilize” and its various grammatical forms as used herein refers to putting into motion or use; becoming ready; being capable of being moved quickly and with relative ease.

The term “modulate” as used herein means to regulate, alter, adapt, or adjust to a certain measure or proportion.

The term “mucosa-associated lymphoid tissue” or MALT, as used herein is a generic term for all organized lymphoid tissue found at mucosal surfaces in which an adaptive immune response can be initiated. It comprises GALT, NALT and BALT (when present). The term “mucosa-associated invariant T cells (MAIT)” as used herein refers primarily to γδT cells with limited diversity present in the mucosal immune system that respond to bacterially derived folate derivatives presented by the nonclassical MHC class 1b molecule MR1.

The term “mucosal epithelia” as used herein refers to mucus-coated epithelia lining the body's internal cavities that connect with the outside (e.g., the gut, airways, and vaginal tract).

The term “mucosal mast cells” as used herein refers to specialized mast cells present in mucosa. They produce little histamine but large amounts of prostaglandins and leukotrienes.

As used herein, the term “mutation” refers to a change of the DNA sequence within a gene or chromosome of an organism resulting in the creation of a new character or trait not found in the parental type, or the process by which such a change occurs in a chromosome, either through an alteration in the nucleotide sequence of the DNA coding for a gene or through a change in the physical arrangement of a chromosome. Three mechanisms of mutation include substitution (exchange of one base pair for another), addition (the insertion of one or more bases into a sequence), and deletion (loss of one or more base pairs).

The term “myeloid” as used herein means of or pertaining to bone marrow. Granulocytes and monocytes, collectively called myeloid cells, are differentiated descendants from common progenitors derived from hematopoietic stem cells in the bone marrow. Commitment to either lineage of myeloid cells is controlled by distinct transcription factors followed by terminal differentiation in response to specific colony-stimulating factors and release into the circulation. Upon pathogen invasion, myeloid cells are rapidly recruited into local tissues via various chemokine receptors, where they are activated for phagocytosis as well as secretion of inflammatory cytokines, thereby playing major roles in innate immunity. [Kawamoto, H., Minato, N. Intl J. Biochem. Cell Biol. (2004) 36 (8): 1374-9].

The abbreviation “NFκB” as used herein refers to a proinflammatory transcription factor that switches on multiple inflammatory genes, including cytokines, chemokines, proteases, and inhibitors of apoptosis, resulting in amplification of the inflammatory response [Barnes, P J, (2016) Pharmacol. Rev. 68: 788-815]. The molecular pathways involved in NF-κB activation include several kinases. The classic (canonical) pathway for inflammatory stimuli and infections to activate NF-κB signaling involve the IKK (inhibitor of κB kinase) complex, which is composed of two catalytic subunits, IKK-α and IKK-β, and a regulatory subunit IKK-γ (or NFκB essential modulator [Id., citing Hayden, M S and Ghosh, S (2012) Genes Dev. 26: 203-234]. The IKK complex phosphorylates Nf-κB-bound IκBs, targeting them for degradation by the proteasome and thereby releasing NF-κB dimers that are composed of p65 and p50 subunits, which translocate to the nucleus where they bind to κB recognition sites in the promoter regions of inflammatory and immune genes, resulting in their transcriptional activation. This response depends mainly on the catalytic subunit IKK-β (also known as IKK2), which carries out IκB phosphorylation. The noncanonical (alternative) pathway involves the upstream kinase NF-κB-inducing kinase (NIK) that phosphorylates IKK-a homodimers and releases RelB and processes p100 to p52 in response to certain members of the TNF family, such as lymphotoxin-β [Id., citing Sun, S C. (2012) Immunol. Rev. 246: 125-140]. his pathway switches on different gene sets and may mediate different immune functions from the canonical pathway. Dominant-negative IKK-β inhibits most of the proinflammatory functions of NF-κB, whereas inhibiting IKK-a has a role only in response to limited stimuli and in certain cells, such as B-lymphocytes. The noncanonical pathway is involved in development of the immune system and in adaptive immune responses. The coactivator molecule CD40, which is expressed on antigen-presenting cells, such as dendritic cells and macrophages, activates the noncanonical pathway when it interacts with CD40L expressed on lymphocytes [Id., citing Lombardi, V et al. (2010) Int. Arch. Allergy Immunol. 151: 179-89].

The term “natural killer (NK) cells” as used herein is meant to refer to lymphocytes in the same family as T and B cells, classified as group I innate lymphocytes. They have an ability to kill tumor cells without any priming or prior activation, in contrast to cytotoxic T cells, which need priming by antigen presenting cells. NK cells secrete cytokines such as IFNγ and TNFα, which act on other immune cells, like macrophages and dendritic cells, to enhance the immune response. Activating receptors on the NK cell surface recognize molecules expressed on the surface of cancer cells and infected cells and switch on the NK cell. Inhibitory receptors act as a check on NK cell killing. Most normal healthy cells express MHCI receptors, which mark them as “self.” Inhibitory receptors on the surface of the NK cell recognize cognate MHCI, which switches off the NK cell, preventing it from killing. Once the decision is made to kill, the NK cell releases cytotoxic granules containing perforin and granzymes, which leads to lysis of the target cell. Natural killer reactivity, including cytokine secretion and cytotoxicity, is controlled by a balance of several germ-line encoded inhibitory and activating receptors such as killer immunoglobulin-like receptors (KIRs) and natural cytotoxicity receptors (NCRs). The presence of the MHC Class I molecule on target cells serves as one such inhibitory ligand for MHC Class I-specific receptors, the Killer cell Immunoglobulin-like Receptor (KIR), on NK cells. Engagement of KIR receptors blocks NK activation and, paradoxically, preserves their ability to respond to successive encounters by triggering inactivating signals. Therefore, if a KIR is able to sufficiently bind to MHC Class I, this engagement may override the signal for killing and allows the target cell to live. In contrast, if the NK cell is unable to sufficiently bind to MHC Class I on the target cell, killing of the target cell may proceed. Consequently, those tumors which express low MHC Class I and which are thought to be capable of evading a T-cell-mediated attack may be susceptible to an NK cell-mediated immune response instead.

The term “natural killer T cell” or “NKT” as used herein, is meant to refer to invariant natural killer T (iNKT) cells, also known as type-I NKT cells, as well as all subsets of non-invariant (Vα24− and Vα24+)natural killer T cells, which express CD3 and an αβ T cell receptor (TCR) (herein termed “natural killer αβ T cells”) or γδ TCR (herein termed “natural killer γδ T cells”), all of which have demonstrated capacity to respond to non-protein antigens presented by CD1 antigens. The non-invariant NKT cells share in common with type-I NKT cells the expression of surface receptors commonly attributed to natural killer (NK) cells, as well as a TCR of either αβ or γδ TCR gene locus rearrangement/recombination. Accordingly, as used herein, the term “NKT cells” refers to a population of cells that includes CD3+Vα24+ NKT cells, CD3+Vα24− NKT cells, CD3+Vα24− CD56+ NKT cells, CD3+Vα24−CD161+ NKT cells, CD3+γδ− TCR+ T cells, and mixtures thereof.

The term “invariant natural killer T cell” as used herein, is meant to be used interchangeably with the term “iNKT,” and is meant to refer to a subset of T-cell receptor (TCR)c-expressing cells that express a restricted TCR repertoire that, in humans, is composed of a Vα24-Jα18 TCRα chain, which is, for example, coupled with a Vβ11 TCRβ chain. iNKT is meant to encompass all subsets of CD3+Vα24+ type-I NKT cells (CD3+CD4+CD8−Vα24+, CD3+CD4−CD8+Vα24−+, and CD3+CD4−CD8−Vα24+) as well as those cells, which can be confirmed to be type-I NKT cells by gene expression or other immune profiling, but have down-regulated surface expression of Vα24 (CD3+Vα24−). This includes cells which either do or do not express the regulatory transcription factor FOXP3. Unlike conventional T cells, which mostly recognize peptide antigens presented by MHC molecules, iNKT cells recognize glycolipid antigens presented by the non-polymorphic MHC class 1-like CD1d.

The term “nebulizer” as used herein refers to a device used to administer liquid medication in the form of a mist inhaled into the lungs.

The term “neutrophils” or “polymorphonuclear neutrophils (PMNs)” as used herein refers to the most abundant type of white blood cells in mammals, which form an essential part of the innate immune system. They form part of the polymorphonuclear cell family (PMNs) together with basophils and eosinophils. Neutrophils are normally found in the blood stream. During the beginning (acute) phase of inflammation, particularly as a result of bacterial infection and some cancers, neutrophils are one of the first-responders of inflammatory cells to migrate toward the site of inflammation. They migrate through the blood vessels, then through interstitial tissue, following chemical signals such as interleukin-8 (IL-8) and C5a in a process called chemotaxis, meaning the directed motion of a motile cell or part along a chemical concentration gradient toward environmental conditions it deems attractive and/or away from surroundings it finds repellent.

The term “non-expanded” as used herein, is meant to refer to a cell population that has not been grown in culture (in vitro) to increase the number of cells in the cell population.

The term “non-replicating” or “replication-impaired” virus refers to a virus that is not capable of replication to any significant extent in the majority of normal mammalian cells or normal primary human cells.

The term “normal healthy subject” as used herein refers to a subject having no symptoms or other evidence of a viral infection.

The term “Nucleotide-binding Oligomerization Domain (NOD)-like receptors (NLRs)” as used herein refers to innate sensors that detect microbial products or cellular damage in the cytoplasm or activate signaling pathways, and are expressed in cells that are routinely exposed to bacteria, such as epithelial cells, macrophages and dendritic cells. Some NLRs activate NFκB to initiate the same inflammatory responses as the TLRs, while others trigger a distinct pathway that induces cell death and the production of pro-inflammatory cytokines. [Janeway's Immunobiology. 9th ed., GS, Garland Science, Taylor & Francis Group, 2017, at 96].

Subfamilies of NLRs can be distinguished based on the other protein domains they contain. For example the NOD subfamily has an amino-terminal caspase recruitment domain (CARD), which is structurally related to the T1R death domain in MyD88, and can dimerize with CARD domains on other proteins to induce signaling. NOD proteins recognize fragments of bacterial cell wall peptidoglycans, although it is not known if they do so through direct binding or through accessory proteins. Id. At 96. NOD1 senses γ-glutamyl diaminopimelic acid (iE-DAP), a breakdown product of peptidoglycans of Gram negative and some Gram positive bacteria, whereas NOD2 recognizes muramyl dipeptide (MDP), which is present in the peptidoglycans of most bacteria. Id. Other members of the NOD family, including NLRX1 and NLRC5, have been identified, but their function is less well understood. [Janeway's Immunobiology. 9th ed., GS, Garland Science, Taylor & Francis Group, 2017, at 96-98]

When NOD1 or NOD2 recognizes its ligand, it recruits the CARD-containing serine-threonine kinase RIP2 (also known as RICK and RIPK2), which associates with the E3 ligases cIAP1, CIAP2, and XIAP, whose activity generates a polyubiquitin scaffold, which recruits TAK1 and IKK and results in activation of NFκB. NFκB then induces the expression of genes for inflammatory cytokines and for enzymes involved in the production of NO. [Janeway's Immunobiology. 9th ed., GS, Garland Science, Taylor & Francis Group, 2017, at 97].

Macrophages and dendritic cells express both TLRs and NOD1 and NOD2, and are activated by both pathways. In epithelial cells, NOD1 may also function as a systemic activator of innate immunity. NOD2 is strongly expressed in the Paneth cells of the gut where it regulates the expression of potent anti-microbial peptides such as the α- and β-defensins. [Janeway's Immunobiology. 9th ed., GS, Garland Science, Taylor & Francis Group, 2017, at 97].

Other members of the NOD family, including NLRX1 and NLRC5, have been identified, but their function is less well understood. [Janeway's Immunobiology. 9th ed., GS, Garland Science, Taylor & Francis Group, 2017, at 96-98]

The NLRP family, another subfamily of NLR proteins, has a pyrin domain in place of the CARD domain at their amino termini. Humans have 14 NLR proteins containing pyrin domains, of which NLRP3 (also known as NAPL3 or cryopyrin) is the best characterized. NLRP3 resides in an inactive form in the cytoplasm, where its leucine rich repeat (LRR) domains are thought to bind the head-shock chaperone protein HSP90 and the co-chaperone SGT1. NRLP3 signaling is induced by reduced intracellular potassium, the generation of reactive oxygen species, or the disruption of lysosomes by particulate or crystalline matter. For example, death of nearby cells can release ATP into the extracellular space, which would activate the purinergic receptor P2X7, which is a potassium channel, and allow potassium ion efflux. A model proposed for ROS-induced NLRP3 activation involves intermediate oxidation of sensor proteins collectively called thioredoxin (TRX). Normally TRX proteins are bound to thioredoxin-interacting protein (TXNIP). Oxidation of TRX by ROS causes dissociation of TXNIP from TRX. The free TXNIP may then displace HSP90 and SGT1 from NLRP3, again causing its activation. In both cases, NLRP3 activation involves aggregation of multiple monomers via their leucine-rich repeat (LRR) and NOD domains to induce signaling. Phagocytosis of particulate matter (e.g. the adjuvant alum), may lead to the rupture of lysosomes and release of the active protease cathepsin B, which can activate NLRP3. [Janeway's Immunobiology. 9th ed., GS, Garland Science, Taylor & Francis Group, 2017, at 98-99].

NLR signaling, as exemplified by NLRP3, leads to the generation of pro-inflammatory cytokines and to cell death through formation of an inflammasome, a multiprotein complex. Activation of the inflammasome proceeds in several stages. Aggregation of NLRP molecules triggers autocleavage of procaspase I, which releases active caspase 1—Aggregation of LRR domains of several NLRP3 molecules, or other NLRP molecules by a specific trigger or recognition event, which induces the pyrin domains of NLRP3 to interact with pyrin domains of ASC (also called PYCARD), an adaptor protein composed of an amino terminal pyrin domain and a carboxy terminal CARD domain, which further drives the formation of a polymeric ASC filament, with the pyrin domains in the center and the CARD domains facing outward; the CARD domains then interact with CARD domains of the inactive protease pro-caspase 1, initiating its CARD-dependent polymerization into discrete caspase 1 filaments. Active caspase 1 then carries out ATP-dependent proteolytic processing of proinflammatory cytokines, particularly 1L-1β and IL-18, into their active forms, and induces a form of cell death (pyroptosis) associated with inflammation because of the release of these pro-inflammatory cytokines upon cell rupture. [Janeway's Immunobiology. 9th ed., GS, Garland Science, Taylor & Francis Group, 2017, at 99-1001.

A priming step, which can result from TLR signaling, must first occur in which cells inducer and translate the mRNAs that encode the pro-forms of IL-1, IL-18 or other cytokines for inflammasome activation to produce inflammatory cytokines. For example, the TLR-3 agonist poly I:C can be used experimentally to prime cells for triggering of the inflammasome. Janeways Immunobiology. 9th ed., GS, Garland Science, Taylor & Francis Group, 2017, at 1001.

Inflammasome activation also can involve proteins of the PYHIN family, which have an H inversion (HIN) domain in place of an LRR domain. There are four PYIN proteins in humans. Id. At 100. A noncanonical inflammasome (caspase I-independent) pathway uses the protease caspase 11, which therefore is both a sensor and an effector molecule, to detect intracellular LPS. [Janeway's Immunobiology. 9th ed., GS, Garland Science, Taylor & Francis Group, 2017, at 1011.

Besides activating effector functions and cytokine production, another outcome of the activation of innate sensing pathways is the induction of co-stimulatory molecules on tissue dendritic cells and macrophages. B7.1 (CD80) and B7.2 (CD86), for example, which are induced on macrophages and tissue dendritic cells by innate sensors such as TLRs in response to pathogenic recognition, are recognized by specific co-stimulatory receptors expressed by cells of the adaptive immune response, particularly CD4 T cells, and their activation by B7 is an important step in activating adaptive immune responses. [Janeway's Immunobiology. 9th ed., GS, Garland Science, Taylor & Francis Group, 2017, at 105].

The term “overall survival” (OS) as used herein, is meant to refer to the length of time from either the date of diagnosis or the start of treatment for a disease that patients diagnosed with the disease are still alive.

The term “oxygen saturation” (SpO₂) as used herein refers to a measurement of how much oxygen the blood is carrying as a percentage of the maximum it could carry. For a healthy individual, the normal SpO₂ should be between 96% to 99%.

The term “parenteral” as used herein refers to introduction into the body by means other than through the digestive tract, for example, without limitation, by way of an injection (i.e., administration by injection), including, for example, subcutaneously (i.e., an injection beneath the skin), intramuscularly (i.e., an injection into a muscle), intravenously (i.e., an injection into a vein), or infusion techniques.

The term “pathogenesis” as used herein refers to the development of a disease and the chain of events leading to that disease and its sequelae.

The term “pathological” as used herein refers to indicative of or caused by disease.

The term “pathophysiology” and its various grammatical forms as used herein refers to derangement of function in an individual or organ due to a disease.

The term “pattern recognition receptors” or “PRRs” as used herein, is meant to refer to receptors that are present at the cell surface to recognize extracellular pathogens; in the endosomes where they sense intracellular invaders, and finally in the cytoplasm. They recognize conserved molecular structures of pathogens, called pathogen associated molecular patterns (PAMPs) specific to the microorganism and essential for its viability. PRRs are divided into four families: toll-like receptors (TLR); nucleotide oligomerization receptors (NLR); C-type leptin receptors (CLR), and RIG-1 like receptors (RLR).

The term “peptide” is used herein to designate a series of amino acid residues, connected one to the other typically by peptide bonds between the alpha-amino and carbonyl groups of the adjacent amino acids. Peptides are typically 9 amino acids in length, but can be as short as 8 amino acids in length, and as long as 14 amino acids in length. A series of amino acids are considered an “oligopeptide” when the amino acid length is greater than about 14 amino acids in length, typically up to about 30 to 40 residues in length. When the amino acid residue length exceeds 40 amino acid residues, the series of amino acid residues is termed a “polypeptide”.

As used herein, the term “perforin” is meant to refer to a molecule that can insert into the membrane of target cells and promote lysis of those target cells. Perforin-mediated lysis is enhanced by enzymes called granzymes.

The terms “peripheral blood mononuclear cells” or “PBMCs” are used interchangeably herein to refer to blood cells having a single round nucleus such as, for example, a lymphocyte or a monocyte. When a Ficoll fractionation of peripheral blood method is used, PBMCs remain at the less dense, upper interface of the Ficoll layer, often referred to as the buffy coat, and are the cells collected. These cells consist of lymphocytes (T cells, B cells, NK cells) and monocytes. In humans, lymphocytes make up the majority of the PBMC population, followed by monocytes, and only a small percentage of dendritic cells.

The term “pharmaceutical composition” is used herein to refer to a composition that is employed to prevent, reduce in intensity, cure or otherwise treat a target condition or disease.

The term “pharmaceutically acceptable carrier” as used herein is meant to refer to any substantially non-toxic carrier conventionally useable for administration of pharmaceuticals in which the isolated polypeptide of the present disclosure will remain stable and bioavailable. The pharmaceutically acceptable carrier must be of sufficiently high purity and of sufficiently low toxicity to render it suitable for administration to the mammal being treated. It further should maintain the stability and bioavailability of an active agent. The pharmaceutically acceptable carrier can be liquid or solid and is selected, with the planned manner of administration in mind, to provide for the desired bulk, consistency, etc., when combined with an active agent and other components of a given composition.

The term “pharmaceutically acceptable salt” as used herein is meant to refer to those salts which are, within the scope of sound medical judgment, suitable for use in contact with the tissues of humans and lower animals without undue toxicity, irritation, allergic response and the like and are commensurate with a reasonable benefit/risk ratio. When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts may be prepared as alkaline metal or alkaline earth salts, such as sodium, potassium or calcium salts of the carboxylic acid group. By “pharmaceutically acceptable salt” is meant those salts which are, within the scope of sound medical judgment, suitable for use in contact with the tissues of humans and lower animals without undue toxicity, irritation, allergic response and the like and are commensurate with a reasonable benefit/risk ratio. Pharmaceutically acceptable salts are well-known in the art. For example, P. H. Stahl, et al. describe pharmaceutically acceptable salts in detail in “Handbook of Pharmaceutical Salts: Properties, Selection, and Use” (Wiley VCH, Zurich, Switzerland: 2002). The salts may be prepared in situ during the final isolation and purification of the compounds described within the present disclosure or separately by reacting a free base function with a suitable organic acid. Representative acid addition salts include, but are not limited to, acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups may be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid. Basic addition salts may be prepared in situ during the final isolation and purification of compounds described within the disclosure by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Pharmaceutically acceptable salts also may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium or magnesium) salts of carboxylic acids may also be made.

The term “plasma cell” as used herein refers to terminally differentiated B cells that secrete antibody. They may be short-lived, with no isotype switching or somatic hypermutation, or long lived, meaning they undergo isotype switching and somatic hypermutation.

The term “plasmablasts” as used herein refer to proliferating progeny of an activated B cell. Plasmablasts become plasma cells. Antigen binding to the BCR triggers activation of Src family kinases such as Lyn and Fyn leading to phosphorylation of Igα (CD79a) and Igβ (CD79b), recruitment of Syk kinase and subsequent recruitment and phosphorylation of BLNK, Btk and PLCγ [Luo, W. et al. J. Immunol. (2014) 193(2): 909-20, citing Packard, T A & Cambier, J C. F1000 prime reports (2013) 5: 40]. These events activate the Ras pathway, PKC pathway and calcium flux, eventually triggering the activation of NF-κB, Erk and JNK. These positive signals are normally counterbalanced by negative signals that limit B cell activation and prevent spontaneous B cell proliferation and differentiation to plasma cells [Id., citing Nitschke, L. Curr. Opin. Immunol. (2005) 17: 2990-97]. Negative signals are generated by a series of membrane receptors (CD22, CD72, FcγRIIb, PIR-B, Siglec-G, etc.) that are phosphorylated by Lyn. This allows them to recruit phosphatases such as SHP1 and SHIP1 that reverse phosphorylation of signaling molecules in the BCR pathway and dampen BCR signaling [Id., citing Poe, J C & Tedder, T F, Trends Immunol. (2012) 33: 413-20; Tsubata, T. Infectious disorders drug targets (2012) 12: 181-90; Vang, T. et al. Annu. Rev. Immunol. (2008) 26: 29-55].

The term “Plasmalyte A” as used herein refers to a sterile, nonpyrogenic isotonic solution in a single dose container for intravenous administration. Each 100 mL contains 526 mg of Sodium Chloride, USP (NaCl); 502 mg of Sodium Gluconate (C₆H₁₁NaO₇); 368 mg of Sodium Acetate Trihydrate, USP (C₂H₃NaO₂.3H₂O); 37 mg of Potassium Chloride, USP (KCl); and 30 mg of Magnesium Chloride, USP (MgCl₂.6H₂O). It contains no antimicrobial agents. The pH is adjusted with sodium hydroxide. The pH is 7.4 (6.5 to 8.0).

The term “potentiate” and its other grammatical forms as used herein means to increase the power, effect, or potency, of; to enhance, to augment the activity of.

The term “prevention” as used herein, is meant to refer to a process of prophylaxis in which an animal (e.g., a mammal, and most especially a human) is exposed to an immunogen of the present disclosure prior to the induction or onset of the disease process. This could be done where an individual is at high risk for any viral infection based on the living or travel to the virus pandemic areas. Alternatively, the immunogen could be administered to the general population as is frequently done for any infectious diseases. Alternatively, the term “suppression” is often used to describe a condition wherein the disease process has already begun but obvious symptoms of said condition have yet to be realized. Thus, the cells of an individual may have been infected but no outside signs of the disease have yet been clinically recognized. In either case, the term prophylaxis can be applied to encompass both prevention and suppression.

The term “priming” as used herein refers to the process whereby T cells and B cell precursors encounter the antigen for which they are specific. The term “unprimed cells” (also referred to as virgin, naïve, or inexperienced cells) as used herein refers to T cells and B cells that have generated an antigen receptor (TCR for T cells, BCR for B cells) of a particular specificity, but have never encountered the antigen. For example, before helper T cells and B cells can interact to produce specific antibody, the antigen-specific T cell precursors must be primed.

Priming involves several steps: antigen uptake, processing, and cell surface expression bound to class II MHC molecules by an antigen presenting cell, recirculation and antigen-specific trapping of helper T cell precursors in lymphoid tissue, and T cell proliferation and differentiation. [Janeway, C A, Jr., “The priming of helper T cells, Semin. Immunol. (1989) 1(1): 13-20]. Helper T cells express CD4, but not all CD4 T cells are helper cells. Id. The signals required for clonal expansion of helper T cells differ from those required by other CD4 T cells. The critical antigen-presenting cell for helper T cell priming appears to be a macrophage; and the critical second signal for helper T cell growth is the macrophage product interleukin 1 (IL-1). Id. If the primed T cells and/or B cells receive a second, co-stimulatory signal, they become activated T cells or B cells.

The term “progression” as used herein refers to the course of a disease as it becomes worse or spreads in the body.

The term “proliferate” and its various grammatical forms as used herein is meant to refer to the process that results in an increase of the number of cells, and is defined by the balance between cell division and cell loss through cell death or differentiation.

The term “protect” or “protection of” a subject from developing a disease or from becoming susceptible to an infection as referred herein means to partially or fully protect a subject. As used herein, to “fully protect” means that a treated subject does not develop a disease or infection caused by an agent such as a virus, bacterium, fungus, protozoa, helminth, and parasites, or caused by a cancer cell. To “partially protect” as used herein means that a certain subset of subjects may be fully protected from developing a disease or infection after treatment, or that the subject does not develop a disease or infection with the same severity as an untreated subject.

The term “protective immune response” or “protective response” as used herein, is meant to refer to an immune response mediated by antibodies against an infectious agent, which is exhibited by a vertebrate (e.g., a human), that prevents or ameliorates an infection or reduces at least one symptom thereof. Vaccines of the present disclosure can stimulate the production of antibodies that, for example, neutralize infectious agents, block infectious agents from entering cells, block replication of said infectious agents, and/or otherwise protect host cells from infection and destruction. The term can also refer to an immune response that is mediated by T-lymphocytes and/or other white blood cells against an infectious agent, exhibited by a vertebrate (e.g., a human), that prevents or ameliorates a viral infection or reduces at least one symptom thereof.

As used herein, the term “purify” is meant to refer to freeing from extraneous or undesirable elements.

The term “pyroptosis” as used herein refers to a form of programmed cell death that is associated with abundant pro-inflammatory cytokines, such as IL-1β and IL-18 produced through inflammasome activation.

The term “reduce” and its various grammatical forms as used herein refers to a diminution, a decrease, an attenuation or abatement of a degree, intensity, extent, size, amount, density or number.

The Renin-Angiotensin-aldosterone System (RAAS) or renin-angiotensin system (RAS) is a critical regulator of blood volume and systemic vascular resistance. It is composed of three major compounds: renin, angiotensin II, and aldosterone, which act to elevate arterial pressure in response to decreased renal blood pressure, decreased salt delivery to the distal convoluted tubule, and/or beta agonism.

Angiotensin II (Ang II), the primary physiological product of the RAAS system, is a potent vasoconstrictor. Angiotensin converting enzyme (ACE) catalyzes the transformation of angiotensin I (Ang I) to Ang II. Ang II elicits its effects by activating two receptors: type 1 angiotensin II (AT1) receptor and type 2 angiotensin II (AT2) receptor [Ingraham, N E, et al. Eur. Respir. J. (2020); DOI: 10.1183/13993003.00912-2020, citing Balakumar, P. & Jagadeesh, G. Cell Signal (2014) 26: 2147-60]. Ang II action through AT1 receptor causes a cascade with resultant inflammation, vasoconstriction, and atherogenesis [Id., citing Strawn, W B & Ferrario, C M., Curr Opin. Lipido. (2002) 13: 505-12]. These effects also promote insulin resistance and thrombosis [Id., citing Dandona, P. et al. J. Hum. Hypertens. (2007) 21: 20-27]. In contrast, AT2 receptor stimulation causes vasodilation, decreased platelet aggregation, and the promotion of insulin action. However, the expression of AT2 receptor is low in healthy adults [Id., citing Dandona, P. et al. J. Hum. Hypertens. (2007) 21: 20-27]. As such, Ang II's effects in adults are modulated and balanced indirectly by angiotensin II converting enzyme (ACE2), which converts Ang II into lung-protective Angiotensin-(1-7) (Ang-[1-7]), similar to effects seen from AT2 receptor stimulation [Id., citing Ghazi, L. & Grawz, P. F1000Research 2017; 6: F1000, Faculty Rev-1297. doi:10.12688/f1000research.9692.1; Warner, F J et al. Cell Mol. Life Sci. (2004) 61: 2704-13].

The term “restore” and its various grammatical forms as used herein refers to bringing back to a former or normal condition, to recover or renew.

The term “retinoic acid receptor (RAR) and “retinoid X receptor” as used herein refer to nuclear hormone receptors that mediate both the organismal and cellular effects of intracellular retinoic acids and their synthetic analogs. The terms RORγt and RORα as used herein refer to transcription factors of the RAR-related orphan nuclear receptor (ROR) family. They are expressed in T_{H}17 cells and have been suggested to play a role in T_{H}17 differentiation.

The term “secondary lymphoid tissues” as used herein refers to sites where lymphocytes interact with each other and nonlymphoid cells to generate immune responses to antigens. These include the spleen, lymph nodes, and mucosa-associated lymphoid tissues (MALT).

As used herein, the term “secretion” and its various grammatical forms is meant to refer to production by a cell of a physiologically active substance and its movement out of the cell in which it is formed.

The term “senescence” as used herein refers to a biological process by which cells undergo growth arrest after extensive replication.

The term “sequelae” and its various grammatical forms as used herein means a pathological condition resulting from a prior disease, injury or attack.

The term “shock” as used herein refers to a critical condition brought on by a sudden drop in blood flow through the body, where the circulatory system fails to maintain adequate blood flow, sharply curtailing the delivery of oxygen and nutrients to vital organs.

The term “sign” as used herein refers to a healthcare provider's evidence of disease.

The term “specification” as used herein refers to a list of tests, references to analytical procedures, and appropriate acceptance criteria that are numerical limits, ranges or other criteria for the test described that establishes the set of criteria to which material should conform to be considered acceptable for its intended use. The term “conformance to specification” means that the material, when tested according to the listed analytical procedures, will meet the listed acceptance criteria.

As used herein, the term “stimulate” in any of its grammatical forms as used herein is meant to refer to inducing activation or increasing activity.

The term “stimulate an immune cell” or “stimulating an immune cell” as used herein is meant to refer to a process (e.g., involving a signaling event or stimulus) causing or resulting in a cellular response, such as activation and/or expansion, of an immune cell, e.g. a CD8+ T cell.

The term “subject” as used herein is meant to refer to any member of the subphylum chordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The present disclosure above is intended for use in any of the above vertebrate species, since the immune systems of all of these vertebrates operate similarly.

As used herein, the phrase “subject in need thereof” is meant to refer to a patient that (i) will be administered an immunogenic composition (e.g. a population of SCKTCs) according to the described invention, (ii) is receiving an immunogenic composition (e.g. a population of SCKTCs) according to the described invention; or (iii) has received an immunogenic composition (e.g. a population of SCKTCs) according to the described invention, unless the context and usage of the phrase indicates otherwise.

As used herein, the term “sufficient to stimulate cytokine killer T cell (CKTC) cell expansion” refers to an amount or level of a signaling event or stimulus, e.g. an amount of alpha-galactosylceramide (αGalCer), or an analog or functional equivalent thereof, that promotes preferential expansion of a CKT cell.

As used herein, the term “sufficient to stimulate CKT cell activation” refers to an amount or level of a signaling event or stimulus, e.g. an amount of IL-2, IL-7, IL-15 and IL-12, that promotes cytokine secretion or cell-killing activity of a CKT cell.

As used herein, the term “superactivated cytokine killer T cells” (or SCKTCs) refers to cells derived from cytokine killer T cells (CKTCs) by contacting CKTCs in vitro with cytokines IL-2, IL-7, IL-15 and IL-12 in a predetermined order and time of addition.

The term “susceptible subject” as used herein refers to an individual vulnerable to developing infection when their body is invaded by an infectious agent. Examples of individuals vulnerable to developing a serious lung infection include, without limitation, the very young, the elderly, those who are ill; those who are receiving immunosuppressants; those with long term health conditions; those that are obese; and those who are physically weak, e.g., due to malnutrition or dehydration.

The term “symptom” as used herein refers to a patient's subjective evidence of disease.

The term “Tbet” as used herein refers to a T_{H}1 cell transcription factor. Differential expression of the T_{H}1 cell transcription factor T bet and a closely related T-box family transcription, factor particularly in CD8+ T cells, Eomesodermin (Eomes) facilitates the cooperative maintenance of the pool of antiviral CD8+ T cells during chronic viral infection. [Paley, M A et a., Science (2012) 338: 1220-125]. During chronic infections, T-bet is reduced in virus-specific CD8+ T cells; this reduction correlates with T cell dysfunction. In contrast, Eomes mRNA expression is up-regulated in exhausted CD8+ T cells during chronic infection. [Id.]

The terms “T cell” or “T lymphocyte” or are used interchangeably to refer to cells that mediate a wide range of immunologic functions, including the capacity to help B cells develop into antibody-producing cells, the capacity to increase the microbicidal action of monocytes/macrophages, the inhibition of certain types of immune responses, direct killing of target cells, and mobilization of the inflammatory response. These effects depend on their expression of specific cell surface molecules and the secretion of cytokines. T cells recognize antigens on the surface of antigen presenting cells (APCs) and mediate their functions by interacting with, and altering, the behavior of these APCs. T cells can also be classified based on their function as helper T cells; T cells involved in inducing cellular immunity; suppressor T cells; and cytotoxic T cells. T-cell activation is dependent on the interaction of the TCR/CD3 complex with its cognate ligand, a peptide bound in the groove of a class I or class II MHC molecule. The molecular events set in motion by receptor engagement are complex. Among the earliest steps appears to be the activation of tyrosine kinases leading to the tyrosine phosphorylation of a set of substrates that control several signaling pathways. These include a set of adapter proteins that link the TCR to the ras pathway, phospholipase Cγ1, the tyrosine phosphorylation of which increases its catalytic activity and engages the inositol phospholipid metabolic pathway, leading to elevation of intracellular free calcium concentration and activation of protein kinase C, and a series of other enzymes that control cellular growth and differentiation. Full responsiveness of a T cell requires, in addition to receptor engagement, an accessory cell-delivered costimulatory activity, e.g., engagement of CD28 on the T cell by CD80 and/or CD86 on the antigen presenting cell (APC).

Although the lineage relationship between T cell subsets remains controversial, T cells cluster in populations that can be arranged as a progressive continuum on the basis of phenotypic, functional and transcriptional attributes. T lymphocytes transition through progressive stages of differentiation that are characterized by a stepwise loss of functional and therapeutic potential in the order from naive T (T_{N}) cells to T memory stem cells (T_{SCM}) (the most immature antigen experienced T cells), to T central memory (T_{CM}) cells, which patrol central lymphoid organs, to Teffector memory (T_{EM}) cells, which patrol peripheral tissues. In contrast to T_{N} cells, memory T cells are capable of rapidly releasing cytokines on restimulation. T_{CM} cells more efficiently secrete IL-2 and T_{EM} have an increased capacity for IFNγ release and cytotoxicity. All antigen-experienced T cells upregulate the common IL-2 and IL-15β receptor (IL-2RD) conferring the ability to undergo homeostatic proliferation in response to IL-15, and also display high amounts of CD95 (also known as FAS), a receptor that provides either costimulatory or pro-apoptotic signals depending on the efficiency of CD95 signaling complex formation and on which particular intracellular signaling proteins are part of the complex. [Gattinoni, L. et al. Natur Revs. Cancer 12: 671-684].

The term “T cell antigen” as used herein is meant to refer to a protein or fragment thereof which can be processed into a peptide that can bind to either Class I MHC, Class II MHC, non-classical MHC, or CD1 family molecules (collectively antigen presenting molecules), and in this combination can engage a T cell receptor on a T cell.

The term “T cell epitope” as used herein is meant to refer to a short peptide molecule that binds to a class I or II MHC molecule and that is subsequently recognized by a T cell. T cell epitopes that bind to class I MHC molecules are typically 8-14 amino acids in length, and most typically 9 amino acids in length. T cell epitopes that bind to class II MHC molecules are typically 12-20 amino acids in length. In the case of epitopes that bind to class II MHC molecules, the same T cell epitope may share a common core segment, but differ in the length of the carboxy- and amino-terminal flanking sequences due to the fact that ends of the peptide molecule are not buried in the structure of the class II MHC molecule peptide-binding cleft as they are in the class I MHC molecule peptide-binding cleft.

The term “T cell exhaustion” as used herein refers to a state of T cell dysfunction that arises during many chronic infections and cancer. It is defined by poor effector function, sustained expression of inhibitory receptors and a transcriptional state distinct from that of functional effector or memory T cells. Modulating pathways overexpressed in exhaustion—for example, by targeting programmed cell death protein 1 (PD1) and cytotoxic T lymphocyte antigen 4 (CTLA4)—can reverse this dysfunctional state and reinvigorate immune responses [Wherry E J and Kurachi, M. Nature (2015) 15: 486-99, citing Wherry E J. Nat. Immunol. (2011) 131:492-499; Schietinger A, Greenberg P D. Trends Immunol. (2014) 35:51-60; Barber D L, et al. Restoring function in exhausted CD8 T cells during chronic viral infection. Nature. (2006) 439:682-687; Nguyen L T, Ohashi P S. Nat. Rev. Immunol. (2014) 15:45-56]. The level and duration of chronic antigen stimulation and infection seem to be key factors that lead to T cell exhaustion and correlate with the severity of dysfunction during chronic infection. Examples of inhibitory receptors include the inhibitory pathways mediated by PD1 in response to binding of PD1 ligand 1 (PDL1) and/or PDL2. [Id., citing Okazaki T, et al., Nature Immunol. (2013) 14:1212-1218, Odorizzi P M, Wherry E J. J. Immunol. (2012) 188:2957-2965, Araki K, et al. Cold Spring Harb. Symp. Quant. Biol. (2013) 78:239-247]. Exhausted T cells can co-express PD1 together with lymphocyte activation gene 3 protein (LAG3), 2B4 (also known as CD244), CD160, T cell immunoglobulin domain and mucin domain-containing protein 3 (TIM3; also known as HAVCR2), CTLA4 and many other inhibitory receptors [Id., citing Blackburn S D, et al. Nat. Immunol. (2009) 10:29-37]. Typically, the higher the number of inhibitory receptors co-expressed by exhausted T cells, the more severe the exhaustion. It has been suggested that inhibitory receptors such as PD1 might regulate T cell function in several ways [Id., citing Schietinger A, Greenberg P D. Trends Immunol. (2014) 35:51-60; Odorizzi P M, Wherry E J. J. Immunol. (2012) 188:2957-29651, e.g., by ectodomain competition, which refers to inhibitory receptors sequestering target receptors or ligands and/or preventing the optimal formation of microclusters and lipid rafts (for example, CTLA4); second, through modulation of intracellular mediators, which can cause local and transient intracellular attenuation of positive signals from activating receptors such as the TCR and co-stimulatory receptors [Id., citing Parry R V, et al. Molec. Cell. Biol. (2005) 25:9543-9553; Yokosuka T, et al. J. Exp. Med. (2012) 209:1201-1217; Clayton K L, et al. J. Immunol. (2014) 192:782-7911; and third, through the induction of inhibitory genes [Id., citing Quigley M, et al. Nat. Med. (2010) 16:1147-1151]. Co-stimulatory receptors also are involved in T cell exhaustion [Id., citing Odorizzi P M, Wherry E J. J. Immunol. (2012) 188:2957-29651. For example, desensitization of co-stimulatory pathway signaling through the loss of adaptor molecules can serve as a mechanism of T cell dysfunction during chronic infection. The signaling adaptor tumor necrosis factor receptor (TNFR)-associated factor 1 (TRAF1) is downregulated in dysfunctional T cells in HIV progressors, as well as in chronic LCMV infection [Id., citing Wang C, et al. J. Exp. Med. (2012) 209:77-911. Adoptive transfer of CD8+ T cells expressing TRAF1 enhanced control of chronic LCMV infection compared with transfer of TRAF1-deficient CD8+ T cells, which indicates a crucial role for TRAF1-dependent co-stimulatory pathways in this setting [Id., citing Wang C, et al. J. Exp. Med. (2012) 209:77-911. It has also been possible to exploit the potential beneficial role of co-stimulation to reverse exhaustion by combining agonistic antibodies to positive co-stimulatory pathways with blockade of inhibitory pathways. 4-1BB (also known as CD137 and TNFRSF9) is a TNFR family member and positive co-stimulatory molecule that is expressed on activated T cells. Combining PD1 blockade and treatment with an agonistic antibody to 4-1BB dramatically improved exhausted T cell function and viral control [Id, citing Vezys V, et al. J. Immunol. (2011) 187:1634-16421. Soluble molecules are a second class of signals that regulate T cell exhaustion; these include immunosuppressive cytokines such as IL-10 and transforming growth factor-β (TGFβ) and inflammatory cytokines, such as type I interferons (IFNs) and IL-6. [Id.]

The term “T cell mediated immune response” as used herein is meant to refer to a response that occurs as a result of recognition of a T cell antigen bound to an antigen presenting molecule on the cell surface of an APC, coupled with other interactions between costimulatory molecules on the T cell and APC. This response serves to induce T cell proliferation, migration, and production of effector molecules, including cytokines and other factors that can injure cells.

The term “T cell receptor” (TCR) as used herein, is meant to refer to a complex of integral membrane proteins that participate in the activation of T cells in response to an antigen. The TCR expressed by the majority of T cells consisting of α and β chains. A small group of T cells express receptors made of γ and δ chains. Among the α/β T cells are two sublineages: those that express the coreceptor molecule CD4 (CD4+ cells), and those that express CD8 (CD8+ cells). These cells differ in how they recognize antigen and in their effector and regulatory functions. CD4+ T cells are the major regulatory cells of the immune system. Their regulatory function depends both on the expression of their cell-surface molecules, such as CD40 ligand whose expression is induced when the T cells are activated, and the wide array of cytokines they secrete when activated. The cytokines can be directly toxic to target cells and can mobilize potent inflammatory mechanisms. CD8+ T cells, can develop into cytotoxic T-lymphocytes (CTLs) capable of efficiently lysing target cells that express antigens recognized by the CTLs.

Naive conventional CD4 T cells can differentiate into four distinct T cell populations, a process that is determined by the pattern of signals they receive during their initial interaction with antigen. These 4 T cell populations are T_{H}1, T_{H}2, T_{H}17, and induced regulatory T (iTreg) cells. Th1 cells, which are effective inducers of cellular immune responses, mediate immune responses against intracellular pathogens, and are responsible for the induction of some autoimmune diseases. Their principal cytokine products are IFNγ (which enhances several mechanisms important in activating macrophages to increase their microbiocidal activity), lymphotoxin α (LTα), and IL-2, which is important for CD4 T cell memory. Th2 cells, which are effective in helping B cells develop into antibody producing cells, mediate host defense against extracellular parasites, are important in the induction and persistence of asthma and other allergic disease, and produce IL-4, IL-5, IL-9, IL-10 (which suppresses T_{H}1 cell proliferation and can suppress dendritic cell function), IL-13, IL-25 (signaling through IL-17RB, enhances the production of IL-4, IL-5, and IL-13 by a c-kit-FcεRI-nonlymphocyte population, serves as an initiation factor as well as an amplification factor for T_{H}2 responses) and amphiregulin. IL-4 and IL-10 produced by T_{H}2 cells block IFNγ production by T_{H}1 cells. T_{H}17 cells produce IL-17a, IL-17f, IL-21, and IL-22. IL-17a can induce many inflammatory cytokines, IL6 as well as chemokines such as IL-8 and plays an important role in inducing inflammatory responses. Treg cells play a critical role in maintaining self-tolerance and in regulating immune responses. They exert their suppressive function through several mechanisms, some of which require cell-cell contact. The molecular basis of suppression in some cases is through their production of cytokines, including TGFβ, IL-10, and IL-35. TGFβ produced by T reg cells may also result in the induction if iTreg cells from naïve CD4 T cells. CD4+ T-cells bear receptors on their surface specific for the B-cell's class II/peptide complex. B-cell activation depends not only on the binding of the T cell through its T cell receptor (TCR), but this interaction also allows an activation ligand on the T-cell (CD40 ligand) to bind to its receptor on the B-cell (CD40) signaling B-cell activation. Zhu, J. and Paul, W E, Blood (2008) 112: 1557-69). Resting naïve CD8+ T cells, when primed by antigen presenting cells that have acquired antigens from the infected macrophages through direct infection or cross-presentation in secondary lymphoid organs, such as lymph nodes and spleen, react to pathogens by massive expansion and differentiation into cytotoxic T lymphocyte effector cells that migrate to all corners of the body to clear the infection. In the majority of viral infections, however, CD8 T cell activation requires CD4 effector T cell help to activate dendritic cells for them to become able to stimulate a complete CD8 T cell response. CD4 T cells that recognize related antigens presented by the APC can amplify the activation of naïve CD8 T cells by further activating the APC. B7 expressed by the dendritic cell first activates the CD4 T cells to express IL-2 and CD40 ligand. CD40 ligand binds CD40 on the dendritic cell, delivering an additional signal that increases the expression of B7 and 4-1BBL by the dendritic cell, which in turn provides additional co-stimulation to the naïve CD8 T cell. The IL-2 produced by activated CD4 T cells also acts to promote effector CD T cell differentiation.

The CD3 (TCR complex) is a protein complex composed of four distinct chains. In mammals, the complex contains a CD3γ chain, a CD3δ chain, and two CD3ε chains, which associate with the T cell receptor (TCR) and the ζ-chain to generate an activation signal in T lymphocytes. Together, the TCR, the ζ-chain and CD3 molecules comprise the TCR complex. The intracellular tails of CD3 molecules contain a conserved motif known as the immunoreceptor tyrosine-based activation motif (ITAM), which is essential for the signaling capacity of the TCR. Upon phosphorylation of the ITAM, the CD3 chain can bind ZAP70 (zeta associated protein), a kinase involved in the signaling cascade of the T cell.

The term “T follicular helper (T_{FH}) cells” as used herein refers to a distinct subset of CD4+ T lymphocytes, specialized in B cell help and in regulation of antibody responses. They develop within secondary lymphoid organs (SLO) and can be identified based on their unique surface phenotype, cytokine secretion profile, and signature transcription factor. They support B cells to produce high-affinity antibodies toward antigens, in order to develop a robust humoral immune response and are crucial for the generation of B cell memory. They are essential for infectious disease control and optimal antibody responses after vaccination. Stringent control of their production and function is critically important, both for the induction of an optimal humoral response against thymus-dependent antigens but also for the prevention of self-reactivity. [Gensous, N. et al. Front. Immunol. (2018) doi.org/10.3389/finmmu.2018.01637).

The term “T_{H}1 cells” as used herein refers to a lineage of CD4+ effector T cells that promotes cell-mediated immune responses and is required for host defense against intracellular viral and bacterial pathogens. They are mainly involved in activating macrophages but can also help stimulate B cells to produce antibody. T_{H1} cells secrete IFN-gamma, IL-2, IL-10, and TNF-alpha/beta. IL-12 and IFN-γ make naive CD4+ T cells highly express T-bet and STAT4 and differentiate to T_{H}1 cells. (Zhang, Y. et al. Adv. Exp. Med. Bio. (2014) 841: 15-44)/

The term “T_{H}2 cells” as used herein refers to a lineage of CD4+ effector T cells that secrete IL-4, IL-5, IL-9, IL-13, and IL-17E/IL-25. These cells are required for humoral or antibody-mediated immunity and play an important role in coordinating the immune response to large extracellular pathogens. IL-4 makes naive CD4+ T cells highly express STAT6 and GATA3 and differentiate to T_{H}2 cells. (Zhang, Y. et al. Adv. Exp. Med. Bio. (2014) 841: 15-44)/

The term “T_{H}17 cells” as used herein refers to a CD4+ T-cell subset characterized by production of interleukin-17 (IL-17). IL-17 is a highly inflammatory cytokine with robust effects on stromal cells in many tissues, resulting in production of inflammatory cytokines and recruitment of leukocytes, especially neutrophils, thus creating a link between innate and adaptive immunity. [Tesmer, L A, et al., Immunol. Rev. (2008) 223: 87-113]. The key transcription factor in T_{H}17 cell development is RORγt.

The term “Treg” or “regulatory T cells” as used herein refers to effector CD4 T cells that inhibit T cell responses and are involved in controlling immune reactions and preventing autoimmunity. The natural regulatory T cell lineage that is produced in the thymus is one subset. The induced regulatory T cells that differentiate from naïve CD4 T cells in the periphery in certain cytokine environments is another subset. Tregs are most commonly identified as CD3+CD4+CD25+FoxP3+ cells in both mice and humans. Additional cell surface markers include CD39, 5′ Nucleotidase/CD73, CTLA-4, GITR, LAG-3, LRRC32, and Neuropilin-1. Tregs can also be identified based on the secretion of immunosuppressive cytokines including TGF-beta, IL-10, and IL-35. Cell surface molecules CTLA-4, LAG-3, and neuropilin-1 (Nrp1) impair dendritic cell (DC)-mediated Tconv activation: CTLA-4 and LAG-3 outcompete CD28 and T cell receptor expressed on conventional T cells for binding to CD80/86 and MHC class II on DCs, and Nrp1 stabilizes DC-Treg contact, thereby preventing antigen presentation to conventional T cells [Ikebuchi, R. et al. Front. Immunol. (2019) doi.org/10.3389/finmmu.2019.01098].

The terms “therapeutic amount”, “effective amount”, an “amount effective”, or “pharmaceutically effective amount” of an active agent are used interchangeably to refer to an amount that is sufficient to provide the intended benefit of treatment. However, dosage levels are based on a variety of factors, including the type of injury, the age, weight, sex, medical condition of the patient, the severity of the condition, the route of administration, and the particular active agent employed. Thus the dosage regimen may vary widely, but can be determined routinely by a physician using standard methods. Additionally, the terms “therapeutic amount”, “effective amounts” and “pharmaceutically effective amounts” include prophylactic or preventative amounts of the compositions of the described disclosure. In prophylactic or preventative applications of the described disclosure, pharmaceutical compositions or medicaments are administered to a patient susceptible to, or otherwise at risk of, a disease, disorder or condition in an amount sufficient to eliminate or reduce the risk, lessen the severity, or delay the onset of the disease, disorder or condition, including biochemical, histologic and/or behavioral symptoms of the disease, disorder or condition, its complications, and intermediate pathological phenotypes presenting during development of the disease, disorder or condition. It is generally preferred that a maximum dose be used, that is, the highest safe dose according to some medical judgment. The terms “dose” and “dosage” are used interchangeably herein.

The term “therapeutic effect” as used herein is meant to refer to a consequence of treatment, the results of which are judged to be desirable and beneficial. A therapeutic effect can include, directly or indirectly, the arrest, reduction, or elimination of a disease manifestation. A therapeutic effect can also include, directly or indirectly, the arrest reduction or elimination of the progression of a disease manifestation.

For any therapeutic agent described herein the therapeutically effective amount may be initially determined from preliminary in vitro studies and/or animal models. A therapeutically effective dose may also be determined from human data. The applied dose may be adjusted based on the relative bioavailability and potency of the administered compound. Adjusting the dose to achieve maximal efficacy based on the methods described above and other well-known methods is within the capabilities of the ordinarily skilled artisan.

General principles for determining therapeutic effectiveness, which may be found in Chapter 1 of Goodman and Gilman's The Pharmacological Basis of Therapeutics, 10th Edition, McGraw-Hill (New York) (2001), incorporated herein by reference, are summarized below.

Pharmacokinetic principles provide a basis for modifying a dosage regimen to obtain a desired degree of therapeutic efficacy with a minimum of unacceptable adverse effects. In situations where the drug's plasma concentration can be measured and related to the therapeutic window, additional guidance for dosage modification can be obtained.

Drug products are considered to be pharmaceutical equivalents if they contain the same active ingredients and are identical in strength or concentration, dosage form, and route of administration. Two pharmaceutically equivalent drug products are considered to be bioequivalent when the rates and extents of bioavailability of the active ingredient in the two products are not significantly different under suitable test conditions.

The term “therapeutic window” as used herein is meant to refer to a concentration range that provides therapeutic efficacy without unacceptable toxicity. Following administration of a dose of a drug, its effects usually show a characteristic temporal pattern. A lag period is present before the drug concentration exceeds the minimum effective concentration (“MEC”) for the desired effect. Following onset of the response, the intensity of the effect increases as the drug continues to be absorbed and distributed. This reaches a peak, after which drug elimination results in a decline in the effect's intensity that disappears when the drug concentration falls back below the MEC. Accordingly, the duration of a drug's action is determined by the time period over which concentrations exceed the MEC. The therapeutic goal is to obtain and maintain concentrations within the therapeutic window for the desired response with a minimum of toxicity. Drug response below the MEC for the desired effect will be subtherapeutic, whereas for an adverse effect, the probability of toxicity will increase above the MEC. Increasing or decreasing drug dosage shifts the response curve up or down the intensity scale and is used to modulate the drug's effect. Increasing the dose also prolongs a drug's duration of action but at the risk of increasing the likelihood of adverse effects. Accordingly, unless the drug is nontoxic, increasing the dose is not a useful strategy for extending a drug's duration of action.

The term “thrombosis” as used herein refers to the formation of a blood clot (thrombus) within a blood vessel, which prevents blood from flowing normally through the circulatory system. For example, endothelial infection with influenza virus has been shown to increase the adhesion of human platelets to primary human lung microvascular endothelial cells via fibronectin, contributing to mortality from acute lung injury. [Sugiyama, M G et al. J. Virol. (2016) 90 (4): 1812-21] A blood clot that forms in the veins (a venous thromboembolism) can cause deep vein thrombosis and pulmonary embolisms. Deep vein thrombosis (DVT) occurs when a blood clot forms in a major vein, usually in the leg, which stops blood from flowing easily through the vein, which can lead to swelling, discoloration and pain. Patients with DVT are at risk for developing post-thrombotic syndrome (PTS), which can involve chronic leg swelling, calf pain calf heaviness/fatigue, skin discoloration and/or venous ulcers. A pulmonary embolism (PE) is a blood clot that has traveled to the lungs. It often starts as a DVT where a piece of the clot breaks off and is carried to the lungs. PE can block the flow of blood to the lungs, causing serious damage to the lungs and affecting a person's ability to breath, which can lead to serious injury and death A blood clot that forms in the arteries (atherothrombosis) can lead to heart attack and stroke.

The term “tissue-resident memory T cell” or “T_{RM}” as used herein refers to memory lymphocytes that do not migrate after taking up residence in barrier tissues, where they are retained long term. They appear to be specialized for rapid effector function after restimulation with antigen or cytokines at sites of pathogen entry.

The term “toll-like receptor (TLR)” as used herein refers to innate receptors on macrophages, dendritic cells, and some other cells, that recognize pathogens and their products, such as bacterial lipopolysaccharide (LPS). Recognition stimulates the receptor-bearing cells to produce cytokines that help initiate immune responses. For example, TLR-1 is a cell surface toll-like receptor that acts in a heterodimer with TLR-2 to recognize lipoteichoic acid and bacterial lipoproteins. TLR-2 is a cell surface toll-like receptor that acts in a heterodimer with either TLR-1 or TLR-6 to recognize lipoteichoic acid and bacterial lipoproteins. TLR-4 is a cell surface toll-like receptor that, in conjunction with accessory proteins MD-2 and CD14, recognizes bacterial lipopolysaccharide and lipoteichoic acid. TLR5 is a cell surface toll-like receptor that recognizes the flagellin protein of bacterial flagella. TLR 6 is a cell surface toll-like receptor that acts in a heterodimer with TLR2 to recognize lipoteichoic acid and bacterial lipoproteins. TLR3 is an endosomal toll-like receptor that recognizes double-stranded viral RNA. TLR-7 is an endosomal toll-like receptor that recognizes single-stranded viral RNA. TLR-8 is an endosomal toll-like receptor that recognizes single-stranded viral RNA. TLR-9 is an endosomal toll-like receptor that recognizes DNA containing unmethylated CpG.

The term “TRAIL” as used herein refers to tumor necrosis factor-related apoptosis-inducing ligand, a member of the TNF cytokine family expressed on the cell surface of some cells, e.g., NK cells, that induces cell death in target cells by ligation of the “death” receptors DR4 and DR5.

The term “treat” or “treating” includes abrogating, substantially inhibiting, slowing or reversing the progression of a disease, condition or disorder, substantially ameliorating clinical or esthetical symptoms of a condition, substantially preventing the appearance of clinical or esthetical symptoms of a disease, condition, or disorder, and protecting from harmful or annoying symptoms. Treating further refers to accomplishing one or more of the following: (a) reducing the severity of the disorder; (b) limiting development of symptoms characteristic of the disorder(s) being treated; (c) limiting worsening of symptoms characteristic of the disorder(s) being treated; (d) limiting recurrence of the disorder(s) in patients that have previously had the disorder(s); and (e) limiting recurrence of symptoms in patients that were previously asymptomatic for the disorder(s).

The term “treatment” as used herein is meant to refer to one or more of (i) the prevention of infection or reinfection, (ii) the reduction or elimination of symptoms, and (iii) the substantial or complete elimination of the pathogen in question. Treatment may be effected prophylactically (prior to infection) or therapeutically (following infection).

The term “TRIM21” as used herein refers to tripartite motif-containing 21, a cytosolic Fc receptor and E3 ligase that is activated by IgG and can ubiquitinate viral proteins after an antibody coated virus enters the cytoplasm.

The term “TRIM25” as used herein refers to an E3 ubiquitin ligase involved in signaling by RIG-1 and MDA-5 for the activation of MAVs.

The terms “variants”, “mutants”, and “derivatives” are used herein to refer to nucleotide or polypeptide sequences with substantial identity to a reference nucleotide or polypeptide sequence. The differences in the sequences may be the result of changes, either naturally or by design, in sequence or structure. Natural changes may arise during the course of normal replication or duplication in nature of the particular nucleic acid sequence. Designed changes may be specifically designed and introduced into the sequence for specific purposes. Such specific changes may be made in vitro using a variety of mutagenesis techniques. Such sequence variants generated specifically may be referred to as “mutants” or “derivatives” of the original sequence. A skilled artisan likewise can produce polypeptide variants having single or multiple amino acid substitutions, deletions, additions or replacements, but biologically equivalent to the wild type sequence. These variants may include inter alia: (a) variants in which one or more amino acid residues are substituted with conservative or non-conservative amino acids; (b) variants in which one or more amino acids are added; (c) variants in which at least one amino acid includes a substituent group; (d) variants in which amino acid residues from one species are substituted for the corresponding residue in another species, either at conserved or non-conserved positions; and (d) variants in which a target protein is fused with another peptide or polypeptide such as a fusion partner, a protein tag or other chemical moiety, that may confer useful properties to the target protein, for example, an epitope for an antibody. The techniques for obtaining such variants, including, but not limited to, genetic (suppressions, deletions, mutations, etc.), chemical, and enzymatic techniques, are known to the skilled artisan.

The term “vascular permeability” as used herein means the net amount of a solute, typically a macromolecule, that has crossed a vascular bed and accumulated in the interstitium in response to a vascular permeabilizing agent or at a site of pathological angiogenesis. [Nagy, J A, et al. Angiogenesis (2008) 11(2): 1009-119].

The term “virus immune escape” or “virus escape” as used herein refers to mechanisms by which viruses evade the immune system of the host.

The terms “viral load” or “viral burden” as used herein refer to a measurement of the amount of a virus in an organism, typically in the bloodstream, usually stated in virus particles per milliliter.

The term “wild-type” as used herein refers to the most common phenotype of an organism, strain, gene, protein, nucleic acid, or characteristic as it occurs in nature. The terms “wild-type” and “naturally occurring” are used interchangeably.

### EMBODIMENTS

### 1. Method of Preparing a Cell Product

According to one aspect, the present disclosure describes a method for preparing a pharmaceutical composition comprising an enriched population of superactivated cytokine killer T cells (SCKTCs) comprising, in order

(a) isolating a population of mononuclear cells (MCs) comprising a population of cytokine killer T cells (CKTCs);

(b) transporting the preparation of (a) to a processing facility under sterile conditions;

(c) on Day 0, placing the population of MCs in a suspension culture system in a serum free culture medium;

(d) on Day 6, contacting the culture system of step (c) with the serum free culture medium containing IL-2 and IL-7,wherein the contacting stimulates CKTC activation;

(e) on Day 7, pulsing the CKTCs of step (d) with an enriched population of CD1d− expressing antigen presenting cells (APCs) derived from the MCs in (a) loaded with a-GalCer;

(f) on Day 8-13, replenishing the culture medium every 1-3 days from day 7 to day 14 with fresh serum-free culture medium;

(g) on Day 14, adding CD1d expressing APCs loaded with α-GalCer;

(h) on Day 14+1 to Day 14+6, replenishing the culture medium of the cells with fresh serum-free culture medium every 1-3 days;

(i) on Day 14+7 replenishing the culture medium of the culture with fresh serum-free culture medium and pulsing with CD1d expressing APCs loaded with α-GalCer;

(j) on Day 14+8 to Day 14+13, replenishing the culture medium of the culture with fresh serum-free culture medium;

on Day 14+14, replenishing the culture medium of the culture with fresh serum-free culture medium and pulsing with CD1d-expressing APCs loaded with α-GalCer;

(k) on Day 14+15 to Day 14+20, replenishing the culture medium of the culture with fresh serum-free culture medium;

on day 14+21 replenishing the culture medium of the culture with fresh serum-free culture medium and adding IL-12;

(l) on Day 14+22 harvesting the amplified enriched superactivated population of SCKTCs from the culture system to form a SCKTC cell product; and

(m) filling and finishing aliquots of the SCKTC cell product comprising 2×10⁸-1×10⁹ SCKTCs into a container;

(n) optionally cryopreserving the SCKTC cell product in the vapor phase of a liquid nitrogen freezer in a serum-free cryo freezing medium.

According to some embodiments, the method further comprises transporting the SCKTC cell product from the processing facility to a treatment facility. According to some embodiments, the transporting step is initiated within at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, or at least 24 hours of the addition of IL-12.

According to some embodiments, in step (a) the frequency of the population of CKTCs from the donor represents <0.5% of the total MNC population.

According to some embodiments, activation and expansion steps of the method are performed in tissue culture flasks. According to some embodiments, activation and expansion steps of the method are performed in gas permeable cell culture bags. According to some embodiments, activation and expansion steps of the method are performed in a closed system. According to some embodiments, the closed system is fully automated.

According to some embodiments, the population of PBMCs comprises subpopulations of T lymphocytes, NK cells, B lymphocytes, and monocytes. According to some embodiments, the subpopulation of T lymphocytes comprises NKT cells, CD4+ T cells, and CD8+ T cells.

According to some embodiments, the SCKTC cell product in step (l) is proliferation competent.

According to some embodiments, a source of the mononuclear cells (MCs) is blood. According to some such embodiments, the blood is peripheral blood and the MCs are peripheral blood MCs (PBMCs). According to some embodiments, the PBMCs are derived from a human subject. According to some embodiments, the donor of the MCs is autologous to the recipient. According to some embodiments, the donor of the MCs is allogeneic to the recipient.

According to some embodiments, leukapheresis is performed at a blood collection center. PBMCs then are isolated using an apparatus containing a spinning chamber (e.g., a Sepax c-Pro System (Cytiva)). The blood separates into its components (plasma, platelet-rich plasma, leukocytes and red blood cells) by gravity along the wall of the chamber. Mononuclear cells are sorted out and collected.

According to some embodiments, the MCs can be isolated from whole peripheral blood at room temperature as follows. 2 ml of defibrinated or anti-coagulin-treated blood and an equal volume of balanced salt solution is added to a 10 ml centrifuge tube. The blood and buffer are mixed. Ficoll-Paque media (3 ml—Cytiva) is added to the centrifuge tube. The diluted blood sample (4 ml) is layered onto the Ficoll-Paque media solution and centrifuged at 400 g for 30-40 min with the brake off. The upper layer containing plasma and platelets is drawn off, leaving the mononuclear cell layer undisturbed at the interface. The layer of mononuclear cells is transferred to a sterile centrifuge tube using a sterile pipette and washed with centrifugation.

According to some embodiments, the population of MCs comprising a population of CKTCs can be derived from stem cells. The term “stem cells” as used herein refers to undifferentiated cells having high proliferative potential with the ability to self-renew that can generate daughter cells that can undergo terminal differentiation into more than one distinct cell phenotype. Stem cells are distinguished from other cell types by two characteristics. First, they are unspecialized cells capable of renewing themselves through cell division, sometimes after long periods of inactivity. Second, under certain physiologic or experimental conditions, they can be induced to become tissue- or organ-specific cells with special functions.

Embryonic stem cells (EmSC) are stem cells derived from an embryo that are pluripotent, i.e., they are able to differentiate in vitro into endodermal, mesodermal and ectodermal cell types. Induced pluripotent stem cells (iPSCs) offer an extensive capacity for self-renewal without the ethical concerns faced by EmSCs. iPSCs can be induced and redifferentiated to cells in the immune system, specifically to HSCs and fully differentiated immune cells, including NIT cells [Jiang, Z. et al. Cellular & Molec. Immunol. (2014) 11: 17-24].

Adult (somatic) stem cells are undifferentiated cells found among differentiated cells in a tissue or organ. Their primary role in vivo is to maintain and repair the tissue in which they are found. Adult stem cells have been identified in many organs and tissues, including brain, bone marrow, peripheral blood, blood vessels, skeletal muscles, skin, teeth, gastrointestinal tract, liver, ovarian epithelium, and testis. Adult stem cells are thought to reside in a specific area of each tissue, known as a stem cell niche, where they may remain quiescent (non-dividing) for long periods of time until they are activated by a normal need for more cells to maintain tissue, or by disease or tissue injury.

According to some embodiments, the stem cells comprise hematopoietic stem cells. Hematopoietic stem cells (also known as the colony-forming unit of the myeloid and lymphoid cells (CFU-M,L), HSCs, or CD34+ cells) are rare pluripotential cells within the blood-forming organs that are responsible for the continued production of blood cells during life. While there is no single cell surface marker exclusively expressed by hematopoietic stem cells, it generally has been accepted that human HSCs have the following antigenic profile: CD34+, CD59+, Thy1+(CD90), CD38low/−, C-kit−/low and, lin−. HSCs can generate a variety of cell types, including erythrocytes, neutrophils, basophils, eosinophils, platelets, mast cells, monocytes, tissue macrophages, osteoclasts, and the T and B lymphocytes.

According to some embodiments, the HSCs can be derived from adult bone marrow, umbilical cord, umbilical cord blood, placental tissue, or fetal liver. According to some embodiments, the HSCs can be purified by positive or negative selection cell separation methods. Positive selection cell separation methods involve directly labeling desired cells for selection with an antibody or a ligand that targets a specific cell surface protein. In immunomagnetic separation methods, the antibody or ligand is linked to a magnetic particle, allowing the labeled cells to be retained in the final isolated fraction after incubation of the same in a magnetic field. Negative selection cell separation methods involve laveling unwanted cell types for removal with antibodies or ligands targeting specific cell surface proteins. In immunomagnetic separation methods, the antibodies or ligands are linked to magnetic particles, allowing the labeled, unwanted cells to be depleted from the final isolated fraction by incubating the sample in a magnetic field. Since the desired cells are not specifically targeted by antibodies or ligands, they remain unbound by particles. According to some embodiments, magnetic bead activated cell sorting, a positive selection technique, can be used for purifying the CD34+ cell population from the mononuclear cells. According to some embodiments, negative selection protocols can be employed to reduce the risk of decreasing the quantity and activity of the desired cells such protocols. According to some embodiments a pure SCKTC population is achieved from HSCs by the method without positive or negative cell separation methods.

### Antigen Presenting Cells

An antigen presenting cell is a class of cell capable of displaying on its surface one or more antigens in the form of a peptide-MHC complex recognizable by specific effector cells of the immune system, and thereby inducing an effective cellular immune response against the antigen or antigens being presented. Examples of professional APCs are dendritic cells and macrophages, although any cell expressing MHC Class I molecules or MHC Class II molecules can potentially present peptide antigen. According to some embodiments, an APC can be a cell or population of cells that is engineered to present one or more antigens (i.e. an artificial APC (aAPC). According to some embodiments, an APC can be irradiated population of PBMCs. According to some embodiments, the irradiated population of pBMCs comprises a subpopulation of cells expressing CD1d.

According to some embodiments, the antigen is a non-peptide antigen. According to some embodiments, the antigen is a lipid antigen. According to some embodiments, the antigen is alpha-GalCer. According to some embodiments, the population of APCs loaded with alpha-GalCer is a population of dendritic cells. According to some embodiments, a population of dendritic cells loaded with αGalCer is prepared by a method comprising (a) isolating a population of CD14+ mononuclear cells (MCs); (b) culturing the population of CD14+ MCs in a culture system; thereby inducing differentiation of the CD14+ MCs into dendritic cells; (c) contacting the culture system with αGalCer, wherein the contacting is sufficient to load the dendritic cells with αGalCer.

### (a) Isolating a Population of CD14+ MCs

Monocytes are circulating blood leukocytes with a fundamental capacity to differentiate into macrophages. In the right environment, monocytes can also differentiate into specialized antigen-presenting dendritic cells (moDCs). [Qu, C. et al. Intl J. Infectious Disease (2014) 19: 1-5]. The major subset of monocytes consists of CD14^{high} CD16^{negative} (CD14⁺⁺CD16⁻). The CD16 expressing monocytes are usually divided into CD14^{high} CD16^{low} (CD14⁺⁺CD16⁺) and CD14^{low} CD16^{high}(CD¹⁴⁺CD16⁺⁺) subsets [Id., citing Ziegler-Heitbrock, L. et al. Blood (2010) 116: e74-e80]; both subsets of monocytes can differentiate into dendritic cells (DCs) in the presence of granulocyte-macrophage colony-stimulating factor (GM-CSF) and IL-4 when cultured in vitro. They internalize soluble and particulate antigens similarly, and both are able to stimulate T cell proliferation in autologous and allogeneic cultures [Id., citing Sanchez-Torres, C. et al., Int'l Immunol. (2001) 13: 1571-81; Sallusto, F. and Lanzavecchia, A. J. Exp. Med. (1994) 179: 1109-18; Romani, N. et al. J. Exp. Med. (1994) 180: 83-93]. However, CD16⁺ moDCs express higher levels of CD86, CD11a, and CD11c, and show lower expression of CD1a and CD32 compared to CD16⁻ moDCs. LPS-stimulated CD16⁻ moDCs express increased levels of IL-12 p40 mRNA and secrete greater amounts of IL-12 p70 than CD16⁺ moDCs, whereas levels of transforming growth factor beta 1 (TGF-β1) mRNA are higher in CD16⁺ moDCs. Moreover, CD4+ T cells stimulated with CD16⁺ moDCs secrete increased amounts of IL-4 compared to those stimulated by CD16⁻ moDCs [Id., citing Sanchez-Torres, C. et al. Intl. Immunol. (2001) 13: 1571-81]. Using an in vitro transendothelial migration model, monocytes were demonstrated to migrate across an endothelial barrier in vitro and differentiate into DCs, which reverse-migrate back across the endothelial layer, or into macrophages, which remain in the subendothelial matrix [Id., citing Randolph, G J et al. Blood (1998) 92: 4167-77]. In this model, the CD14+CD16+ monocytes were found to be more likely to become DCs than the CD14⁺CD16⁻ monocytes [Id., citing Randolph, G J et al. J. Exp. Med. (2002) 196: 517-27], indicating that the CD14⁺CD16⁺ monocytes might be precursors of DCs. The classical CD14+ monocytes develop the non-classical CD14⁺CD16⁺ monocytes; CD14⁺CD16⁺ monocytes may represent a more mature version. Id., citing Randolph, G J J. Exp. Med. (2002) 196: 517-27].

According to some embodiments, CD14+ monocytes are sorted out of a population of PBMCs using CD14+ microbeads (e.g., Miltenyi, Dynabeads™). For MACS separation, cells are magnetically labeled with CD14 microbeads and separated on a column which is placed in the magnetic field of a MACS separator. The magnetically labeled CD14+ cells are retained in the column while the unlabeled CD14− cells, which are depleted of CD14+ cells run through. After removal of the column from the magnetic field, the magnetically retained CD14+ cells can be eluted as a positively selected cell fraction. According to some embodiments, the eluted CD14+ monocytes are viable.

### (b) Culturing the CD14+ MCs to Induce Differentiation of DCs

According to some embodiments, a viable enriched population of DCs is prepared from about 5×10⁸ to 5×10⁹ MCs, i.e., about 5.0×10⁸, about 5.1×10⁸, about 5.2×10⁸, about 5.3×10⁸, about 5.4×10⁸, about 5.5×10⁸, about 5.6×10⁸, about 5.7×10⁸, about 5.8×10⁸, about 5.9×10⁸, about 6.0×10⁸, about 6.1×10⁸, about 6.2×10⁸, about 6.3×10⁸, about 6.4×10⁸, about 6.5×10⁸, about 6.6×10⁸, about 6.7×10⁸, about 6.8×10⁸, about 6.9×10⁸, about 7.0×10⁸, about 7.1×10⁸, about 7.2×10⁸, about 7.3×10⁸, about 7.4×10⁸, about 7.5×10⁸, about 7.6×10⁸, about 7.7×10⁸, about 7.8×10⁸, about 7.9×10⁸, about 8.0×10⁸, about 8.1×10⁸, about 8.2×10⁸, about 8.3×10⁸, about 8.4×10⁸, about 8.5×10⁸, about 8.6×10⁸, about 8.7×10⁸, about 8.8×10⁸, about 8.9×10⁸, about 9.0×10⁸, about 9.1×10⁸, about 9.2×10⁸, about 9.3×10⁸, about 9.4×10⁸, about 9.×10⁸, about 9.6×10⁸, about 9.7×10⁸, about 9.8×10⁸, about 9.9×10⁸, about 1×10⁹, about 1.1×10⁹, about 1.2×10⁹, about 1.3×10⁹, about 1.4×10⁹, about 1.5×10⁹, about 1.6×10⁹, about 1.7×10⁹, about 1.8×10⁹, about 1.9×10⁹, about 2.0×10⁹, about 2.1×10⁹, about 2.2×10⁹, about 2.3×10⁹, about 2.4×10⁹, about 2.5×10⁹, about 2.6×10⁹, about 2.7×10⁹, about 2.8×10⁹, about 2.9×10⁹, about 3.0×10⁹, about 3.1×10⁹, about 3.2×10⁹, about 3.2×10⁹, about 3.3×10⁹, about 3.4×10⁹, about 3.5×10⁹, about 3.6×10⁹, about 3.7×10⁹, about 3.8×10⁹, about 3.9×10⁹, about 4.0×10⁹, about 4.1×10⁹, about 4.2×10⁹, about 4.3×10⁹, about 4.4×10⁹, about 4.5×10⁹, about 4.6×10⁹, about 4.7×10⁹, about 4.8×10⁹, about 4.9×10⁹, about 5.0×10⁹ MCs.

According to some embodiments, the culturing of CD14+ monocytes induces differentiation of the monocytes to DCs.

According to some embodiments, at least 30% of the monocyte derived population of DCs constitutively expresses CD1d. According to some embodiments, at least 35% of the monocyte derived population of DCs constitutively expresses CD1d. According to some embodiments, at least 40% of the monocyte derived population of DCs constitutively expresses CD1d. According to some embodiments, at least 45% of the monocyte derived population of DCs constitutively expresses CD1d. According to some embodiments, at least 50% of the monocyte derived population of DCs constitutively expresses CD1d. According to some embodiments, at least 55% of the monocyte derived population of DCs constitutively expresses CD1d. According to some embodiments, at least 60% of the dendritic cell population constitutively expresses CD1d. According to some embodiments, at least 65% of the monocyte derived population of DCs constitutively expresses CD1d. According to some embodiments, at least 70% of the monocyte derived population of DCs constitutively expresses CD1d. According to some embodiments, at least 75% of the monocyte derived population of DCs constitutively expresses CD1d. According to some embodiments, at least 80% of the monocyte derived population of DCs constitutively expresses CD1d. According to some embodiments, at least 85% of the monocyte derived population of DCs constitutively expresses CD1d. According to some embodiments, at least 90% of the monocyte derived population of DCs constitutively expresses CD1d. According to some embodiments, at least 95% of the monocyte derived population of DCs constitutively expresses CD1d.

### (c) Loading αGalCer

According to some embodiments, the enriched population of DCs is contacted and loaded with α-GalCer or a derivative or analog thereof. According to some embodiments the enriched population of DCs is contacted and loaded with α-GalCer up to 2 hrs before pulsing the CKTCs. According to some embodiments, the DC population loaded with α-GalCer is a mixture of adherent and suspension cells.

According to some embodiments, the concentration of αGalCer, or an analog or functional equivalent thereof, ranges from about 50 ng/ml to about 500 ng/ml, from about 100 ng/ml to about 500 ng/ml, from about 150 ng/ml to about 500 ng/ml, from about 200 ng/ml to about 500 ng/ml, from about 250 ng/ml to about 500 ng/ml, from about 300 ng/ml to about 500 ng/ml, from about 350 ng/ml to about 500 ng/ml, from about 400 ng/ml to about 500 ng/ml, or from about 450 ng/ml to about 500 ng/ml. According to some embodiments, the concentration of αGalCer, or an analog or functional equivalent thereof, is maintained at a concentration of about 50 ng/ml, about 60 ng/ml, about 70 ng/ml, about 80 ng/ml, about 90 ng/ml, about 100 ng/ml, about 110 ng/ml, about 120 ng/ml, about 130 ng/ml, about 140 ng/ml, about 150 ng/ml, about 160 ng/ml, about 170 ng/ml, about 180 ng/ml, about 190 ng/ml, about 200 ng/ml, about 210 ng/ml, about 220 ng/ml, about 230 ng/ml, about 240 ng/ml, about 250 ng/ml, about 260 ng/ml, about 270 ng/ml, about 280 ng/ml, about 290 ng/ml, about 300 ng/ml, about 310 ng/ml, about 320 ng/ml, about 330 ng/ml, about 340 ng/ml, about 350 ng/ml, about 360 ng/ml, about 370 ng/ml, about 380 ng/ml, about 390 ng/ml, about 400 ng/ml, about 410 ng/ml, about 420 ng/ml, about 430 ng/ml, about 440 ng/ml, about 450 ng/ml, about 460 ng/ml, about 470 ng/ml, about 480 ng/ml, about 490 ng/ml, or about 500 ng/ml. According to some embodiments, the αGalCer, or an analog or functional equivalent thereof is maintained at a constant concentration. According to some embodiments, the concentration of α-Gal Ser is about 200 ng/ml.

α-GalCer, also known as KRN7000, is a simplified glycolipid analogue of agelasphin, which was originally isolated from a marine sponge Agelas mauritianus (Kobayahi et al., Oncol Res. α-GalCer is composed of an a-linked galactose, a phytosphingosine and an acyl chain. Alpha-GalCer is composed of a galactose head group that is linked through the a-hydroxyl to the sphingosine chain (18 carbons). The sphingosine chain is further linked to the fatty acyl chain (26 carbons). The structural chemical formula (A) and ball and stick formula (B) for alpha-galactosyl ceramide (α-GalCer, also known as N-[(2S,3S,4R)-3,4-dihydroxy-1-[(2S,3R,4S,5R,6R)-3,4,5-trihydroxy-6-(hydroxymethyl)oxan-2-yl]oxyoctadecan-2-yl]hexacosanamide; Krn7000; KRN7000, alpha-GalCer, PubChem CID 2826713, molecular formula C₅₀H₉₉N0₉, molecular weight 858.3 g/mol) are shown below.

### A.

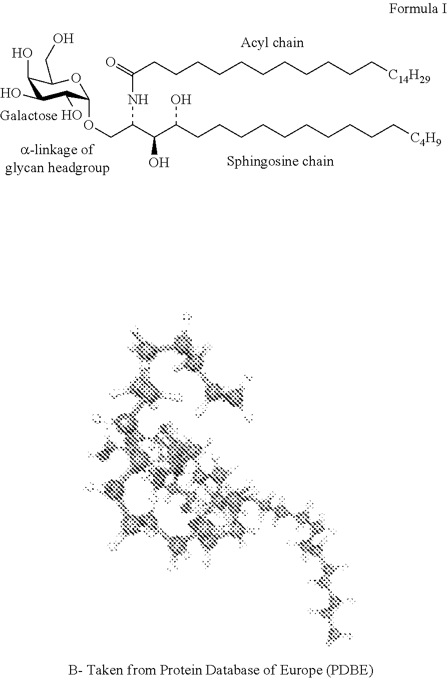

Recognition of the α-GalCer-CD1d complex by the type-I NKT cell TCR results in the secretion of a range of cytokines, and the initiation of a powerful immune response.

Several analogs of α-GalCer have been prepared and described, e.g.,

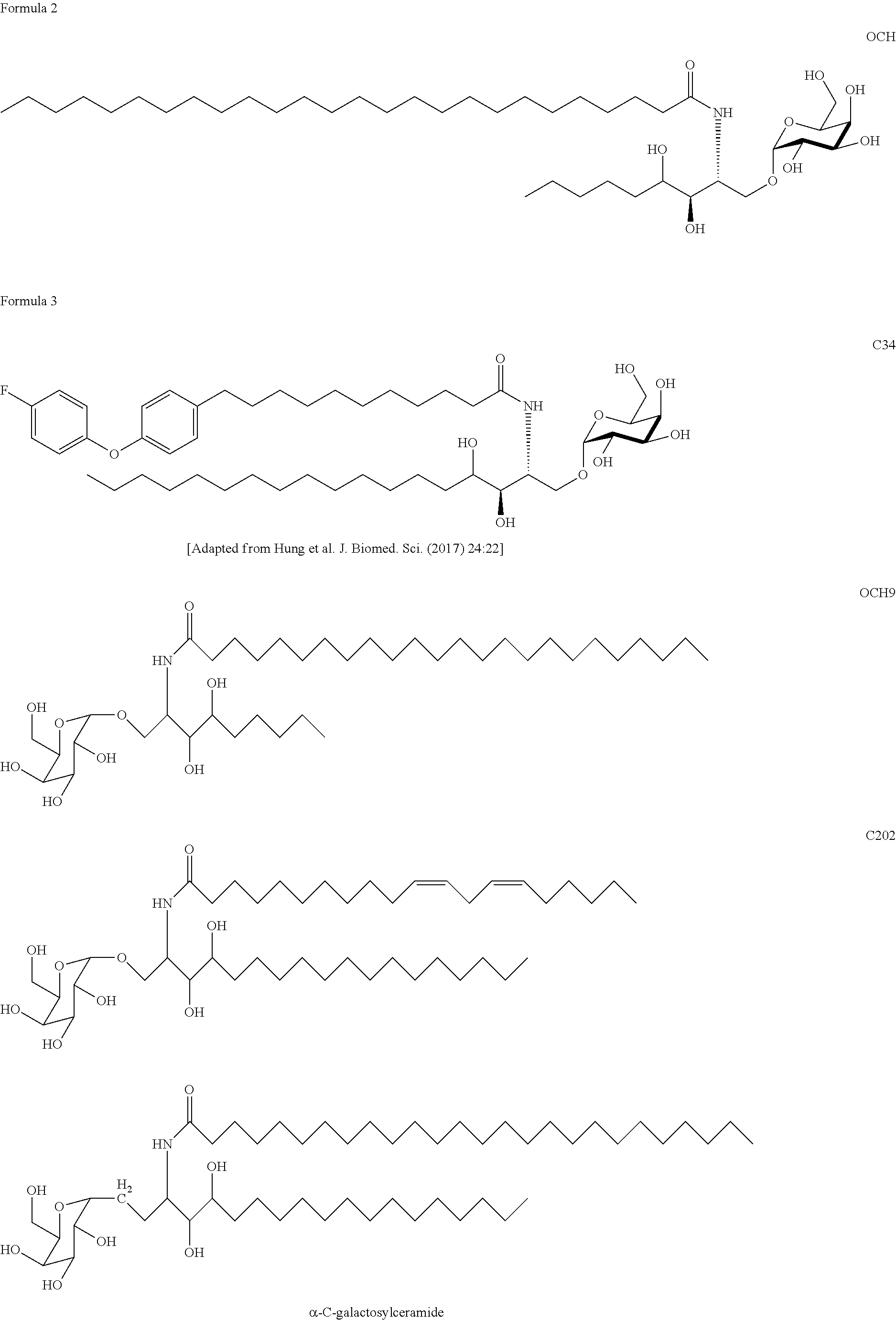

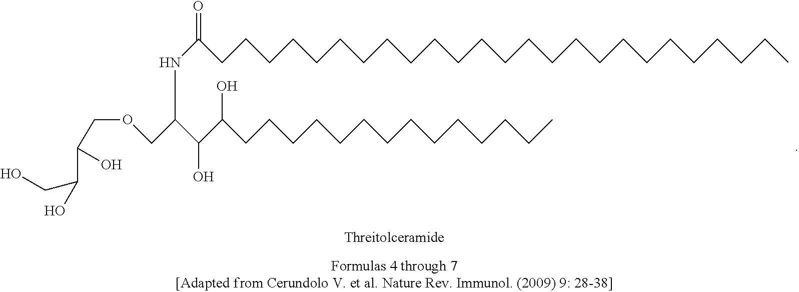 [SMILES: CCCCCCCCCCCCCCCCCCCCCCCCCC(=O)NC(COCC(O)C(O)CO)C(O)C(O)CCCCCCCCCCCCCC]

Of these, OCH, an alpha-GalCer analogue with a shorter phytosphingosine chain, stimulates type-I NKT cells to secrete higher amounts of IL-4 than IFN-γ, triggering the immune response toward T_{H}2 [Hung, J T et al. Journal of Biomedical Science 2017, 24:221, while alpha-C-GalSer is a T_{H}1-biasing CD1d agonist. [Wojno, J. et al. ACS Biology (2012) 7: 847-55]. Other synthetic glycolipids or alpha-GalCer analogs chemically modified to induce more precise and predictable cytokine profile than alpha-GalCer also have been synthesized and tested. [See, e.g., Hung, J-T et al. Journal of Biomedical Science (2017) 24:22]. U.S. Pat. Nos. 9,365,496, and 10,765,648, each of which is incorporated by reference in its entirety herein, also describes various alpha-GalCer analogs with the general structural formula:

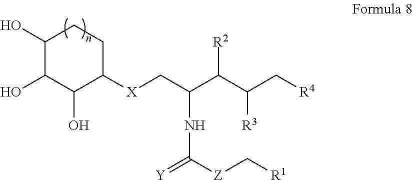

where in some embodiments, n is 1, 2, or 3.

Beta-ManCer, another class of type-I NKT cell agonist, also has been described [O'Konek, J J et al., J Clin Invest. 2011 February; 121(2):683-94].

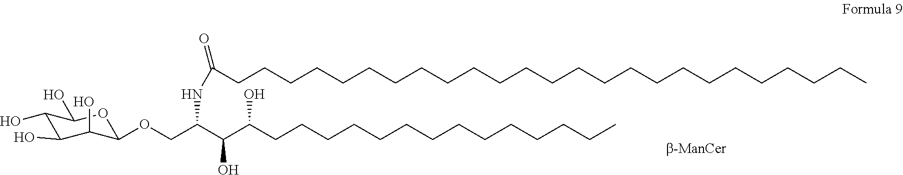 [SMILES: CCCCCCCCCCCCCCCCCCCCCCCCCC(=O)N[C@@H](COC1OC(O)C(O)C(O)C1O)[C@H](O)[C@H](O)CCCCCCCCCCCCCC]

This compound has an identical ceramide structure to that of alpha-GalCer, which contributes to the binding with CD1d, with a beta-linked mannose instead of alpha-linked galactose. Because it had been believed in the field that the alpha-linked sugar moiety was a critical feature of alpha-GalCer to elicit tumor immunity, the discovery of the relatively strong anti-tumor activity of beta-ManCer was unexpected. While the protection induced by beta-ManCer was type-I NKT cell-dependent, the protection was independent of IFN-γ but dependent on TNF-α and nitric oxide synthase (NOS). Furthermore, consistent with their distinct mechanisms of protection, alpha-GalCer and beta-ManCer synergize to induce tumor immunity when suboptimal doses were used. In addition, beta-ManCer has much weaker ability to induce long-term anergy in type-I NKT cells than alpha-GalCer [O'Konek, J J et al, Clin Cancer Res. 2013 Aug. 15; 19(16):4404-11]. Similar to alpha-GalCer, beta-ManCer can enhance the effect of a tumor vaccine [Mattarollo, S R et al., Blood. (2012) Oct. 11; 120(15):3019-29].

### Activation of the CKTC Population

According to some embodiments, a fresh population of DCs is added to the IL-2 and IL-7 stimulated CKTC culture. According to some embodiments, the population of DCs is cryopreserved, thawed and then added to the CKTC culture. According to some embodiments the population of DCs derived from PBMCs that is added to the 1-1.5×10⁶ CKTCs ranges from about 1×10⁶ to about 1×10⁷ DCs, i.e., about 1.0×10⁶, about 1.1×10⁶, about 1.2×10⁶, about 1.3×10⁶, about 1.4×10⁶, about 1.5×10⁶, about 1.6×10⁶, about 1.7×10⁶, about 1.8×10⁶, about 1.9×10⁶, about 2.0×10⁶, about 2.1×10⁶, about 2.2×10⁶, about 2.3×10⁶, about 2.4×10⁶, about 2.5×10⁶, about 2.6×10⁶, about 2.7×10⁶, about 2.8×10⁶, about 2.9×10⁶, about 3.0×10⁶, about 3.1×10⁶, about 3.2×10⁶, about 3.3×10⁶, about 3.4×10⁶, about 3.5×10⁶, about 3.6×10⁶, about 3.7×10⁶, about 3.8×10⁶, about 3.9×10⁶, about 4.0×10⁶, about 4.1×10⁶, about 4.2×10⁶, about 4.3×10⁶, about 4.4×10⁶, about 4.5×10⁶, about 4.6×10⁶, about 4.7×10⁶, about 4.8×10⁶, about 4.9×10⁶, about 5.0×10⁶, about 5.1×10⁶, about 5.2×10⁶, about 5.3×10⁶, about 5.4×10⁶, about 5.5×10⁶, about 5.6×10⁶, about 5.7×10⁶, about 5.8×10⁶, about 5.9×10⁶, about 6.0×10⁶, about 6.1×10⁶, about 6.2×10⁶, about 6.3×10⁶, about 6.4×10⁶, about 6.5×10⁶, about 6.6×10⁶, about 6.7×10⁶, about 6.8×10⁶, about 6.9×10⁶, about 7.0×10⁶, about 7.1×10⁶, about 7.2×10⁶, about 7.3×10⁶, about 7.4×10⁶, about 7.5×10⁶, about 7.6×10⁶, about 7.7×10⁶, about 7.8×10⁶, about 7.9×10⁶, about 8.0×10⁶, about 9×10⁶, or about 1×10⁷ DCs.

According to some embodiments of the methods describe herein, the concentration of IL-2 (Recombinant Human IL-2 GMP Protein (R&D Systems, cat #202-GMP) is between about 10 U/ml to about 100 U/ml, for example between about 10 U/ml to about 100 U/ml, about 15 U/ml to about 100 U/ml, about 20 U/ml to about 100 U/ml, about 25 U/ml to about 100 U/ml, about 30 U/ml to about 100 U/ml, about 35 U/ml to about 100 U/ml, about 40 U/ml to about 100 U/ml, about 45 U/ml to about 100 U/ml, about 50 U/ml to about 100 U/ml, about 55 U/ml to about 100 U/ml, about 60 U/ml to about 100 U/ml, about 65 U/ml to about 100 U/ml, about 70 U/ml to about 100 U/ml, about 75 U/ml to about 100 U/ml, about 80 U/ml to about 100 U/ml, about 85 U/ml to about 100 U/ml, about 90 U/ml to about 100 U/ml, or about 95 U/ml to about 100 U/ml. According to some embodiments, the concentration of IL-2 is about 10 U/ml, about 15 U/ml, about 20 U/ml, about 25 U/ml, about 30 U/ml, about 35 U/ml, about 40 U/ml, about 45 U/ml, about 50 U/ml, about 55 U/ml, about 60 U/ml, about 65 U/ml, about 70 U/ml, about 75 U/ml, about 80 U/ml, about 85 U/ml, about 90 U/ml, about 95 U/ml, or about 100 U/ml.

According to some embodiments of the methods describe herein, the concentration of IL-7 (Recombinant Human IL-7 GMP Protein (R&D Systems, cat #207-GMP) is between about 10 ng/ml to about 200 ng/ml, for example between about 10 ng/ml to about 200 ng/ml, about 20 ng/ml to about 200 ng/ml, about 30 ng/ml to about 200 ng/ml, about 40 ng/ml to about 200 ng/ml, about 50 ng/ml to about 200 ng/ml, about 60 ng/ml to about 200 ng/ml, about 70 ng/ml to about 200 ng/ml, about 80 ng/ml to about 200 ng/ml, about 90 ng/ml to about 200 ng/ml, about 100 ng/ml to about 200 ng/ml, about 110 ng/ml to about 200 ng/ml, about 120 ng/ml to about 200 ng/ml, about 130 ng/ml to about 200 ng/ml, about 140 ng/ml to about 200 ng/ml, about 150 ng/ml to about 200 ng/ml, about 160 ng/ml to about 200 ng/ml, about 170 ng/ml to about 200 ng/ml, about 180 ng/ml to about 200 ng/ml, or about 190 ng/ml to about 200 ng/ml. According to some embodiments, the concentration of IL-7 is about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, about 100 ng/ml, about 110 ng/ml, about 15 ng/ml, about 120 ng/ml, about 125 ng/ml, about 130 ng/ml, about 135 ng/ml, about 140 ng/ml, about 145 ng/ml, about 150 ng/ml, about 155 ng/ml, about 160 ng/ml, about 165 ng/ml, about 170 ng/ml, about 175 ng/ml, about 180 ng/ml, about 185 ng/ml, about 190 ng/ml, about 195 ng/ml, or about 200 ng/ml.

According to some embodiments, IL-15 is added between about day 13 and day 15 of culture. According to some embodiments, IL-15 is added at about day 13 of culture. According to some embodiments, IL-15 is added at about day 14 of culture. According to some embodiments, IL-15 is added at about day 15 of culture.

According to some embodiments, the concentration of IL-15 (Recombinant Human IL-15 GMP Protein (R&D Systems, cat #247-GMP) is between about 10 ng/ml to about 100 ng/ml, for example between about 10 ng/ml to about 100 ng/ml, about 15 ng/ml to about 100 ng/ml, about 20 ng/ml to about 100 ng/ml, about 25 ng/ml to about 100 ng/ml, about 30 ng/ml to about 100 ng/ml, about 35 ng/ml to about 100 ng/ml, about 40 ng/ml to about 100 ng/ml, about 45 ng/ml to about 100 ng/ml, about 50 ng/ml to about 100 ng/ml, about 55 ng/ml to about 100 ng/ml, about 60 ng/ml to about 100 ng/ml, about 65 ng/ml to about 100 ng/ml, about 70 ng/ml to about 100 ng/ml, about 75 ng/ml to about 100 ng/ml, about 80 ng/ml to about 100 ng/ml, about 85 ng/ml to about 100 ng/ml, about 90 ng/ml to about 100 ng/ml, or about 95 ng/ml to about 100 ng/ml. According to some embodiments, the concentration of IL-15 is about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, or about 100 ng/ml.

According to some embodiments, IL-12 (Recombinant Human IL-12 GMP Protein (R&D Systems, cat #219-GMP) is added about one day before cell harvest. According to some embodiments, the concentration of IL-12 is between about 10 ng/ml to about 100 ng/ml, for example between about 10 ng/ml to about 100 ng/ml, about 15 ng/ml to about 100 ng/ml, about 20 ng/ml to about 100 ng/ml, about 25 ng/ml to about 100 ng/ml, about 30 ng/ml to about 100 ng/ml, about 35 ng/ml to about 100 ng/ml, about 40 ng/ml to about 100 ng/ml, about 45 ng/ml to about 100 ng/ml, about 50 ng/ml to about 100 ng/ml, about 55 ng/ml to about 100 ng/ml, about 60 ng/ml to about 100 ng/ml, about 65 ng/ml to about 100 ng/ml, about 70 ng/ml to about 100 ng/ml, about 75 ng/ml to about 100 ng/ml, about 80 ng/ml to about 100 ng/ml, about 85 ng/ml to about 100 ng/ml, about 90 ng/ml to about 100 ng/ml, or about 95 ng/ml to about 100 ng/ml. According to some embodiments, the concentration of IL-12 is about 10 ng/ml, about 15 ng/ml, about 20 ng/ml, about 25 ng/ml, about 30 ng/ml, about 35 ng/ml, about 40 ng/ml, about 45 ng/ml, about 50 ng/ml, about 55 ng/ml, about 60 ng/ml, about 65 ng/ml, about 70 ng/ml, about 75 ng/ml, about 80 ng/ml, about 85 ng/ml, about 90 ng/ml, about 95 ng/ml, or about 100 ng/ml.

According to some embodiments, the SCKTCs can be purified by positive or negative selection cell separation methods. Positive selection cell separation methods involve directly labeling desired cells for selection with an antibody or a ligand that targets a specific cell surface protein. In immunomagnetic separation methods, the antibody or ligand is linked to a magnetic particle, allowing the labeled cells to be retained in the final isolated fraction after incubation of the same in a magnetic field. Negative selection cell separation methods involve laveling unwanted cell types for removal with antibodies or ligands targeting specific cell surface proteins. In immunomagnetic separation methods, the antibodies or ligands are linked to magnetic particles, allowing the labeled, unwanted cells to be depleted from the final isolated fraction by incubating the sample in a magnetic field. Since the desired cells are not specifically targeted by antibodies or ligands, they remain unbound by particles. According to some embodiments, magnetic bead activated cell sorting, a positive selection technique, can be used for purifying a specific cell population from the mononuclear cells. According to some embodiments, negative selection protocols can be employed to reduce the risk of decreasing the quantity and activity of the desired cells such protocols.

According to some embodiments a pure SCKTC population is achieved without positive or negative cell separation methods. According to some embodiments, the pulsing with DCs loaded with alpha-GalCer enables the increased purity of the SCKTC population without positive or negative selection cell separation methods. According to some embodiments, the SCKTCs prepared by the process are at least 80% pure SCKTCs. According to some embodiments, the SCKTCs prepared by the process are at least 81% pure SCKTCs. According to some embodiments, the SCKTCs prepared by the process are at least 82% pure SCKTCs. According to some embodiments, the SCKTCs prepared by the process are at least 83% pure SCKTCs. According to some embodiments, the SCKTCs prepared by the process are at least 84% pure SCKTCs. According to some embodiments, the SCKTCs prepared by the process are at least 85% pure SCKTCs. According to some embodiments, the SCKTCs prepared by the process are at least 86% pure SCKTCs. According to some embodiments, the SCKTCs prepared by the process are at least 87% pure SCKTCs. According to some embodiments, the SCKTCs prepared by the process are at least 88% pure SCKTCs. According to some embodiments, the SCKTCs prepared by the process are at least 89% pure SCKTCs. According to some embodiments, the SCKTCs prepared by the process are at least 90% pure SCKTCs. According to some embodiments, the SCKTCs prepared by the process are at least 91% pure SCKTCs. According to some embodiments, the SCKTCs prepared by the process are at least 92% pure SCKTCs. According to some embodiments, the SCKTCs prepared by the process are at least 93% pure SCKTCs. According to some embodiments, the SCKTCs prepared by the process are at least 94% pure SCKTCs. According to some embodiments, the SCKTCs prepared by the process are at least 95% pure SCKTCs. According to some embodiments, the SCKTCs prepared by the process are at least 96% pure SCKTCs, According to some embodiments, the SCKTCs prepared by the process are at least at least 97% pure SCKTCs.

According to some embodiments, the method comprises replenishing the culture medium in the culture system with fresh serum-free culture medium every 1 to 3 days, i.e., at least every 3 days, at least every 2 days, or every day. According to some embodiments cells are counted to about 0.8-1.5×10⁶ cells/ml and then fed with the fresh serum-free culture medium based on the cell count. According to some embodiments, the replenishing step includes adding to the culture system a pulse comprising an enriched population of DCs derived from PBMCs that are loaded with αGalCer or an analog or functional equivalent thereof. According to some embodiments, the number of pulses of DCs loaded with α-GalCer added to the SCKTC culture is at least 1, at least 2, at least 3, at least 4, at least 5, at least 6, at least 7, at least 8, at least 9, or at least 10 pulses.

According to some embodiments, the serum-free culture medium comprises X-VIVO-15 serum-free medium.

### CKTC Activation

According to some embodiments, the population of CKTCs of the described invention comprises a subpopulation of CD3+ T cells. According to some embodiments, the population of CKTCs comprises a subpopulation of NKT cells. According to one embodiment, the subpopulation of NKT cells comprises CD3+Vα24+ cells. According to one embodiment, the subpopulation of NKT cells comprises CD3+Vα24− cells. According to one embodiment, the subpopulation of NKT cells comprises CD3+CD56+ cells. According to some embodiments, the subpopulation of NKT cells comprise a subpopulation of type 1 NKT cells. According to some embodiments, the T cell receptor of the subpopulation of NKT cells comprises a Vα24-Jα18 TCRα chain. According to some embodiments, the T cell receptor of the subpopulation of NKT cells comprises a Vα24-Jα18 TCRα chain and a Vβ11 β chain. According to some embodiments, the subpopulation of NKT cells recognize glycolipid antigens presented by CD1d. According to some embodiments, the glycolipid antigen is αGalCer or an analog or functional equivalent thereof.

In nature, when type-I NKT cells are stimulated with α-GalCer, they produce IFN-γ. Simultaneously, they activate antigen-presenting cells (APCs) through CD40-CD40L interaction, especially inducing DCs to mature and up-regulate co-stimulatory receptors such as CD80 and CD86. DCs also produce IL-12 upon their interaction with type-I NKT cells. IL-12 induces more IFN-γ production by other T cells and plays a critical role together with IFN-γ in the activation of downstream effectors such as NK cells, CD8+ T cells and γδ T cells (Paget et al., J Immunol. 2012 Apr. 15; 188(8):3928-39). The interaction of type-I NKT cells with APCs offers activation signals to (i.e., licenses) APCs to render them able to cross-prime to CD8+ T cells through the induction of CD70 and CCL17 (Taraban et al., J Immunol. 2008 Apr. 1; 180(7):4615-20; Fujii et al., Immunol Rev. 2007 December; 220( ):183-98).

According to some embodiments, the activating of the population of CKTCs can comprise one or more of inducing secretion of a cytokine by the population of CKTCs, stimulating proliferation of the population of CKTCs, or modulating expression of one or more markers on the cell surface of the CKTCs. According to some embodiments, the cytokine whose expression is modulated is one or more cytokine selected from the group consisting of IFNγ, IL-4, IL-5, IL-6, or IL-10.

Activation of the population of CKTCs can be measured by various assays as described herein. Exemplary activities that may be measured include the induction of proliferation, the induction of expression of activation markers in the population of CKTCs, the induction of cytokine secretion by the population of CKTCs, the induction of signaling in the population of CKTCs, and an increase in the cytotoxic activity of the population of CKTCs.

### Cytokine Secretion

The activation of CKTCs to form SCKTCs may be assessed or measured by determining secretion of cytokines, including one or more of gamma interferon (IFNγ), interleukin 4 (IL-4), interleukin 5 (IL-5), interleukin 6 (IL-6) or interleukin-10 (IL-10). According to some embodiments, an ELISA is used to determine cytokine secretion, for example secretion of gamma interferon (IFNγ), IL-4, IL-5, IL-6 or IL-10. According to some embodiments, the ELISPOT (enzyme-linked immunospot) technique may be used to detect CKTCs and SCKTCs that secrete a given cytokine (e.g., gamma interferon (IFNγ)) in response to the methods described herein. For example, a culture system can be set up whereby a population of CKTCs or SCKTCs produced by the methods described herein are cultured within wells that have been coated with anti-IFNγ antibodies. The secreted IFNγ is captured by the coated antibody and then revealed with a second antibody coupled to a chromogenic substrate. Locally secreted cytokine molecules form spots, with each spot corresponding to one IFNγ-secreting cell. The number of spots allows one to determine the frequency of IFNγ-secreting cells in the analyzed sample. The ELISPOT assay has also been described for the detection of tumor necrosis factor alpha (TNFα), IL-4, IL-5, IL-6, IL-10, IL-12, granulocyte-macrophage colony-stimulating factor (GM-CSF), and granzyme B-secreting lymphocytes (Klinman D, Nutman T. Current protocols in immunology. New York, N.Y: John Wiley & Sons, Inc.; 1994. pp. 6.19.1-6.19.8, incorporated by reference in its entirety herein).

According to some embodiments, cytokine secretion is quantified by cytokine bead assay. Bead populations with distinct fluorescence intensities are coated with capture antibodies specific for IFN-γ and IL4 and mixed together to form a bead array that is resolved in a flow cytometer. During the assay procedure, the inflammatory cytokine capture beads are mixed with recombinant standards or SCKTCs and incubated with PE-detection antibodies. The intensity of PE fluorescence of each complex reveals the concentration of that cytokine.

Flow cytometric analyses of intracellular cytokines may be used to measure the cytokine content in culture supernatants, but provide no information on the number of NKT cells that actually secrete the cytokine. When lymphocytes are treated with inhibitors of secretion, such as monensin or brefeldin A, they accumulate cytokines within their cytoplasm upon activation. After fixation and permeabilization, intracellular cytokines can be quantified by cytometry. This technique allows the determination of the cytokines produced, the type of cells that produce these cytokines, and the quantity of cytokine produced per cell.

According to some embodiments, cytokine production by the enriched population of SCKTCs is characterized as IL-4 low, IL-5 low, IL-6 low, IL-10 low, IFNγ high.

According to one embodiment, the amount of IFN-γ produced by the population of cells into the culture supernatant is at least about 500 pg/ml; 1000 pg/ml; 1500 pg/ml; 2000 pg/ml, at least about 2500 pg/ml, at least about 3000 pg/ml, at least about 3500 pg/ml, at least about 4000 pg/ml, at least about 4500 pg/ml, at least about 5000 pg/ml, at least about 5500 pg/ml, at least about 6000 pg/ml, at least about 6500 pg/ml, at least about 7000 pg/ml, at least about 7500 pg/ml, at least about 8000 pg/ml, at least about 8500 pg/ml, at least about 9000 pg/ml, at least about 9500 pg/ml, at least about 10,000 pg/ml, at least about 10,500 pg/ml, at least about 11,000 pg/ml, at least about 11,500 pg/ml, at least about 12,000 pg/ml, at least about 12,500 pg/ml, at least about 13,000 pg/ml, at least bout 13,500 pg/ml, or at least about 14,0000 pg/ml.

According to some embodiments, the amount of IL-4 produced by the population of cells and secreted into the culture supernatant is less than 1000 pg/ml; less than 900 pg/ml; less than 800 pg/ml, less than 700 pg/ml, less than 600 pg/ml; less than 500 pg/ml; less than 400 pg/ml; less than 300 pg/ml; less than 200 pg/ml; less than 100 pg/ml; less than 90 pg/ml; less than 80 pg/ml; less than 70 pg/ml; less than 60 pg/ml; less than 50 pg/ml; less than 40 pg/ml; less than 30 pg/ml; less than 20 pg/ml; less than 10 pg/ml; less than 9 pg/ml; less than 8 pg/ml; less than 7 pg/ml; less than 6 pg/ml; less than 5 pg'ml; or 4 pg/ml, 3 pg/ml; 2 pg/ml or 1 pg/ml. According to some embodiments, the amount of IL-4 produced by the population of SCKTC cells and secreted into the culture supernatant ranges from 1-5 pg/ml; 5-6 pg/ml; 6-7 pg/ml; 7-8-pg/ml; 8-9 pg/ml; 9-10 pg/ml, 10-15 pg/ml; 10-20 pg/ml; 20-30 pg/ml; 30-40 pg/ml; 40-50 pg/ml; 50-60 pg/ml; 60-70 pg/ml; 70-80 pg/ml; 80-90 pg/ml; or 90-100 pg/ml, inclusive.

According to some embodiments, the ratio of IFNγ to IL-4 is an indicator of one or more T cell effector functions (such as cell killing and cell activation), of the CKTCs and SCKTCs.

According to one embodiment, the ratio of IFN-γ:IL-4 in culture supernatants is at least 500. According to one embodiment, the ratio of IFN-γ:IL-4 in culture supernatants is at least 600. According to one embodiment, the ratio of IFN-γ:IL-4 in culture supernatants is at least 700. According to one embodiment, the ratio of IFN-γ:IL-4 in culture supernatants is at least 800. According to one embodiment, the ratio of IFN-γ:IL-4 in culture supernatants is at least 900. According to one embodiment, the ratio of IFN-γ:IL-4 in culture supernatants is at least 1000. According to one embodiment, the ratio of IFN-γ:IL-4 in culture supernatants is at least 1100. According to one embodiment, the ratio of IFN-γ:IL-4 in culture supernatants is at least 1200. According to one embodiment, the ratio of IFN-γ:IL-4 in culture supernatants is at least 1300. According to one embodiment, the ratio of IFN-γ:IL-4 in culture supernatants is at least 1400. According to one embodiment, the ratio in culture supernatants of IFN-γ:IL-4 is at least 1500. According to one embodiment, the ratio of IFN-γ:IL-4 in culture supernatants is at least 1550. According to one embodiment, the ratio of IFN-γ:IL-4 in culture supernatants is at least 1600. According to one embodiment, the ratio of IFN-γ:IL-4 in culture supernatants is at least 1650. According to one embodiment, the ratio of IFN-γ:IL-4 in culture supernatants is at least 1700. According to one embodiment, the ratio of IFN-γ:IL-4 in culture supernatants is at least 1750. According to one embodiment, the ratio of IFN-γ:IL-4 in culture supernatants is at least 1800. According to one embodiment, the ratio of IFN-γ:IL-4 in culture supernatants is at least 1850. According to one embodiment, the ratio of IFN-γ:IL-4 in culture supernatants is at least 1900. According to one embodiment, the ratio of IFN-γ:IL-4 in culture supernatants is at least 1950. According to one embodiment, the ratio of IFN-γ:IL-4 in culture supernatants is at least 2000. According to one embodiment, the ratio of IFN-γ:IL-4 is at least 2050. According to one embodiment, the ratio of IFN-γ:IL-4 in culture supernatants is at least 2100. According to one embodiment, the ratio of IFN-γ:IL-4 in culture supernatants is at least 2150. According to one embodiment, the ratio of IFN-γ:IL-4 in culture supernatants is at least 2200. According to one embodiment, the ratio of IFN-γ:IL-4 in culture supernatants is at least 2250. According to one embodiment, the ratio of IFN-γ:IL-4 in culture supernatants is at least 2300. According to one embodiment, the ratio of IFN-γ:IL-4 in culture supernatants is at least 2350. According to one embodiment, the ratio of IFN-γ:IL-4 in culture supernatants is at least 2400. According to one embodiment, the ratio of IFN-γ:IL-4 in culture supernatants is at least 2450. According to one embodiment, the ratio of IFN-γ:IL-4 in culture supernatants is at least 2500. According to one embodiment, the ratio of IFN-γ:IL-4 in culture supernatants is at least 2550. According to one embodiment, the ratio of IFN-γ:IL-4 in culture supernatants is at least 2600. According to one embodiment, the ratio of IFN-γ:IL-4 in culture supernatants is at least 2650. According to one embodiment, the ratio of IFN-γ:IL-4 in culture supernatants is at least 2700. According to one embodiment, the ratio of IFN-γ:IL-4 in culture supernatants is at least 2750. According to one embodiment, the ratio of IFN-γ:IL-4 in culture supernatants is at least 2800. According to one embodiment, the ratio of IFN-γ:IL-4 in culture supernatants is at least 2850. According to one embodiment, the ratio of IFN-γ:IL-4 in culture supernatants is at least 2900. According to one embodiment, the ratio of IFN-γ:IL-4 in culture supernatants is at least 2950. According to one embodiment, the ratio of IFN-γ:IL-4 in culture supernatants is at least 3000.

### Cytotoxicity

The activation of CKTCs to form SCKTCs may be assessed by assaying cytotoxic activity of the CKTCs at each step of the described method.

The cytotoxic activity may be assessed by any suitable technique known to those of skill in the art. For example, a sample comprising a population of CKTCs or SCKTCs produced by the methods described herein can be assayed for cytotoxic activity after an appropriate period of time, in a standard cytotoxicity assay. Such assays may include, but are not limited to, the chromium release CTL assay and the ALAMAR BLUE fluorescence assay known in the art. According to some embodiments, cytotoxicity can be assayed in a lactate dehydrogenase (LDH) assay. LDH, a well-established and reliable indicator of cellular toxicity, is a cytosolic enzyme that is released into the cell culture medium upon damage to the plasma membrane. The extracellular LDH is then quantified by a coupled enzymatic reaction in which LDH catalyzes the conversion of lactate to pyruvate via NAD+ reduction to NADH, which then reduces a tetrazolium salt to a red formazan product that can be measured at 490 nm. The level of formazan formation is directly proportional to the amount of LDH released into the medium.

According to some embodiments, a population of SCKTC cells is collected by centrifugation and their cytotoxicity against A549 cells (human lung epithelial cell line) assessed. According to some embodiments, cytotoxicity is assessed against a genetically modified cell line that expresses increased amounts of CD1d, e.g., a genetically modified A549 cells or Panc-1 (pancreatic carcinoma) cells. According to some embodiments, a population of cells is collected by centrifugation and cytotoxicity against K562 cells (highly undifferentiated and of the granulocytic series, derived from a patient with chronic myeloid leukemia) is assessed. The K562 cell line, derived from a chronic myeloid leukemia (CML) patient and expressing B3A2 bcr-abl hybrid gene, is known to be particularly resistant to apoptotic death. (Luchetti, F. et al, Haematologica (1998) 83: 974-980). According to some embodiments, K562 target cells and SCKTCs are allocated into wells at one or more effector: target ratios, e.g. 5:1, 6:1, 7:1, 8:1, 9:1, 10:1, 11:1, 12:1, 13:1, 14:1, 15:1, 16:1, 17:1, 18:1, 19:1 or 20:1. After incubation, absorbance is detected by an enzyme-linked immunosorbent assay reader, and the killing rate can be calculated. According to some embodiments, the same assay can be carried out, where cytotoxicity against Jurkat cells (acute T leukemia) is assessed (Somanchi et al., PLoS ONE 10(10): e0141074. https://doi.org/10.1371/journal.pone.0141074).

According to some embodiments, killing rate can be represented by the following formula:

$$Killing Rate : ( \% ) = \frac{( {OD}_{490 experimental well} - {OD}_{490 negative well} )}{( {OD}_{490 experimental well} - {OD}_{490 negative well} )} \times 100$$

According to some embodiments, the killing rate of the CKTC population comprising SCKTCs against a target cell ranges from about 20% to about 85%, inclusive. According to some embodiments, the killing rate of the CKTC population comprising SCKTCs ranges from about 50% to about 75%, inclusive. According to some embodiments, the killing rate of the CKTC population comprising SCKTCs is at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%,at least 75%, at least 76%, at least 77%, at least 78%, at least 79%, at least 80%, at least 81%, at least 82%, at least 83%, at least 84%, at least 85%.

### Proliferation/Expansion

The ability of the described methods of the invention to induce expansion of the SCKTCs can be evaluated by staining using the fluorescent cell staining dye carboxyfluorescein syccinimidyl ester (CFSE). To compare the initial rate of cell expansion, the cells are stained with CFSE to determine how well the various steps of the described method (i.e. steps (b)-(e)) induced the proliferation of the SCKTCs. CFSE staining provides a quantitative endpoint and allows simultaneous phenotyping of the expanded cells. Every day after stimulation, an aliquot of cells is removed from each culture and analyzed by flow cytometry. CFSE staining makes cells highly fluorescent. Upon cell division, the fluorescence is halved and thus the more times a cell divides the less fluorescent it becomes. The ability of the described method to induce proliferation of the SCKTCs is quantitated by measuring the number of cells that divided once, twice, three times and so on.

To determine how well the described method promotes long-term growth of the SCKTCs, cell growth curves can be generated. These experiments are set up as are the foregoing CFSE experiments, but no CFSE is used. Every 2-3 days of culture, cells are removed from the respective cultures and counted using a Coulter counter, which measures how many cells are present and the mean volume of the cells. The mean cell volume is the best predictor of when to restimulate the cells. In addition, the phenotypes of the cells that are expanded can be characterized to determine whether a particular subset is preferentially expanded.

Prior to each restimulation, a phenotypic analysis of the expanding cell populations is performed to determine the presence of particular markers that define the SCKTC population. According to some embodiments, prior to each restimulation, an aliquot of cells is removed from each culture and analyzed by flow cytometry, using Forward Scatter (FS) vs 90° Light Scatter to bitmap the intact lymphocyte population. Gating (rectangular) on this bitmap, CD56 vs CD3 was measured. Gating on the double positives, Vα24 vs. Vβ11 was measured. Perforin and Granzyme B intracellular staining can be used to perform a gross measure to estimate cytolytic potential.

According to some embodiments, the population of SCKTCs is expanded from about 100- to about 1,000,000-fold, or from about 1,000- to about 1,000,000-fold, e.g., from about 1,000-fold to about 100,000-fold based on the population of starting CKTC cells, i.e., at least about 100-, at least about 200-, at least about 300-, at least about 400-, at least about 500-, at least about 600-, at least about 700-, at least about 800-, at least about 900-, at least about 1000-, at least about 2000-, at least about 3000-, at least about 4000-, at least about 5000-, at least about 6000-, at least about 7000-, at least about 8000-, at least about 9000-, at least about 10,000-, at least about 11,000-, at least about 12,000-, at least about 13,000-, at least about 14,000-, at least about 15,000-, at least about 16,000-, at least about 17,000-, at least about 18,000-, at least about 19,000-, at least about 20,000-, at least about 21,000-, at least about 22,000-, at least about 23,000-, at least about 24,000-, at least about 25,000-, at least about 26,000-, at least about 27,000-, at least about 28,000-, at least about 29,000-, at least about 30,000-, at least about 31,000-, at least about 32,000-, at least about 33,000-, at least about 34,000-, at least about 35,000-, at least about 36,000-, at least about 37,000, at least about 38,000-, at least about 39,000-, at least about 40,000-, at least about 41,000-, at least about 42,000-, at least about 43,000-, at least about 44,000-, at least about 44,000-, at least about 45,000-, at least about 46,000-, at least about 47,000-, at least about 48,000-, at least about 49,000-, at least about 50,000-, at least about 51,000-, at least about 52,000-, at least about 53,000-, at least about 54,000-, at least about 55,000-, at least about 56,000-, at least about 57,000-, at least about 58,000-, at least about 59,000-, at least about 60,000-, at least about 61,000-, at least about 62,000-, at least about 63,000-, at least about 64,000-, at least about 65,000-, at least about 66,000-, at least about 67,000-, at least about 68,000-, at least about 69,000-, at least about 70,000, at least about 71,000-, at least about 72,000-, at least about 73,000-, at least about 74,000-, at least about 75,000-, at least about 76,000-, at least about 77,000-, at least about 78,000-, at least about 79,000-, at least about 80,000-, at least about 81,000-, at least about 82,000-, at least about 83,000-, at least about 84,000-, at least about 85,000-, at least about 86,000-, at least about 87,000-, at least about 88,000-, at least about 89,000-, at least about 90,000-, at least about 91,000-, at least about 92,000-, at least about 93,000-, at least about 94,000-, at least about 95,000-, at least about 96,000-, at least about 97,000-, at least about 98,000-, at least about 99,000-, at least about 100,000-, at least about 200,000-, at least about 300,000-, at least about 400,000-, at least about 500,000-, at least about 600,000-, at least about 700,000-, at least about 800,000-, at least about 900,000-, or at least about 1,000,000-fold.

### 2. Pharmaceutical Composition Comprising the Cell Product Comprising SCKTCs

According to some embodiments, the cell product prepared by the method comprises at least about 5×10⁸ to about 5×10¹⁰ SCKTCs, inclusive, i.e., at least 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6.0×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7.0×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8.0×10⁸, 9.0×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸, 1×10⁹, 1.1×10⁹, 1.2×10⁹, 1.3×10⁹, 1.4×10⁹, 1.5×10⁹, 1.6×10⁹, 1.7×10⁹, 1.8×10⁹, 1.9×10⁹, 2.0×10⁹, 2.1×10⁹, 2.2×10⁹, 2.3×10⁹, 2.4×10⁹, 2.5×10⁹, 2.6×10⁹, 2.7×10⁹, 2.8×10⁹, 2.9×10⁹, 3.0×10⁹, 3.1×10⁹, 3.2×10⁹, 3.3×10⁹, 3.4×10⁹, 3.5×10⁹, 3.6×10⁹, 3.7×10⁹, 3.8×10⁹, 4.9×10⁹, 5.0×10⁹, 5.1×10⁹, 5.2×10⁹, 5.3×10⁹, 5.4×10⁹, 5.5×10⁹, 5.6×10⁹, 5.7×10⁹, 5.8×10⁹, 5.9×10⁹, 6.0×10⁹, 6.1×10⁹, 6.2×10⁹, 6.3×10⁹, 6.4×10⁹, 6.5×10⁹, 6.6×10⁹, 6.7×10⁹, 6.8×10⁹, 6.9×10⁹, 7.0×10⁹, 6.1×10⁹, 6.2×10⁹, 6.3×10⁹, 6.4×10⁹, 6.5×10⁹, 6.6×10⁹, 6.7×10⁹, 6.8×10⁹, 6.9×10⁹, 7.0×10⁹, 7.1×10⁹, 7.2×10⁹, 7.3×10⁹, 7.4×10⁹, 7.5×10⁹, 7.6×10⁹, 7.7×10⁹, 7.8×10⁹, 7.9×10⁹, 8.0×10⁹, 8.1×10⁹, 8.2×10⁹, 8.3×10⁹, 8.4×10⁹, 8.5×10⁹, 8.6×10⁹, 8.7×10⁹, 8.8×10⁹, 8.9×10⁹, 9.0×10⁹, 9.1×10⁹, 9.2×10⁹, 9.3×10⁹, 9.4×10⁹, 9.5×10⁹, 9.6×10⁹, 9.7×10⁹, 9.8×10⁹, 9.9×10⁹, 1.0×10¹⁰, 1.1×10¹⁰, 1.2×10¹⁰, 1.3×10¹⁰, 1.4×10¹⁰, 1.5×10¹⁰, 1.6×10¹⁰, 1.7×10¹⁰, 1.8×10¹⁰, 1.9×10¹⁰, 2.0×10¹⁰, 2.2×10¹⁰, 2.3×10¹⁰, 2.4×10¹⁰, 2.5×10¹⁰, 2.6×10¹⁰, 2.7×10¹⁰, 2.8×10¹⁰, 2.9×10¹⁰, 3.0×10¹⁰, 3.1×10¹⁰, 3.2×10¹⁰, 3.3×10¹⁰, 3.4×10¹⁰, 3.5×10¹⁰, 3.6×10¹⁰, 3.7×10¹⁰, 3.8×10¹⁰, 3.9×10¹⁰, 4.0×10¹⁰, 4.1×10¹⁰, 4.2×10¹⁰, 4.3×10¹⁰, 4.4×10¹⁰, 4.5×10¹⁰, 4.6×10¹⁰, 4.7×10¹⁰, 4.8×10¹⁰, 4.9×10¹⁰, or about 5.0×10¹⁰ SCKTCs. According to some embodiments the SCKTC cell product further contains about 0.8%, 0.9%, 1.0%, 1.1%, 1.2%, 1.3%, 1.4%, or about 1.5% DCs.

According to some embodiments, the SCKTC cell product prepared by the method is formulated with a pharmaceutically acceptable carrier. According to some embodiments the pharmaceutically acceptable carrier can contain one or more of Human Serum Albumin (HSA), Plasmalyte injection ((Multiple Electrolytes Injection), glucose/dextrose, or dextran 40. According to some embodiments, the SCKTCs can be cryopreserved in a freezing medium comprising 10% DM (e.g., cryoStor CS 10) and stored in the vapor phase of a liquid nitrogen freezer (−130° C. or lower). According to some embodiments, the freezing medium may comprise 31.25% (v/v) of Plasmia-Lyte A, 31.25% (v/v) of 5% Dextrose/0.45% sodiumchloride, 10% Dextran 40 (LMD)/5% Dextrose, 20% (v/v) of 25% Human Serum Albumin (HSA), and 7.5% (v/v) Cryoserv® dimethylsulfoxide (DMSO).

Target quality attributes of the amplified enriched population of SCKTCs of (g) prepared by the described method are shown in Table 4 below.

TABLE 4
Preliminary minimum acceptable target quality attributes

| | SCKTCs |
|---|---|
| | (preliminary minimum |
| Target attribute | acceptable specifications) |
| % Va24 + Vβ11 expression | [symbol] 80% after 3 weeks (2^{nd} DC pulse |
| (range) | [symbol] 1.5 × 10⁹ total yield |
| IFN-γ secretion: (range) | At least 2500 pg/ml w/ IL-12 stim; |
| | 200-750 pg/mL w/o IL-12 stim. |
| IL-4 secretion: (range) | 4-5 pg/mL w/ IL-12 stim. |
| IFN-γ: IL-4 ratio: (range) | [symbol] At least 500 w/ IL-12 stim. |
| | [symbol] 20-200 w/o IL-12 stim. |
| Cytotoxicity against A549 | At an Effector Target Ratio of 20: |
| cells (range): | 1, ≥50% cytotoxicity (A549) |
| Viability after freeze thaw | At least 80% |
| (range) | |
| Sterile, endotoxin negative | Negative |
| mycoplasma | |
| Bacterial and fungus | Negative |
| Therapeutic dose (range) | at least 0.2 × 10⁹ SCKTCs per |
| | treatment cycle (30 days) |

According to some embodiments, the properties of the SCKTC cell product are stable and reproducible from batch to batch. According to some embodiments, the fresh activated and expanded SCKTC cell product prepared by the process is characterized by at least 80% SCKTC viability and stability for at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, or at least 20 hours at room temperature. According to some embodiments, the fresh activated and expanded SCKTC cell product prepared by the process is characterized by identity of the SCKTCs, as confirmed by expression of cell surface markers by flow cytometry. According to some embodiments, the fresh activated and expanded SCKTC cell product prepared by the process is characterized by a purity of at least 80% SCKTCs. According to some embodiments, the fresh activated and expanded SCKTC cell product prepared by the process is characterized by secretion into the culture medium of at least 2500 pg/ml IFN-γ. According to some embodiments, the fresh activated and expanded SCKTC cell product prepared by the process is characterized by secretion of about 4-5 pg/mL IL-4 into the culture medium. According to some embodiments, the fresh activated and expanded SCKTC cell product prepared by the process is characterized by an IFN γ:IL4 ratio of at least 500 with IL-12 stimulation. According to some embodiments, the fresh activated and expanded SCKTC cell product prepared by the process is characterized by at least 50% cytotoxicity on A549 target cells at an effector:target ratio of 20:1.

According to some embodiments, the properties of the cryofrozen and thawed SCKTC cell product are stable and reproducible from batch to batch. According to some embodiments, the cryofrozen and thawed activated and expanded SCKTC cell product prepared by the process after thawing is pulsed with at least 1×10⁶ DCs loaded with α-GalCer. According to some embodiments, the cryofrozen, thawed and pulsed SCKTC product is characterized by at least 70% SCKTC viability and stability for at least 8 hours, at least 9 hours, at least 10 hours, at least 11 hours, at least 12 hours, at least 13 hours, at least 14 hours, at least 15 hours, at least 16 hours, at least 18 hours, or at least 18 hours, at least 19 hours, at least 20 hours, at least 21 hours, at least 22 hours, at least 23, hours, or at least 24 hours at room temperature. According to some embodiments, the cryofrozen and thawed activated and expanded SCKTC cell product prepared by the process is characterized by IFN-γ secretion into the culture medium of at least 2500 pg/mL with IL-12 stimulation. According to some embodiments, the cryofrozen and thawed activated and expanded SCKTC cell product prepared by the process is characterized by an IFN γ:IL4 ratio of at least 500 with IL-12 stimulation. According to some embodiments, the cryofrozen and thawed activated and expanded SCKTC cell product prepared by the process is characterized by at least 50% cytotoxicity on A549 target cells at an effector:target ratio of 20:1.

### Markers

According to some embodiments of the present disclosure, expansion of the SCKTCs using the methods as described herein can be determined by assessing the presence of markers that characterize the SCKTCs, and thereby determining the percent of the SCKTCs in the cell population. According to some embodiments, flow cytometry can be used to determine the presence of a subpopulation of SCKTCs expressing NKT cell markers using Forward Scatter (FS) vs 90° Light Scatter bitmap of the lymphocyte intact lymphocyte population. According to some embodiments, gating (rectangular) on this bitmap, CD56 vs CD3 is measured. According to some embodiments, gating on the double positives, Vα24 vs. Vβ11 is measured. According to some embodiments, a sub population of NKT cells can be determined by the presence of CD3 and CD56 markers (CD3+CD56+ NKT cells). According to some embodiments, binding of an anti-CD3 antibody labeled with a first fluorescent label (e.g. a commercially available fluorescently labeled anti-CD3 antibody, such as anti-CD3-pacific blue (PB) (BD Pharmingen, clone #SP34-2) and an anti-CD56 antibody labeled with a second fluorescent label (e.g. a commercially available fluorescently labeled anti-CD56 antibody, such as anti-CD56-Phycoerythrin (PE)-Cy7 (BD Pharmingen, clone #NCAM16.2)) can be used to determine expression of CD3 and CD56 in the cell population, where binding of the antibody is measured by flow cytometry for, e.g., PB fluorescence or PE fluorescence, and a gate is set based on CD3+CD56+ cells.

According to some embodiments, a subpopulation of type-I NKT cells can be determined by the presence of TCR Vα and TCR Vβ markers. According to one embodiment, binding of an anti-TCR Vα24 antibody labelled with a first fluorescent label (e.g. a commercially available fluorescently labeled anti-TCR Vα24 antibody, such as anti-TCR Vα24-PE (Beckman Coulter, clone # C15)) and an anti-TCR Vβ11 antibody labeled with a second fluorescent label (e.g. a commercially available fluorescently labeled anti-TCR Vβ11 antibody, such as anti-TCR Vβ-Fluorescein isothiocyanate (FITC) (Beckman Coulter, clone #C21)) can be used to determine expression of Vα24 and Vβ11 in the cell population, where binding of the antibody is measured by flow cytometry for, e.g., PE fluorescence or FITC fluorescence, and a gate is set based on Vα24+Vβ+11 cells.

According to some embodiments, a subpopulation of NKT cells can be characterized by expression of the markers CD3+Vα24+. According to some embodiments, a subpopulation of NKT cells is characterized by expression of the markers CD3+Vα24−. According to some embodiments, the subpopulation of type-I NKT cells includes cells characterized by the markers CD3+CD56+. According to some embodiments, the subpopulation of type-I NKT cells includes cells characterized by expression of the markers CD3+Vα24+, CD3+Vα24−, CD3+CD56+ and mixtures thereof.

### Additional Compatible Actives

According to some embodiments, the pharmaceutical composition of the described invention can further include one or more compatible active ingredients to provide the composition with another pharmaceutical effect in addition to that provided by the cell product of the described invention. “Compatible” as used herein means that the active ingredients of such a composition are capable of being combined with each other in such a manner so that there is no interaction that would substantially reduce the efficacy of each active ingredient or the composition under ordinary use conditions.

According to some embodiments, the pharmaceutical composition comprising the SCKTC cell product further comprises an enriched population of NK cells. According to some embodiments, the population of NK cells can be acquired by apheresis of peripheral blood from PBMCs. According to some embodiments, stem cell mobilization, a process whereby CD34+ hematopoietic stem cells are stimulated out of the bone marrow into the blood stream, may be used to harvest the PBMCs. According to some embodiments, Plerixafor in combination with G-CSF may be used to mobilize the CD34+ stem cells into the blood before collection. According to some embodiments, the PBMCs are depleted of CD3+ T cells and/or CD19 B cells with magnetic beads. According to some embodiments, CD3−CD56+NK cells are positively selected with magnetic beads. According to some embodiments, the selected CD3−CD56+ cells or T cell and/or B cell depleted cells are differentiated to NK cells by culturing in NK cell medium containing high IL-2 (2813 U/mL) for 14 days. According to some embodiments, the population of enriched NK cells can be expanded using static cell culture bags or an automated bioreactor. [e.g., see Saito, S. et al. Human Gene Therapy Methods (2013) 24 (4): 241-52; Spanholtz, J. et al. PLoS One (2011) 6 (6): e20740]. According to some embodiments, the NK cells are characterized by flow cytometry for identity/activation markers, for IFN-γ expression and secretion; and for their cytolytic potential against an MHC class I null cell line, e.g., K562.

According to some embodiments, a pharmaceutical composition comprising a-GalCer may be administered intranasally to activate the infused SCKTCs in situ [See, e.g., Artiaga, Bl et al. Sci Reports (2016) 6: 37999]. For example, excipients that offer mucosal bioadhhesion, in situ gelling tendency, ability to control the rate of drug clearance from the nasal cavity as well as protect the drug from enzymatic degradation are well suited for intranasal delivery. [Remington. The Science and Practice of Pharmacy, 23rd Ed. Adejare, A. Ed. In Chief, Academic (Cambridge, Mass. (2021) at p. 640, citing Upadhyay, S. et al. J. App. Pharm. Sci. (2011) 01 (03): 34-44; Ghori, M U et al. Am. J. Pharmacol. Sci. (2015) 3 (5): 110-19; Alnasser, S. Asian J. Pharm. Clin. Res. (2019) 12 (1): 40-45). Suspending agents act by retarding the agglomeration of particles by minimizing interparticle interaction and/or increasing viscosity of continuous medium (acting as thickeners or viscosity modifiers), thereby decreasing the settling rate of particles. These include inorganic materials, synthetic compounds of polysaccharides. Exemplary suspending agents and thickeners for intranasal administration include colloidal microcrystalline cellulose (MCC) (MCC and sodium carboxymethylcellulose (Na CMC), mesoporous methylcellulose (MPMC), methylcellulse (MC), Na CMC, pectin and polyethylene glycols (PEGs). Preservatives are added to pharmaceuticals to inhibit or prevent microbialgrowth and consequently ensure stability during shelf life. Exemplary preservatives for intranasal administration include methyl paraben, propyl paraben, and benzalkonium chloride. Penetration enhancers promote the transport of the drug across the nasal membrane, thereby improving nasal absorption of the drug. Examples include polysorbates, poloxamers, PEGs, propylene glycol and EDTA. Tonicity agents are used to adjust the osmolality of parenteral, ophthalmic and nasal solutions that directly come in contact with biological fluids. Exemplary tonicity agents include dextrose, glycerin, mannitol, potassium/sodium chloride and sorbitol/sorbitol solution. Buffering agents are weak acids or weak bases that are used to adjust, maintain or prevent rapid changes in the pH of a solution. Commonly used buffers include acetate, citrate, tartarate, phosphate and triethanolamide (TRIS) buffers.

According to some embodiments, the pharmaceutical composition comprising the SCKTC cell product containing the population of SCKTCs may be administered with a supportive therapy or an additional therapeutic agent, e.g., one or more of an immunomodulatory agent, an anti-inflammatory agent, an anti-infective agent, an anti-malarial agent, an anti-viral agent or an anti-fibrotic agent.

According to some embodiments, the supportive therapy is therapeutic apheresis comprising a virion removing step. According to some embodiments, the therapeutic apheresis reduces viral load.

According to some embodiments, the additional agent regulates immune cell activation. According to some embodiments, the additional agent modulates T cell exhaustion pathways.

### Immunomodulatory Agents

According to some embodiments, the immunomodulatory agent may comprise methotrexate; a glucocorticoid, cyclosporine, tacrolimus and sirolimus; a recombinant interferon selected from IFN-α; IFN-α-2b, IFN-β, IFN-γ, IFN-κ, IFN-ω; a recombinant IL-2 receptor inhibitor; a PDE4 inhibitor; a hyperimmune globulin prepared from a donor with high titers of a desired antibody; a TNFα inhibitor/antagonist; an IL-1β inhibitor; a chimeric IL-1Ra; an IL-6 inhibitor; an IL-12/IL-23 inhibitor selected from ustekinumab, briakinumab; an IL-23 inhibitor selected from guselkumab, tildrakizumab; a compound that targets TLR4 signaling; a p38 MAPK inhibitor, a compound that targets Janus kinase signaling; a compound that targets cell adhesion molecules to reduce leukocyte recruitment; a checkpoint inhibitor or a recombinant anti-inflammatory cytokine.

According to some embodiments, a physiologic or supraphysiological dose of the recombinant interferon comprising IFN-α; IFN-α-2b, IFN-β, IFN-γ, IFN-κ, and IFN-ω or a PEGylated form thereof boosts immune defenses of the subject.

According to some embodiments, the glucocorticoid comprises prednisone, dexamethasone, azathioprine, mycophenolate, mycophenolate mofetil, or combinations thereof; or the recombinant IL-2 inhibitor comprises denileukin diftitox; or the PDE4 inhibitor comprises cilomilast; or the TNFα inhibitor/antagonist comprises etanercept; adalimumab; infliximab, certolizumab pegol, or golimumab; or the IL-1β inhibitor comprises rilonacept; canakinumab; or Anakinra; or the IL-6 inhibitor comprises tocilizumab, siltuximab, sarilumab, olokizumab, or sirukumab; or the compound that targets TLR4 signaling comprises (ethyl 4-(4′-chlorophenyl) amino-6 methyl-2-oxocyclohex-3-en-1-aote (enamionone E121), JODI 18b; JODI 19, resatorvid, TLR-C34; or C35; or the p38 MAPK inhibitor comprises 4-(4′-fluorophenyl)-2-(4′-methylsulfinylphenyl)-5-(4′-pyridyl)-imidazole (SB203580), trans-4-[4-(4-fluorophenyl)-5-(2-methoxy-4-pyrimidinyl)-1H-imidazol-1-yl]cyclohexanol (SB239063), ord 4-[4-(4-fluorophenyl)-1-(3-phenylpropyl)-5-(4-pyridinyl)-1H-imidazol-2-yl]-3-butyn-1-ol (RWJ 67657); or the compound that targets Janus kinase signaling comprises tofacitinub, baricitinib, or upadacitinib; or the compound that targets a cell adhesion molecule to reduce leukocyte recruitment comprises an a4 integrin inhibitor comprising vedolizumab or natalizumab; or the recombinant anti-inflammatory cytokine comprises IL-4, IL-10, or IL-11; or the interferon is in a PEGylated form.

The term “immune checkpoint molecules” as used herein refers to ligand-receptor pairs that exert inhibitory or stimulatory effects on immune responses. Examples include programmed cell death 1 receptor (PD-1, also known as CD279), thought to regulate T cell proliferation later in the immune response, and its ligand programmed cell death ligand 1 (PD-L1), lymphocyte-activation gene 3 (LAG3), which suppresses T cells activation and cytokine secretion, thereby ensuring immune homeostasis and shows synergy with PD-1 to inhibit immune responses (Long, L. et al. Genes Cancer (2018) 9 (5-6): 176-89. and cytotoxic T-lymphocyte-associated antigen 4 (CTLA4; also known as CD152), a negative regulator of T cell immune function thought to regulate T cell proliferation early in an immune response [Buchbinder, E I, and Desai, A. Am. J. Clin. Oncol. (39) (10: 98-106). In addition, glucocorticoid-induced TNFR family related gene (GITR), a member of the TNFR superfamily (TNFRSF) that is expressed in different cell types, including T lymphocytes activation; GITR activation by its ligand (GITRL) influences the activity of effector and regulatory T cells, thus participating in the development of immune response against tumors and infectious agents, as well as in autoimmune and inflammatory diseases. [Nocentini, G. et al. Br. J. Pharmacol. (2012) 165 (7): 2089-99] T-cell immunoglobulin and mucin domain 3 (Tim-3) is a checkpoint receptor expressed by a wide variety of immune cells as well as leukemic stem cells. [Acharya, N. et al. J. Immunother. Cancer (2020) 8(10: e000911). T-cell immunoreceptor with immunoglobulin and ITIM domains (TIGIT) is an immune checkpoint receptor that can suppress T-cell activation and promote T-cell exhaustion. Inhibition of TIGIT may increase cytotoxic T-cell proliferation and function. Inducible T cell costimulator (ICOS, cluster of differentiation (CD278)) is an activating costimulatory immune checkpoint expressed on activated T cells. Its ligand, ICOSL is expressed on antigen-presenting cells and somatic cells, including tumour cells in the tumour microenvironment. [Solinas, C. et al. ESMO Open. (2020) 5(1): e000544].

According to some embodiments, the immunomodulator is an immune checkpoint inhibitor. The term “immune checkpoint inhibitor” as used herein refers to a molecule that can block immune checkpoint molecules. Specific immune checkpoint inhibitors, including antibodies against CTLA-4, PD-1 receptor or its ligand PD-L1 include YERVOY™ (Ipilimumab; CTLA-4 antagonist), OPDIVO™ (Nivolumab; PD-1 antagonist) and KEYTRUDA™ (Pembrolizumab; PD-1 antagonist) in multiple tumor indications, with ongoing registration trials in many more.

According to some embodiments, the immunomodulatory agent comprises recombinant IL-37. According to some embodiments, the immunomodulatory agent comprises recombinant CD24. According to some embodiments, the immunomodulatory agent comprises rIL-37 and rCD24.

IL-37 is a member of the IL-1 family, which includes IL-1α, IL-1β, IL-18, IL33, IL36α, IL-36β, IL-36γ, IL-37 and IL-38. [Mantovani, A. et al. Immunity (2019) doi.10.1016/j.immuni.2019.03.012]. The IL-1 family of cytokines is divided into three subgroups on the basis of the Il-1 consensus sequence and the signaling receptor chain. These include secreted molecules with agonistic activity [IL-1α, IL-1β, IL-18, IL33, IL36α, IL-36β, IL-36γ], receptor antagonists [IL-1Ra, IL36Ra, and IL-38] and an anti-inflammatory cytokine (IL-37) [Id., citing Dinarello, C A Immunol. Rev. (2018) 281: 8-27]. There is no murine counterpart to human IL-37. [Id.].

In humans, production of IL-37 is activated by pro-inflammatory stimuli, including cytokines, as a protective mechanism to prevent runaway inflammation and excessive damage. Five transcripts for the human IL-37 gene have been identified (IL-37 a-e). IL-37b is the most complete of these isoforms, is the most abundant and studied, and includes 5 of the 6 exons of the IL-37 gene (all but exon 3). Exons 4,5, and 6 encode for the sequence required for the beta-fold barrel structure and account for the extracellular activity of recombinant IL-37. Conversely, exons 1 2, and 3 may be cleaved in the extracellular environment by unknown proteases. IL-37 isoforms a, b and d share exons 4, 5, and 6 and encode functional proteins. The IL-37 isoforms c and e lack one or more of these exons and likely encode non-functional proteins. [Cavalli, G. and Dinarello, C A. Immunological Revs. (2018) 281 (1): 179-90].

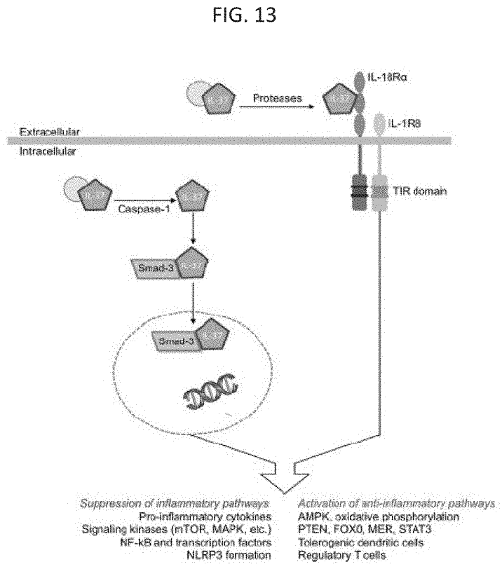

Low concentrations of recombinant IL-37 most effectively suppress cytokine production in vitro. In nature, IL-37 is a dual function cytokine exerting potent anti-inflammatory effects via two distinct mechanisms, either extracellular (receptor-mediated) or intracellular (nuclear function). As shown in FIG. 13, extracellular IL-37 forms a complex with cell surface IL-18 receptor α (IL-18Rα) and IL-1 receptor 8 (IL-1R8), which transduces anti-inflammatory signals. Intracellular IL-37 produced upon proinflammatory stimuli interacts with Smad3 and traffics to the nucleus, where it regulates gene expression and dampens transcription of pro-inflammatory genes.

Intracelluar/endogenous activity of IL-37. The IL-37 precursor is synthesized in human blood monocytes following stimulation by IL-1 or TLR agonists. in human blood monocytes, Pro-inflammatory stimuli induce an increase in intracellular IL-37 precursor while also triggering the activation of caspase-1, which cleaves the carboxyldomain of the IL-37 precursor. Mature Il-37 then associates with phosphorylated Smad-3, which enables nuclear translocation and regulation of gene transcription. Both the mature and precursor forms of IL-37 are released into the extracellulular space upon cell death or secreted by an unknown mechanism.

Extracellular/exogenous activity of IL-37. Both the mature and precursor forms of IL-37 are released into the extracellular space upon cell each or secreted by an unknown mechanism. Extracellular proteases process IL-37 precursor into the mature form. IL-37 binds to the IL18Rα and recruits IL1R8. IL-1R8 has a mutated TIR domain, which functions as a sink for MyD88; as a result, there is a weak or no transduction of pro-inflammatory signals, while anti-inflammatory pathways are activated. [Cavalli, G. and Dinearello, C A. Immunological Revs. (2018) 281(1): 179-90].

Li, et al. (accepted manuscript) examined early response of IL-37 in 254 SARS-CoV-2 infected patients prior to any clinical intervention and determined that higher early IL-37 plasma responses correlated with earlier viral RNA negative conversion, chest CT image improvement and cough relief, resulting in earlier hospital discharge. Higher IL-37 was associated with lower IL-6 and IL-8 and higher IFN-α in these patients. In contrast, low early IL-37 plasma responses predicted severe clinical prognosis in combination with IL-8 and C-reactive protein (CRP), a blood test for inflammation. They reported that Il-37 administration attenuated lung inflammation and alleviated respiratory tissue damage in human angiotensin-converting enzyme 2 (hACE2)-transgenic mice infected with SARS-CoV2.

CD24, also known as Heat Stable Antigen (HSA) or Small Cell Lung Carcinoma Cluster 4 Antigen, is a heavily glycosylated glycophosphatidylinositol (GPI)-anchored surface protein [Barkal, A. A. et al. Nature (2019) 572 (7769): 392-96, citing Pirruccello S J, LeBien T W The human B cell-associated antigen CD24 is a single chain sialoglycoprotein. J. Immunol. 136, 3779-3784 (1986), Chen G Y, Brown N K, Zheng P, Liu Y Siglec-G/10 in self-nonself discrimination of innate and adaptive immunity. Glycobiology 9, 800-806 (2014)]. Several signal transduction proteins are associated with CD24 activity, including the Src-family protein tyrosine kinases Lyn, Fyn, Fgr, Lck snf Hck, but how these are activated is unknown. [Ayre, D C and Christian, S L, Front. Cell & Devel. Biol. (2016) 4: 1146]. Many ligands have been identified for CD24, including P-, L- and E-Selectin, High Mobility Group Box 1 (HMGB1), Li cell adhesion molecule (L1CAM), Neural cell adhesion molecule (NCAM1) and Siglec-G. [Id., citing Aigner, et al. 1995; Myung, et al. 2011; Tan et al 2016]. To explain the contradictory nature of the processes regulated by CD24, its apparent lack of intrinsic signaling capability or its diverse collection of reported ligands, It has been proposed that CD24 functions as a rheostat to modulate responses transduced by partnered cell surface receptor(s), and that these partners define the biological outcomes observed [Ayre, D C and Christian, S L, Front. Cell & Devel. Biol. (2016) 4: 1146]. Mechanistically, the variable nature of CD24-mediated effects can be explained by its in cis association with unique cell-type specific signaling partners through direct physical interaction mediated by its modifiable glycosylations. [Ayre, D C and Christian, S L, Front. Cell & Devel. Biol. (2016) 4: 1146].

It has been hypothesized that the DAMPS released during cell death in viral infection may cause a self-propagating inflammatory response with lasting lung damage. [Tian, R-R et al. Cellular & Molec. Immunol. (2020) 17: 887-888]. Therefore, it has been proposed that the CD24-mediated Siglec10/G interaction is an immune checkpoint that regulates inflammation caused by DAMPS. [Id., citing Chen, G. et al. Science (2009) 323: 1722-25; Liu, Y. et al. Trends Immunol. (2009) 30: 557-61; Fang, X. et al. Cell Mol. Immunol. (2010) 7: 100-103].

CD24 is known to interact with Sialic Acid Binding Ig Like Lectin 10 (Siglec-10) on innate immune cells in order to dampen damaging inflammatory responses to infection [Id., citing Chen W et al. Induction of Siglec-G by RNA viruses inhibits the innate immune response by promoting RIG-I degradation. (2013) Cell 152(3), 467-478], sepsis [(Id, citing Chen G Y et al. Amelioration of sepsis by inhibiting sialidase-mediated disruption of the CD24-SiglecG interaction. Nature Biotechnology (2011) 29, 428-435), liver damage [Id., citing Chen G Y et al. CD24 and Siglec-10 selectively repress tissue damage-induced immune responses. Science (2009) 323 (5922), 1722-1725], and chronic graft v. host disease [Id., citing Toubai T et al. Siglec-G-CD24 axis controls the severity of graft-versus-host disease in mice. Blood (2014) 123(22), 3512-3513]. The binding of CD24 to Siglec-10 elicits an inhibitory signaling cascade mediated by SHP-1 and/or SHP-2 phosphatases associated with the two immunoreceptor tyrosine-based inhibition motifs (ITIMS) in the cytoplasmic tail of Siglec-10, thereby blocking TLR-mediated inflammation and the cytoskeletal rearrangement required for cellular engulfment by macrophages [Id., citing Crocker P R, Paulson J C, Varki A Siglecs and their roles in the immune system. Nature Reviews Immunology (2007) 7, 255-266; Abram C L, Lowell C A Shp1 function in myeloid cells. J. Leukoc. Biol (2017) 102(3), 657-675 Dietrich J, Cella M, Colonna M Ig-Like Transcript 2 (ILT2)/Leukocyte Ig-Like Receptor 1 (LIR1) Inhibits TCR Signalling and Actin Cytoskeleton Reorganization. J. Immunol. (2001) 166(4), 2514-2521].

It has been reported that a recombinant fusion protein CD24-Fc (an agonist of Sioglecs, which can fortify the CD24-Siglec innate immune checkpoint) had a therapeutic effect on SIV-induced lung inflammatory lesions. [Tian, R-R et al., Cellular & Molec. Immunol. (2020) 17: 887-88]. Chinese rhesus macaques were infected with simian immunodeficiency virus SIVmac239 via intravenous infusion received either three injections of a recombinant fusion protein CD24-Fc or normal saline on day 56 of infection. Five months later, another cycle of treatment was given to the surviving animals, which were terminated one week after the last dosing. Previous studies had shown that lung lesions developed within 2-4 weeks in SIV-infected rhesus monkeys. By 8 weeks, essentially all monkeys developed lung pathology. The data showed that CD24Fc not only reduced the incidence of viral pneumonia but also qualitatively altered the nature of the pathology in the lung.

It has also been reported that CD24 is important in regulating T cell survival. T cells must regulate their proliferation to support a long-lived cell population, but can expand their numbers during immune activation. [Ayre, D C and Christian, S L, Front. Cell & Devel. Biol. (2016) 4: 1146, citing Boyman, O. et al Eur. J. Immunol. (2009) 39: 2088-94]. In the absence of CD24, homeostatic proliferation of T cells is markedly reduced, however immune-driven proliferation is less affected [Id., citing Li. O. et al. J. Exp. Med. (2004) 200: 1083-89], likely because it depends on TCR co-receptors [Id., citing Chen, L. and Flies, D B Nat. Rev. Immunol. (2013) 13: 227-42]. When CD24+ T cells are transferred to CD24-knockout mice, excessive and destructive homeostatic T cell proliferation occurs, but CD24 expressed on dendritic cells is sufficient to ameliorate this effect [Id., citing Li, O. et al J. Exp. Med. (2006) 203: 1713-20]. This suggested that CD24 can act in cis on the T cell to regulate TCR signaling, or in trans, where DC-expressed Cd24 can bind and modulate its partner(s) on the T cell.

### Other Compatible Actives

According to some embodiments, the anti-inflammatory agent comprises aspirin, celecoxib, diclofenac, diflunisal, etodolac, ibuprofen, indomethacin, infliximab, ketoprofen, ketorolac nabumetone, naproxen, nintedanib, oxaprozin, pirfenidone, piroxicam, salsalate, sarilumab (Kevzara®) sulindac, tolmetin, or combinations thereof.

According to some embodiments, the anti-infective agent comprises amoxicillin, doxycycline, demeclocycline; eravacycline, minocycline, ormadacycline, tetracycline, cephalexin, defotaxime, cetazidime, cefuroxime, ceftaroline; ciprofloxacin, levofloxacin, moxifloxacin, clindamycin, lincomycin, metronidazole, azithromycin; clarithromycin, erythromycin, sulfamethoxazle and trimethoprim; sulfasalazine, amoxicillin and clavulanate; vancomycin, dalbavancin, oritavancin, telavancin, gentamycin, tobramycin, amikacin, imipenem and cilastatin, meropenem, doripenem, or ertapenem.

According to some embodiments, the anti-malarial agent comprises quinine, quinidine, chloroquine, hydroxychloroquine, amodiaquine, mefloquine, halofantrine, lumefantrine, piperaquine, and tafenoquine; an antifolate compound selected from pyrimethamine, proguanil, chlorproguanil, trimethoprim; an artemisinin compound selected from artemisinin, dihydroartemisinin, artemether, artesunate; or atovaquone.

According to some embodiments, the anti-viral agent comprises acyclovir, gancidovir, foscamet; ribavirin; amantadine, azidodeoxythymidine/zidovudine), nevirapine, a tetrahydroimidazobenzodiazepinone (TIBO) compound; efavirenz; remdecivir, lopinavir/ritonavir, umifenovir, favipiravir, ivermectin, or delavirdine. According to some embodiments, the anti-viral agent is an agent that inhibits viral entry and decreases viral load.

According to some embodiments, the anti-fibrotic agent comprises nintedanib, pirfenidone, ord combinations thereof.

### Formulations

Formulations of the pharmaceutical composition suitable for parenteral administration comprise the active ingredient combined with a pharmaceutically acceptable carrier, such as sterile water or sterile isotonic saline. Exemplary carrier solutions also can contain buffers, diluents and other suitable additives. The term “buffer” as used herein refers to a solution or liquid whose chemical makeup neutralizes acids or bases without a significant change in pH. Examples of buffers envisioned by the described invention include, without limitation, Dulbecco's phosphate buffered saline (PBS), Ringer's solution, 5% dextrose in water (D5W), normal/physiologic saline (0.9% NaCl). In some embodiments, the infusion solution is isotonic to subject tissues.

Exemplary pharmaceutical compositions of the described invention may comprise a suspension or dispersion of cells in a nontoxic parenterally acceptable diluent or solvent. A solution generally is considered as a homogeneous mixture of two or more substances; it is frequently, though not necessarily, a liquid. In a solution, the molecules of the solute (or dissolved substance) are uniformly distributed among those of the solvent. A dispersion is a two-phase system, in which one phase (e.g., particles) is distributed in a second or continuous phase. A suspension is a dispersion in which a finely-divided species is combined with another species, with the former being so finely divided and mixed that it does not rapidly settle out. Among the acceptable vehicles and solvents that may be employed are water, Ringer's solution, and isotonic sodium chloride (saline) solution.

Additional compositions of the present disclosure can be readily prepared using technology which is known in the art such as described in Remington's Pharmaceutical Sciences, 18th or 19th editions, published by the Mack Publishing Company of Easton, Pa., which is incorporated herein by reference.

Formulations of the pharmaceutical composition may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Pharmaceutical compositions that are useful in the methods of the disclosure may be prepared/formulated, packaged, or sold in formulations suitable for oral, rectal, vaginal, parenteral, topical, pulmonary, intranasal, intra-lesional, buccal, ophthalmic, intravenous, intra-organ or another route of administration. Other contemplated formulations include projected nanoparticles, liposomal preparations, resealed erythrocytes containing the active ingredient, and immunologically-based formulations.

According to some embodiments, the pharmaceutical compositions of the described invention may be administered initially, and thereafter maintained by further administrations. For example, according to some embodiments, the pharmaceutical compositions of the described invention may be administered by one method of injection, and thereafter further administered by the same or by different method.

According to some embodiments, a protein stabilizing agent can be added to the cell product comprising the expended and enriched population of SCKTCs after manufacturing, for example albumin, which may act as a stabilizing agent. According to some embodiments, the albumin is human albumin. According to some embodiments, the albumin is recombinant human albumin. According to some embodiments, the minimum amounts of albumin employed in the formulation may be about 0.5% to about 25% w/w, i.e., about 0.5%, about 1.0%, about 2.0, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25% w/w, including intermediate values, such as about 12.5% w/w.

According to some embodiments, the pharmaceutical composition may comprise a stabilizing amount of serum. The term “stabilizing amount” as used herein refers to the amount of serum that, when included in the formulation of the pharmaceutical composition of the described invention comprising enriched SCKTCs, enables these cells to retain their T cell effector activity. According to some embodiments, the serum is human serum autologous to a human patient. According to some embodiments, the serum is synthetic serum. According to some embodiments the stabilizing amount of serum is at least about 1.0% (v/v).

According to some embodiments, the methods of the present disclosure comprise the further step of preparing the pharmaceutical composition by adding a pharmaceutically acceptable excipient, in particular an excipient as described herein, for example a diluent, stabilizer and/or preservative.

The term “excipient” as employed herein is a generic term to cover all ingredients added to the SCKTC population that do not have a biological or physiological function, which are nontoxic and do not interact with other components.

Once the final formulation of the pharmaceutical composition has been prepared it will be filled into a suitable container, for example an infusion bag or cryovial.

According to some embodiments, the methods according to the present disclosure comprises the further step of filling the pharmaceutical composition comprising the cell product containing the expanded and enriched population of SCKTCs or a pharmaceutical formulation thereof into a suitable container, such as an infusion bag and sealing the same to form the cell product.

According to some embodiments, the product comprising the container filled with the pharmaceutical composition comprising the cell product comprising the expanded and enriched population of SCKTCs of the present disclosure is frozen for storage and transport, for example at about −135° C., for example in the vapor phase of liquid nitrogen. According to some such embodiments, the formulation may also contain a cryopreservative, such as DMSO. The quantity of DMSO generally is from about 5% to about 10%, inclusive, i.e., at least 5%, at least 6%, at least 7%, at least 8%, at least 9% or 10% v/v.

According to some embodiments, the process of the present disclosure comprises the further step of freezing the pharmaceutical composition, or the cell product comprising the expanded and enriched population of SCKTCs of the present disclosure. According to one embodiment, freezing occurs by a controlled rate freezing process, for example reducing the temperature by 1° C. per minute to ensure the crystals formed are small and do not disrupt cell structure. This process may be continued until the sample has reached at least −80° C.

Controlled- or sustained-release formulations of the pharmaceutical composition of the disclosure may be made by adapting otherwise conventional technology. The term “controlled release” as used herein is intended to refer to any drug-containing formulation in which the manner and profile of drug release from the formulation are controlled. This includes immediate as well as non-immediate release formulations, with non-immediate release formulations including, but not limited to, sustained release and delayed release formulations. The term “sustained release” (also referred to as “extended release”) is used herein in its conventional sense to refer to a drug formulation that provides for gradual release of a drug over an extended period of time, and that preferably, although not necessarily, results in substantially constant levels of a drug over an extended time period. The term “delayed release” is used herein in its conventional sense to refer to a drug formulation in which there is a time delay between administration of the formulation and the release of the drug therefrom. “Delayed release” may or may not involve gradual release of drug over an extended period of time, and thus may or may not be “sustained release.” The term “long-term” release, as used herein, means that the drug formulation is constructed and arranged to deliver therapeutic levels of the active ingredient over a prolonged period of time, e.g., days.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations may include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt. For parenteral application, suitable vehicles consist of solutions, e.g., oily or aqueous solutions, as well as suspensions, emulsions, or implants. Aqueous suspensions may contain substances, which increase the viscosity of the suspension and include, for example, sodium carboxymethyl cellulose, sorbitol and/or dextran.

According to some embodiments, the present disclosure provides a method of transporting a cell product comprising the expanded and enriched population of SCKTCs according to the present disclosure from the place of manufacture, or a convenient collection point, to a therapeutic facility. According to some embodiments, the temperature of the cell product is maintained during such transporting. According to some embodiments, for example, the pharmaceutical composition can be stored below 0° C., such as −135° C. during transit. According to some embodiments, temperature fluctuations of the pharmaceutical composition are monitored during storage and/or transport.

### 3. Administering the Pharmaceutical Composition Comprising the Cell Product

According to another aspect, the present disclosure provides a method of treating a virus infection, comprising administering to a subject in need thereof a pharmaceutical composition comprising a therapeutic amount of the cell product comprising superactivated cytokine killer T cells of the present disclosure.

According to some embodiments, the virus infection is an infection with a respiratory virus. According to some embodiments the respiratory virus is a respiratory syncytial virus (RSV), an Ebola virus, a cytomegalovirus, a Hanta virus, an influenza virus, a coronavirus, a Zika virus, a West Nile virus, a dengue virus, a Japanese encephalitis virus, a tick-borne encephalitis virus, a yellow fever virus, a rhinovirus, an adenovirus, a herpes virus, an Epstein Barr virus, a measles virus, a mumps virus, a rotavirus, a coxsackie virus, a norovirus, or an encephalomyocarditis virus (EMCV). According to some embodiments, the coronavirus is SARS-CoV-1, SARS-CoV-2 or MERS.

According to some embodiments, the respiratory virus infection is a severe viral infection. According to some embodiments, symptoms of the severe respiratory virus infection include one or more of: primary viral pneumonia; superimposed bacterial pneumonia; disruption or injury to alveolar epithelium, endothelium or both; acute lung injury (ALI); acute respiratory distress syndrome (ARDS); symptoms of shock; excessive complement activation; a pathological increase in vascular permeability; endothelial activation, loss of barrier function and consequent microvascular leak; thrombotic complications; kidney damage; or elevated concentrations of one or more inflammatory mediators in plasma (hypercytokinemia), compared to a normal healthy subject. 1%

According to some embodiments, symptoms of shock include low blood pressure, lightheadedness, shortness of breath, and rash. According to some embodiments, the thrombotic complications include one or more of formation of pulmonary microthrombi, acute pulmonary embolism, deep-vein thrombosis, ischemic stroke, myocardial infarction, or systemic arterial embolism. According to some embodiments, the inflammatory mediator includes one or more of interferon α, interferon β, interferon-κ, interferon-γ, complement, prostaglandin D2, vasoactive intestinal peptide (VIP), interleukin-1-beta (IL-1β), interleukin-6 (IL-6), interleukin-8 (IL-8), interleukin-12 (IL-12), IL-17, or tumor necrosis factor-alpha (TNF-α).

According to some embodiments, the severe viral infection is characterized by viral pathogen-infected cells.

According to some embodiments, the therapeutic amount reduces risk of the virus infection. According to some embodiments, the therapeutic amount reduces signs, symptoms, or both signs and symptoms of the viral infection. According to some embodiments, the therapeutic amount reduces extent of the viral infection where symptoms are not yet clinically recognized. According to some embodiments, the therapeutic amount reduces worsening or progression of the viral infection. According to some embodiments, the therapeutic amount reduces severity of the viral infection developed compared to an untreated subject. According to some embodiments, the therapeutic amount decreases viral burden. According to some embodiments, the therapeutic amount improves progression-free survival. According to some embodiments, the therapeutic amount improves overall survival.

According to some embodiments, the therapeutic amount destroys the infected cells through direct lysis or by effecting destruction of the infected cells indirectly, e.g., by mobilizing attracting cell cytotoxicity agents through secretion of cytokines.

According to some embodiments, the therapeutic amount mobilizes the patient's immune response to the viral pathogen, where the term “mobilizes” as used herein means to put into motion or use, become ready or capable of being moved quickly and with relative ease. stimulates activation of the patient's lymphocyte populations.

According to some embodiments, the term “a therapeutically effective amount” or dose does not necessarily mean an amount that is immediately therapeutically effective, but includes a dose which is capable of expansion in vivo (after administration) to provide a therapeutic effect. Thus, there is provided a method of administering to a patient a sub-therapeutic dose that nonetheless becomes a therapeutic amount after expansion and activation of SCKTCs in vivo to provide the desired therapeutic effect.

According to some aspects, the pharmaceutical composition of the present disclosure supplements a biologically insufficient immune response of the subject at risk for a virus infection by stimulating one or more immune cell population of the subject. According to some embodiment, the immune cell population comprises a dendritic cell population. According to some embodiments, the immune cell population comprises a CD8+ T cell population. According to some embodiments, the immune cell population comprises an NK cell population. According to some embodiments, the immune cell population comprises an MHC-restricted T cell population. According to some embodiments, the MNC-restricted T cell population comprises an invariant NKT population.

According to some embodiments, the therapeutic amount stimulates an effector function of the patient's immune cells. According to some embodiments, the effector function of the immune cell includes one or more of cytokine secretion, cytotoxicity, or antibody-mediated clearance of the pathogen.

### Additional Compatible Actives

According to some embodiments, the pharmaceutical compositions of the described invention can further include one or more compatible active ingredients which are aimed at providing the composition with another pharmaceutical effect in addition to that provided by the cell product of the described invention. “Compatible” as used herein means that the active ingredients of such a composition are capable of being combined with each other in such a manner so that there is no interaction that would substantially reduce the efficacy of each active ingredient or the composition under ordinary use conditions.

According to some embodiments, the pharmaceutical composition comprising the SCKTC cell product further comprises an enriched population of NK cells. According to some embodiments, the population of NK cells can be acquired by apheresis of peripheral blood from PBMCs. According to some embodiments, stem cell mobilization, a process whereby CD34+ hematopoietic stem cells are stimulated out of the bone marrow into the blood stream, may be used to harvest PBMCs. According to some embodiments, Plerixafor in combination with G-CSF may be used to mobilize the CD34+ stem cells into the blood before collection. According to some embodiments, the PBMCs are depleted of CD3+ T cells and/or CD19 B cells with magnetic beads. According to some embodiments, CD3−CD56+NK cells are positively selected with magnetic beads. According to some embodiments, the selected CD3−CD56+ cells or T cell and/or B cell depleted cells are differentiated to NK cells by culturing in NK cell medium containing high IL-2 (2813 U/mL) for 14 days. According to some embodiments, the population of enriched NK cells can be expanded using static cell culture bags or an automated bioreactor. [e.g., see Saito, S. et al. Human Gene Therapy Methods (2013) 24 (4): 241-52; Spanholtz, J. et al. PLoS One (2011) 6 (6): e20740]. According to some embodiments, the NK cells are characterized by flow cytometry for identity/activation markers, for IFN-γ expression and secretion; and for their cytolytic potential against an MHC class I null cell line, e.g., K562.

According to some embodiments, the pharmaceutical composition comprising the cell product containing the population of SCKTCs may be administered with a supportive therapy or an additional therapeutic agent, e.g., one or more of an immunomodulatory agent, an anti-inflammatory agent, an anti-infective agent, an anti-malarial agent, an anti-viral agent or an anti-fibrotic agent.

According to some embodiments, the supportive therapy is therapeutic apheresis comprising a virion removing step. According to some embodiments, the therapeutic apheresis reduces viral load.

According to some embodiments, the additional agent regulates immune cell activation. According to some embodiments, the additional agent modulates T cell exhaustion pathways.

### Immunomodulatory Agents

According to some embodiments, the immunomodulatory agent may comprise methotrexate; a glucocorticoid, cyclosporine, tacrolimus and sirolimus; a recombinant interferon selected from IFN-α; IFN-α-2b, IFN-β, IFN-γ, IFN-κ, IFN-ω; a recombinant IL-2 receptor inhibitor; a PDE4 inhibitor; a hyperimmune globulin prepared from a donor with high titers of a desired antibody; a TNFα inhibitor/antagonist; an IL-1β inhibitor; a chimeric IL-1Ra; an IL-6 inhibitor; an IL-12/IL-23 inhibitor selected from ustekinumab, briakinumab; an IL-23 inhibitor selected from guselkumab, tildrakizumab; a compound that targets TLR4 signaling; a p38 MAPK inhibitor, a compound that targets Janus kinase signaling; a compound that targets cell adhesion molecules to reduce leukocyte recruitment; a checkpoint inhibitor, or a recombinant anti-inflammatory cytokine.

According to some embodiments, a physiologic or supraphysiological dose of the recombinant interferon selected from IFN-α; IFN-α-2b, IFN-β, IFN-γ, IFN-κ, and IFN-ω or a PEGylated form thereof boosts immune defenses of the subject.

According to some embodiments, the glucocorticoid comprises a corticosteroid comprising prednisone, dexamethasone, azathioprine, mycophenolate, mycophenolate mofetil, or combinations thereof; or the recombinant IL-2 inhibitor comprises denileukin diftitox; or the PDE4 inhibitor comprises cilomilast; or the TNFα inhibitor/antagonist comprises etanercept; adalimumab; infliximab, certolizumab pegol, or golimumab; or the IL-1β inhibitor comprises rilonacept; canakinumab; or Anakinra; or the IL-6 inhibitor comprises tocilizumab, siltuximab, sarilumab, olokizumab, or sirukumab; or the compound that targets TLR4 signaling comprises (ethyl 4-(4′-chlorophenyl) amino-6 methyl-2-oxocyclohex-3-en-1-aote (enamionone E121), JODI 18b; JODI 19, resatorvid, TLR-C34; or C35; or the p38 MAPK inhibitor comprises 4-(4′-fluorophenyl)-2-(4′-methylsulfinylphenyl)-5-(4′-pyridyl)-imidazole (SB203580), trans-4-[4-(4-fluorophenyl)-5-(2-methoxy-4-pyrimidinyl)-1H-imidazol-1-yl]cyclohexanol (SB239063), or 4-[4-(4-fluorophenyl)-1-(3-phenylpropyl)-5-(4-pyridinyl)-1H-imidazol-2-yl]-3-butyn-1-ol (RWJ 67657); or the compound that targets Janus kinase signaling comprises tofacitinub, baricitinib, or upadacitinib; or the compound that targets a cell adhesion molecule to reduce leukocyte recruitment comprises an a4 integrin inhibitor comprising vedolizumab or natalizumab; or the recombinant anti-inflammatory cytokine comprises IL-4, IL-10, or IL-11; or the interferon is in a PEGylated form.

The term “immune checkpoint molecules” as used herein refers to ligand-receptor pairs that exert inhibitory or stimulatory effects on immune responses. Examples include programmed cell death 1 receptor (PD-1, also known as CD279), thought to regulate T cell proliferation later in the immune response, and its ligand programmed cell death ligand 1 (PD-L1), lymphocyte-activation gene 3 (LAG3), which suppresses T cells activation and cytokine secretion, thereby ensuring immune homeostasis and shows synergy with PD-1 to inhibit immune responses (Long, L. et al. Genes Cancer (2018) 9 (5-6): 176-89. and cytotoxic T-lymphocyte-associated antigen 4 (CTLA4; also known as CD152), a negative regulator of T cell immune function thought to regulate T cell proliferation early in an immune response [Buchbinder, E I, and Desai, A. Am. J. Clin. Oncol. (39) (10: 98-106). In addition, glucocorticoid-induced TNFR family related gene (GITR), a member of the TNFR superfamily (TNFRSF) that is expressed in different cell types, including T lymphocytes activation; GITR activation by its ligand (GITRL) influences the activity of effector and regulatory T cells, thus participating in the development of immune response against tumors and infectious agents, as well as in autoimmune and inflammatory diseases. [Nocentini, G. et al. Br. J. Pharmacol. (2012) 165 (7): 2089-99] T-cell immunoglobulin and mucin domain 3 (Tim-3) is a checkpoint receptor expressed by a wide variety of immune cells as well as leukemic stem cells. [Acharya, N. et al. J. Immunother. Cancer (2020) 8(10: e000911). T-cell immunoreceptor with immunoglobulin and ITIM domains (TIGIT) is an immune checkpoint receptor that can suppress T-cell activation and promote T-cell exhaustion. Inhibition of TIGIT may increase cytotoxic T-cell proliferation and function. Inducible T cell costimulator (ICOS, cluster of differentiation (CD278)) is an activating costimulatory immune checkpoint expressed on activated T cells. Its ligand, ICOSL is expressed on antigen-presenting cells and somatic cells, including tumour cells in the tumour microenvironment. [Solinas, C. et al. ESMO Open. (2020) 5(1): e000544].

According to some embodiments, the immunomodulator is an immune checkpoint inhibitor. The term “immune checkpoint inhibitor” as used herein refers to a molecule that can block immune checkpoint molecules. Specific immune checkpoint inhibitors, including antibodies against CTLA-4, PD-1 receptor or its ligand PD-L1 include YERVOY™ (Ipilimumab; CTLA-4 antagonist), OPDIVO™ (Nivolumab; PD-1 antagonist) and KEYTRUDA™ (Pembrolizumab; PD-1 antagonist) in multiple tumor indications, with ongoing registration trials in many more.

According to some embodiments, the immunomodulatory agent comprises recombinant IL-37. According to some embodiments, the immunomodulatory agent comprises recombinant CD24. According to some embodiments, the immunomodulatory agent comprises rIL-37 and rCD24.

IL-37 is a member of the IL-1 family, which includes IL-1α, IL-1β, IL-18, IL33, IL36α, IL-36β, IL-36γ, IL-37 and IL-38. [Mantovani, A. et al. Immunity (2019) doi.10.1016/j.immuni.2019.03.012]. The IL-1 family of cytokines is divided into three subgroups on the basis of the Il-1 consensus sequence and the signaling receptor chain. These include secreted molecules with agonistic activity [IL-1α, IL-1β, IL-18, IL33, IL36α, IL-36β, IL-36γ], receptor antagonists [IL-1Ra, IL36Ra, and IL-38] and an anti-inflammatory cytokine (IL-37) [Id., citing Dinarello, C A Immunol. Rev. (2018) 281: 8-27]. There is no murine counterpart to human IL-37. [Id.].

In humans, production of IL-37 is activated by pro-inflammatory stimuli, including cytokines, as a protective mechanism to prevent runaway inflammation and excessive damage. Five transcripts for the human IL-37 gene have been identified (IL-37 a-e). IL-37b is the most complete of these isoforms, is the most abundant and studied, and includes 5 of the 6 exons of the IL-37 gene (all but exon 3). Exons 4,5, and 6 encode for the sequence required for the beta-fold barrel structure and account for the extracellular activity of recombinant IL-37. Conversely, exons 1 2, and 3 may be cleaved in the extracellular environment by unknown proteases. IL-37 isoforms a, b and d share exons 4, 5, and 6 and encode functional proteins. The IL-37 isoforms c and e lack one or more of these exons and likely encode non-functional proteins. [Cavalli, G. and Dinarello, C A. Immunological Revs. (2017) 281: 1-12].

Low concentrations of recombinant IL-37 most effectively suppress cytokine production in vitro. In nature, IL-37 is a dual function cytokine exerting potent anti-inflammatory effects via two distinct mechanisms, either extracellular (receptor-mediated) or intracellular (nuclear function). As shown in FIG. 13, extracellular IL-37 forms a complex with cell surface IL-18 receptor α (IL-18Rα) and IL-1 receptor 8 (IL-1R8), which transduces anti-inflammatory signals. Intracellular IL-37 produced upon proinflammatory stimuli interacts with Smad3 and traffics to the nucleus, where it regulates gene expression and dampens transcription of pro-inflammatory genes.

Intracelluar/endogenous activity of IL-37. The IL-37 precursor is synthesized in human blood monocytes following stimulation by IL-1 or TLR agonists. in human blood monocytes, Pro-inflammatory stimuli induce an increase in intracellular IL-37 precursor while also triggering the activation of caspase-1, which cleaves the carboxyldomain of the IL-37 precursor. Mature Il-37 then associates with phosphorylated Smad-3, which enables nuclear translocation and regulation of gene transcription. Both the mature and precursor forms of IL-37 are released into the extracellulular space upon cell death or secreted by an unknown mechanism.

Extracellular/exogenous activity of IL-37. Both the mature and precursor forms of IL-37 are released into the extracellular space upon cell death or secreted by an unknown mechanism. Extracellular proteases process IL-37 precursor into the mature form. IL-37 binds to the IL18Rα and recruits IL1R8. IL-1R8 has a mutated TIR domain, which functions as a sink for MyD88; as a result, there is a weak or no transduction of pro-inflammatory signals, while anti-inflammatory pathways are activated. [Cavalli, G. and Dinearello, C A. Immunological Revs. (2018) 281 (1): 179-90].

Li, et al. (accepted manuscript) examined early response of IL-37 in 254 SARS-CoV-2 infected patients prior to any clinical intervention and determined that higher early IL-37 plasma responses correlated with earlier viral RNA negative conversion, chest CT image improvement and cough relief, resulting in earlier hospital discharge. Higher IL-37 was associated with lower IL-6 and IL-8 and higher IFN-α in these patients. In contrast, low early IL-37 plasma responses predicted severe clinical prognosis in combination with IL-8 and C-reactive protein (CRP), a blood test for inflammation. They reported that IL-37 administration attenuated lung inflammation and alleviated respiratory tissue damage in human angiotensin-converting enzyme 2 (hACE2)-transgenic mice infected with SARS-CoV2.

CD24, also known as Heat Stable Antigen (HSA) or Small Cell Lung Carcinoma Cluster 4 Antigen, is a heavily glycosylated glycophosphatidylinositol (GPI)-anchored surface protein [Barkal, A. A. et al. Nature (2019) 572 (7769): 392-96, citing Pirruccello S J, LeBien T W The human B cell-associated antigen CD24 is a single chain sialoglycoprotein. J. Immunol. 136, 3779-3784 (1986), Chen G Y, Brown N K, Zheng P, Liu Y Siglec-G/10 in self-nonself discrimination of innate and adaptive immunity. Glycobiology 9, 800-806 (2014)]. Several signal transduction proteins are associated with CD24 activity, including the Src-family protein tyrosine kinases Lyn, Fyn, Fgr, Lck snf Hck, but how these are activated is unknown. [Ayre, D C and Christian, S L, Front. Cell & Devel. Biol. (2016) 4: 1146]. Many ligands have been identified for CD24, including P-, L- and E-Selectin, High Mobility Group Box 1 (HMGB1), Li cell adhesion molecule (L1CAM), Neural cell adhesion molecule (NCAM1) and Siglec-G. [Id., citing Aigner, et al. 1995; Myung, et al. 2011; Tan et al 2016]. To explain the contradictory nature of the processes regulated by CD24, its apparent lack of intrinsic signaling capability or its diverse collection of reported ligands, It has been proposed that CD24 functions as a rheostat to modulate responses transduced by partnered cell surface receptor(s), and that these partners define the biological outcomes observed [Ayre, D C and Christian, S L, Front. Cell & Devel. Biol. (2016) 4: 1146]. Mechanistically, the variable nature of CD24-mediated effects can be explained by its in cis association with unique cell-type specific signaling partners through direct physical interaction mediated by its modifiable glycosylations. [Ayre, D C and Christian, S L, Front. Cell & Devel. Biol. (2016) 4: 1146].

It has been hypothesized that the DAMPS released during cell death in viral infection may cause a self-propagating inflammatory response with lasting lung damage. [Tian, R-R et al. Cellular & Molec. Immunol. (2020) 17: 887-888]. Therefore, it has been proposed that the CD24-mediated Siglec10/G interaction is an immune checkpoint that regulates inflammation caused by DAMPS. [Id., citing Chen, G. et al. Science (2009) 323: 1722-25; Liu, Y. et al. Trends Immunol. (2009) 30: 557-61; Fang, X. et al. Cell Mol. Immunol. (2010) 7: 100-103].

CD24 is known to interact with Sialic Acid Binding Ig Like Lectin 10 (Siglec-10) on innate immune cells in order to dampen damaging inflammatory responses to infection [Id., citing Chen W et al. Induction of Siglec-G by RNA viruses inhibits the innate immune response by promoting RIG-I degradation. (2013) Cell 152(3), 467-478], sepsis [(Id, citing Chen G Y et al. Amelioration of sepsis by inhibiting sialidase-mediated disruption of the CD24-SiglecG interaction. Nature Biotechnology (2011) 29, 428-435), liver damage [Id., citing Chen G Y et al. CD24 and Siglec-10 selectively repress tissue damage-induced immune responses. Science (2009) 323 (5922), 1722-1725], and chronic graft v. host disease [Id., citing Toubai T et al. Siglec-G-CD24 axis controls the severity of graft-versus-host disease in mice. Blood (2014) 123(22), 3512-3513]. The binding of CD24 to Siglec-10 elicits an inhibitory signaling cascade mediated by SHP-1 and/or SHP-2 phosphatases associated with the two immunoreceptor tyrosine-based inhibition motifs (ITIMS) in the cytoplasmic tail of Siglec-10, thereby blocking TLR-mediated inflammation and the cytoskeletal rearrangement required for cellular engulfment by macrophages [Id., citing Crocker P R, Paulson J C, Varki A Siglecs and their roles in the immune system. Nature Reviews Immunology (2007) 7, 255-266; Abram C L, Lowell C A Shp1 function in myeloid cells. J. Leukoc. Biol (2017) 102(3), 657-675 Dietrich J, Cella M, Colonna M Ig-Like Transcript 2 (ILT2)/Leukocyte Ig-Like Receptor 1 (LIR1) Inhibits TCR Signalling and Actin Cytoskeleton Reorganization. J. Immunol. (2001) 166(4), 2514-2521].

It has been reported that a recombinant fusion protein CD24-Fc (an agonist of Sioglecs, which can fortify the CD24-Siglec innate immune checkpoint) had a therapeutic effect on SIV-induced lung inflammatory lesions. [Tian, R-R et al., Cellular & Molec. Immunol. (2020) 17: 887-88].Chinese rhesus macaques were infected with simian immunodeficiency virus SIVmac239 via intravenous infusion received either three injections of a recombinant fusion protein CD24-Fc or normal saline on day 56 of infection. Five months later, another cycle of treatment was given to the surviving animals, which were terminated one week after the last dosing. Previous studies had shown that lung lesions developed within 2-4 weeks in SIV-infected rhesus monkeys. By 8 weeks, essentially all monkeys developed lung pathology. The data showed that CD24Fc not only reduced the incidence of viral pneumonia but also qualitatively altered the nature of the pathology in the lung.

It has also been reported that CD24 is important in regulating T cell survival. T cells must regulate their proliferation to support a long-lived cell population, but can expand their numbers during immune activation. [Ayre, D C and Christian, S L, Front. Cell & Devel. Biol. (2016) 4: 1146, citing Boyman, O. et al Eur. J. Immunol. (2009) 39: 2088-94]. In the absence of CD24, homeostatic proliferation of T cells is markedly reduced, however immune-driven proliferation is less affected [Id., citing Li. O. et al. J. Exp. Med. (2004) 200: 1083-89], likely because it depends on TCR co-receptors [Id., citing Chen, L. and Flies, D B Nat. Rev. Immunol. (2013) 13: 227-42]. When CD24+ T cells are transferred to CD24-knockout mice, excessive and destructive homeostatic T cell proliferation occurs, but CD24 expressed on dendritic cells is sufficient to ameliorate this effect [Id., citing Li, O. et al J. Exp. Med. (2006) 203: 1713-20]. This suggested that CD24 can act in cis on the T cell to regulate TCR signaling, or in trans, where DC-expressed Cd24 can bind and modulate its partner(s) on the T cell.

### Other Compatible Actives

According to some embodiments, the anti-inflammatory agent may comprise aspirin, celecoxib, diclofenac, diflunisal, etodolac, ibuprofen, indomethacin, ketoprofen, ketorolac nabunetone, naproxen, nintedanib, oxaprozin, pirfenidone, piroxicam, salsalate, sarilumab (Kevzara®) sulindac, tolmetin, or combinations thereof.

According to some embodiments, the anti-infective agent may comprise amoxicillin, doxycycline, demeclocycline; eravacycline, minocycline, ormadacycline, tetracycline, cephalexin, defotaxime, cetazidime, cefuroxime, ceftaroline; ciprofloxacin, levofloxacin, moxifloxacin, clindamycin, lincomycin, metronidazole, azithromycin; clarithromycin, erythromycin, sulfamethoxazle and trimethoprim; sulfasalazine, amoxicillin and clavulanate; vancomycin, dalbavancin, oritavancin, telavancin, gentamycin, tobramycin, amikacin, imipenem and cilastatin, meropenem, doripenem, or ertapenem.

According to some embodiments, the anti-malarial agent may comprise quinine, quinidine, chloroquine, hydroxychloroquine, amodiaquine, mefloquine, halofantrine, lumefantrine, piperaquine, and tafenoquine; an antifolate compound selected from pyrimethamine, proguanil, chlorproguanil, trimethoprim; an artemisinin compound selected from artemisinin, dihydroartemisinin, artemether, artesunate; or atovaquone.

According to some embodiments, the anti-viral agent may comprise acyclovir, gancidovir, foscamet; ribavirin; amantadine, azidodeoxythymidine/zidovudine), nevirapine, a tetrahydroimidazobenzodiazepinone (TIBO) compound; efavirenz; remdecivir, lopinavir/ritonavir, umifenovir, favipiravir, ivermectin, or delavirdine. According to some embodiments, the anti-viral agent is an agent that inhibits viral entry and decreases viral load.

According to some embodiments, the anti-fibrotic agent may comprise nintedanib, pirfenidone, or combinations thereof.

### Treating Regimens

The quantity and frequency of administration will be determined by such factors as the condition of the patient, and the type and severity of the patient's disease, although appropriate dosages may be determined by clinical trials.

The administration of the pharmaceutical compositions containing the cell product may be carried out in any manner appropriate to the particular disease, including by aerosol inhalation, injection, ingestion, transfusion, implantation or transplantation. The pharmaceutical compositions of the present disclosure may be administered to a patient parenterally, e.g., subcutaneously, intradermally, intramuscularly, by intravenous (i.v.) injection, intraperitoneally, or by infusion techniques. According to some embodiments, the pharmaceutical compositions of the described invention also can be administered to a subject by direct injection to a desired site, or systemically.

According to some embodiments, the pharmaceutical composition containing the population of SCKTCs can be administered to a patient daily. According to some embodiments, the pharmaceutical composition containing the population of SCKTCs can be administered to a patient by continuous infusion. According to some embodiments, the pharmaceutical composition containing the population of SCKTCs can be administered to a patient twice daily. According to some embodiments, the pharmaceutical composition containing the population of SCKTCs can be administered to a patient more than twice daily. According to some embodiments, the pharmaceutical composition containing the population of SCKTCs can be administered to a patient every other day. According to some embodiments, the pharmaceutical composition containing the population of SCKTCs can be administered to a patient twice a week. According to some embodiments, the pharmaceutical composition containing the population of SCKTCs can be administered to a patient every other week. According to some embodiments, the pharmaceutical composition containing the population of SCKTCs can be administered to a patient every 30 days, or every 1, 2, 3, 4, 5, or 6 months.

According to some embodiments, the pharmaceutical composition comprising a cell product containing the population of SCKTCs can be administered to a patient in a dosing regimen (dose and periodicity of administration) sufficient to maintain function of the administered SCKTCs in the bloodstream of the patient over a period of 2 weeks to a year or more, e.g., one month to one year or longer, e.g., at least 2 weeks, 4 weeks, 6 weeks, 8 weeks, 3 months, 6 months, a year, 2 years.

The frequency of the required dose will be readily apparent to the skilled artisan and will depend upon any number of factors, such as, but not limited to, the type and severity of the disease being treated, the type and age of the animal, etc.

Alternatively, the additional therapeutic agent(s) may be administered an hour, a day, a week, a month, or even more, in advance of the pharmaceutical composition, or any permutation thereof. Further, the additional therapeutic agent(s) may be administered an hour, a day, a week, or even more, after administration of the pharmaceutical composition, or any permutation thereof. The frequency and administration regimen will be readily apparent to the skilled artisan and will depend upon any number of factors such as, but not limited to, the type and severity of the disease being treated, the age and health status of the animal, the identity of the additional therapeutic agent or agents being administered, the route of administration and the pharmaceutical composition comprising the population of SCKTCs, and the like.

According to some embodiments, a “subject having an infection” is a subject that has been exposed to an infectious pathogen with acute or chronic detectable levels of the microorganism in his/her body or has signs and symptoms of the infectious pathogen. Methods of assessing and detecting infections in a subject are known by those of ordinary skill in the art. A “subject at risk of an infection” is a subject that may be expected to come in contact with an infectious pathogen. Examples of such subjects are medical workers or those traveling to parts of the world where the incidence of infection is high. According to some embodiments, the subject is at an elevated risk of an infection because the subject has one or more risk factors to have an infection. Examples of risk factors to have an infection include, for example, immunosuppression, immunocompromise, age, trauma, burns (e.g., thermal burns), surgery, foreign bodies, cancer, newborns especially newborns born prematurely. The degree of risk of an infection depends on the multitude and the severity or the magnitude of the risk factors that the subject has. Risk charts and prediction algorithms are available for assessing the risk of an infection in a subject based on the presence and severity of risk factors. Other methods of assessing the risk of an infection in a subject are known by those of ordinary skill in the art. According to some embodiments, the subject who is at an elevated risk of an infection may be an apparently healthy subject. An “apparently healthy subject” is a subject who has no signs or symptoms of disease.

According to some embodiments, factors other than age associated with the target population for treatment are considered. These factors include, but are not limited to, comorbidities, geographic factors (including microbial endemicity), nutritional status, and iatrogenic immune suppression.

### Subjects

The methods described herein are intended for use with any subject that may experience the benefits of these methods. Thus, “subjects,” “patients,” and “individuals” (used interchangeably) include humans as well as non-human subjects, particularly domesticated animals.

According to some embodiments, the subject and/or animal is a mammal, e g., a human, mouse, rat, guinea pig, dog, cat, horse, cow, pig, rabbit, sheep, or non-human primate, such as a monkey, chimpanzee, or baboon. Although the descriptions of pharmaceutical compositions provided herein are principally directed to pharmaceutical compositions, which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation.

In other embodiments, the subject and/or animal is a non-mammal. According to some embodiments, the subject and/or animal is a human. According to some embodiments, the human is a pediatric human. According to other embodiments, the human is an adult human. According to other embodiments, the human is a geriatric human. According to other embodiments, the human may be referred to as a patient.

According to certain embodiments, the human has an age in a range of from about 0 months to about 6 months old, from about 6 to about 12 months old, from about 6 to about 18 months old, from about 18 to about 36 months old, from about 1 to about 5 years old, from about 5 to about 10 years old, from about 10 to about 15 years old, from about 15 to about 20 years old, from about 20 to about 25 years old, from about 25 to about 30 years old, from about 30 to about 35 years old, from about 35 to about 40 years old, from about 40 to about 45 years old, from about 45 to about 50 years old, from about 50 to about 55 years old, from about 55 to about 60 years old, from about 60 to about 65 years old, from about 65 to about 70 years old, from about 70 to about 75 years old, from about 75 to about 80 years old, from about 80 to about 85 years old, from about 85 to about 90 years old, from about 90 to about 95 years old or from about 95 to about 100 years old.

According to some embodiments, the subject is a non-human animal, and therefore the disclosure pertains to veterinary use. According to some such embodiments, the non-human animal is a household pet. According to some such embodiments, the non-human animal is a livestock animal.

According to some embodiments, the susceptible subject includes a very young subject, an elderly subject, a subject who is ill; an immunocompromised subject, a subject with long term health conditions, a subject who is obese, or a subject that is physically weak due to malnutrition or dehydration.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges which may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present disclosure, exemplary methods and materials have been described.

The publications discussed herein are provided solely for their disclosure prior to the filing date of the present application and each is incorporated by reference in its entirety. Nothing herein is to be construed as an admission that the present disclosure is not entitled to antedate such publication by virtue of prior invention. Further, the dates of publication provided may be different from the actual publication dates which may need to be independently confirmed.

All publications mentioned herein are incorporated herein by reference to disclose and described the methods and/or materials in connection with which the publications are cited.

It is also to be understood that throughout this disclosure where the singular is used, the plural may be inferred and vice versa and use of either is not to be considered limiting.

### EXAMPLES

The following examples are put forth so as to provide those of ordinary skill in the art with a complete disclosure and description of how to make and use the described invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1. Process Flow, Run 5 (Tissue Culture Flasks)

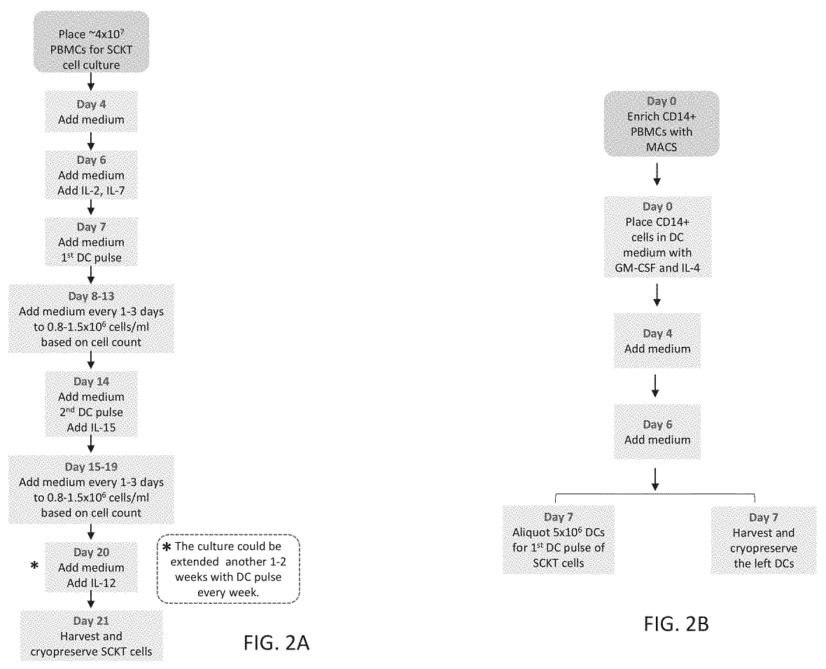

A flow chart depicting the process flow in Run 5 for stimulation of superactivated cytokine killer cells in tissue culture flasks is shown in FIG. 2A. A flow chart depicting the process flow for dendritic cell culture is shown in FIG. 2B.

As shown in FIG. 2A, on day 0, 4×10⁷ PBMCs are placed in 20 ml X-VIVO-15 medium (hereinafter “serum free culture medium”). On day 4, the medium is replenished by adding serum-free culture medium. On day 6, serum-free culture medium containing IL-2 (R & D Systems, about 10U/ml to about 100U/ml) and IL-7 (R & D Systems, about 10 ng/ml to about 200 ng/ml) is added to replenish the cell culture. On day 7, monocyte-derived dendritic cells (DCs) are pre-incubated up to 2 hr with α-GalCer to load the monocyte-derived DCs with α-GalCer, and the cultures then are pulsed with serum-free culture medium containing a first round of fresh 5×10⁶ α-GalCer-loaded DCs; the remainder of the DCs are cryopreserved. From day 8 through day 13, the cultures are replenished with serum-free culture medium every 1-3 days based on cell count to 0.8-1.5×10⁶ cells/ml. On day 14, 5×10⁶ DCs are thawed; before the DC pulse, the DCs are pre-incubated up to 2 hr with α-GalCer to load the DCs with α-GalCer and the cultures then replenished with serum-free culture medium containing the thawed 5×10⁶ α-GalCer-loaded dendritic cells and IL-15 (R & D Systems, about 10 ng/ml to about 100 ng/ml) is added to the serum-free culture medium. Between day 14 and day 20, the cultures are replenished every 1-3 days based on cell count to 0.8-1.5×10⁶ cells/ml. The cultures can be extended another 1-2 weeks by pulsing them with α-GalCer-loaded DCs every week. The day before harvest (at least day 20), cells are fed with serum-free culture medium to which IL-12 (R & D Systems, about 10 ng/ml to about 100 ng/ml) has been added. The SCKTCs are harvested at least on D21 and cryopreserved.

The dendritic process flow is shown in FIG. 2B. On day 0, CD14+ monocytes are sorted out of the PBMCs with CD14 MACS beads. On days 4 and 6 the culture medium is replenished with serum-free culture medium. On day 7, fresh 5×10⁶ DCs are collected, loaded with α-GalCer- and added to the SCKTC cultures with serum-free culture medium as a first DC pulse. The remaining DCs are harvested and cryopreserved. On day 14, about 5×10⁶ DCs are thawed, loaded with α-GalCer and added to the SCKTC cultures as a second DC pulse as shown.

### Characterization of SCKTCs, Run 5

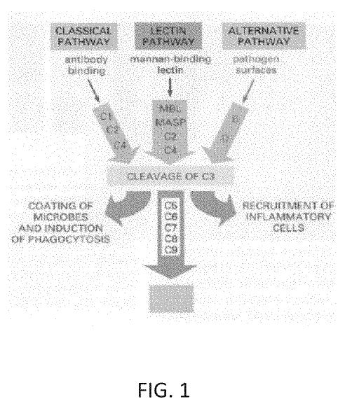

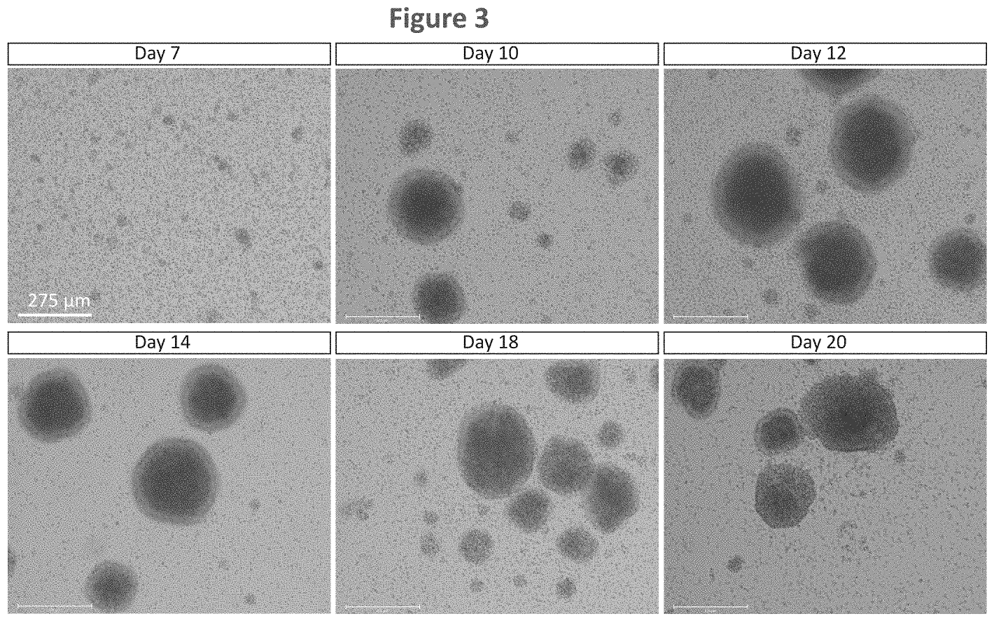

Characteristics of representative SCKTCs produced by the process of FIG. 1 in tissue culture flasks are shown in FIG. 3 through FIG. 7.

Cell Morphology. FIG. 3 shows representative morphology of the Run 5 cultures at day 7, day 10, day 12, day 14, day 18 and day 20. Cell morphology shows obvious cell colonies starting from day 7.

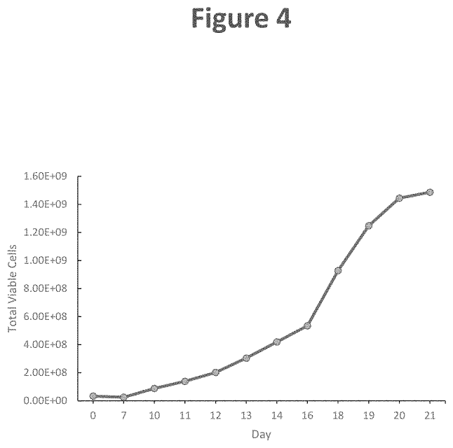

Growth curves. FIG. 4 shows representative growth curves of total viable cells vs. days in culture. After 21 days in culture, total number of cells is about 1.5×10⁹.

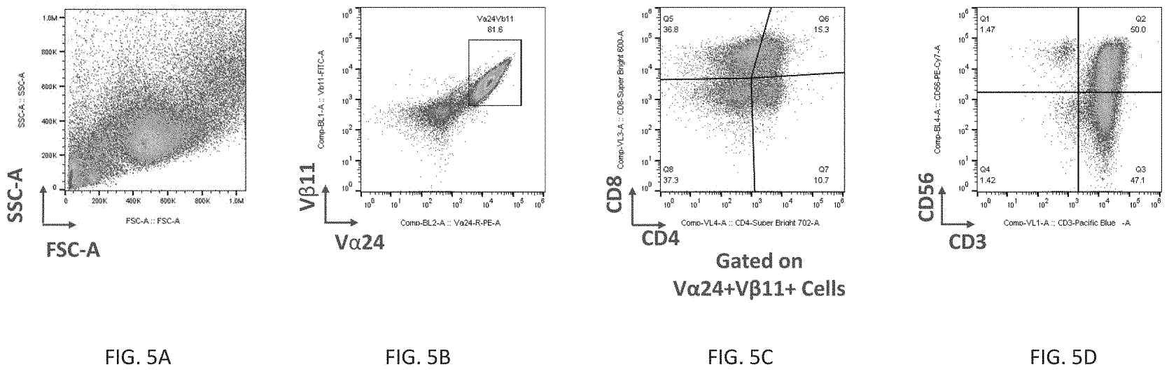

Cell Identity. FIG. 5 shows forward (FSC) and side scatter (SSC) plots for size and granularity from multicolor flow cytometry experiments on day 20. Fresh cells were used for staining with antibodies/dyes. FIG. 5A shows an FSC/SSC plot of the total cell population: FIG. 5B shows Vβ11 v. Vα24; FIG. 5C shows CD8 v. CD4; Gating was on Vα24+Vβ11+ cells; FIG. 5D shows CD56 v. CD3. SCKTC purity achieved was about 81.6% of total viable cells.

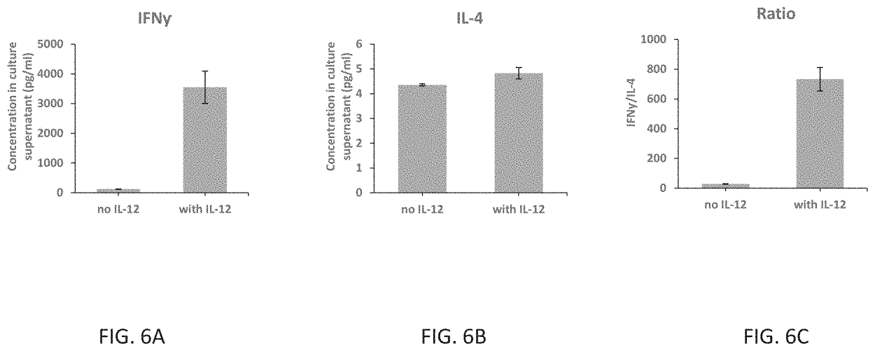

Cytokine production. FIG. 6 shows representative bar graphs depicting cytokine production plotting concentration in Run 5 culture supernatant (pg/ml), for IFN-γ (FIG. 6A), IL-4 (FIG. 6B) and the ratio of IFNγ to IL-4 (FIG. 6C). FIG. 6A and FIG. 6B, y-axis is concentration in culture supernatant (pg/ml). X-axis for FIG. 6A, FIG. 6B and FIG. 6C is no IL-12 or with IL-12. Cytokines were measured by Cytometric Bead Array (CBA) assay [BD, Human IFNγ Flex Set; Human IL-4 Flex Set]. The data show that IL-12 can strongly stimulate IFN-γ secretion, while having no obvious effect on IL-4 secretion. IFNγ:IL-4 ratio with IL-12 is about 750.

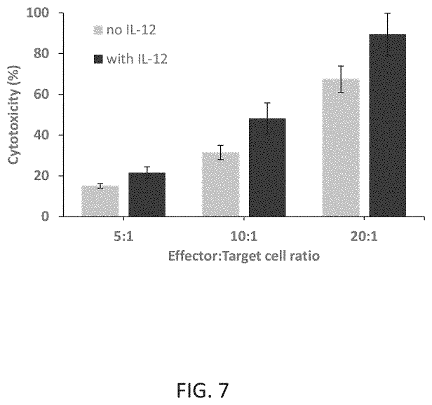

In vitro cytotoxicity. FIG. 7 shows in vitro cytotoxicity of Run 5 SCKTCs on A549 cells. Cytotoxicity was determined by LDH cytotoxicity assay kit (Dojindo Molecular Technologies (#CK12-05) Cytotoxicity (%) is plotted against Effector:Target cell ratio with and without IL-12. The results show that IL-12 stimulation slightly increased in vitro cytotoxicity of SCKTCs from Run 5 on A549 target cells.

### Example 2. Superactivated Cytokine Killer Cell Amplification and Stimulation

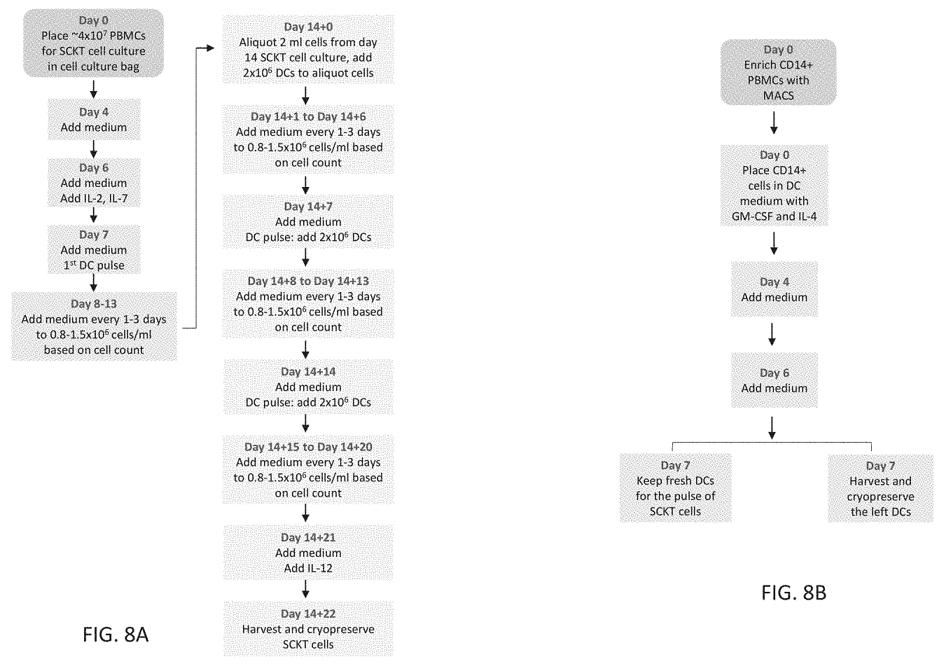

FIG. 8 shows the process flow for stimulation of superactivated cytokine killer cells (SKTCs) and for generating dendritic cell cultures exemplified by Run 14. MCs are derived from peripheral blood by apheresis. Cultures in Run 14 were grown in gas permeable cell culture bags.

The process flow for the SCKTC cells is shown in FIG. 8A. 4×10⁷ MCs are placed in 20 ml SCKTC culture medium in gas permeable cell culture bag. On Day 4, the cell medium is replenished with serum-free culture medium by syringe. On Day 6, the cell medium is replenished with serum-free culture medium by syringe and IL-2 [R & D Systems; 100 IU/ml] and IL-7 [R & D Systems; 20 ng/ml] are added to the cultures. On Day 7, the culture is pulsed with an enriched population of α-GalCer-loaded monocyte-derived DCs (5×10⁶). Between days 7 and 13, the serum-free culture medium is replenished every 1-3 days to a cell count of 0.8-1.5×10⁶ cells/ml. On Day 14, 2 ml of cells were aliquoted from the culture; and 2×10⁶ α-GalCer-loaded monocyte-derived DCs were added to the aliquot. IL-15 [R & D Systems; 20 ng/ml] is added to the cultures. The rest of the cells were cultured according to the protocol of FIG. 2 in order to compare the stimulation effect. The serum-free culture medium is replenished every 1-3 days to 0.8-1.5×10⁶ cells/ml based on cell count. On day 14+7, the serum-free culture medium is replenished and 2×10⁶ α-GalCer-loaded monocyte-derived DCs are added. The serum-free culture medium is replenished every 1-3 days based on cell count to 0.8-1.5×10⁶ cells/ml. On day 14+14, the serum-free culture medium is replenished and 2×10⁶ α-GalCer-loaded monocyte-derived DCs are added. The serum-free culture medium is replenished every 1-3 days based on cell count to 0.8-1.5×10⁶ cells/ml. On day 14+21, the serum-free culture medium is replenished and IL-12 [R & D Systems; 10 ng/ml-200 ng/ml] is added. On day 14+22, the SCKTCs derived from the aliquot culture are harvested and optionally cryopreserved.

The process flow for the DCs is shown in FIG. 8B. CD14 MACS is used to sort out CD14+ monocyte cells derived from 1×10¹ MCs. The CD14+ cells are placed in SCKTC medium in a gas permeable cell culture bag (80 ml). The serum-free DC culture medium is replenished on day 4 and day 6. On day 7, fresh DCs (5×10⁶) are withdrawn for the first DC pulse. Before the DC pulse, the DCs are pre-incubated up to 2 hr with α-GalCer to load the DCs with α-GalCer. The remaining DCs are harvested and cryopreserved.

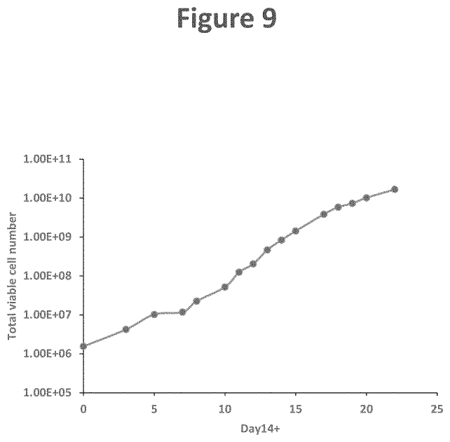

Characteristics of representative SCKTCs produced by the process of FIG. 7 are demonstrated in FIG. 9-12.

Cell morphology. The morphology of the Run 14 cultures produced by the process flow of FIG. 8 was similar to the morphology of the Run 5 cultures shown in FIG. 3 (data not shown).

Growth curve. FIG. 9 shows a representative growth curve of total viable cells vs. days in culture for Run 14 supercell cultures. On day 14+22, the total viable cell number is about 1.68×10¹⁰.

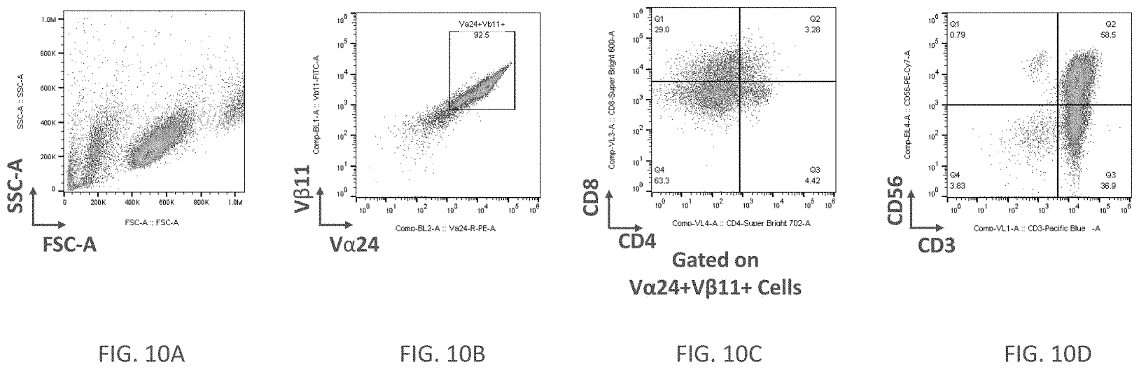

Cell identity. FIG. 10 shows forward (FSC) and side scatter (SSC) plots for size and granularity from multicolor flow cytometry experiments for cell identity of representative Run 14 supercell cultures. Fresh cells were used for staining with the antibodies/dyes. FIG. 10A shows an FSC/SSC plot of the total cell population; FIG. 10B shows Vβ11 c. Vα24; FIG. 10C shows CD8 v. CD4; Gating was on Vα24+Vβ11+ cells; FIG. 10D shows CD56 v. CD3.

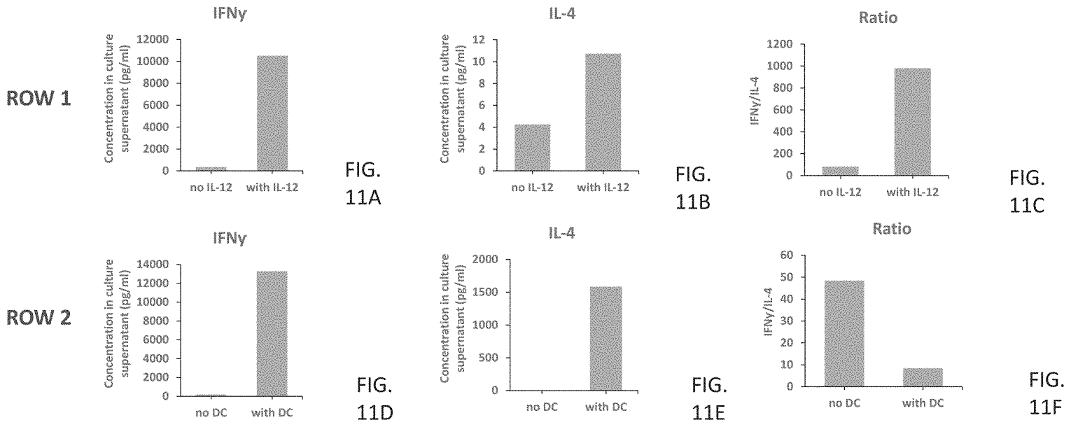

Cytokine production. FIG. 11 shows representative bar graphs depicting cytokine production by the Run 14 supercell cultures. Row 1 shows supercell stimulation with IL12. Row 2 shows supercell stimulation with DCs. The bar graphs plot concentration in culture supernatant (pg/ml), y-axis for IFN-γ (FIG. 11A, FIG. 11 D)), IL-4 (FIG. 11B, FIG. 11 E) and the ratio of IFNγ to IL-4 (FIG. 11C, FIG. 11F). Cytokines were measured by Cytometric Bead Array (CBA) assay [BD, Human IFNγ Flex Set; Human IL-4 Flex Set]. The results show that both IL-12 (FIG. 11A) and DCs (FIG. 11D) can strongly stimulate IFNγ secretion. As for IL-4, IL-12 stimulated IL-4 secretion (FIG. 11B) with an increase in the ratio of IFNγ/IL-4 (FIG. 11C). While DCs could also robustly stimulate IL-4 secretion (FIG. 11E), this stimulation caused a decrease in the ratio of IFNγ/IL-4 (FIG. 11F). The DCs increase the amount of IL4 secreted therefore deceasing the IFN:IL-4 ratio.

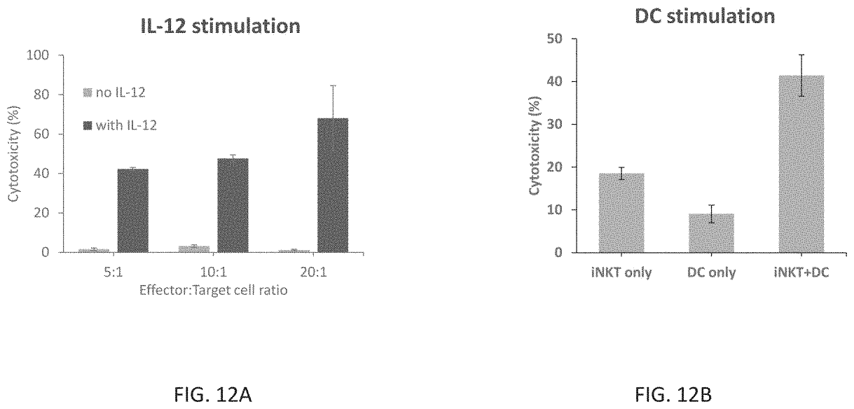

Cytotoxicity. FIG. 12 shows representative bar graphs depicting in vitro cytotoxicity of the Run 14 supercell cultures on A549 target cells. FIG. 12A shows cytotoxicity with and without IL-12 stimulation at an effector:target cell ratio of (from left to right) 5:1, 10:1, and 20:1. FIG. 12B shows cytotoxicity of Run 14 supercell cultures on A549 target cells comparing SCKTCs only (SCKTC:A549 cell ratio: 10:1), DCs only (DC:A549 cell ratio, 1:1), and DC-stimulated SCKTCs+ DCs (SCKTC:DC=10:1). Cytotoxicity was determined by an LDH cytotoxicity assay kit (Dojindo Molecular Technologies (#CK12-05)). The results show that both IL-12 and DC stimulation can strongly activate in vitro cytotoxicity of SCKTCs on target A549 cells.

### Example 3. Use of K18-hACE2 Mice as a Model of SARS-CoV2 Infection

A transgenic mouse model that expresses the hACE2 gene under the control of the human cytokeratin 18 promoter will be used to test the efficacy of the pharmaceutical composition of the present disclosure as described by Moreau, G B et al. Am. J. Trop. Med. Hyg. (2020) 103 (3): 1215-19. Mice (K18-hACE2Prlmn/J, Jax #034860; available from Jackson Laboratories) will be infected with median tissue culture infected dose (TCID50) of 104 plaque-forming units (PFUs) of SARSCoV-2. The pharmaceutical composition comprising a cell product containing SSCKTCs will be administered by an intranasal route, intravenously, and/or intramuscularly in groups of 5 mice. Five mock-infected mice will receive 50 μl DMEM. Mice will be followed twice daily for clinical symptoms until day 5. Categories included in clinical scoring will include weight loss; posture and appearance of fur (piloerection), activity; eye closure, and respiratory rate.

Blood samples will be collected by standard procedures. Neutralizing and immunogen-specific antibody titers and isotypes produced by vaccinated mice in serum will be determined by measuring inhibition of SARS-CoV infection of Vero cells and by ELISA, respectively.

For histology, the tissues of euthanized mice will be fixed in formaldehyde. Histopathological scoring for lung tissue will be performed according to the guidelines of the American Thoracic Society. Statistical significance will be determined by standard methods.

Viral titers will be determined by homogenizing the left lobe of the lung in 1 mL serum-free DMEM with a disposable tissue grinder and plaque assays performed. In brief, Vero cells grown in DMEM with fetal bovine serum will be seeded into multiwell plates at a concentration of 2×10⁵ cells/well the night before the assay. Serial dilutions will be added to the wells. The plate will be incubated at 37° C., 5% CO₂ for 2 hr, shaking the plates every 15 minutes. After 2 hr the plate media will be replaced with a liquid overlay of DMEM, 2.5% FBS containing 1.2% Avicel PH-101 (Signa-Aldrich, St. Louis, Mo.) and incubated at 37° C., 5% CO₂. After 3 days, the overlay will be removed, wells will be fixed with 10% formaldehyde and stained with 0.1% crystal violet to visualize plaques. Plaques will be counted, and PFUs calculated according to the following equation: average # plaques/dilution factor x volume diluted virus added to the well.

### Example 4. Use of NSG Mice Reconstituted with Human Immune System Components for Evaluation of the Cell Product of the Present Disclosure

NSG (NOD-scid 11.2 Rγnull) mice (from The Jackson Laboratory, jax.org/jax-mice-and-services/find-and-order-jax-mice/nsg-portfolio) will be engrafted with human PBMC as follows. Fresh whole blood from healthy adult donors collected with preservative free heparin will be diluted (1:3) with low endotoxin PBS (PBSle) (Biochrom) and the leukocyte fraction enriched using standard ficoll gradient centrifugation. The interface will be harvested and washed twice with PBSle. For a 9 week reconstitution protocol, mice will be irradiated with a sub-lethal dose of 100 cGy one day before intravenous injection of 1×10⁶ human PBMCs; a 4-week protocol will use a single intravenous injection of 10×10⁶ PBMC, without irradiation.

### Example 5. Evaluation of Immune Response and Selective Expansion of Immune Cell Subtypes and Cytokines

A therapeutic amount of the pharmaceutical composition comprising the cell product comprising human SKCTs will be administered to the reconstituted NSG mice and the response to this administration will be evaluated.

Briefly, PBMCs, splenocytes, or bone marrow cells of human or murine origins will be isolated and stained for 1 hr at 4° C. in the dark with the appropriate antibody cocktail. Following washing (1% (v/v) FBS in PBS), cells will be fixed with fixation buffer (1% (v/v) FBS, 4% (w/v) PFA in PBS) for 30 min at 4° C. in the dark. Flowcytometric analysis will be performed, and flow cytometry data will be analyzed using FlowJo software (TreeStar, Ashland, Oreg.). Chimerism of all humanized mice model will be assessed prior to each experiment by quantifying the following human populations: Human CD45+, human CD45+ murine CD45−; T-cells, CD45+CD3+; CD4+ T cells, CD45+CD3+CD4+; CD8+ T cells, CD45+CD3+CD8+; CD45+CD16+ leukocytes; B-cells, CD45+CD19; conventional dendritic cells, CD45+CD11c+; NK/NKT cells, CD45+CD56+; Monocytes, CD45+CD14+. Mouse immune cell subsets will be gated as followed: Murine CD45+, Human CD45− Murine CD45+; Conventional dendritic cells, CD45+CD3−CD19−NK1.1−TER119−Ly-6G/Gr1−CD11c+; Plasmacytoid dendritic cells, CD45+CD3−CD19−NK1.1−TER 119−Ly-6G/Gr1−CD317+; Monocytes, CD45+CD3−CD19−NK1.1−TER 119−Ly-6G/Gr1−CD11b+CD11c−F4/80−; Macrophages, CD45+CD3−CD19−NK1.1−TER119−Ly-6G/Gr1−CD11b+F4/80+. Human immune cell subsets will be gated as follows: Human CD45+, human CD45+ murine CD45−; T-cells, CD45+CD3+; CD4+ T cells, CD45+CD3+CD4+; CD8+ T cells, CD45+CD3+CD8+; Myeloid cells, CD45+CD3−CD19−(CD56+) CD33+; Granulocytes, CD45+CD66b+; B cells, CD45+CD3−CD19+; Natural Killer cells, CD45+CD3−(CD19−) CD56+; Natural Killer T cells and γδ T cells, CD45+CD3+(CD19−) CD56+; Conventional dendritic cells, CD45+CD3−CD19−(CD56−) (CD33+) CD11c+(BDCA1/3+); CD45+CD3−CD19 CD123+, group composed of monocytes, plasmacytoid dendritic cells, basophils and myeloid precursors; Plasmacytoid dendritic cells, CD45+CD3−CD19−(CD56−) BDCA-2+CD123+; Monocytes, CD45+CD3−CD19−(CD56−) CD14+; Macrophages, CD45+CD3−CD19−(CD56−) CD68+.

Flow cytometry fluorophor compensation for antibodies will be performed using AbC™ Anti-Mouse Bead Kit (Life Technologies, Invitrogen, Foster City, Calif., USA). Counting beads will be added to each sample prior to flow-cytometry analysis (AccuCheck Counting Beads, Life Technologies, Invitrogen, Foster City, Calif., USA).

The frequency of each cell fraction will be shown as a percentage of CD45+ cells, with the exception of CD4+ and CD8+ T cells, which will be shown as a percentage of CD3+ T cells. The frequencies of myeloid subsets (e.g., CD14+ monocytes and CD11c+ dendritic cells) and CD56+NK cells will also be determined.

### IFN-γ ELISpot Assay

An exemplary ELISPOT assay protocol is as follows. Enzyme-linked immunosorbent spot (ELISpot) assays are conducted using mouse IFN-γ ELISpot kit (BD Bioscience, Cat #551083). Control animals or animals receiving the SCKTCs of the present disclosure are sacrificed and bronchoalveolar lavage cells and splenocytes were isolated. 2×10⁵ splenocytes are plated in triplicate in 96-well plates pre-coated with 5 pg/ml of purified anti-mouse IFN-γ and subsequently stimulated with a peptide specific for a viral immunogen at a final 5 pg/ml concentration. After 24 hours of stimulation, the cells are washed with deionized water and exposed to 100 μl biotinylated anti-mouse IFN-γ (2 μg/ml) for 2 hours at room temperature, followed by extensive washing prior to the addition of 100 μl Streptavidin-HRP. After 1 hour incubation at room temperature, the cells are washed and 100 μl of substrate solution is added to develop spots. The reaction is stopped with water and the number of spot-forming cells (SFCs) is determined using an automated ELISPOT software.

### Cytokine Bead Assay.

Bead populations with distinct fluorescence intensities are coated with capture antibodies specific for IFN-γ and IL4 and mixed together to form a bead array that is resolved in a flow cytometer. During the assay procedure, the inflammatory cytokine capture beads are mixed with recombinant standards or SCKTCs and incubated with PE-detection antibodies. The intensity of PE fluorescence of each complex reveals the concentration of that cytokine.

### Example 6. Infection of NSG Mice Reconstituted with Human Immune System Components with Highly Pathogenic H7N9 Influenza Virus and Low Pathogenicity/Mild H9N2 Influenza Virus

Reconstituted NSG mice are anesthetized with ketamine (40 μl/mouse) before infection, and then infected with an influenza virus H7N9 virus strain (3.5×10⁵ of 50% tissue culture infective dose TCID50/50 ul of volume) and an H9N2 virus strain (1.7×10⁷ of 50% egg infective dose EID50/50 μl of volume) at a high dose by nasal drip. The mice are placed in an IVC cage. For 14 consecutive days, the mice are weighed, and the survival and survival status of the mice is observed. Lung tissues are taken at 6 hours, 1 day, 2 days, 3 days, 7 days, and 14 days after infection, and quick-frozen in liquid nitrogen for use. Weight and mortality will be determined.

Reconstituted NSG mice in groups of 6 will be dosed once by nasal dripping 2 hours before infection with as well as 3 days and 8 days after infection, respectively with the cell product of the present disclosure comprising superactivated cytokine killer T cells or a control carrier) supernatant. Mice will be weighed continuously, and the survival status of the mice observed.

For histology, the tissues of euthanized mice will be fixed in formaldehyde. Histopathological scoring for lung tissue will be performed according to the guidelines of the American Ihoracic Society. Statistical significance will be determined by standard methods.

Viral titers will be determined by homogenizing the left lobe of the lung in 1 mL serum-free DMEM with a disposable tissue grinder and plaque assays performed. In brief, Vero cells grown in DMEM with fetal bovine serum will be seeded into multiwell plates at a concentration of 2×10⁵ cells/well the night before the assay. Serial dilutions will be added to the wells. The plate will be incubated at 37° C., 5% CO₂ for 2 hr, shaking the plates every 15 minutes. After 2 hr the plate media will be replaced with a liquid overlay of DMEM, 2.5% FBS containing 1.2% Avicel PH-101 (Signa-Aldrich, St. Louis, Mo.) and incubated at 37° C., 5% CO₂. After 3 days, the overlay will be removed, wells will be fixed with 10% formaldehyde and stained with 0.1% crystal violet to visualize plaques. Plaques will be counted, and PFUs calculated according to the following equation: average # plaques/dilution factor x volume diluted virus added to the well.

While the present disclosure has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the present disclosure. All such modifications are intended to be within the scope of the claims appended hereto.

What is claimed is:

1. A method for treating a viral infection in a recipient subject suffering from or at risk of the viral infection comprising
a. administering to the recipient subject a pharmaceutical composition comprising a cell product containing a therapeutic amount of superactivated cytokine killer T cells (SCKTCs) and a pharmaceutically acceptable carrier, and
b. mobilizing an immune response of the recipient subject to the viral pathogen;
wherein the therapeutic amount is at least 0.2×10⁹ SCKTCs per 30 day treatment cycle; and
wherein when tested in vitro,
the SCKTCs predominantly produce T_{H}1 dominant cytokines including IFN-γ; or
an IFN-γ:IL-4 ratio of the SCKTC population when tested in vitro is at least 500:1 with IL-12 stimulation; and
at an effector:target ratio of 20:1, cytotoxicity against A549 target cells is >50%.

2. The method according to claim 1, wherein the immune response of the recipient subject comprises stimulating activation of one or more immune cell population of the recipient subject.

3. The method according to claim 2, wherein the immune cell population of the recipient subject comprises one or more of a dendritic cell population; a CD8+ T cell population; an NK cell population; or an MHC-restricted T cell population.

4. The method according to claim 3, wherein the MHC-restricted T cell population comprises an invariant NKT population.

5. The method according to claim 3, wherein the therapeutic amount stimulates an effector function of the immune cells of the recipient subject.

6. The method according to claim 5, wherein the effector function includes one or more of cytokine secretion, cytotoxicity, or antibody-mediated clearance of the pathogen.

7. The method according to claim 1, wherein the viral infection is characterized by virus-infected cells.

8. The method according to claim 7, wherein the therapeutic amount destroys virus-infected cells through direct lysis, by effecting destruction of the infected cells indirectly or both.

9. The method according to claim 8, wherein destruction of the infected cells indirectly comprises mobilizing attracting cell cytotoxicity agents through secretion of cytokines.

10. The method according to claim 1, wherein the virus infection is an infection with a respiratory virus.

11. The method according to claim 10, wherein the respiratory virus is a respiratory syncytial virus (RSV), an Ebola virus, a cytomegalovirus, a Hanta virus, an influenza virus, a coronavirus, a Zika virus, a West Nile virus, a dengue virus, a Japanese encephalitis virus, a tick-borne encephalitis virus, a yellow fever virus, a rhinovirus, an adenovirus, a herpes virus, an Epstein Barr virus, a measles virus, a mumps virus, a rotavirus, a coxsackie virus, a norovirus, or an encephalomyocarditis virus (EMCV).

12. The method according to claim 11, wherein the coronavirus is SARS-CoV-1, SARS-CoV-2 or MERS.

13. The method according to claim 1, wherein
a. the therapeutic amount reduces risk of the virus infection; or
b. the therapeutic amount reduces signs, symptoms, or both signs and symptoms of the viral infection; or
c. the therapeutic amount reduces extent of the viral infection where symptoms are not yet clinically recognized; or
d. the therapeutic amount reduces worsening or progression of the viral infection; or
e. the therapeutic amount reduces severity of the viral infection, compared to an untreated subject; or
f. the therapeutic amount improves progression-free survival; or
g. the therapeutic amount improves overall survival.

14. The method according to claim 1, wherein
a. the superactivated cytokine killer T cells (SCKTCs) are derived from blood; or
b. The SCKTCs are derived from a leukapheresis; or
c. The SCKTCs are derived from hematopoietic stem cells; or
d. The SCKTCs are derived from hematopoietic stem cells derived from adult bone marrow, umbilical cord, umbilical cord blood, placental tissue or fetal liver.

15. The method according to claim 1, wherein the pharmaceutical composition further comprises an enriched differentiated and expanded population of NK cells.

16. The method according to claim 1,
a. wherein the population of SCKTCs is autologous to the recipient subject; or
b. wherein the population of SCKTCs is allogeneic to the recipient subject.

17. The method according to claim 15, wherein the NK cells are derived from CD34+ hematopoietic stem cells of a donor.

18. The method according to claim 15, wherein the population of NK cells is depleted of CD3+ T cells, CD19 B cells or both.

19. The method according to claim 17,
(a) wherein the population of NK cells of the donor is autologous to the recipient subject. or
(b) wherein the population of NK cells of the donor is allogeneic to the recipient subject.

20. The method according to claim 1, further comprising administering the pharmaceutical composition comprising the cell product containing the population of SCKTCs with a supportive therapy or an additional compatible therapeutic agent.

21. The method according to claim 20, wherein the supportive therapy reduces viral load.

22. The method according to claim 20 wherein the additional compatible therapeutic agent is one or more of an immunomodulatory agent, an anti-inflammatory agent, an anti-infective agent, an anti-malarial agent, an anti-viral agent or an anti-fibrotic agent.

23. The method according to claim 22 wherein
a. the immunomodulatory agent comprises one or more of methotrexate; a glucocorticoid, cyclosporine, tacrolimus and sirolimus; a recombinant interferon selected from IFN-α; IFN-α-2b, IFN-β, IFN-γ, IFN-κ, IFN-ω; a recombinant IL-2 receptor inhibitor; a PDE4 inhibitor; a hyperimmune globulin prepared from a donor with high titers of a desired antibody; a TNFα inhibitor/antagonist; an IL-1β inhibitor; a chimeric IL-1Ra; an IL-6 inhibitor; an IL-12/IL-23 inhibitor selected from ustekinumab, briakinumab; an IL-23 inhibitor selected from guselkumab, tildrakizumab; a compound that targets TLR4 signaling; a p38 MAPK inhibitor, a Janus kinase signaling inhibitor; a compound that targets cell adhesion molecules to reduce leukocyte recruitment; a checkpoint inhibitor, or a recombinant anti-inflammatory cytokine; or
b. the anti-infective agent is amoxicillin, doxycycline, demeclocycline; eravacycline, minocycline, ormadacycline, tetracycline, cephalexin, defotaxime, cetazidime, cefuroxime, ceftaroline; ciprofloxacin, levofloxacin, moxifloxacin, clindamycin, lincomycin, metronidazole, azithromycin; clarithromycin, erythromycin, sulfamethoxazle and trimethoprim; sulfasalazine, amoxicillin and clavulanate; vancomycin, dalbavancin, oritavancin, telavancin, gentamycin, tobramycin, amikacin, imipenem and cilastatin, meropenem, doripenem, or ertapenem; or
c. the anti-viral agent is selected from acyclovir, gancidovir, foscamet; ribavirin; amantadine, azidodeoxythymidine/zidovudine), nevirapine, a tetrahydroimidazobenzodiazepinone (TIBO) compound; efavirenz; remdecivir, lopinavir/ritonavir, umifenovir, favipiravir, ivermectin, and delavirdine; or
d. the anti-fibrotic agent is selected from nintedanib, pirfenidone, and combinations thereof.

24. The method according to claim 22, wherein the immunomodulatory agent comprises recombinant IL-37, recombinant CD24, or both.

25. The method according to claim 23, the anti-viral agent is an agent that inhibits viral entry and decreases viral load.

26. The method according to claim 23, wherein the checkpoint inhibitor is YERVOY™ (Ipilimumab; CTLA-4 antagonist), OPDIVO™ (Nivolumab; PD-1 antagonist) or KEYTRUDA™ (Pembrolizumab; PD-1 antagonist).

27. A method for preparing a pharmaceutical composition comprising an enriched population of superactivated cytokine killer T cells (SCKTCs) comprising, in order
(a) isolating a population of mononuclear cells (MCs) comprising a population of cytokine killer T cells (CKTCs);
(b) transporting the preparation of (a) to a processing facility under sterile conditions;
(c) on day 0, placing the population of MCs in a suspension culture system comprising a serum-free culture medium;
(d) on day 6, contacting the culture system of step (c) with the serum-free culture medium containing IL-2 and IL-7, wherein the contacting stimulates CKTC activation;
(e) on day 7, pulsing the CKTCs of step (d) with an enriched population of CD1d-expressing antigen presenting cells (APCs) derived from the MCs in (a) loaded with α-GalCer;
(f) replenishing the serum-free culture medium every 1-3 days from day 7 to day 14;
(g) on day 14, adding CD1d expressing APCs loaded with α-GalCer;
(h) replenishing the serum-free culture medium of the cells every 1-3 days;
(i) On day 14+7 days, replenishing the culture medium of the culture and pulsing with CD1d expressing APCs loaded with α-GalCer;
(j) On day 14+14 days, a replenishing the culture medium of the culture and pulsing with CD1d-expressing APCs loaded with α-GalCer;
(k) On day 14+21 days, replenishing the culture medium of the culture and adding IL-12;
(l) On Day 14+22 harvesting the amplified enriched superactivated population of SCKTCs from the culture system to form a SCKTC cell product; and
(m) filling and finishing the SCKTC cell product into a container; and
(n) optionally cryopreserving the SCKTC cell product in the vapor phase of a liquid nitrogen freezer in a serum-free cryo freezing medium.

28. The method according to claim 27, wherein the population of MCs comprising the population of CKTCs:
(a) is derived from hematopoietic stem cells derived from adult bone marrow, umbilical cord, umbilical cord blood, placental tissue, or fetal liver; or
(b) is derived from leukapheresis of a donor subject allogeneic to a recipient subject; or
(c) is derived from leukapheresis of a donor subject autologous to a recipient subject.

29. The method according to claim 27, wherein in step (a) frequency of the population of CKTCs from the donor represents <0.5% of the total MNC population.

30. The method according to claim 27, wherein the population of MCs comprises subpopulations of T lymphocytes, NK cells, B lymphocytes, and monocytes.

31. The method according to claim 30, wherein the subpopulation of T lymphocytes comprises NKT cells, CD4+ T cells, and CD8+ T cells.

32. The method according to claim 27, wherein
a) the CD1d− expressing antigen presenting cells (APCs) derived from the MCs comprise CD14+ monocytes; or
b) the CD1d− expressing antigen presenting cells (APCs) derived from the MCs comprise an irradiated population of PBMCs.

33. The method according to claim 27, wherein the CD1d-expressing population of APCs loaded with alpha-GalCer is a population of monocyte-derived dendritic cells.

34. The method according to claim 33, wherein at least 30% of the monocyte derived population of DCs constitutively expresses CD1d.

35. The method according to claim 27, wherein the pulsing steps with DCs loaded with alpha-GalCer achieve at least an 80% pure population of SCKTCs without positive or negative cell separation methods.

36. The method according to claim 33, wherein the population of dendritic cells loaded with αGalCer is prepared by a method comprising
(i) isolating a population of mononuclear cells (MCs) comprising CD14+ monocytes;
(ii) inducing differentiation of the CD14+ monocytes into dendritic cells by culturing the population of CD14+ monocytes in a culture system; and
(iii) contacting the culture system with αGalCer, wherein the contacting is sufficient to load the monocyte-derived dendritic cells with αGalCer.

37. The method according to claim 27, wherein minimum acceptable specifications of the SCKTC cell product when tested in vitro include:
(i) cytokine production comprising IL-4 low, IL-5 low, IL-6 low, IL-10 low, IFNγ high, and
(ii) a ratio of IFN-γ:IL-4 in culture supernatants of at least 500: 1; and
(iii) at an effector:target cell ratio of 20:1 greater than or equal to 50% cytotoxicity against A549 cells; and
(iv) a therapeutic dose of the cell product per treatment cycle of 30 days comprising about 0.2×10¹ activated SCKTCs.
